(19) United States
(12) Patent Application Publication
(10) Pub. No.: US 2012/0101007 A1
(43) Pub. Date: Apr. 26, 2012

(54) SILVER NANOPLATES

(75) Inventors: Damian Ahern, Dublin (IE); Margaret Brennan Fournet, County Galway (IE); Denise Charles, Dublin (IE); Stephen Michael Cunningham, County Louth (IE); Patrick Fournet, County Galway (IE); John Moffat Kelly, County Dublin (IE); Deirdre Marie Ledwith, County Offaly (IE); Muriel Celine Voisin, County Galway (IE)

(73) Assignees: PROVOST FELLOWS AND SCHOLARS OF THE COLLEGE OF THE HOLY AND UNDIVIDED TRINITY OF QUEEN ELIZEBETH NEA, Dublin (IE); NATIONAL UNIVERSITY OF IRELAND, GALWAY, Galway (IE)

(21) Appl. No.: 13/263,645

(22) PCT Filed: Apr. 8, 2010
(86) PCT No.: PCT/IE2010/000020

§ 371 Date: Oct. 7, 2011

Related U.S. Application Data
(60) Provisional application No. 61/202,817, filed on Apr. 8, 2009.
(60) Provisional application No. 61/202,816, filed on Apr. 8, 2009.
(60) Provisional application No. 61/202,815, filed on Apr. 8, 2009.

(51) Int. Cl. C40B 30/10; C40B 40/12; C07H 1/00; C40B 40/06; C07K 17/14; C40B 40/10; C40B 40/00

(52) U.S. Cl. 506/12; 506/18; 506/19; 506/13; 506/16; 530/345; 530/391.1; 536/25.3; 536/123.1

(57) ABSTRACT

A sensor for detecting of an analyte in a solution phase comprises a plurality of functionalised silver nanoplates wherein a functionalising agent is directly bonded to the surfaces of the nanoplates. The nanoplates provide a detectable wavelength shift change in their local surface plasmon resonance spectrum in response to the binding of an analyte. Two or more of the nanoplates may be electromagnetically coupled.

This invention relates to silver nanoplates. In one aspect the invention relates to a sensor, especially a biosensor comprising silver nanoplates.

The systematic tunability of the optical properties of noble metal nanoparticles including nanoplates has received increasing fundamental and technological interest due to the many uses of noble metal nanoparticles such as in photonic devices, as spectroscopic and imaging labels, in sensing applications and in biomedicine.

The optical properties of noble metal nanostructures are governed by their unique localized surface plasmon resonance (LSPR). The LSPR is the collective oscillation of the nanostructure conduction band electrons in resonance with the incident electromagnetic field¹. This occurs for diameters smaller than the wavelength of the incident light and has two primary consequences. Firstly the resonance frequency of this surface plasmon induces wavelength dependent absorption of light and secondly the local electromagnetic field surrounding the particles is greatly enhanced. It is these two unique properties which have lead to the development of noble metal nanoparticle based sensor technologies. The spectrum of these LSPR oscillations are strongly reliant upon the nanostructure size² shape³ and spacing, while the spectral response is strongly dependant on the dielectric constant⁴⁻⁷ and the dielectric constant of the surrounding environment⁸⁻¹⁰. The sensitivity of the LSPR to changes in these parameters has potential for a diverse range of technologies resulting in the development of noble nanostructures for applications including waveguides, molecular rulers¹¹ bio-imaging agents¹² and chemical and biological sensing¹³⁻¹⁵. In particular harnessing LSPR shifts induced by local medium refractive index (RI) changes caused by specific binding of analyte molecules to capture-ligand functionalized nanostructures opens a route to ultra sensitive biosensors.

The sensitivity of the LSPR shifts induced by local medium refractive index (RI) changes caused for example by the specific binding of analyte molecules to capture-ligand functionalized nanostructures can be enhanced by tuning the geometry of the nanostructures. Non-spherical nanoparticles (e.g. nanoprisms, nanorods, or nanoshells) have been postulated to exhibit increased LSPR sensitivities due to their support of large surface charge polarisability and increased local field enhancement at their sharp geometries¹⁶. A variety of single substrate bound shaped nanostructures with increased LSPR sensitivity have been reported including single silver nanoprisms¹⁷ silver nanocubes¹⁸, gold nanostars¹⁹, and gold nanorings²⁰. Significantly increased LSPR sensitivity have been reported for more complex coupled plasmonic nanostructures such as; 801 nm/RIU for hematite core/Au shell Nanorice²¹ and 880 nm/RIU for gold nanorings²², however these are at longer NIR wavelengths than are suitable for biosensing applications. Silver nanoparticles have the advantage over other noble metals such as gold and copper in that the LSPR energy of silver is removed from interband transitions (3.8 eV ˜327 nm)²³ resulting in a narrow LSPR which exhibits a much stronger shift with increasing local dielectric constant compared to that for gold or copper^{23,24}.

### STATEMENTS OF INVENTION

According to the invention there is provided a sensor for detecting of an analyte in a solution phase, the sensor comprising a plurality of functionalised silver nanoplates wherein a functionalising agent is directly bonded to the surfaces of the nanoplates and whereby the nanoplates provide a detectable wavelength shift change in their local surface plasmon resonance spectrum in response to the binding of an analyte.

Two or more of the nanoplates may be electromagnetically coupled. At least three or more of the nanoplates may be electromagnetically coupled. At least four or more of the nanoplates may be electromagnetically coupled.

In the invention at least some of the plurality of nanoplates form electromagnetically coupled groups such as dimers, and/or trimers, and/or multimers or are otherwise proximally clustered, wherein the nanoplates in a coupled group remain discrete, unaggregated, and do not physically touch or chemically bond, but their electromagnetic fields overlap or strongly couple to a degree which permits the sharing of the electromagnetic field among the individual nanoplates within the coupled group, and/or the exhibition of electromagnetic modes of the nanoplates in the coupled group which add or multiply together or subtract (both modes of which may be exhibited within a single coupled group.

The electromagnetic coupling or other proximal clustering of the functionalised nanoplates results in an increased optical extinction, or an increased optical reflection and/or scattering and/or emission signal, wherein the sensor may comprise a smaller number of nanoplates in a given optical illumination and spectroscopy arrangement than if the said coupling or clustering were not exhibited, and/or wherein the sensitivity of the sensor to a species is improved as a result of the said coupling or clustering.

In one embodiment the coupled nanoplates form a chain-like structure.

In one case the nanoplates are dispersed in a solvent system.

The nanoplates may be tethered to a support substrate such that substantially all of the surfaces of the nanoplate are available for interaction with an analyte. The functionalised nanoplates may be tethered to a substrate by means of one or more tethering molecules, which are attached to the functionalised nanoplates at locations among the functionalizing agent (receptor) molecules, wherein substantially all of the surfaces remain available for interaction with an analyte species. The tethering molecules may tether the functionalised molecules indirectly to the substrate by means of one or more other linking molecules, either by the formation of a complex with these other linking molecules or otherwise.

The linking molecules may be selected in order to avoid or reduce steric hindrances between the functionalised nanoplates and in particular to avoid or reduce steric hindrances between the functionalizing agent (receptor) molecules, to improve the specificity and sensitivity of the sensor.

The sensor may comprise from 10¹ to 10¹³ nanoplates, at least 10⁹ to 10¹³ nanoplates, from 10¹ to 10⁹ nanoplates, from 10² to 10⁴ nanoplates.

We have found that the functionalised nanoplates remain stable in the solvent system for a period of at least one week at atmospheric pressure and at a temperature of 20° C. Indeed the nanoplates remain stable for at least several weeks.

In one embodiment when the functionalised nanoplates are exposed to a light source at a wavelength range within the ultraviolet-visible-infrared spectrum or part thereof, and an optical spectrum of an ensemble of the functionalised nanoplates is measured over a wavelength range within the ultraviolet-visible-infrared spectrum or part thereof, at least one optical spectral peak is observed due to the local surface plasmon resonance (LSPR) of the functionalised nanoplates with incident light from said light source, and the said functionalised nanoplates have, for a specific method of light exposure and optical spectrum measurement, a specified minimum sensitivity or ensemble sensitivity figure of merit (FOM) (defined as the ratio of the linear local surface plasmon resonance (LSPR) refractive index sensitivity or ensemble sensitivity, to the local surface plasmon resonance linewidth being the full width at half peak maximum (FWHM) of the optical spectral peak due to the local surface plasmon resonance (LSPR)) at least at one specified wavelength in the spectrum.

The said optical spectrum of the functionalised nanoplates or an ensemble thereof is measured, after the functionalised nanoplates have been exposed to one or a plurality of analyte species of a type which is capable of attaching to the said functionalised nanoplates or to the functionalising agent which is directly bonded to the functionalised nanoplates, such that attachment of analyte species occurs to the functionalising agent (the receptor) which is directly bonded to the functionalised nanoplates, increasing the local refractive index inducing the local surface plasmon resonance (LSPR) of the functionalised nanoplates and causing their said optical spectral peak as observed with incident light from said light source, to change from that of functionalised nanoplates which have not been exposed to said species, in a manner consistent with a wavelength shift in the said optical spectral peak, due to changes in the local surface plasmon resonance of the functionalised nanoplates consequent on the said attachment of a species to the said functionalised nanoplates.

The light from the light source may traverse a volume or part thereof containing the functionalised nanoplates and the optical spectrum measured is an optical extinction spectrum of the functionalised nanoplates or an ensemble thereof.

In one embodiment the ensemble sensitivity figure of merit is at least 1.75 at a wavelength of 450 nm the ensemble sensitivity figure of merit is at least 1.75 at wavelengths between 450 nm and 930 nm; the ensemble sensitivity figure of merit is at least 2.25 at wavelengths above 900 nm; the ensemble sensitivity figure of merit is at least 3.0 at wavelengths above 1100 nm.

In one embodiment the nanoplates have an ensemble sensitivity value of between 281 nm and 1400 nm per unit change in the (dimensionless) refractive index and with a local surface plasmon resonance (LSPR) peak in the 400 nm to 1200 nm wavelength region of the spectrum when measured by optical extinction spectroscopy.

In one case the nanoplates have an ensemble sensitivity value of at least 300 nm per unit change in the (dimensionless) refractive index with a local surface plasmon resonance (LSPR) peak in the 600 nm region of the spectrum when measured by optical extinction spectroscopy.

In one case when a light from the light source traverses a volume or part thereof containing the functionalised nanoplates in a dark field imaging or light collection arrangement, and the optical spectrum measured is an optical reflection and/or scattering and/or emission spectrum of the functionalised nanoplates or an ensemble thereof measured by dark field spectroscopy.

In one embodiment the ensemble sensitivity figure of merit is greater than 1.9 at a wavelength of 450 nm when measured by dark field spectroscopy; the ensemble sensitivity figure of merit is greater than 3.0 at a wavelength of 600 nm when measured by dark field spectroscopy; the ensemble sensitivity figure of merit is greater than 3.5 at a wavelength of 750 nm when measured by dark field spectroscopy.

In one embodiment the ensemble sensitivity figure of merit of the functionalised nanoplates when measured by dark field spectroscopy is greater than the sensitivity or ensemble sensitivity figure of merit (respectively) of the functionalised nanoplates when measured by optical extinction spectroscopy performed at a wavelength range within the ultraviolet-visible-infrared spectrum or part thereof.

In some embodiments the functionalising agent is selected from a ligand, a peptide, a polypeptide, a glycan, an antibody, or a nucleic acid.

The functionalising agent may be selected from a mono-species, a di-species, and a multi-species functionalising agent.

The silver nanoplates may have an aspect ratio of between 2 and 20.

The nanoplates may be triangular in shape.

The nanoplates may have an edge length between about 10 nm and about 200 nm.

The nanoplates may have an aspect ratio between about 2 to about 13.

In one embodiment the nanoplates may have a truncated triangular shape.

The apices of the triangles may be snipped with a chemical agent or by deprivation of a passivation agent. The chemical agent may be one or more of an acid, a base, a salt, a polymer, or a biological agent. The acid may be ascorbic acid or citric acid. The base may be an amine. The salt may be selected from one or more of sodium chloride, sodium bromide, sodium iodide, potassium chloride, potassium bromide, or potassium iodide. The polymer may be polyvinyl alcohol or polyvinylpyrrolidone. The biological agent may be selected from one or more of an amino acid or biological medium.

In another embodiment the corners of the triangle may have been snipped by centrifugation or sonication.

In one embodiment the nanoplates may be blocked with a blocking agent. The blocking agent may be selected from a mercapto based agent, such as mercaptobenzoic acid or mercaptohexadecanoic acid or 16-mercaptohexadecanoic acid, or a serum, or an immuno stripped serum, or a non-immuno antibody or a non-specific protein, or a nucleic acid sequence or styrene, or polyethylene glycol.

In one embodiment the wavelength shift in the optical spectral peak due to the local surface plasmon resonance (LSPR) peak wavelength may be a red shift (a shift to a longer wavelength) within the 300 nm to 1200 nm spectral window

In one case the wavelength shift in the optical spectral peak due to the local surface plasmon resonance (LSPR) peak wavelength may be a blue shift (a shift to a shorter wavelength) within the 300 nm to 1150 nm spectral window as a result of the attachment of analyte species to the said functionalised nanoplates or to the functionalising agent which is directly bonded to the functionalised nanoplates. There can be a small blue shift which makes the red shift smaller than it might otherwise be.

In some embodiments the full width at half peak maximum (FWHM) of the optical spectral peak due to the local surface plasmon resonance (LSPR) of the functionalised nanoplate may be between about 50 nm and about 300 nm, preferably between about 60 nm to about 160 nm.

In some embodiments the full width at half peak maximum (FWHM) of the optical spectral peak due to the local surface plasmon resonance (LSPR) of the functionalised nanoplate may have a local surface plasmon resonance (LSPR) peak in the 300 nm to 1200 nm region.

In one aspect when the functionalised nanoplates are applied in solution to one or more analyte species molecules which are bonded to a substrate, either directly, or else indirectly by means of one or more linking molecules, such that at least some of the functionalised nanoplates become tethered to the substrate by means of one or more of the analyte species molecules, with a resultant change in the local surface plasmon resonance (LSPR)

In some embodiments the functionalised nanoplates are exposed to a light source, and a Raman spectrum of the functionalised nanoplates or an ensemble thereof is measured, wherein at least one Raman spectral peak is sensitive to and changes, either in spectral position or in magnitude or relative magnitude, as a result of the attachment of a species to some of the functionalised nanoplates. The Raman spectrum may be measured by Surface Enhanced Raman Spectroscopy. In some cases the Raman response at least one spectral position is enhanced by at least a factor of 10³, preferably by a factor of 10⁶.

The invention also provides an assay comprising a sensor of the invention.

In another aspect the invention provides the use of a sensor of the invention in a solution phase assay.

In another aspect the invention provides the use of a sensor of the invention in an assay based on the principle of local surface plasmon resonance (LSPR) optical spectral peak wavelength shift due to a refractive index change or other optical property change in response to the attachment of a species to at least some of the functionalised nanoplates.

In another aspect the invention provides the use of a sensor of the invention in an assay based on Raman Spectroscopy. The assay may be based on Surface Enhanced Raman Spectroscopy

In a further aspect the invention provides the use of a sensor of the invention as a contrast agent for cellular imaging.

In a further aspect the invention provides a process for functionalising the surface of a silver nanoplate with a functionalising agent comprising the steps of:
a. forming silver seeds from an aqueous solution comprising a reducing agent, a stabilising agent, a water soluble polymer and a silver source; and
b. growing the thus formed seeds into silver nanoplates in an aqueous solution comprising silver seeds, a reducing agent, a silver source, and a functionalising agent selected from a ligand, a peptide, a polypeptide, a glycan, an antibody, or a nucleic acid.

In one embodiment step (a) and/or step (b) are performed at a shear flow rate between about 1×10¹ s⁻¹ and about 9.9×10⁵ s⁻¹. Step (a) and/or step (b) may be performed at a shear flow rate between about 1×10¹ s⁻¹ and 2×10⁵ s⁻¹.

The reducing agent, stabilising agent and water soluble polymer of step (a) may be mixed prior to the addition of a silver source.

The reducing agent, stabilising agent and water soluble polymer may be mixed for at least 2 minutes.

The silver source may be added to the reducing agent, stabilising agent and water soluble polymer mixture at a rate of less than about 10% by volume/min.

In one case the water soluble polymer is a polyanionic polymer. The polymer may be a derivative of polysulphonate. The polymer may be a derivative of polystyrene sulphonate such as an inorganic salt of polystyrene sulphonate. The derivative may be a monovalent salt of polystyrene sulphonate.

The water soluble polymer may be poly (sodium styrenesulphonate) (PSSS). The PSSS may have a molecular weight between about 3 kDa to about 1,000 kDa, typically about 1,000 kDa.

The water soluble polymer may be present at a concentration of at least 0.5 mg/mL.

The reducing agent of step (a) may be sodium borohydride. The reducing agent of step (a) may be present at a concentration of at least 3 mM.

The silver source of step (a) may be a silver salt, such as silver nitrate. The silver source of step (a) may be present at a concentration of at least 0.1 mM, this concentration may be about 0.25 mM.

The stabilization agent in step (a) may be TSC. The stabilization agent in step (a) may be present at a molar ratio of at least 1:1 relative to the concentration of the silver salt in step (a), this molar ratio may be about 5:1.

In one case the reducing agent of step (b) is ascorbic acid. The reducing agent of step (b) may be present at a concentration of half the concentration of the silver source.

The silver source of step (b) may be a silver salt such as silver nitrate. The silver source of step (b) may be present at a concentration of at least 0.01 mM, this concentration may be about 0.15 mM and can range up to 10 mM.

In one case the silver seeds of step (b) are present at a mole ratio of silver seeds: silver ion in the silver source may range from 1:500 to 1:100000

The silver seeds and reducing agent of step (b) may be mixed prior to the addition of a silver source. The silver seeds and reducing agent may be mixed for at least 2 minutes.

In one case the silver source is added to the silver seeds and reducing agent mixture at a rate of at least 10% by volume/min.

The silver seeds formed in step (a) may be aged prior to growing the seeds in step (b). The silver seeds may be aged for at least one hour.

In one case step (a) is performed at room temperature.

The process may be a batch process.

The process may be a continuous flow process.

In one embodiment the functionalising agent may be added after the addition of the silver source.

In one embodiment the process comprises the step of blocking the functionalised nanoplate with a blocking agent. The blocking agent may be selected from a mercapto based agent, such as mercaptobenzoic acid or mercaptohexadecanoic acid or 16-mercaptohexadecanoic acid, or a serum, or an immuno stripped serum, or a non-immuno antibody or a non-specific protein, or a nucleic acid sequence or styrene, or polyethylene glycol.

According to the invention there is provided a sensor comprising a silver nanoplate wherein the silver nanoplate has an aspect ratio of between 2 and 20.

The nanoplate may be triangular in shape. The nanoplate may have an edge length between about 10 nm and about 200 nm. The nanoplate may have an aspect ratio between about 2 to about 13. The nanoplate may have a FWHM of between about 0.297 eV and about 0.6 eV. The nanoplate may have an LSPR peak in the 300 nm to 1150 nm region. The nanoplate may have an ensemble sensitivity value of between 281 nm/RIU and 420 nm/RIU with an LSPR peak in the visible region. The nanoplate may have an ensemble sensitivity value of at least 300 nm/RIU with an LSPR peak in the 600 nm region.

The nanoplate may be a truncated triangle. The corners of the triangle may have been snipped with a chemical agent. The chemical agent may be one or more of an acid, a salt, a polymer, or a biological agent. The acid may be mercaptobenzoic acid or mercaptohexadecanoic acid. The salt may be selected from one or more of sodium chloride, sodium bromide, or sodium iodide. The polymer may be polyvinyl alcohol or polyvinylpyrrolidone. The biological agent may be selected from one or more of sucrose, bovine serum albumin, an antibody, or a protein such as C-reactive protein. Alternatively, the corners of the triangle may have been snipped by centrifugation or sonication. The LSPR peak wavelength of the nanoplate may be blue shifted within the 300 nm to 1150 nm spectral window.

Substantially all of the surfaces of the nanoplate may be available for interation with an analyte or for functionalisation. The surface of the nanoplate may be functionalised with a functionalising agent. The functionalising agent may be selected from a ligand, a peptide, a polypeptide, a glycan, an antibody, and a nucleic acid. The functionalising agent may be selected from a mono-species, a di-species, and a multi-species functionalising agent. The LSPR peak wavelength of the nanoplate may be red shifted within the 320 nm to 1200 nm spectral window. The nanoplate may be stabilised with a stabilising agent such as trisodium citrate. Alternatively, the stabilising agent may be the functionalising agent.

The nanoplates may be blocked with a blocking agent. The blocking agent may be a mercapto based agent. Alternatively, the blocking agent may be selected from one or more of 16-mercaptohexadecanoic acid, styrene, polyethylene glycol, serum, immuno stripped serum and a nucleic acid sequence

The nanoplates of the sensor may be discrete. Alternatively, the nanoplates may be dimerised, and/or clustered.

The invention also provides for the use of a sensor described herein in a solution phase assay.

The invention further provides for the use of a sensor described herein in a Raman based assay. The Raman based assay may be surface enhanced Raman spectroscopy. The sensor may have a SERS enhancement factor of the order of 5.3×10⁶.

The invention also provides for the use of a sensor described herein as a contrast agent for cellular imaging.

The invention further provides a process for functionalising the surface of a silver nanoplate with a functionalising agent comprising the steps of:
a) forming silver seeds from an aqueous solution comprising a reducing agent, a stabiliser, a water soluble polymer and a silver source;
b) growing the thus formed seeds into silver nanoplates in an aqueous solution comprising silver seeds, a reducing agent and a silver source; and
c) incubating the thus formed silver nanoplates with a functionalising agent.

The functionalising agent may be one or more of a ligand, a peptide, a polypeptide, an antibody, or a nucleic acid. The nanoplates may be incubated with the functionalising agent for at least 8 hours. The nanoplates may be incubated with the functionalising agent at about 4° C. The nanoplates may be incubated with the functionalising agent in the dark.

The process may comprise the step of
d) centrifuging the functionalised nanoplates of step (c) to remove excess functionalising agent.

The process may comprise the step of stabilising the functionalised nanoplate with a stabilising agent such as trisodium citrate.

The process may comprise the step of blocking the functionalised nanoplate with a blocking agent. The blocking agent may be a mercapto based blocking agent. Alternatively, the blocking agent may be selected from one or more of 16-mercaptohexadecanoic acid, styrene, polyethylene glycol serum, immune stripped serum and a nucleic acid sequence.

Nanoparticles including nanoplates can be synthesised from a range of materials, including noble metals such as gold or silver. Nanoparticles have been utilised in a number of different fields of technology ranging from paints to biomolecular devices. The wide range of application and uses of nanoparticles has resulted in a need to produce nanoparticles in large quantities while maintaining batch reproducibility. WO04/086044 describes a two-step wet chemistry batch process for synthesising silver seeds to produce a range of silver nanoparticles. Whilst the silver nanoparticles produced by the wet chemistry batch method are high quality nanoparticles, the quantity of nanoparticles produced is limited as each batch is restricted to a maximum volume of about 100 ml.

We describe a process for producing high quality nanoplates on an industrial scale. According to a further aspect of the invention there is provided a process for synthesising silver nanoplates comprising the steps of:
(i) forming silver seeds from an aqueous solution comprising a reducing agent, a stabiliser, a water soluble polymer and a silver source; and
(ii) growing the thus formed seeds into silver nanoplates in an aqueous solution comprising silver seeds, a reducing agent and a silver source.
   wherein step (i) and/or step (ii) are performed at a shear flow rate between about 1×10¹ s⁻¹ and about 9.9×10⁵ s⁻¹. Step (i) and/or step (ii) may be performed at a shear flow rate between about 1×10¹ s⁻¹ and 2×10⁵ s⁻¹.

The reducing agent, stabiliser and water soluble polymer of step (i) may be mixed prior to the addition of a silver source. The reducing agent, stabiliser and water soluble polymer may be mixed for at least 2 minutes. The silver source of step (i) may be added to the reducing agent, stabiliser and water soluble polymer mixture at a rate of less than about 10% by volume/min.

The water soluble polymer may be a polyanionic polymer. The polymer may be a derivative of polysulphonate. The polymer may be a derivative of polystyrene sulphonate. The derivative may be an inorganic sort of polystyrene sulphonate. The derivative may be a monovalent salt of polystyrene sulphonate. The water soluble polymer may be poly (sodium styrenesulphonate) (PSSS). The PSSS may have a molecular weight between about 3 kDa to about 1,000 kDa. The PSSS may have a molecular weight of about 1,000 kDa. The water soluble polymer may be present at a concentration of at least 25 mg/mL.

The reducing agent of step (i) may be sodium borohydride. The reducing agent of step (i) may be present at a concentration of at least 3 mM.

If a stabiliser is used in step (i) it may be trisodium citrate. The stabiliser of step (i) may be present at a concentration of at least 0.3 mM and preferable at 1.25 mM.

The stabiliser may also be a functionalisation agent.

The silver source of step (i) may be a silver salt. The silver salt may be silver nitrate. The silver source of step (i) may be present at a concentration of at least 2.5 mM.

The reducing agent of step (ii) may be ascorbic acid. The reducing agent of step (ii) may be present at a concentration of at least 7.5 mM.

The silver source of step (ii) may be a silver salt. The silver salt may be silver nitrate. The silver source of step (ii) may be present at a concentration of at least 15 mM.

The silver seeds of step (ii) may be present at a mole ratio of silver seeds: silver ion in the silver source of at least 1:500 and up to 1:10000.

The silver seeds and reducing agent of step (ii) may be mixed prior to the addition of a silver source. The silver seeds and reducing agent may be mixed for at least 2 minutes. The silver source may be added to the silver seeds and reducing agent mixture at a rate of at least 10% by volume/min.

The silver seeds formed in step (ii) may be aged prior to growing the seeds in step (ii). The silver seeds may be aged for at least one hour.

Step (i) may be performed at room temperature.

The process may be a batch process. Alternatively, the process may be a continuous flow process.

The invention also provides a process for synthesising silver nanoplates comprising the steps of
c. forming silver seeds from an aqueous solution comprising a reducing agent, a stabilising agent, a water soluble polymer and a silver source; and
d. growing the thus formed seeds into silver nanoplates in an aqueous solution comprising silver seeds, a reducing agent, a silver source
   wherein step (a) and/or step (b) are performed at a shear flow rate between about 1×10⁵ s⁻¹ and about 9.9×10⁵ s⁻¹.

In one case step (a) and/or step (b) are performed at a shear flow rate between about 1×10¹ s⁻¹ and 2×10⁵ s⁻¹.

In one embodiment the reducing agent, stabilising agent and water soluble polymer of step (a) are mixed prior to the addition of a silver source. The reducing agent, stabilising agent and water soluble polymer may be mixed for at least 2 minutes.

In one embodiment the silver source is added to the reducing agent, stabilising agent and water soluble polymer mixture at a rate of less than about 10% by volume/min.

The water soluble polymer may be a polyanionic polymer. The polymer may be a derivative of polysulphonate such as a derivative of polystyrene sulphonate. The derivative may be an inorganic salt of polystyrene sulphonate. The derivative may be a monovalent salt of polystyrene sulphonate. In one embodiment the water soluble polymer is poly (sodium styrenesulphonate) (PSSS). The PSSS may have a molecular weight between about 3 kDa to about 1,000 kDa, especially about 1,000 kDa. The water soluble polymer may be present at a concentration of at least 0.5 mg/mL.

In one embodiment the silver source of step (a) is a silver salt. The silver salt of step (a) may be silver nitrate. The silver salt of step (a) may be present at a concentration of at least 0.1 mM, and typically at a concentration of 0.25 mM

The reducing agent of step (a) may be sodium borohydride. The reducing agent of step (a) may be present at a molar ratio of at least 1:1 relative to the concentration of the silver salt in step (a), this molar ratio may be about 1.2:1.

In one embodiment the stabiliser of step (a) is trisodium citrate. The stabiliser of step (a) may be present at a molar ratio of at least 1:1 relative to the concentration of the silver salt in step (a), and this molar ratio may be about 5:1.

In one embodiment the silver source of step (b) is a silver salt. The silver salt may be silver nitrate. The silver source of step (b) may be present at a concentration of at least 0.01 mM, this concentration may be about 0.15 mM and can range up to 10 mM.

In one embodiment the silver seeds of step (b) are present at a mole ratio of silver seeds: silver ion in the silver source of from 1:500 to 1:100000

In one case the reducing agent of step (b) is ascorbic acid. In one embodiment the reducing agent of step (b) is present at a concentration of half the concentration of the silver source.

In one case the silver seeds and reducing agent of step (b) are mixed prior to the addition of a silver source. The silver seeds and reducing agent may be mixed for at least 2 minutes. The silver source may be added to the silver seeds and reducing agent mixture at a rate of at least 10% by volume/min. The silver seeds formed in step (a) may be aged prior to growing the seeds in step (b). The silver seeds may be aged for at least one hour.

Step (a) may be performed at room temperature.

The process may be a batch process or a continuous flow process.

In one embodiment step (b) is carried out without a stabilising agent.

In another embodiment step (b) is carried out in the presence of a stabilising agent. In this case the stabiliser of step (b) may be trisodium citrate. The stabiliser of step (b) may be present at a concentration of from 12.5 μM to 12.5 mM.

In one embodiment the process comprises concentrating an aqueous solution or suspension of the silver nanoplates. A solution or suspension of the nanoplates may be concentrated by cross-flow filtration. The process may comprise a plurality of cross-flow filtration steps. Typically each cross-flow filtration step increases the amount of silver by weight in the solution or suspension by at least a factor of 10.

In one embodiment the process comprises the further step after step (b) of adding a chemical and/or a biological functionalising agent. The functionalising agent may be selected from: a ligand (such as cytidine 5′-diphosphocholine, diethylene glycol, or beta-carotene), a thiolated ligand (such as long chain mercapto-based compounds, mercapto-hexanoic acid, and mecapto-benzoic acid), an aromatic ligand, an aromatic thiolated ligand (such as 2-aminothiophenol, thiophenol, 4-methylthiophenol, or 4-aminothiophenol), or a polymer (such as polyvinyl alcohol or polyvinyl pyrrolidone), or a conjugated polymer (such as polythiophenes, polyphenylene-vinylenes (PPV), poly(2-methoxy-5-(2′-ethyl-hexyloxy)-1,4-phenylene-vinylene) (MEH-PPV)), or a conductive polymer (such as Poly(3,4-ethylenedioxythiophene) poly(styrenesulfonate) (PEDOT:PSS)).

In one embodiment the process comprises the addition of one or more other chemical additives in one or more further process steps after step (b).

In one case a viscosity modifying agent is added in a further process step after step (b). The viscosity modifying agent may be a viscosity increasing agent. The viscosity modifying agent may be a polymer such as polyvinyl alcohol or polyvinyl pyrrolidone or glycerol. Up to 5%, up to 10%, up to 20% by weight of the viscosity modifying agent is present in the product formulation on completion of the process.

In one case a surface tension modifying agent is added in a further process step after step (b). The surface tension modifying agent may be a surface tension lowering agent such as diethylene glycol. Up to 50% by weight of the surface tension modifying agent may be present in the product formulation on completion of the process.

In one case a chemical agent, which can promote bonding, linkage, electrical conduction, electromagnetic coupling or plasmonic coupling between two or more nanoplates, is added in a further process step after step (b). The chemical agent may be selected from a ligand (such as cytidine 5′-diphosphocholine, diethylene glycol, or beta-carotene), a thiolated ligand (such as long chain mercapto-based compounds, mercapto-hexanoic acid, and mecapto-benzoic acid), an aromatic ligand, an aromatic thiolated ligand (such as 2-aminothiophenol, thiophenol, 4-methylthiophenol, or 4-aminothiophenol), or a polymer (such as polyvinyl alcohol or polyvinyl pyrrolidone), or a conjugated polymer (such as polythiophenes, polyphenylene-vinylenes (PPV), poly(2-methoxy-5-(2′-ethyl-hexyloxy)-1,4-phenylene-vinylene) (MEH-PPV)), or a conductive polymer (such as Poly(3,4-ethylenedioxythiophene) poly(styrenesulfonate) (PEDOT:PSS))

In one case the process parameters in either or both of steps (a) and (b) are selected such as to produce polygonal nanoplates. The process parameters in either or both of steps (a) and (b) may be selected such as to produce polygonal nanoplates having six or less sides. The process parameters (in either or both of steps (a) and (b) a may be selected such as to produce hexagonal nanoplates. The process parameters (in either or both of steps (a) and (b) may be selected such as to produce triangular nanoplates.

In one embodiment the concentration of the stabilising agent in step (b), if present, is reduced for the purpose of truncating or rounding the apices or corners of the polygonal nanoplates.

An additional chemical agent may be added either in, or after, step (b), for the purpose of truncating or rounding the apices or corners of the polygonal nanoplates. The chemical agent may be one or more of an acid, a base, a salt, a polymer, or a biological agent. The acid may be ascorbic acid or citric acid. The base may be an amine. The salt may be selected from one or more of sodium chloride, sodium bromide, sodium iodide, potassium chloride, potassium bromide, or potassium iodide. The polymer may be polyvinyl alcohol or polyvinylpyrrolidone. The biological agent may be selected from one or more of an amino acid or biological medium.

In one case the apices or corners of the polygonal nanoplates have been truncated or rounded by centrifugation or sonication.

The invention also provides a formulation comprising a plurality of silver nanoplates in an aqueous solution or suspension wherein the nanoplates are dispersed in the aqueous solution or suspension. Two or more of the nanoplates may be electromagnetically coupled. At least three or more of the nanoplates may be electromagnetically coupled. At least four or more of the nanoplates may be electromagnetically coupled. The coupled nanoplates may form a chain-like structure.

The nanoplates remain stable in the solvent system for a period of at least one week at atmospheric pressure and at a temperature of 20° C.

The silver nanoplates may have an aspect ratio of between 2 and 20. The nanoplates may be triangular in shape. The nanoplates may have an edge length between about 10 nm and about 200 nm. The nanoplates may have an aspect ratio between about 2 to about 13.

In one case the nanoplates are of a polygonal shape and may have six or less sides. In one case the nanoplates are of a triangular shape.

The apices or corners of the polygonally shaped nanoplates may have been truncated or rounded.

The apices or corners of the polygonally shaped nanoplates may have been truncated or rounded by a chemical agent or by deprivation of a stabilising agent as described above.

Alternatively or additionally the apices or corners of the polygonally shaped nanoplates may have been truncated or rounded by centrifugation or sonication.

In some embodiments at least greater than 50%, 80%, 90%, 95% of the silver nanoplates are substantially triangular or truncated triangular in shape.

In some embodiments at least greater than 50%, 80%, 90% of the silver nanoplates are substantially hexagonal or truncated hexagonal in shape.

In some embodiments at least 90% of the silver nanoplates have an aspect ratio which is greater than 2. At least 90% of the silver nanoplates may have an aspect ratio which is between 2 and 20. At least 90% of the silver nanoplates may have an aspect ratio which is between 2 and 13. At least 80% of the silver nanoplates may have an aspect ratio which is greater than 10.

In some embodiments the formulation exhibits a local surface plasmon resonance optical spectral peak in the visible or infrared regions of the spectrum, when observed by an appropriate optical spectroscopic detector.

The aspect ratio of at least 80% of the silver nanoplates may be between 5.5 and 6.5 and the local surface plasmon resonance optical spectral peak is between 650 nm and 750 nm

The aspect ratio of at least 80% of the silver nanoplates may be between 7 and 8 and the local surface plasmon resonance optical spectral peak is between 840 nm and 880 nm

The aspect ratio of at least 80% of the silver nanoplates may be between 9 and 10 and the local surface plasmon resonance optical spectral peak is between 900 nm and 940 nm

In some embodiments the formulation comprises between 1000 ppm (0.1%) and 10000 ppm (1%) of silver by weight.

In some cases the formulation comprises between 1% and 2% of silver by weight, between 2% and 10% of silver by weight, up to 30% of silver by weight, up to 70% of silver by weight.

The formulation may comprise a viscosity modifying agent such as a viscosity increasing agent which may be a polymer such as polyvinyl alcohol or polyvinyl pyrrolidone. The formulation may comprise up to 20% by weight of the viscosity modifying agent, up to 10% by weight of the viscosity modifying agent. The formulation may comprise about 5% by weight of the viscosity modifying agent.

In some cases the formulation comprises a surface tension modifying agent such as a surface tension lowering agent, for example diethylene glycol. The formulation may comprise up to 50% by weight of the surface tension lowering agent.

In some cases the nanoplates are surface functionalised with a chemical and/or a biological functionalising agent. The functionalising agent may be selected from one or more of: cytidine 5′-diphosphocholine, mercapto-hexanoic acid, and mecapto-benzoic acid.

The formulation may comprise a stabilising agent such as trisodium citrate.

In one case the formulation is capable of delivery to a substrate by means of a printing device, such as an ink-jet printing device. The ink-jet printing device may be a piezo-electrically actuated ink-jet device or a thermal ink-jet printing device.

In some embodiments the silver nanoplates are of a thickness and/or length which reduces their melting point below that of the temperature of operation of the thermal ink-jet printing device.

In one embodiment the formulation is capable of delivery to a substrate by means of a gravure printing device.

The formulation may be capable of delivery to a flexible substrate. The flexible substrate may be delivered to the printing device from a reel or a roll, and may be withdrawn from the printing device into a reel or a roll.

In one case the silver nanoplates have a surface enhanced resonance spectroscopy enhancement factor of at least 1×10⁶.

The invention also provides a substrate having a formulation of the invention thereon. The substrate with the formulation applied thereto may be subsequently cured by any method including one or more methods selected from aging time, natural evaporation, thermally assisted evaporation, thermal curing, ultraviolet curing, other photoexposure curing, cooling, sintering, or firing.

The curing may be thermal curing at a temperature of less than 130° C.

The invention further provides a substrate on which a solid film or wire or conductive network of wires or assembly of nanoplates have been made from the formulation of the invention applied thereto.

The sheet resistance of the solid film or wire or conductive network of wires or assembly of nanoplates may be about 0.5 Ohms per dimensionless square.

The resistivity of the solid film or wire or conductive network of wires or assembly of nanoplates may be less than 1×10⁻⁴ Ω·cm. The resistivity of the solid film or wire or conductive network of wires or assembly of nanoplates may be less than 1.4×10⁻⁵ Ω·cm.

The silver content by weight of the formulation used may be less than 10% by weight, less than 1% by weight.

The solid film or wire or conductive network of wires or assembly of nanoplates may be thermally stable at temperatures above 100° C., above 150° C., above 200° C., above 220° C., above 260° C., above 320° C.

The solid film or wire or conductive network of wires or assembly of nanoplates is at least 40% translucent over at least a wavelength range of 300 nm within the spectral wavelength range 400 nm to 2000 nm

The solid film or wire or conductive network of wires or assembly of nanoplates may be at least 80%, at least 90% translucent over at least a wavelength range of 300 nm within the spectral wavelength range 400 nm to 2000 nm.

The solid film or wire or conductive network of wires or assembly of nanoplates may be at least 40% transparent over at least a wavelength range of 300 nm within the spectral wavelength range 400 nm to 2000 nm.

The solid film or wire or conductive network of wires or assembly of nanoplates may be at least 80%, at least 90% transparent over at least a wavelength range of 300 nm within the spectral wavelength range 400 nm to 2000 nm.

The solid film or wire or conductive network of wires or assembly of nanoplates may be at least 80% transparent over at least 80% of the spectral wavelength range 400 nm to 700 nm.

The invention also provides an optically transparent electrical conductor device comprising a substrate and a solid film or wire or conductive network of wires or assembly of nanoplates of the invention. The device may be a part of a photovoltaic device, panel or cell device.

Also provided are
a display device comprising an optically transparent electrical conductor device of the invention
a light emitting diode device (which may be semiconductor or organic material based) comprising an optically transparent electrical conductor device of the invention
an electrical or electronic circuit or device comprising a substrate and a solid film or wire or conductive network of wires or assembly of nanoplates of the invention
an optoelectronic device comprising a substrate and a solid film or wire or conductive network of wires or assembly of nanoplates of the invention
a plasmonic device comprising a substrate and a solid film or wire or conductive network of wires or assembly of nanoplates of the invention

The invention also provides a device comprising a substrate and a solid film or wire or conductive network of wires or assembly of nanoplates wherein at least some of the silver nanoplates are electromagnetically coupled to the substrate or to another layer in the device.

At least some of the silver nanoplates may be electromagnetically coupled to other particles or nanoparticles.

At least some of the silver nanoplates may be electromagnetically coupled to particles, nanoparticles or quantum dots of at least one material selected from: silicon, germanium, carbon in any of its allotropic forms, carbon nanotubes, copper indium gallium diselenide, compounds of at least one of (Al, Ga, In, Hg, Cd) with at least one of (As, P, Sb, N, Te), metal oxides.

The electromagnetic coupling may improve the absorption or coupling of electromagnetic radiation to either the nanoplate, the entity to which the nanoplate is coupled, the coupled entity-nanoplate, or any layer or device made from them.

In one case the charge carrier generation is increased by the action of the nanoplates.

In one case the formulation further comprises particles, nanoparticles or quantum dots of at least one material selected from: silicon, germanium, carbon in any of its allotropic forms, carbon nanotubes, copper indium gallium diselenide, compounds of at least one of (Al, Ga, In, Hg, Cd) with at least one of (As, P, Sb, N, Te), metal oxides.

The electromagnetic coupling may improve the absorption or coupling of electromagnetic radiation to either the nanoplate, the entity to which the nanoplate is coupled, the coupled entity-nanoplate, or any layer or device made from them.

In one case the efficiency of conversion of solar electromagnetic radiation to electrical power, of device made comprising them is increased as a result of the electromagnetic coupling and/or surface plasmons associated with the silver nanoplates.

In one embodiment at least some of the silver nanoplates are tethered to the substrate or to another layer in the device by means of another chemical entity such as a molecule or chain of molecules.

In one case the solid film or wire or conductive network of wires or assembly or distribution of silver nanoplates functions as an optical filter.

According to the invention there is provided a silver nanoplate having an ensemble average local surface plasmon sensitivity which increases as the local surface plasmon resonance peak wavelength position is tuned from the UV to Visible to the NIR spectral regions. The silver nanoplate may have an ensemble average local surface plasmon sensitivity value of at least 130 nm/RIU in the 500 nm spectral region. The silver nanoplate may have a solution phase ensemble average local surface plasmon sensitivity value of at least 200 nm/RIU in the 500 nm spectral region. The silver nanoplate may have a ensemble average local surface plasmon sensitivity value of at least 500 nm/RIU in the 950 nm spectral region. The silver nanoplate may have a a solution phase ensemble average local surface plasmon sensitivity value of at least 400 nm/RIU in the 700 nm spectral region The silver nanoplate may have an ensemble average local surface plasmon sensitivity value of at least 600 nm/RIU in the 1000 nm spectral region. The silver nanoplate may have an ensemble average local surface plasmon sensitivity value of at least 800 nm/RIU in the 1100 nm spectral region. The silver nanoplate may have an ensemble average local surface plasmon resonance value of up to 1093 nm/RIU.

The nanoplate may have an aspect ratio of at least 2. The nanoplate may have an aspect ratio of between 2 and 25 such as between 2 and 13.

The invention also provides a silver nanoplate comprising an aspect ratio of at least 12. The nanoplate may have a local surface plasmon resonance in the 1070 nm region. The nanoplate may have an ensemble average local surface plasmon resonance sensitivity of 1070/RIU in the 1093 nm spectral range.

The invention further provides a silver nanoplate comprising an aspect ratio of about 6 and a local surface plasmon resonance peak in the 700 nm region.

The invention also provides a silver nanoplate comprising an aspect ratio of about 7.4 and a local surface plasmon resonance peak in the 868 nm region.

The invention further still provides a silver nanoplate comprising an aspect ratio of about 9.6 and a local surface plasmon resonance peak in 919 nm region.

The nanoplate may have a surface enhanced resonance spectroscopy enhancement factor of at least 5.3×10⁶.

The nanoplate may be triangular in shape. The nanoplate may be a snipped triangular nanoplate.

We have outlined above and below various aspects of the invention. It will be appreciated that details given in relation to one aspect may also be applicable to other aspects and the specification should be read in this way.

### BRIEF DESCRIPTION OF THE DRAWINGS

The invention will be more clearly understood from the following description of an embodiment thereof, given by way of example only, with reference to the accompanying drawings, in which:

FIG. 1 is a schematic of a shear mixer used in a shear mixing process in accordance with an embodiment of the invention;

FIG. 2 is a UV-vis spectra of 200 ml of silver seeds prepared in a shear mixer in accordance with Example 3A;

FIG. 3 is a UV-vis spectra of 1 L of 17 ppm silver nanoplates prepared in a shear mixer in accordance with Example 3B;

FIG. 4 is a UV-vis spectra of 5 L of 17 ppm silver nanoplates prepared in a shear mixer in accordance with Example 3C;

FIG. 5 is a UV-vis spectra of 1 L of 34 ppm silver nanoplates prepared in a shear mixer in accordance with Example 3D;

FIG. 6 is a UV-vis spectra of 1 L of 17 ppm silver nanoplates prepared in a shear mixer in accordance with Example 3E;

FIG. 7 is a UV-vis spectrum of sol prepared by batch methods on 1 L scale in accordance with Example 3F;

FIG. 8 is a UV-vis spectra of 200 ml silver seeds prepared using batch method (solid line) in accordance with Example 3F and shear mixer (dash line) in accordance with Example 3A;

FIG. 9 is a schematic of an inline continuous flow shear mixer used in a process of the invention;

FIG. 10 (A) is a set of UV-vis spectra demonstrating the tunability of the LSPR λₘₐₓ of triangular silver nanoprism (TSNP) solutions; (B) is a graph showing the linear increase in the TSNP aspect ratio with increasing edge length (R=0.98); and (C) is a plot depicting the dependence of the ensembles peak wavelength on the mean aspect ratio measured for the various samples, a linear fit (R=0.94) has been applied to the collected data;

FIG. 11 (A) are TEM images showing some of the various sized TSNP samples fabricated; (D) is AFM analysis from a typical TSNP sample with a mean thickness of 11±2 nm. The two samples in the filtered AFM height images shown have measurements of 7 nm (B) and 9 nm (C); (E) is a linear fit (R=0.96) of the structural data depicting the linear relationship between the nanoparticle ensembles mean edge length (nm) and mean thickness (nm);

FIG. 12 is a UV-vis spectra and a transmission electron micrograph of a TSNP ensemble with an aspect ratio of 6 and a λₘₐₓ of 700 nm;

FIG. 13 is a UV-vis spectra and a transmission electron micrograph of a TSNP ensemble with an aspect ratio of 7.4 and a λₘₐₓ of 868 nm;

FIG. 14 is a UV-vis spectra and a transmission electron micrograph of a TSNP ensemble with an aspect ratio of 9.6 and a λₘₐₓ of 919 nm;

FIG. 15 is a UV-vis spectra and a transmission electron micrograph of a TSNP ensemble with an aspect ratio of 12.3 and a λₘₐₓ of 1070 nm;

FIG. 16 is a UV-vis spectra and a transmission electron micrograph of a TSNP ensemble with an aspect ratio of 13.3 and a λₘₐₓ of 1093 nm;

FIG. 17 is a plot of the LSPR sensitivity of three different TSNP ensemble sample sets with an aspect ratio of 3.99 to 6.95 as function of percentage surface area;

FIG. 18 is a plot of LSPR sensitivity of the TSNP ensemble samples of FIG. 17;

FIG. 19 is a plot showing the percentage surface area of the TSNP ensemble samples of FIG. 17;

FIG. 20 (A) is a graph showing the dependence of the localised surface plasmon resonance (LSPR) peak wavelength sensitivity on the edge length of TSNP; (B) is a graph showing the dependence of the localised surface plasmon resonance (LSPR) peak wavelength sensitivity on the aspect ratio of TSNP; and (C) is a graph illustrating that the shape factor for nanostructures increases with increasing aspect ratio;

FIG. 21 (A) and (B) are graphs showing linewidth calculations to determine the dominant contribution of LSPR bandwidth and resonance for TSNPs. In (A) the linewidth equation has been fitted to the experimentally measured linewidths minus the bulk value for silver (72 meV); Also shown is the relative contribution of surface electron scattering and volume induced radiation damping and the linewidth data has been plotted against the reciprocal of the TEM measured edge length showing the fit of the linewidth equation with values of A=2 and κ=1.2; in (B) the experimentally measured linewidth data and the experimentally measured aspect ratio data have been plotted, also shown are fits where the aspect ratio is reduced to half and a quarter of the experimental values;

FIG. 22 (A) is a UV-vis Spectral shift observed for mean edge length 82 nm mean height 11.1 nm TSNP sample with original LSPR peak wavelength of 868 nm, in varying sucrose solution concentrations of different refractive indices. (B) is a plot showing the linear dependence of the shift recorded in the LSPR peak wavelength upon the refractive index of the corresponding sucrose solution;

FIG. 23 is a plot of the peak LSPR wavelength of twenty TSNP solution samples plotted against the corresponding ensemble average LSPR sensitivity measured using the sucrose refractive index analysis. As the peak wavelength approaches the NIR the sensitivity increases significantly, the inset shows the highest LSPR sensitivities recorded for the four most sensitive ensemble samples tested in ascending order;

FIG. 24 is a set of UV-vis spectra showing the red and blue optical tuning of LSPR in-plane dipole peak about the original LSPR peak position using Bovine serum albumin (BSA) at pH 5.8 for the purposes for red shifting and sucrose and a range of concentrations of C-reactive protein for systematic blue shifting;

FIG. 25 is a set of UV-Vis-NIR spectra of sequential blue shifting of high aspect ratio triangular silver nanoprisms treated using C-reactive protein in aqueous solution;

FIG. 26 is a set of UV-Vis-NIR spectra of sequential blue shifting of high aspect ratio TSNP in the presence of 50% w/v sucrose where A is un coated, unfunctionalised TSNP, B is in situ phosphocholine (PC) functionalised TSNP, C is in situ hydrolysed-PC and un-hydrolysed PC functionalised TSNP where the hydrolysed-PC has been exposed to water vapour and allowed to hydrolyse, and D is in situ hydrolysed-PC functionalised TSNP;

FIG. 27 are UV-vis spectra for samples of the PVA nanoparticles of Tables 3 and 4 in which (A) is sample S22.2; (B) is sample S31.2; (C) is sample 7; (D) is sample 6; (E) is sample 2; (F) is sample S21.1; (G) is sample 521.2; (H) is sample 522.1; and (I) is sample S22.3;

FIG. 28 is a graph showing a comparison of the figure of merit (FOM) for refractive index local surface plasmon resonance (LSPR) sensing of TSNP prepared in accordance with the methods of Examples 1 to 3 and PVA nanoparticles prepared in accordance with the method described in PCT/1E2004/000047;

FIG. 29 is a graph showing a comparison of refractive index local surface plasmon resonance (LSPR) sensitivity of TSNP prepared in accordance with the methods of Examples 1 to 3 and PVA nanoparticles prepared in accordance with the method described in PCT/IE2004/000047;

FIG. 30 is a graph showing a comparison of full width half maximum (FWHM) of TSNP prepared in accordance with the methods of Examples 1 to 3 and PVA nanoparticles prepared in accordance with the method described in PCT/IE2004/000047;

FIG. 31 is a transmission electron micrograph of a single snipped high aspect ratio triangular silver nanoprism;

FIG. 32 is a transmission electron micrograph of a mixture of snipped and unsnipped high aspect ratio triangular silver nanoprisms;

FIG. 33 is a UV-vis spectrum of TSNP in-situ functionalised and stabilised by IgG;

FIG. 34 is a UV-vis spectrum of TSNP stabilised by TSC (solid line) and TSNP in-situ functionalised and stabilised by IgG(dashed line);

FIG. 35 is a UV-vis spectrum of TSNP in-situ functionalised and stabilised by cytidine 5′-diphosphocholine (PC);

FIG. 36 is a UV-vis spectrum of TSNP in-situ functionalised and stabilised by TSC (solid line), phosphocholine (PC) (dashed line) and TSC+PC (dotted line);

FIG. 37 is a UV-vis spectrum of TSNP in-situ functionalised and stabilised by oligonucleotide that have been modified to contain a positively charged headgroup;

FIG. 38 is a schematic of a total solution phase ensemble average biosensor detection system where in the in situ-receptor functionalised TSNP remain in solution phase throughout the detection process;

FIG. 39 (A) is a UV-vis spectrum of a CRP Assay using total solution phase in-situ phosphocholine functionalised TSNP ensemble with an ensemble average in-plane dipole LSPR peak in the region of 680 nm. Systematic LSPR peak wavelength shift response on the presence of CRP is observed by the ensemble average LSPR of the in-situ phosphocholine functionalised TSNP; (B) is a UV-vis spectrum of a CRP assay using in-situ phosphocholine functionalised TSNP and chemically blocked using 0.2 μM MHA. A systematic LSPR peak wavelength shift response on the presence of CRP is observed by the ensemble average LSRP sensitivity of the in-situ phosphocholine functionalised TSNP; (C) is a UV-vis spectrum of a CRP assay using in-situ phosphocholine functionalised TSNP, chemically blocked using 0.2 μM MHA in the presence of human serum. An LSPR peak wavelength shift response on the presence of CRP is observed by the ensemble average LSRP of the in-situ phosphocholine functionalised TSNP in human sera; and (D) is a dose response curve for CRP in the range 0 ng/ml to 250 ng/ml;

FIG. 40 is a set of UV-Visible spectra of unfunctionalised (solid line) and in-situ nucleic acid probe functionalised and stabilised TSNP (dotted line);

FIG. 41 are optical extinction spectra measured using UV-visible-NIR spectroscopy of silver nanoplates produced in accordance with Example 7 with (i) 1.25 mM TSC stabilisation, (ii) in-situ functionalisation with 423 ng/ml anti-CRP antibody followed by the addition of 0.3 mM TSC, (iii0 in-situ functionalisation with 1.27 μg/ml anti-CRP antibody followed by the addition of 0.3 mM TSC, (iv) 2 mM cytidine stabilisation, and (v) no stabiliser in which (A) is 30 minutes after production; (B) is 24 hours after production; and (C) is 1 week after production;

FIG. 42 (A) is a set of UV-Vis spectra for in situ PC functionalized TSNP blocked with MHA concentration in the range of 0 to 20 μM; (B) is a UV-Vis spectra for in situ IgG functionalized TSNP blocked with MHA concentration in the range of 0 to 20 μM;

FIG. 43 is an optical extinction spectra of the TSNP samples (TSC stabilised and PC stabilised TSNP) of Example 8B;

FIG. 44 is an optical extinction spectra of MHA blocked TSC stabilised TSNP with an original peak wavelength in the region of 541 nm of Example 8B;

FIG. 45 is a plot showing the LSPR sensitivities and peak wavelength dependence of TSC stabilised TSNP with an original peak wavelength in the region of 541 nm upon the nM concentration of MHA (log scale) in accordance with Example 8B;

FIG. 46 is an optical extinction spectra of MHA blocked PC stabilised TSNP with an original peak wavelength in the region of 545 nm of Example 8B;

FIG. 47 is a plot showing the LSPR sensitivities and peak wavelength dependence of PC stabilised TSNP with an original peak wavelength in the region of 545 nm upon blocking with nM concentration of MHA (log scale) in accordance with Example 8B;

FIG. 48 is an optical extinction spectra of MHA blocked TSC stabilised TSNP with an original peak wavelength in the region of 577 nm of Example 8B;

FIG. 49 is a plot showing the LSPR sensitivities and peak wavelength dependence of TSC stabilised TSNP with an original peak wavelength in the region of 577 nm upon blocking with nM concentration of MHA (log scale) in accordance with Example 8B;

FIG. 50 is an optical extinction spectra of MHA blocked PC stabilised TSNP with an original peak wavelength in the region of 617 nm of Example 8B;

FIG. 51 is a plot showing the LSPR sensitivities and peak wavelength dependence of PC stabilised TSNP with an original peak wavelength in the region of 617 nm upon blocking with nM concentration of MHA (log scale) in accordance with Example 8B;

FIG. 52 is a spectra of TSC stabilised TSNP blocked with 20 μM MHA in the presence and absence of 200 ng CRP in accordance with Example 8C;

FIG. 53 is a spectra of PC stabilised TSNP blocked with 20 μM MHA in the presence and absence of 200 ng CRP in accordance with Example 8C;

FIG. 54 is a spectra of TSC stabilised TSNP blocked with CRP-free serumin the presence and absence of 200 ng CRP in accordance with Example 8C;

FIG. 55 is a spectra of PC stabilised TSNP blocked with CRP-free serumin the presence and absence of 200 ng CRP in accordance with Example 8C;

FIG. 56 is a plot of the time dependence of serum blocked PC stabilised TSNP(CRP sensor) in the presence and absence of 200 ng CRP in accordance with Example 8C;

FIG. 57 (A) is a series of darkfield images of a group of coupled TSNP moving in Brownian motion in solution phase; and (B) is a series of darkfield images of twinned, coupled and grouped TSNP. Note in the case of each group or twin coupled TSNP the entire group or twin appear same colour due to the sharing of the coupled plasmon;

FIG. 58 (A) to (C) are dark field images of (A) individual in-situ probe functionalised TSNP; (B) individual probe in-situ functionalised TSNP and negative target coated substrate; and (C) individual in-situ probe functionalised TSNP and positive target coated substrate;

FIG. 59 (A) is a set of UV-vis spectra of in situ IgG functionalised TSNP in response to a range of concentrations of aIgG; (B) is an aIgG Assay response curve using in-situ IgG antibody functionalised TSNP;

FIG. 60 is a schematic of a total solution phase individual single TSNP assay. The TSNP sensors/labels may exhibit a spectral response such as a shift, increase or decrease in optical scattering or a combination of these features upon the binding of an analyte molecule;

FIG. 61 is a schematic of an assay configuration involving TSNP functionalised with 3 different probes Probe 1 identifies and quantifies the target; Probe 2 recognises allele 1 (wild type); and probe 3 recognises allele 2 (mutant). The TSNP sensors/labels may exhibit a spectral response such as a shift, increase or decrease in optical scattering or a combination of these features upon the binding of an analyte molecule. This change in the optical spectrum may be shared by all of the bound probe functionalised TSNP to a single analyte in that a uniform spectral profile may be exhibited by each of the TSNP in the bound group due to plasmon coupling;

FIG. 62 is a schematic of a twinned or pregrouped probe comprising functionalised TSNP which may facilitate increased LSPR sensitivity and/or enable increased optical extinction cross section than in the case of single probe functionalised TSNP;

FIG. 63 is a schematic of an assay configuration involving dual probe functionalised TSNP. Probe 1 is for target identification e.g. the presence or absence of analyte; and Probe 2 acts to further characterise the analyte for example by subtyping the analyte such as in the case of bacterial or protein isotyping;

FIG. 64 is a schematic of the capturing and tethering or immobilisation of probe functionalised TSNP sensors on the binding of target analyte with the solution phase TSNP sensors and substrate immobilised probes. The TSNP sensors/labels may exhibit a spectral response such as a shift, increase or decrease in optical scattering or a combination of these features upon the binding of an analyte molecule;

FIG. 65 is a schematic of multiplex TNSP sensors wherein two or more different probe functionalised TSNP, each have a distinct and different LSPR peak wavelength for each corresponding probe, Probe functionalised TSNP sensors are captured and tethered or immobilised on the binding of target analyte with the solution phase TSNP sensors and substrate immobilised probes;

FIG. 66 is a schematic of a tethered probe arrangement wherein substantially all of the probe functionalised TSNP surface are available for binding; the TSNP sensors/labels may exhibit a spectral response such as a shift, increase or decrease in optical scattering or a combination of these features upon the binding of an analyte molecule;

FIG. 67 (A) is a dark field image of individual and grouped C-reactive protein receptor in situ functionalised TSNP in the absence of C-reactive protein; (B) is a UV-Vis Spectra of an individual C-reactive protein receptor in situ functionalised TSNP in the absence of C-reactive protein; and (C) is a UV-Vis Spectra of a different individual C-reactive protein receptor in situ functionalised TSNP in the absence of C-reactive protein;

FIG. 68 (A) is a dark field image of individual and grouped C-reactive protein receptor in situ functionalised TSNP in the presence of 100 ng/ml C-reactive protein; (B) is a UV-Vis Spectra of an individual C-reactive protein receptor in situ functionalised TSNP in the presence of 100 ng/ml C-reactive protein; and (C) is a UV-Vis Spectra of a different individual C-reactive protein receptor in situ functionalised TSNP in the presence of 100 ng/ml C-reactive protein An average shift of 38 nm is found for the TSNP CRP sensor in the presence of 100 ng/ml C-reactive protein;

FIG. 69 is a schematic of target functionalised TSNP, targets may be nucleic acids, proteins, antibodies, peptides, ligands. Cancer cell target functionalised TSNP delivered cancer cells in a cancer tumour located within healthy normal cell tissue. A cell with specific protein target functionalised TSNP and specific gene sequence target functionalised TSNP delivered to target locations for in situ detection, monitoring, characterisation, labelling and mapping of events and process of target bodies. The target functionalised TSNP sensors/labels may exhibit a spectral response such as a shift, increase or decrease in optical scattering or a combination of these features upon the binding of an analyte molecule resulting from the activity of the body under surveillance;

FIG. 70 (A) and (B) are darkfield images and the corresponding UV-Vis spectrum of TSNP moving in Brownian motion in solution phase;

FIG. 71 (A) is a Darkfield image at 100× magnification and (B) is the corresponding dark field scattering spectrum of an ensemble collection of circa 5000 nanoparticles solution phase of TSNP moving freely in solution;

FIG. 72 is a Darkfield scattering spectrum of an ensemble collection of solution phase of TSNP moving freely in solution at 100× magnification and corresponding UV-Vis spectrum of nanoplates using a 1 cm path length;

FIG. 73 is a Darkfield scattering spectrum at 100× magnification of another collection of solution phase of TSNP moving freely in solution;

FIG. 74 is a Darkfield scattering spectrum at 100× magnification of the collection of solution phase TSNP moving freely in solution and corresponding UV-Vis spectrum of nanoplates using a 1 cm path length;

FIG. 75 is a Darkfield scattering spectrum at 100× magnification of another collection of solution phase TSNP moving freely in solution and corresponding UV-Vis spectrum of nanoplates using a 1 cm path length;

FIG. 76 is a Darkfield scattering spectrum at 100× magnification of anther collection of solution phase TSNP moving freely in solution in a 1.33 (water) and 1.42 (50% w/v sucrose solution) refractive index medium and corresponding UV-Vis spectrum of nanoplates using a 1 cm path length in a 1.33 (water) and 1.42 (50% w/v sucrose solution) refractive index medium;

FIG. 77 (A) is a set of UV-Vis extinction spectra for another solution phase ensemble of silver nanopolates in water, 25% sucrose and 50% sucrose, while B is a set of darkfield scattering spectrum for a collection of circa 5000 of the same silver nanoplates in solution phase. C shows the a linear plot of the peak wavelength shift as a function of refractive index in the case of both the UV-Vis extinction spectra and the darkfield scattering spectra;

FIG. 78 is a plot showing the difference between the peak wavelength positions of DDA single TSNP calculated and the experimentally measured TSNP ensemble using UV-VIS peak wavelength position (black squares). Difference between the DDA single TSNP calculated and the experimentally measured TSNP ensemble using UV-VIS peak wavelength position (grey stars);

FIG. 79 is a plot showing the difference between the DDA single TSNP calculated and the experimentally measured TSNP ensemble using UV-VIS peak wavelength position (black squares) as a function of TSNP aspect ratio;

FIG. 80 is a plot showing the peak wavelength positions of nanoparticles measured using UV-Vis with a 1 cm optical path length (black squares) and darkfield (grey stars) and calculated using DDA (black circles);

FIG. 81 is a Calculated Spectra using DDA and corresponding UV-Vis experimental measurements of spectra for shape 1 nanoparticles listed in table 6;

FIG. 82 is a Calculated Spectra using DDA and corresponding UV-Vis experimental measurements of spectra for shape 3 nanoparticles listed in table 6;

FIG. 83 is a Calculated Spectra using DDA and corresponding UV-Vis experimental measurements of spectra for shape 5 nanoparticles listed in table 6;

FIG. 84 is a Calculated Spectra using DDA and corresponding UV-Vis experimental measurements of spectra for shape 7 nanoparticles listed in table 6;

FIG. 85 is a Calculated Spectra using DDA and corresponding UV-Vis experimental measurements of spectra for shape 8 nanoparticles listed in table 6;

FIG. 86 is a Calculated Spectra using DDA and corresponding UV-Vis experimental measurements of spectra for shape 9 nanoparticles listed in table 6;

FIG. 87 is a Calculated Spectra using DDA and corresponding UV-Vis experimental measurements of spectra for shape 11 nanoparticles listed in table 6;

FIG. 88 is a Calculated Spectra using DDA and corresponding UV-Vis experimental measurements of spectra for shape 13 nanoparticles listed in table 6;

FIG. 89 is a Calculated Spectra using DDA and corresponding UV-Vis experimental measurements of spectra for shape 15 nanoparticles listed in table 6;

FIG. 90 is a Calculated Spectra using DDA and corresponding UV-Vis experimental measurements of spectra for shape 16 nanoparticles listed in table 6;

FIG. 91 is a Calculated Spectra using DDA and corresponding UV-Vis experimental measurements of spectra for shape 17 nanoparticles listed in table 6;

FIG. 92 is a Calculated Spectra using DDA and corresponding UV-Vis experimental measurements of spectra for shape 19 nanoparticles listed in table 6;

FIG. 93 is a UV-vis spectra showing the optical tuning of LSPR in-plane dipole peak for TSNP grown from various quantities of silver seeds in which from A-G, 1 mL, 800 μL, 750 μL, 600 μL, 500 μL, 400 μL, and 250 μL seeds respectively were used;

FIG. 94(A) is a set of SERS spectra of crystal violet added to each of the TSNP of FIG. 93 after aggregation with MgSO₄; (B) is a plot of the change in intensity of the 1173 cm⁻¹ peak against the initial LSPR λₘₐₓ of each TSNP; and (C) is a UV-vis spectra of each of the TSNP of FIG. 93 after aggregation with MgSO₄; Note the degradation of the out of plane quadrupole @ circa 345 nm indicating actual physical destruction of the nanoplate morphology and aggregation of the nanoplates as distinct from coupling;

FIG. 95(A) is a set of SERS spectra of crystal violet added to the TSNP from FIG. 93 wherein the crystal violet analyte is added prior to MgSO₄ (the lines from top to bottom are G to A respectively); (B) is a plot of the change in intensity of the 1173 cm⁻¹ peak against the initial LSPR λₘₐₓ of each TSNP;

FIG. 96(A) is a set of SERS spectra of 4-mercaptopyridine (30 μM) using TSNP from FIG. 93 as substrates (the lines from top to bottom are G to A respectively); (B) is a plot of the Raman intensity of the 1004 cm⁻¹ band of 4-mercaptopyridine in (A) against LSPR λₘₐₓ;

FIG. 97 is a SERS spectra of adenine using TSNP of FIG. 93 as substrates (the lines from top to bottom are G to A respectively);

FIG. 98 is a set of SERS spectra of 4-mercatopyridine (30 μM) using Lee and Meisel⁴⁵ colloid and TSNP G₆₁₅ from FIG. 93 as the substrates;

FIGS. 99(A) and (B) are normalized UV-vis spectra of TSNP grown from various quantities of silver seeds in which A-K 650 μL, 500 μL, 400 μL, 260 μL, 200 μL, 120 μL, 90 μL, 60 μL, 40 μL, 20 μL, 10 μL seeds respectively were used (the lines from left to right are A to K respectively);

FIG. 100 is a set of UV-vis spectra of TSNP A-H of FIG. 99A after aggregation with MgSO₄;

FIG. 101(A) to (D) are TEM images of TSNP (sample H from FIG. 99A) prior to aggregation (A and C) and after aggregation with MgSO₄ (B and D);

FIG. 102A) and (B) are normalized UV-vis spectra of TSNP used for aggregation studies grown from various quantities of silver seeds in which A-E 650 μL, 400 μL, 200 pt, 60 μL and 10 μL seeds respectively were used (the lines from left to right are A to E respectively);

FIGS. 103(A) to (E) are UV-vis spectra monitoring the coupling process of TSNP from FIG. 102 in the presence of 4-aminothiophenol (30 μM), spectra were recorded every 30 seconds for 15 minutes. a) A₅₀₀ b) B₅₅₀ c) C₅₉₀ d) D₇₆₅ e) E₉₈₉ the vertical line indicates the laser excitation wavelength; (F) is a TEM image of TSNP E₅₉₅ from FIG. 102 in the presence of 30 μM 4-aminothiophenol;

FIG. 104(A) is a set of UV-vis spectra monitoring the aggregation process of TSNP D₅₉₀ in the presence of 4-methylthiophenol (30 μM), spectra were recorded every 30 seconds for 15 minutes the vertical line indicates the laser excitation wavelength; (B) is a TEM image of TSNP E₅₉₅ after aggregation;

FIG. 105(A) is a set of UV-vis spectra monitoring the aggregation process of TSNP D₅₉₀ in the presence of thiophenol (30 μM), spectra were recorded every 30 seconds for 15 minutes the vertical line indicates the laser excitation wavelength (B) is a TEM image of TSNP E₅₉₅ after aggregation;

FIG. 106 is a set of SERS spectra of thiophenol (30 μM) as an analyte using the TSNP solutions from FIG. 99 as substrates (the lines from top to bottom are K to A respectively);

FIG. 107 (A) and (B) are Raman intensities of the band at (A) 1574 and (B) 1000 cm⁻¹ of thiophenol versus the initial LSPR μₘₐₓ of each TSNP;

FIG. 108 is a set of SERS spectra of 4-methylthiophenol (30 μM) as an analyte using the TSNP sols from FIG. 38 as substrates (the lines from top to bottom are K to A respectively);

FIG. 109 (A) and (B) are Raman intensities of the band at (A) 1594 and (B) 1080 cm⁻¹ in 4-methylthiophenol versus the initial LSPR λₘₐₓ of each TSNP;

FIG. 110 is a set of SERS spectra of 4-aminothiophenol (30 μM) as an analyte using the TSNP sol from FIG. 99 as substrates (the lines from top to bottom are K to A respectively);

FIG. 111 (A) and (B) are Raman intensities of the band at (A) 1594 and (B) 1080 cm⁻¹ of 4-aminothiophenol versus the initial LSPR λₘₐₓ of each TSNP;

FIG. 112 is a set of SERS spectra of 4-mercaptopyridine (30 μM) as an analyte using the TSNP sols from FIG. 99 as substrates, TSNP were aggregated with MgSO₄ (0.1M) after the addition of the analyte (the lines from top to bottom are K to A respectively);

FIG. 113 is a plot showing the SERS intensities of the band at 1004 cm⁻¹ of 4-mercaptopyridine versus the initial LSPR λₘₐₓ, of each TSNP;

FIG. 114 is a SERS spectrum for ethanol (3.4M);

FIG. 115 is a set of SERS spectra at a laser excitation wavelength of 785 nm of 4-aminothiophenol (30 μM) when the concentration of substrate was varied from 9.375 μm to 150 μm, Φ denotes the EtOH peaks (the lines from top to bottom are 9.375 μm to 150 μm respectively);

FIG. 116 is a set of SERS spectra at a laser excitation wavelength of 785 nm of 4-mercaptopyridine (30 μM) when the concentration of substrate was varied from 9.375 μm to 150 μm, Φ denotes the EtOH peaks (the lines from top to bottom are 9.375 μm to 150 μm respectively);

FIG. 117 shows the E-field enhancement contours external to a dimer of silver nanoparticles separated by 2 nm for a plane that is along the inter-particle axis and that passes midway through the two particles. In the 3D plots the axis perpendicular to the selected plane represents the amount of E-field enhancement around the dimer (left 430 nm, right 520 nm)⁵⁵;

FIG. 118 is a UV-visible spectra of the sols of Example 20;

FIG. 119 are UV-visible spectra of the coupling of triangular nanoplates with (A) 30 μM 4-ATP; (B) 3 μM 4-ATP; and (C) 0.3 μM 4-ATP;

FIG. 120 are UV-visible spectra of the coupling of hexagonal nanoplates prepared with 12.5 μM TSC with (A1) 30 μM 4-ATP; (B1) 3 μM 4-ATP; and (C1) 0.3 μM 4-ATP; and the coupling of hexagonal nanoplates prepared with 1.25 mM TSC with (A2) 30 μM 4-ATP; (B2) 3 μM 4-ATP; and (C2) 0.3 μM 4-ATP;

FIG. 121 are UV-visible spectra of the coupling of disk shaped nanoplates prepared with 12.5 μM TSC with (A1) 30 μM 4-ATP; (B1) 3 μM 4-ATP; and (C1) 0.3 μM 4-ATP; and the coupling of hexagonal nanoplates prepared with 12.5 μM TSC and 1.25 mM TSC added with (A2) 30 μM 4-ATP; (B2) 3 μM 4-ATP; and (C2) 0.3 μM 4-ATP;

FIG. 122 is a Raman spectra for 4-aminothiophenol and ethanol;

FIG. 123 is SERS of triangular, hexagonal and disk shaped nanoplates in the presence of 4-ATP at a concentration of 100 μM;

FIG. 124 is SERS of triangular, hexagonal and disk shaped nanoplates in the presence of 4-ATP at a concentration of 30 μM;

FIG. 125 is SERS of triangular, hexagonal and disk shaped nanoplates in the presence of 4-ATP at a concentration of 10 μM;

FIG. 126 is SERS of triangular, hexagonal and disk shaped nanoplates in the presence of 4-ATP at a concentration of 3 μM;

FIG. 127 is SERS of triangular, hexagonal and disk shaped nanoplates in the presence of 4-ATP at a concentration of 1 μM;

FIG. 128 is SERS of triangular, hexagonal and disk shaped nanoplates in the presence of 4-ATP at a concentration of 0.3 μM;

FIG. 129 is SERS of triangular, hexagonal and disk shaped nanoplates in the presence of 4-ATP at a concentration of 0.1 μM;

FIG. 130 is SERS of triangular, hexagonal and disk shaped nanoplates in the presence of 4-ATP at a concentration of 0.03 μM;

FIG. 131 is SERS peak intensities of 4-ATP at a concentration range of 100 μM to 0.03 μM on triangular nanoplates;

FIG. 132 is SERS peak intensities of 4-ATP at a concentration range of 100 μM to 0.03 μM on hexagonal nanoplates;

FIG. 133 is SERS peak intensities of 4-ATP at a concentration range from 100 μM to 0.03 μM on disk nanoplates;

FIG. 134 is a schematic of a slide containing hybridisation chambers and nucleic acid array spotted. Oligonucleotide 1=positive nucleic acid Target, complementary to probe sequences functionalised on TSNP. Oligonucleotide 2 and 3 are negative controls. Spot diameter is approximately 200 μm Hybridisation chamber volume is 40 μl;

FIG. 135 shows a dark field image taken at a magnification of 100× of unfunctionalised TSNP on a spot containing immobilized positive target nucleic acid at a concentration of 20 μM. This image confirms negative unspecific binding of bare unfunctionalised TSNP with nucleic acid sequences and a very low background binding signal;

FIG. 136 shows a dark field image as representative of TSNP functionalized with oligonucleotides with are complementary with the immobilized positive target sequence. Specifically this case shows a dark field image taken at a magnification of 100× of thiol functionalised TSNP on a spot containing immobilized positive target nucleic acid at a concentration of 20 μM. This image confirms very low unspecific binding of functionalised TSNP with nucleic acid sequences and a very low background binding signal. Note that the one TSNP observable in the image is a group;

FIG. 137 shows a dark field image taken at a magnification of 10× of DAPA functionalised TSNP on a spot containing immobilized positive target nucleic acid. This image confirms high binding of DAPA functionalised TSNP with complementary nucleic acid sequences;

FIG. 138 shows a dark field image taken at a magnification of 100× of DAPA functionalised TSNP on a spot containing immobilized positive target nucleic acid. This image confirms high binding of DAPA functionalised TSNP with complementary nucleic acid sequences;

FIG. 139 shows a dark field image taken at a magnification of 100× of DAPA functionalised TSNP in a position between spots containing immobilized positive target nucleic acid. This image confirms the very low unspecific binding of DAPA functionalised TSNP and very low background unspecific binding signal;

FIG. 140 shows a dark field image taken at a magnification of 100× of no end group chemistry functionalised TSNP on a spot containing immobilized positive target nucleic acid. This image confirms the efficient binding of TSNP functionalised with complementary oligonucleotides with out any additional end group chemistry with complementary nucleic acid target sequences;

FIG. 141 shows a dark field image taken at a magnification of 10× of IDEA functionalised TSNP on a spot containing immobilized positive target nucleic acid. This image confirms the binding of IDEA functionalised TSNP with complementary nucleic acid target sequences;

FIG. 142 shows a dark field image taken at a magnification of 100× of IDEA functionalised TSNP on a spot containing immobilized positive target nucleic acid. This image confirms the binding of IDEA functionalised TSNP with complementary nucleic acid target sequences;

FIG. 143 shows a dark field image taken at a magnification of 10× of Thiol 20AA functionalised TSNP on a spot containing immobilized positive target nucleic acid. This image confirms high binding of Thiol 20 AA functionalised TSNP with complementary nucleic acid sequences;

FIG. 144 shows a dark field image taken at a magnification of 100× of Thiol 20 AA functionalised TSNP on a spot containing immobilized positive target nucleic acid. This image confirms the very high binding of Thiol 20 AA functionalised TSNP with complementary nucleic acid sequences;

FIG. 145 shows a dark field image taken at a magnification of 10× of Thiol functionalised TSNP on a spot containing immobilized positive target nucleic acid. This image confirms the very high binding of Thiol functionalised TSNP with complementary nucleic acid sequences;

FIG. 145 shows a dark field image taken at a magnification of 100× of Thiol functionalised TSNP on a spot containing immobilized positive target nucleic acid. This image confirms the very high binding of Thiol functionalised TSNP with complementary nucleic acid sequences. In addition the darkfield image shows that the Thiol functionalised TSNP of consist of twinned, grouped and coupled TSNP. Note in the case of each group or twin coupled TSNP the entire group or twin are same colour which is uniformly distributed over the extent of the TNSP group. This is due to the sharing of the coupled plasmon. The TSNP group shows increased optical extinction cross section or brightness than in the case of single functionalised TSNP sensors and facilitates optical detection. To this end live observation of these tethered grouped TSNP sensors shows the vigorous movement of the TSNP group about their tethered position in solution. TSNP grouped sensor may also facilitate increased LSPR refractive index sensitivity over single TSNP sensors;

FIG. 147 shows a darkfield image of a grouped or precoupled TSNP coupled TSNP in solution phase. Note entire TSNP group is the same colour which is uniformly distributed over the extent of the TNSP group. This is due to the sharing of the plasmon among coupled TSNP. The TSNP group shows increased optical scattering which is observed as increase brightness than in the case of single probe functionalised TSNP facilitating optical detection and may also facilitate increased LSPR refractive index sensitivity. Increased LSPR refractive index sensitivity of coupled TSNP may be achieved by presenting the receptors such that they binding with the analyte occurs within the E-field;

FIG. 148 shows a sequence of dark field images taken at a magnification of 10× of DAPA functionalised TSNP corresponding to spots containing immobilized positive target nucleic acid at a concentrations of a) 20 μM, b) 2 μM, c) 200 nM, d) 20 nM and e) 2 nM. These image confirm the high binding of DAPA functionalised TSNP with complementary nucleic acid sequences across the spotting concentration range from 20 μM to 2 nM;

FIG. 149 shows the optical transmission spectra in the ultraviolet-visible-infrared region of the spectrum of stabilised (1.25 mM trisodium citrate—denoted “TSC”) and non stabilised nanoplates at (a) 0 minutes, (b) 18 hours and (c) 1 week post production, indicating the stability of these formulations made using the process;

FIG. 150 shows the optical transmission spectra in the ultraviolet-visible-infrared region of the spectrum of Trisodium citrate (TSC), Polyvinylpyrrolidone (PVP) and gelatine stabilised (capped) silver nanoplates after densification using cross flow ultrafiltration. The stabilising agent was added before cross-flow filtration, demonstrating the compatibility of the cross-flow filtration processes even with stablised formulations made using the process;

FIG. 151 shows the optical transmission spectra in the ultraviolet-visible-infrared region of the spectrum of silver nanoplates before and after densification using cross flow ultrafiltration. Also shown is the spectrum of the dead volume;

FIG. 152 shows a graph of the resistivity of a film made by depositing a 1 wt % aqueous suspension of silver nanoplates on a substrate, as a function of curing temperature. The resistivity drops dramatically between 120° C. and 130° C. and drops gradually at higher temperatures;

FIG. 153 shows a graph of the resistivity of a film made by depositing an aqueous suspension of silver nanoplates on a substrate, at different silver contents by weight, as a function of curing temperature.

FIG. 154 is a micrograph showing the alignment of functionalised triangular nanoplates over 15 microns.

FIG. 155 is a micrograph showing the assembled network of chemically functionalised triangular nanoplates

FIG. 156 is a micrograph showing an assembled network of hexagonal silver nanoplates which result in better packing than triangular nanoplates.

FIG. 157 shows two photographs of silver thin films, post thermal curing, made with (a) 0.1 wt % and (b) 1 wt % of silver nanoplates

FIG. 158 shows a graph of the thin film transmittance of a 0.1 wt % silver nanoplate coated glass substrate, in the ultraviolet-visible-infrared spectral region.

### DETAILED DESCRIPTION

Spectroscopic studies at the individual-particle or single-molecule levels can provide invaluable information on the dynamics of complex systems in fields as different as materials science and molecular cell biology. These measurements can provide a direct record of the time trajectory and reactions of individual molecules that are otherwise hidden in the ensemble average.

The use of LSPR sensing techniques with a single nanoplate limit provides several advantages for example, the absolute detection limit (i.e. number of analyte molecules per nanoplate) is dramatically reduced, and the formation of a molecular monolayer on a nanoparticle array results in a larger LSPR max shift which is of the order of about 100 times greater than the instrumental resolution of typical small-footprint UV-visible spectrophotometer. It has been postulated that the limit of detection for single nanoplate based LSPR sensing is well below 1,000 molecules for small-molecule adsorbates. For larger molecules, such as antibodies and proteins single nanoplate based LSPR sensing may result in a greater change in the local dielectric environment per adsorbed molecule, which will further improve detection limits. Theory suggests that the sensitivity of single-nanoplate LSPR spectroscopy could approach the single-molecule limit of detection for large biomolecules. Additionally, as a result of the high sensitivity of the sensor only a very small sample volume (e.g. attoliters) is required to obtain a measurable response.

The absorbance spectra and images of individual nanoplates and individual nanoplate groups can be recorded using an inverted optical microscope equipped with a dark-field condenser. The dark-field condenser forms a hollow cone of light focused at the sample. Only light that is scattered out of this cone reaches the objective. Thus, nanoplates on the substrate appear as bright, diffraction-limited spots on a dark background. Spectral measurement of multiple nanoplates under dark-field illumination can give statistically valid information for both in vivo and in vitro sensing. An array of individual nanoplates or nanoplate groups can be functionalized for binding to specific target analytes. As the nanoplates are sensitive to the local environment, a shift in the optical spectrum of the nanoplate will take place upon binding, thereby enabling quick identification of multiple proteins in a variety of environments.

Individual-nanoplate sensing platforms offer further advantages because they can be readily implemented in multiplex detection schemes. By controlling the size, shape, and chemical modification of individual nanoplates, several sensing platforms can be fabricated in which each unique nanoplate can be distinguished on the basis of the spectral location of its LSPR. Multiplex sensing can be enabled wherein nanoplates or nanoplate groups of different LSPR peak wavelengths may each be functionalized to target different analytes. Several of these unique nanoplates can then be incorporated into one device, allowing for the rapid, simultaneous, label-free detection of thousands of different chemical or biological targets, and there respective isotypes.

Advantages of utilizing single nanoplates as sensors lies in their non-invasive nature, making them ideal platforms for in vivo quantification of chemical species and monitoring of dynamic processes both in vivo and in vitro inside biological cells. Furthermore, the use of metal nanoplates as contrast agents for in vivo molecular imaging offers a number of advantages over both quantum dots and organic fluorescent dyes including increased half life, non photo-bleaching, signal stability and intensity. The very high scattering cross section of metal nanoplates as compared with the fluorescence cross sections of organic dyes and even quantum dots provides a much brighter source of signal with complete immunity to photobleaching.

Coupled nanoplate systems can show higher LSPR sensitivity compared to an isolated nanoplate. Plasmon coupling between nanoplate partners results in an exponential red shift in the optical resonance but also a near exponential increase in the medium sensitivity in direct correlation. It may therefore be advantageous to employ patterned/nanofabricated nanoplate pair arrays in LSPR sensing applications, in addition to current strategies involving non-interacting nanoplate systems.

Individual nanoparticle assay methods to date mainly rely on surface immobilisation of the metal nanoparticles such that a significant portion of the surface area of the immobilised nanoparticle is unavailable for interaction with a receptor or analyte. In a typical method gold or silver nanoparticles functionalised with receptors bind to target biomolecules which are subsequently immobilised on to a substrate surface such as a glass slide by secondary capture receptors. In certain cases further additional steps to reduce silver ions on the surface to form large silver particles for the purpose as the light scattering signal enhancers is required in what is known as silver-enhanced assays.

The distinct absorption spectra of metal nanoplates in the visible and the near-IR regions of the electromagnetic spectrum provide many excellent opportunities for detection and monitoring of in vitro and in vivo biological processes. The strong scattering of receptor functionalized metal nanoplates delivered to specific biological targets nanoplates enables them to be efficient biomarkers and image contrast agents.

We describe a biosensor comprising silver nanoplates. Nanoplates are a subset of nanoparticles having lateral dimensions (such as edge length) that are larger than their height (thickness). The term nanoplate includes for example nanodisks, nanohexagons and nanoprisms. Nanoprisms have an equilateral triangle shape.

The nanoplates described herein may be monodisperse (discrete), in one embodiment the nanoplates are well-defined triangular silver nanoplates (TSNP) of varying edge length. The TSNP may have an aspect ratio from about 2 to about 20 with increasing edge length wherein aspect ratio is the ratio of the edge length and thickness of a nanoplate and is calculated using equation 1 below.

$$\text{Aspect ratio} = \frac{\text{Edge length}}{\text{Thickness}} \qquad ( Equation 1 )$$

One of the advantages associated with nanoplates having a high aspect ratio is that the aspect ratio enables the preservation of the quantum confinement effects in nanoplates that would otherwise enter the bulk regime due to the size of the nanoplate. Nanoplates having a high aspect ratio retain many of the optical and electronic properties normally only associated with smaller nanoparticles.

Some of the advantages associated with the high aspect ratio TSNP used in the biosensors described herein include:
High aspect ratio TSNP have optimal LSRP sensing sensitivity for ready exhibition of individual TSNP or TSNP group spectral shifts easily detectable using darkfield spectroscopy or another optical reader detection system;
TSNP may be finely optically tuned throughout the Visible-NIR spectrum for use in a multiplex assay;
TSNP may be snipped (for example, chemically treated to remove one or more of the corners (tips) of the TSNP) to blue shift the spectrum in order to maintain the LSPR peak wavelength within the spectral range for which absorption by organic molecules and water does not occur;
TSNP exhibit strong optical extinction which facilitates easy observation and detection using optical reader systems such as darkfield spectroscopy for image based detection configurations;
TSNP may be readily coupled, twinned or grouped in a controlled fashion by chemical treatment or functionalisation in order to exhibit further enhanced LSPR sensitivity and optical extinction;
TSNP may be produced in situ and functionalised with receptor molecules without the need for conjugation chemistry; and
TSNP may operate in a total solution phase sensing format homogeneous with the phase of the biomolecules to be detection.

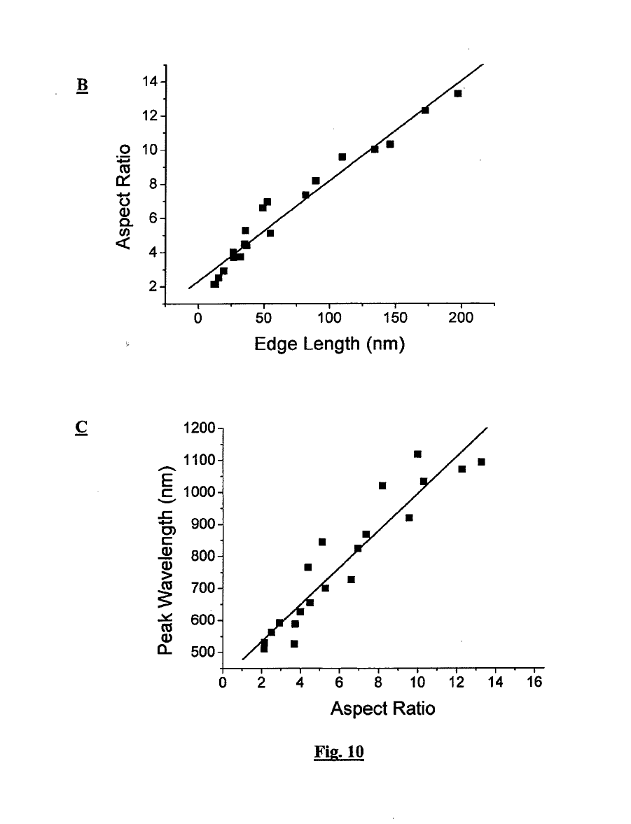

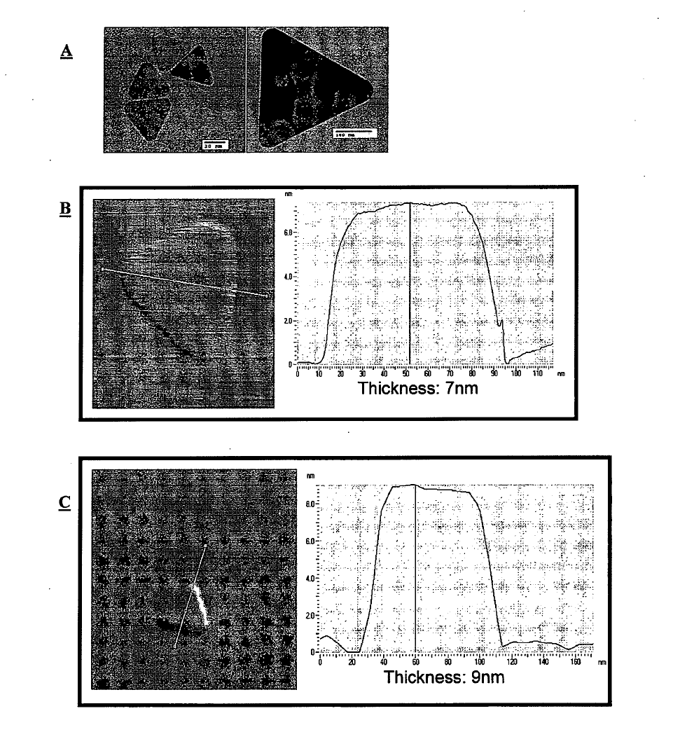

The TSNP used herein have a narrow geometric distribution which results in a highly uniform response upon interaction of a TSNP ensemble with an electromagnetic field. The aspect ratio of the TSNPs is found to increase from values of 2 to 13 with increasing edge length (FIG. 10B). The ensembles LSPR λₘₐₓ is observed to red-shift as the aspect ratio increases (FIG. 10C) for LSPRs within the range 500-1150 nm.

LSPR sensitivity scales with nanoparticle (including nanoplates) size up to the order of the electron mean free path. Larger high aspect ratio TSNP have a longer λₘₐₓ which enables more free-electron like responses and contributes to the enhanced optical and physical properties of high aspect ratio TSNP.

The majority of LSPR sensitivities presented in the literature are for single nanostructures and not ensemble averages as in the case of the TSNP described herein. As a result of the nature of ensemble averaging, LSRP sensitivity values are known to diminish and reduce compared to those calculated for individual single or coupled nanostructures. In the case of ensemble average LSPR sensitivities, Au nanorattles in solution which have an aspect ratio of approximately 2 (length ˜60-65 nm, width ˜30-35 nm depending on initial rod length), were reported to have values ranging from 150 to 285 nm/RIU at wavelength of approximately 600 nm²⁹. In comparison, average LSPR sensitivity values for all TSNP ensemble are all greater than 300 nm/RIU in the 600 nm spectral region. It is also significant that the TSNP ensemble average sensitivity values at LSPR peak wavelengths in the visible range exceed those previously reported for single nanostructures within this wavelength band such as 204 nm/RIU for single Au triangles by Sherry et al¹⁷ (Table 1 below). It is evident that the highest sensitivities of the TSNP ensemble solutions examined here are greater than those recorded to date including those for single nanostructures such as nanorice²¹, gold nanorings²² and gold nanostars¹⁹ (see Table 1 below). Furthermore, unlike other reported high LSPR sensitive nanostructures the TSNP high LSPR sensitivities occur at wavelengths shorter than 1150 nm, this is important if the TSNP are incorporated into a biosensor as the high LSPR sensitivities occur at wavelengths before water and biomolecular absorptions can become limiting factors.

Full width at half maximum (FWHM) calculations were carried out manually. The FWHM calculation involved normalisation of the LSPR spectral peak, intersecting the halfway point and determining the wavelength on either side of the LSPR peak and calculating the difference.

TABLE 1
Comparison between the LSPR sensitivities reported to date in literature
for various different single nanostructures fabricated and tested using
similar refractive index methods.

| | Peak λ (nm)/ | Δλ(nm)/ | FWHM |
|---|---|---|---|
| Sample | Shape | RIU | (eV) |
| Single silver | Pk 1: 459.3 | 93.99 | 0.284 |
| Nanoprisms¹⁷ | Pk 2: 630.6 | 204.9 | 0.246 |
| (2006) | Pk 1: 460.8 | 80.64 | 0.267 |
| | Pk 2: 634.6 | 182.9 | 0.195 |
| | Pk 1: 439.6 | 78.62 | 0.167 |
| | Pk 2: 631.4 | 196.4 | 0.166 |
| Single Silver | Sphere: | 161 | — |
| Nanoparticles | Triangle: | 197 | — |
| ²⁹(~35 nm) | Cube: | 235 | — |
| (2003) | | | |
| Nanorice | Longitudinal | 801/ | — |
| Length~366 nm | Plasmon Peak | FDTD: | |
| Width~80 nm | 1160 nm | 1060 | |
| (Shell Thickness | Transverse | 103/ | — |
| 13.7 nm)²¹ | Plasmon Peak | FDTD: | — |
| (2006) | 860 nm | 115 | |
| Gold Nanoshells²⁰ | ~30 nm | 70.9 | — |
| (2002) | immobilised gold | | |
| | solid colloid | | |
| | ~50 nm gold solid | 60 | — |
| | colloid | | |
| | Nanoshells: Mean | 408.8 | — |
| | size 50 nm Wall | | |
| | thickness~4.5 nm | | |
| Gold Nanorings | Peak at 1545 nm | 880 | — |
| 150 nm Diameter | | | |
| (Gold: 20 nm thick)²² | | | |
| (2007) | | | |
| Au Nanohole Arrays | Infinite hole | 286 | 70 nm |
| 100 nm holes³⁰ | arrays | | |
| (2007) | Finite Hole | 313 | 0.032 |
| | Arrays | | |
| Rod-Shaped Gold | Dark Field | 199 ± 70 | — |
| Nanorattles~30-40 nm | Measurement: | | |
| rods with 3-6 nm shell | 50-100 single | | |
| (2009) | particles per | | |
| | measurement | | |
| Gold NanoBoxes* | Wall thickness | 336 | ~127 nm |
| Inner edge length | 5 nm Pk~600 nm | | for 5.7 nm |
| 30 nm³¹ | | | thickness |
| (*These values were | Varied wall | 210-565 | Peak |
| predicted | thickness 15- | | broadens as |
| computationally) | 1.5 nm | | thickness is |
| | Pk: ~600 nm- | | increased |
| | 1000 nm | | |
| Ag/PVA | Peak: 600 mn | 377 | 0.89 |
| nanoparticles Edge | 55% shaped | | |
| Length 25 nm²⁵ | particles in | | |
| | ensemble, | | |
| | hexagons and | | |
| | triangles | | |
| TSNP Ensembles | Pk: 504 nm- | 178- | 0.297-0.6 |
| Edge Length 11.77- | 1093 nm | 1070 | |
| 197.23 nm | >95% Triangles | | |

Unlike other reported high LSPR sensitive nanostructures the high TSNP sensitivities occur at wavelengths shorter than 1150 nm, before water and biomolecular absorptions can become limiting factors in their suitability as biosensors. Solution phase sensors in which the nanostructure sensor is homogenous with the biological target species, is the most advantageous phase for biosensing applications. Therefore it is also significant that the TSNP ensemble sensitivity values of 281-420 nm/RIU with LSPR peak wavelengths in the visible region exceed those previously reported for nanostructures within this wavelength band such as 204 nm/RIU for single Au triangles by Sherry et al¹⁷ and 285 nm/RIU for Au nanorattles in solution²⁸. Our data demonstrate the versatility of the solution phase TSNP as optimal wavelength and sensitivity tunable local refractive index sensors.

LSPR sensitivity may be further increased by coupling of the TSNP to form dimer, trimers or multimers. This may be used in ensemble averaging mode or in individual, single dimer, trimers or multimers mode.

A number of additional properties render the TSNP suitable for molecular sensing including the nanoplates acting as optical antennas and are exceedingly bright about 10⁷ times brighter than fluorophores. Unlike fluorophores, fluorescent proteins, or even quantum dots, TSNP do not photodecompose during extended illumination. Furthermore the TSNP sensor can potentially be integrated with technology formats such as lab-on-a-chip and microfluidic microarrays to facilitate, for example, multiplex analysis of multiple genetic factors simultaneously in the move away from single-analyte analysis and focus on complex multi-analyte applications. The narrow LSPR peaks of the TSNP located at predetermined wavelengths through out the UV-Vis-NIR spectrum facilitates their application in a multiplex capacity. The nanoplates enable flexible design of assay configurations which may include a combination of imaging, spectral shifts, and optical amplification in picolitre sample volumes. Furthermore, total solution phase sensing enables assay homogeneity with the target analyte. It will be appreciated that the biosensors described herein can be used in individual TSNP solution phase assaying such as dark field imaging and spectroscopy of an in situ capture probe functionalised TSNP detecting of target molecules

We also describe a process for the in situ construction of triangular silver nanoparticles functionalized with ligands, antibodies and nucleic acids. The functionalisation may be mono, di or multi species. The process for the in situ functionalization/stabilization of triangular silver nanoplates provides a facile and versatile route for the surface modification of shaped nanoplates. Furthermore, the functionalisation method is aqueous based and does not result in a significant loss of particles for example through rigorous centrifugation/purification steps. The resultant functionalised shaped silver nanoplates are highly stable for long periods of time in aqueous solution.

The functionalisation process described herein allows for different surface chemistries to be imparted on to silver nanoplates in a one-pot procedure. The method avoids covalent linking chemistries such as EDC and sulfo-NHS coupling which can etch and degrade the nanoparticles and also avoids the use of linker chemicals, coatings and surface monolayers all of which serve to lengthen the path between a bound target molecule and the surface of the nanoparticle thereby reducing the optimal LSPR sensitivity response of the sensor. The functionalisation process is versatile and allows the surface chemistry of the TSNP to be tailored depending on the end use.

In accordance with an embodiment of the invention, silver nanoplates are produced which enable intimate and direct contact of functionalisation agents and stabilization agents with the crystal lattice of the nanoplate surface. Indeed stable silver nanoplates can be produced without any stabilization agent or functionalisation agent. In the case of in-situ functionalisation the surface of the nanoplates function to provide better binding of the functionalisation agents which is stronger, more durable, provided increased stability in harsh environments and is longer lasting. In situ functionalisation importantly means that receptors are also located directly at the nanoplate surface and enable processes such as analyte binding to occur in the regions of maximum E-field intensities which are close to the nanoplate surface and not to permeate into regions further out from the nanoplates where the E-field intensities reduce which occurs with distance from the surface.

We also describe the Perpetuation of Plasmon Resonance Coherence. Preservation of LSPR coherence and ensurance of slow plasmon oscillation dephasing times is essential in obtaining increased electromagnetic field enhancement, particularly in nanostructures of larger dimensions. A direct relationship exists between nanostructures size and the scale of the electromagnetic field enhancement up to the point where the capability of the incident field to homogeneously polarize the nanostructure plasmon resonance becomes limited. In the case of biosensing applications, defining the potential of nanostructures as LSPR refractive index sensors and enhancing the attainable LSPR refractive index sensitivity through perpetuation of LSPR coherence in larger nanostructures enables promotion over other less sensitive nanostructures. High aspect ratio is a means of perpetuating the coherence of the oscillation of the plasmon and confining its electromagnetic field to the surface resulting in enhanced LSPR refractive sensitivity and increased responsiveness of the electromagnetic field at the nanoplate surfaces such as interactions including refractive index induced changes by analyte binding to receptor on the nanoplate surface.

Inhibition of the coherence of the nanostructure LSPR through damping processes can broaden the plasmon resonance linewidth (FWHM) and decrease the intensity of the LSPR peak. In the case of the TSNP radiation damping only begins to contribute at an edge length of approximately 180 nm. This is much larger than the size which quasistatic theory predicts which would be between 20 and 40 nm and can therefore be attributed to the platelet like structure of the TSNP within the sols. The reduced radiation damping observed in TSNPs with sizes above that which theory predicts them to dominant, enables longer plasmon dephasing times and a more coherent oscillation. Using DDA calculated absorption and scattering spectra the above trends are shown to be attributed to the aspect ratios of nanoplates. This demonstrates that high aspect ratio is a means of preserving coherence of the oscillation of the plasmon while confining its electromagnetic field to the surface thereby promoting the scaling of electromagnetic field enhancement with nanoplate size beyond what would be possible at low aspect ratios.

We also describe coupled nanoplates. Coupled nanoplates can be defined as linked individual nanoplates which are discrete and not physically touching but whose electromagnetic fields (E-Field) overlap. The degree of coupling may vary wherein the nanoplates may form simple dimers, trimers or other multimers where the individual nanoplates are spaced at different distances apart. They may form larger chains or groups within which each discrete nanoplate is completely identifiable. They may physically operate as a unit. In all cases electromagnetic fields and LSPR of the coupled nanoplates can combine, may become shared among the individual nanoplates within the coupled group, (note in many cases coupled nanoplates are found to share the same colour and spectrum) or they may exhibit modes which add or multiply together in areas or conversely subtract in other areas.

The enhancement of electromagnetic fields which can occur at areas on the surface of coupled nanostructures is of key importance to phenomena which rely on the local electromagnetic fields surrounding nanostructures such as LSPR refractive index biosensing and SERS.

Coupled TSNP and coupled TSNP sensors show increased optical extinction cross sections or brightness than in the case of single TSNP and single TSNP sensors which improves optical detection. Live observation tethered grouped TSNP sensors show the vigorous movement of the TSNP group about their tethered position in solution. TSNP grouped sensor may also facilitate increased LSPR refractive index sensitivity over single TSNP sensors.

Also described is the presentation of the analyte molecules and analyte molecular interactions with local E-field with an improved configuration and with in E-field hot spots with an improved configuration. In one embodiment of the invention, presentation of the analyte molecules within the E-fields and E-field hot spots in an improved configuration is achieved through the use of under passivated/satbilised/capped nanoplates or through alteration of the surface chemistry of the nanoplates. The under these conditions processes such as receptor analyte binding are presented in an arrangement amenable to generating an increased response such as an LSPR refractive index induced wavelength shift. In the case of analyte molecule presentation in more optimal configuration within the E-field hot spots at the interface region between the coupled nanoplates increased SERS signals and LSPR refractive index response may be produced. In the case of SERS under the conditions of deprived nanoplate passivation/satbilisation/capping the analyte molecules in addition to functioning to complete the passivation of the nanoplates also function to couple the nanoplates. In so doing the analyte molecules present themselves within the E-field hot spots at the interface region between the coupled nanoplates in more optimal configuration for SERS.

One of the advantages associated with high aspect ratio is that it enables the preservation of the quantum confinement effects in nanoplates that would otherwise enter the bulk regime due to the size of the nanoplate. Nanoplates having a high aspect ratio retain many of the optical and electronic properties normally only associated with smaller nanoparticles.

The optical and electronic properties of noble metal nanoparticles (including nanoplates) are intrinsically linked to the optical extinction of incident electromagnetic fields through collective oscillation of the noble metal nanoparticles surface conduction electrons known as the local surface plasmon resonances (LSPR). Size dependence of the optical and electronic properties is observed due to the dominance of intrinsic size effects such as electron surface scattering at sizes below the bulk electron mean free path and extrinsic effects i.e. size dependence responses to external electromagnetic fields at larger dimensions. In general optical and electronic properties of metal nanoparticles, such as localized surface plasmon resonance (LSPR) sensitivity and electromagnetic field (E-field) enhancement, scale with increasing nanoparticle size up to a limit of the order of the length of the bulk metals electron mean free path. In nanoparticles having a radius (length) larger than the electron mean free path, radiative damping of the external electromagnetic field becomes a factor which can diminish the optical and electromagnetic response of the nanoparticles. A high aspect ratio retains at least one of the dimensions of the nanoplate a number of multiples (such as 3 times) below the length of the metals bulk electron mean free path resulting in increased optical and electronic properties without the onset of bulk material behaviour. In the case of silver, the bulk electron mean free path is 52 nm²⁸. In the absence of a high aspect ratio silver nanoplates would be expected to exhibit lower LSPR sensitivity to local refractive index changes compared to nanoparticles housing smaller dimensions. Instead, the high aspect ratio of nanoplates results in LSPR sensitivities which are equal to or greater than the LSPR sensitivities observed for smaller nanoparticles.

The sensitivity of the LSPR response to the local medium refractive index changes can be enhanced by tuning the geometry of the nanostructures. Nonspherical particles show typically larger E² than spheres which is associated with their ability to support plasmon resonances at long wavelengths while keeping the effective nanoparticle radii small. Non-spherical nanostructures (e.g. nanoprisms, nanorods, or nanoshells) have been postulated to exhibit increased LSPR sensitivities due to their support of large surface charge polarisability and increased local field enhancement at their sharp geometries¹⁶.

A variety of single substrate bound shaped nanostructures with increased LSPR sensitivity have been reported including single silver nanoprisms¹⁷, silver nanocubes¹⁸, gold nanostars¹⁹, and gold nanorings²⁰. Sensitivity values have been recorded as large as 0.79 eV/RIU for single silver nanocubes¹⁸, and 1.41 eV/RIU in the case of dielectric substrate coupled single gold nanostars¹⁹. Significantly increased LSPR sensitivities have been reported for more complex coupled single plasmonic nanostructures such as; 801 nm/RIU for hematite core/Au shell nanorice²¹ and 880 nm/RIU for gold nanorings²², however the position of these plasmon resonances are located at Near Infrared (NIR) wavelengths. Silver nanoparticles have the advantage over other noble metals such as gold and copper in that the LSPR energy is removed from that of interband transitions (3.8 eV˜327 nm)²³ resulting in a narrow LSPR which exhibits a much stronger shift with increasing local dielectric constant compared to gold or copper^{23, 24}.

We describe triangular silver nanoplate (TSNP) ensembles as highly sensitive LSPR nanostructures. The TSNP solutions are prepared using a seed mediated approach involving the reduction of silver ions by ascorbic acid that produces over 95% nanoprism populations in a rapid reproducible manner. The TSNP ensembles can be prepared using the methods described in PCT application no. PCT/IE2008/000097, the entire contents of which is incorporated herein by reference. The narrow geometric distribution of the TSNP within the solution leads to a highly uniform response of the ensemble upon interaction with an electromagnetic field.

Geometric parameters of the solution phase TSNP ensembles were defined using AFM and TEM size distribution analysis and the sensitivity of the collective LSPR to changes in the external environment was demonstrated using a sucrose based refractive index method. Solutions of TSNP with different edge lengths, aspect ratios and subsequent LSPR positions have been investigated to determine the influence of the nanoplate structure upon the sensitivity of the LSPR to the surrounding refractive index.

The invention will be more clearly understood from the following examples.

### Example 1

### Synthesis of Nanoplates (Wet Chemistry)

TSNP can be prepared according to the seed mediated methods described in PCT/IE2008/000097, the entire contents of which is incorporated herein by reference.

In this particular example, TSNP were prepared as follows: 5 ml of 2.5 mM trisodium citrate, 250 μl of 500 mg˜L⁻¹ 1,000 kDa poly(sodium styrenesulphonate) (PSSS) and 300 μL of freshly prepared 10 mM NaBH₄ were combined followed by addition of 5 mL of 0.5 mM AgNO₃ at a rate of 2 ml˜min⁻¹ while stirring vigourously.

The triangular silver nanoplates were grown by combining 5 mL distilled water, 75 μl of 10 mM freshly prepared ascorbic acid and various quantities of seed solution followed by addition of 3 mL of 0.5 mM AgNO₃ at a rate of 1 ml˜min⁻¹ followed by the addition of 0.5 ml of 25 mM Trisodium citrate.

The size of the TSNP can be controlled by adjusting the volume of seeds used in the nanoplate growth step.

### Example 2

### Synthesis of Nanoplates (Microfluidics)

TSNP can be prepared according to the seed mediated microfluidics methods described in PCT/IE2008/000097, the entire contents of which is incorporated herein by reference.

Briefly, microfluidic synthesis of TSNP comprises the steps of:
(a) forming silver seeds from a silver source and a reducing agent; and
(b) growing the thus formed silver seeds into TSNP

A generic microfluidic chip system was used for the production of TSNP using the following experimental parameters:

### Step (a)

A mixture of 3 mL of 10 mM sodium borohydride, 2.5 mL of 500 mgL⁻¹ poly(sodiumstyrene sulfonate) and 100 mL of 2.5×10⁻³M trisodium citrate in water (solution 1) was prepared and connected to a pump (pump 1). A solution comprising 100 ml of 5×10⁻⁴ M silver nitrate (solution 2) was prepared and connected to a pump (pump 2). The flow rates of pump 1 and pump 2 were set at 1 ml/min and 1 ml/min respectively. The pump lines were primed with the solution to be used in them and pump 1 and pump 2 were run in succession for about 2 min each such that an initial volume of about 2 mL of each solution was run through the microfluidic chip and discarded. Pump 1 and pump 2 were run together and the first 1 ml of the product solution was discarded. The subsequent 5 ml of seed product was collected and both the pumps were stopped.

### Step (b)

5 mL of water, 75 μL of 10 mM ascorbic acid and 1000, of the seeds from step (a) were stirred together in a beaker using a magnetic flea at a rate of 500 rpm, 3 mL of silver nitrate 5×10⁻⁴ M was added at a rate of 1 mLmin⁻¹. 500 μL 2.5×10⁻²M trisodium citrate was then added to stabilize the particles and the final volume was brought up to 10 mL using water.

The size of the TSNP can be controlled by adjusting the volume of seeds used in the growth step (step (b)).

Step (a) and/or step (b) may be carried out using a high pressure microfluidics device.

### Example 3

### Synthesis of Nanoplates (Shear Mixing)

In this example, we describe a simple, cost effective process for producing large volumes of high quality silver nanoplates with good batch to batch reproducibility. By “large volumes” we mean batches of at least 1 L of silver nanoplates are made. The process may be easily scaled to produce at least 5 L or 10 L or nanoplates in a single batch. By adjusting the quantities of starting materials, it will be possible to make a batch of nanoplates in excess of 10 L. The simplicity and batch reproducibility of the process described herein allow the process to be tailored for industrial production of nanoplates in volumes greater than 10 L, for example up to about 10,000 L.

The physical properties of the resulting silver nanoplates may be modified by altering the processing parameters such as flow rate and stirring speed while maintaining the relative concentrations of precursor materials. The process parameters may be optimised for the production of single shaped, narrow single spectral band monodispersed high aspect ratio triangular nanoplates. Alternatively, the process parameters may be modified to produce nanoplates having a mixture of geometric shapes such as triangles, hexagons, truncated or snipped triangles, ovals, polygons and/or nanoplates having a range of size distribution.

Nanoplates are a subset of nanoparticles having lateral dimensions (such as edge length) that are larger than their height (thickness). The term nanoplate includes for example nanodisks and nanoprisms. Nanoprisms have an equilateral triangle shape. Nanoplates have characteristic surface plasmon resonance bands, and are highly desirable for certain applications such as biosensors. When light is incident on a metal nanoparticle, the oscillating electric field generates a collective oscillation on the mobile conduction electrons in the metal, this collective oscillation of the electrons is called the surface plasmon resonance (SPR) of the nanoparticle and more correctly the dipole plasmon resonance. Higher modes of plasmon excitation can also occur. For example, when half the electron cloud moves parallel to the applied field, and the other half moves antiparallel, this is known as the quadrupole mode. A single plasmon band is indicative of a small (for example 1-10 nm) isotropic nanoparticle for example a spherical nanoparticle. As the degree of anisotropy increases the number of SPR bands increases due to decreasing nanoparticle symmetry. Increasing the size of nanoparticles can lead to high order SPR resonances such as quadrupolar, octupolar, or hexadecapolar resonances resulting in the presence of the corresponding weaker higher order SPR bands in the UV-Vis-NIR spectrum. However the presence of out-plane modes of these surface plasmon resonances are only observed in the case of non-isotropic nanoparticles such as nanoplates.

The effect of silver nanoparticle size and shape therefore gives the nanoparticle characteristic UV-Vis-NIR spectral profiles encompassing the respective SPR peaks located and tuned around designated wavelength positions. In the case of the nanoplates the characteristic peak in the 330 nm to 345 nm range is an out of plane quadrupole resonance which would not be present for spheres of any size. The relative position of the in-plane dipole, in-plane quadrupole and out of plane dipole, both of which may be masked and finally the out of plane quadrupole resonance provide a well known signature UV-VIS-NIR spectrum for triangular silver nanoplates of various edge lengths and aspect ratios. The size, shape and aspect ratio of the nanoplates may therefore be derived from a given spectral profile.

The process described in this example produces nanoplates that are monodisperse (discrete), well-defined silver nanoprisms of varying edge length. The triangular silver nanoplates have an aspect ratio from about 2 to about 20 with increasing edge length wherein aspect ratio is the ratio of the edge length and thickness of a nanoplate.

### Preparing Silver Seeds in a Shear Mixer

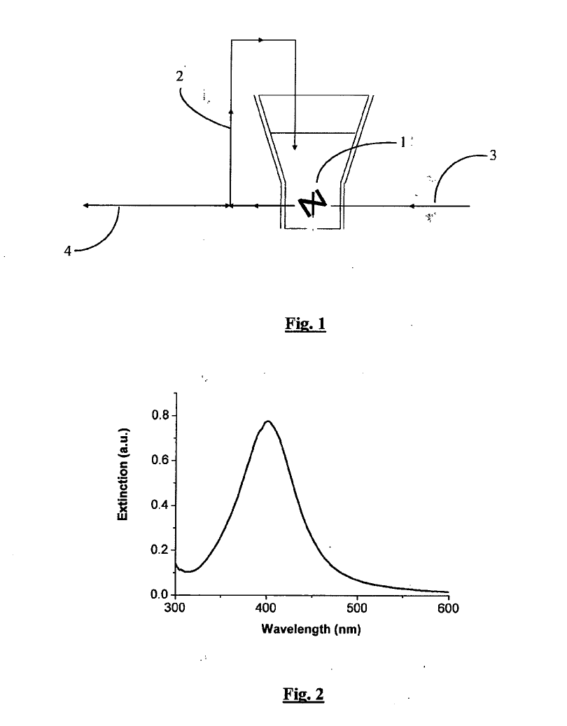

Referring to FIG. 1, an industrial scale shear mixer comprises a mixing chamber 1, in fluid communication with a recirculation line 2. An inlet 3 is in fluid communication with the mixing chamber 1. An outlet 4 is located downstream of the mixing chamber 1.

In general, an aqueous solution of sodium borohydride (a reducing agent), trisodium citrate (a stabilising agent) and PSSS (a water soluble polymer) is introduced into the mixing chamber 1 and is mixed via recirculation for at least 2 minutes at a shear rate between about 1×10¹ s⁻¹ to about 9.9×10⁵ s⁻¹. Such as between about 1×10¹s⁻¹ to about 2×10⁵ s⁻¹. Following premixing of the sodium borohydride, trisodium citrate and PSSS, silver nitrate (a silver source) is introduced into the mixing chamber 1 via inlet 3. The silver nitrate may be pumped into the mixing chamber by a peristaltic pump at a flow rate of up to 10% volume/min. The silver nitrate, sodium borohydride, trisodium citrate and PSSS are mixed for at least 5 minutes at a shear rate between about 1×10⁵ s⁻¹ to about 9.9×10⁵ s⁻¹ such as between about 1×10¹ s⁻¹ to about 2×10⁵ s⁻¹ to form silver seeds, after which the silver seeds solution is discharged from the mixing chamber 1 via the outlet 4.

### Shear Mixing Process

TSNP can be prepared by a shear mixing a process comprising the steps of
(i) forming silver seeds from an aqueous solution comprising a reducing agent, a stabiliser, a water soluble polymer and a silver source; and
(ii) growing the thus formed seeds into silver nanoplates in an aqueous solution comprising silver seeds, a reducing agent and a silver source.
   wherein step (i) and/or step (ii) are performed at a shear flow rate between about 1×10⁵ s⁻¹ and about 9.9×10⁵ s⁻¹.

In one example, silver seeds were produced in a shear mixer having the following parameters: Speed 16,000 rpm Gap size 0.15 mm, Radius of outer gap 15.05 mm, 15 cuttings/360° Shear rate 1.68×10⁵ s⁻¹; Shear frequency 3.36 Mio. Min⁻¹. A suitable shear mixer is sold by IKA process under item Magic Lab UTL 6F.

To produce the silver seeds (step (i)), H₂O (90 mL), TSC (10 mL, 25 mM), NaBH₄ (6 mL, 10 mM) and PSSS (5 mL, 0.5 mg/mL) were combined in a beaker. This solution was transferred into the mixing chamber of a shear mixer. The motor was switched on at a tip speed of 23 m/s and the solution was allowed to circulate for about 2 minutes. AgNO₃ (100 mL, 0.5 mM) was introduced through an adapted inlet at a rate of 40 ml/min using a peristaltic pump. After the AgNO₃ addition was complete, the solution was allowed to circulate for approximately 5 min before being tapped off. During the initial recirculation the cooling system was switched on so that the growth was carried out at about 30° C. The seeds were allowed to age for 1 h before further use.

In one example, silver nanoplates were produced in a shear mixer having the following parameters: Speed 16,000 rpm Gap size 0.15 mm, Radius of outer gap 15.05 mm, 15 cuttings/360° Shear rate 1.68×10⁵ s⁻¹; Shear frequency 3.36 Mio. Min⁻¹. A suitable shear mixer is sold by IKA process under item Magic Lab UTL 6F. A 1 L scale production of silver nanoplates at a concentration of 17 ppm were grown from silver seeds as follows:

To produce silver nanoplates (step (ii)), H₂O (500 mL), seeds (30 mL) and ascorbic acid (7.5 mL, 10 mM) were combined and then added to the mixing chamber of a shear mixer. This solution was then circulated at a shear rate of 1.68×10⁵ s⁻¹ for about 2 min and AgNO₃ (300 mL, 0.5 mM) was added at a rate of 100 mL/min using a peristaltic pump. Two minutes after the addition of AgNO₃ was complete, TSC (200 mL, 25 mM) was added using the peristaltic pump and the sol was allowed to recirculate for a further 2 minutes before being tapped off.

It will be appreciated that the reagent volumes and concentrations and process parameters may be modified. The size of the TSNP can be controlled by adjusting the volume of seeds used in the growth step (step (ii)).

### Further Examples of the Shear Mixing Process are Given Below.

### Example 3A

### Preparing Silver Seeds in a Shear Mixer

In this example, silver seeds were produced in a shear mixer having the following parameters: Speed 16,000 rpm Gap size 0.15 mm, Radius of outer gap 15.05 mm, 15 cuttings/360° Shear rate 1.68×10⁵ s⁻¹; Shear frequency 3.36 Mio. Min⁻¹. A suitable shear mixer is sold by IKA process under item Magic Lab UTL 6F.

To produce the silver seeds, H₂O (90 mL), trisodium citrate (TSC) (10 mL, 25 mM), NaBH₄ (6 mL, 10 mM) and PSSS (5 mL, 0.5 mg/mL) were combined in a beaker. This solution was then transferred into the mixing chamber of a shear mixer. The motor was switched on at a tip speed of 23 m/s and the solution was allowed to circulate for about 2 minutes. AgNO₃ (100 mL, 0.5 mM) was then introduced through an adapted inlet at a rate of 40 ml/min using a peristaltic pump. After the AgNO₃ addition was complete, the solution was allowed to circulate for approximately 5 min before being tapped off. During the initial recirculation the cooling system was switched on so that the growth was carried out at about 30° C. The seeds were allowed to age for 1 h before further use. Referring to FIG. 2, the seeds, produced exhibited a single peak at about 400 nm. The presence of this single plasmon band indicates the presence of isotropic particles which is consistent with the seeds being spherical nanoparticles with a size in the order of about 5 nm.

### Example 3B

### Preparing Silver Nanoplates in a Shear Mixer

In this example, silver nanoplates were produced in a shear mixer having the following parameters: Speed 16,000 rpm Gap size 0.15 mm, Radius of outer gap 15.05 mm, 15 cuttings/360° Shear rate 1.68×10⁵ s⁻¹; Shear frequency 3.36 Mio. Min⁻¹. A suitable shear mixer is sold by IKA process under item Magic Lab UTL 6F

In this example, a 1 L scale production of silver nanoplates at a concentration of 17 ppm were grown from silver seeds produced in accordance with Example 3A above.

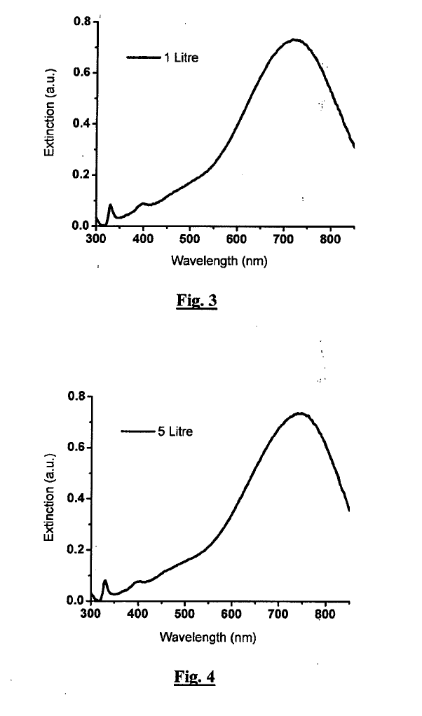

To produce silver nanoplates, H₂O (500 mL), seeds (30 mL) and ascorbic acid (7.5 mL, 10 mM) were combined and then added to the mixing chamber of a shear mixer. This solution was then circulated at a shear rate of 1.68×10⁵ s⁻¹ for about 2 min and AgNO₃ (300 mL, 0.5 mM) was added at a rate of 100 mL/min using a peristaltic pump. Two minutes after the addition of AgNO₃ was complete, TSC (200 mL, 25 mM) was added using the peristaltic pump and the solution was allowed to recirculate for a further 2 minutes before being tapped off. Referring to FIG. 3, the nanoplates exhibited a peak at about 710 nm. The UV-VIS-NIR spectrum shown in FIG. 3 is characteristic of triangular silver nanoplates with the out of plane quadrupole resonance located at 331 nm and the in-plane dipole peak located at 722 nm. The small peak located in the 400 nm region can be assigned to the out-of-plane dipole resonance but may also be indicative of a small number of spheres present in the sample.

### Example 3C

### Preparing Silver Nanoplates in a Shear Mixer

In this example, silver nanoplates were produced in a shear mixer having the following parameters: Speed 8,000 rpm Gap size 0.25 mm, Radius of outer gap 28.5 mm, 14 cuttings/360° Shear rate 9.56×10⁴ s⁻¹; Shear frequency 1.456 Mio. Min⁻¹. A suitable shear mixer is sold by IKA process under item Pilot Process 6F UTL 2000/4

In this example, a 5 L scale production of silver nanoplates at a concentration of 17 ppm were grown from silver seeds produced in accordance with Example 3A above.

To produce silver nanoplates, H₂O (2.5 L), seeds (150 mL) and ascorbic acid (27.5 mL, 10 mM) were combined and then added to the mixing chamber of a shear mixer. This solution was then circulated at a shear rate of 9.56×10⁴ s⁻¹ for about 2 min and AgNO₃(1.5 L, 0.5 mM) was added at a rate of 100 mL/min using a peristaltic pump. In the case of producing unstabilised nanoplates no further reagents are added on the completion of the addition of AgN0₃. In the case of producing TSC stabilised silver nanoplates two minutes after the addition of AgNO₃ was complete, TSC (1 L, 25 mM) was added using the peristaltic pump and the solution was allowed to recirculate for a further 2 minutes before being tapped off. Referring to FIG. 4 the nanoplates exhibited a peak at about 710 nm. The UV-VIS-NIR spectrum shown in FIG. 4 is characteristic of triangular silver nanoplates with the out of plane quadrupole resonance located at 331 nm and the in-plane dipole peak located at 745 nm. The red shifting of the in-plane dipole peak by 23 nm compared to the nanoplates produced in Example 3B above suggests that these nanoplates have a longer edge length. The small peak located in the 400 nm region can be assigned to the out-of-plane dipole resonance but may also be indicative of a small number of spheres present in the sample.

### Example 3D

### Preparing Silver Nanoplates in a Shear Mixer

In this example, silver nanoplates were produced in a shear mixer having the following parameters: Speed 16,000 rpm Gap size 0.15 mm, Radius of outer gap 15.05 mm, 15 cuttings/360° Shear rate 1.68×10⁵ s⁻¹; Shear frequency 3.36 Mio. Min⁻¹. A suitable shear mixer is sold by IKA process under item Magic Lab UTL 6F.

In this example, a 1 L scale production of silver nanoplates at a concentration of 34 ppm were grown from silver seeds produced in accordance with Example 3A above.

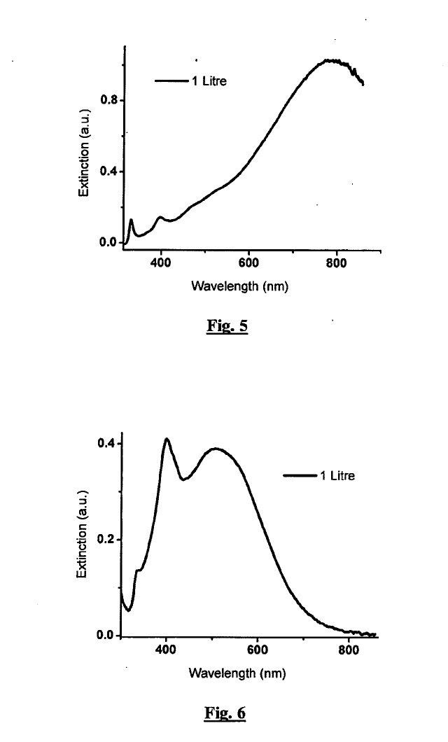

To produce silver nanoplates, H₂O (100 mL), seeds (60 mL) and ascorbic acid (15 mL, 10 mM) were combined and then added to the mixing chamber of a shear mixer. This solution was then circulated at a shear rate of 1.68×10⁵ s⁻¹ for about 2 min and AgNO₃(600 mL, 0.5 mM) was added at a rate of 100 mL/min using a peristaltic pump. In the case of producing TSC stabilised nanoplates two minutes after the addition of AgNO₃ was complete, TSC (300 mL, 25 mM) was added using the peristaltic pump and the solution was allowed to recirculate for a further 2 minutes before being tapped off. In the case of producing unstabilised nanoplates no further reagents are added on the completion of the addition of AgN0₃. Referring to FIG. 5 the nanoplates exhibited a peak at about 780 nm. The UV-VIS-NIR spectrum shown in FIG. 5 is characteristic of triangular silver nanoplates with the out of plane quadrupole resonance located at 331 nm and the in-plane dipole peak located at 790 nm. The red shifting of the in-plane dipole peak by a further 45 nm compared to the nanoplates produced in Example 3C above suggests that these nanoplates have the longest edge length of these three nanoplate samples of Examples 3B to 3D. The weaker peaks observed in between the in-plane dipole and the 400 nm peaks are indicative of higher order multipole resonances which become unmasked as the nanoplate size increases. The small peak located in the 400 nm region may be indicative of a small number of spheres present in the sample.

### Example 3E

### Preparing Silver Nanoplates in a Shear Mixer

In this example, silver nanoplates were produced in a shear mixer having the following parameters: Speed 16,000 rpm Gap size 0.15 mm, Radius of outer gap 15.05 mm, 15 cuttings/360° Shear rate 1.68×10⁵ s⁻¹; Shear frequency 3.36 Mio. Min⁻¹. A suitable shear mixer is sold by IKA process under item Magic Lab UTL 6F.

In this example, a 1 L scale production of silver nanoplates at a concentration of 17 ppm were grown from silver seeds produced in accordance with Example 3A above.

To produce silver nanoplates, H₂O (500 mL), seeds (50 mL) and ascorbic acid (7.5 mL, 10 mM) were combined and then added to the flask of the mixing chamber of the shear mixer. This solution was circulated at a shear rate of 1.68×10⁵ s⁻¹ for about 2 min and AgNO₃(300 mL, 0.5 mM) was added at a rate of 100 mL/min using a peristaltic pump. Two minutes after the addition of AgNO₃ was complete, TSC (200 mL, 25 mM) was added using the peristaltic pump and the sol was allowed to recirculate for a further 2 minutes before being tapped off. Referring to FIG. 6. The UV-VIS-NIR spectrum shown in FIG. 6 is characteristic of a mixture of triangular silver nanoplates and nanospheres with the out of plane quadrupole peak and in-plane dipole peaks associated with the triangular nanoplates located at 337 nm and at 507 nm respectively. The blue shifting of the in-plane dipole peak compared to the triangular nanoplates produced in the previous examples (Examples 3B to 3D) suggests that these nanoplates have the shortest edge lengths of the nanoplate samples. The strong peak located in the 400 nm region may be indicative of a large percentage of spheres present in the sample.

### Example 3F (Comparative Example)

In this Example, a 1 L scale production of silver seeds and silver nanoplates at a concentration of 17 ppm were prepared using magnetic stirring bar and overhead bench top stirrer. 200 mL seeds were prepared by the batch method on a using a standard magnetic stirring bar. These seeds were then used to prepare IL of particles using an over head stirrer @ 6,500 rpm.

An aqueous solution of sodium borohydride (a reducing agent), trisodium citrate (a stabilising agent) and PSSS (a water soluble polymer) was placed in a beaker and set stirring using a magnetic bar. Silver nitrate (a silver source) is introduced into the beaker at a rate of 40 ml˜min using peristaltic pump

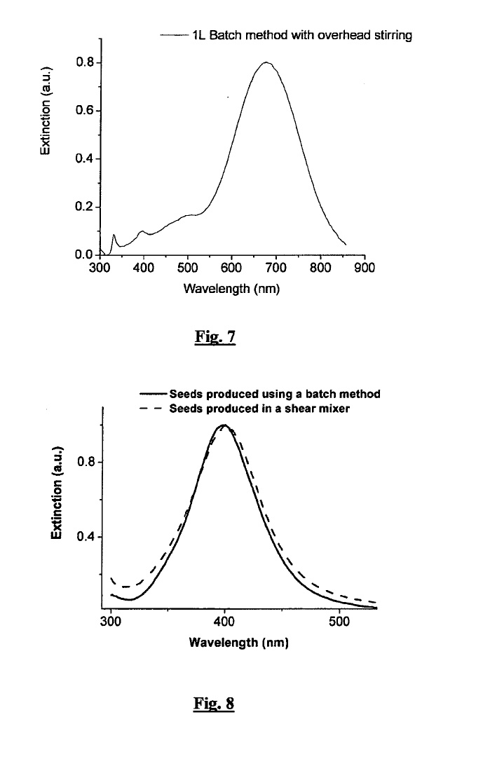

Referring to FIG. 7, the nanoplate solution exhibited a peak at about 676 nm. The UV-VIS-NIR spectrum shown in FIG. 7 is characteristic of triangular silver nanoplates with the out of plane quadrupole resonance located at 330 nm and the in-plane dipole peak located at 676 nm. The small peak located in the 400 nm region can be assigned to the out-of-plane dipole resonance but may also be indicative of a small number of spheres present in the sample.

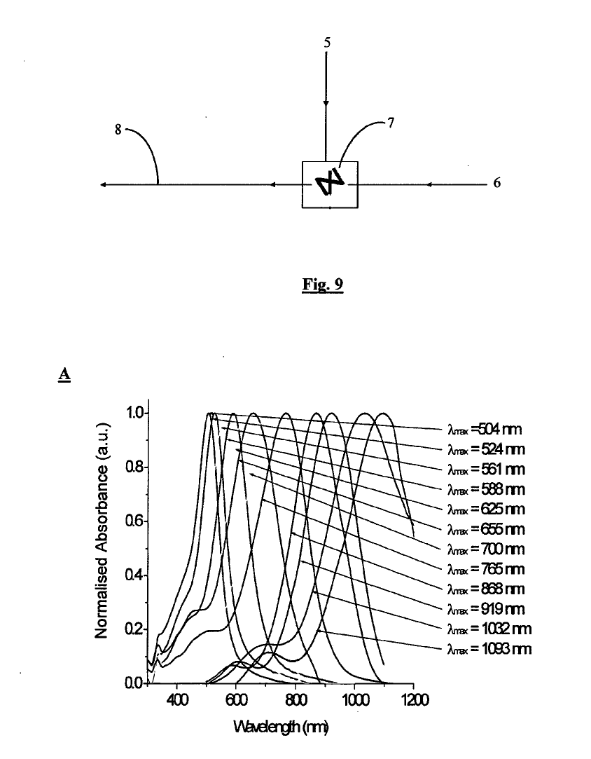

Referring to FIG. 8, the FWHM of the seeds produced in a shear mixer (dash line) in accordance with Example 3A is broader and slightly red shifted compared to that of the batch seeds (solid line). We believe that optimization of the flow rates in the shear mixer method will result in the production of seeds with a smaller FWHM which can be grown into narrower band silver nanoplates.

### Example 3G

### Flow Chemistry/Inline Production of Silver Nanoplates

The shear mixer may be configured to function as an inline/flow chemistry device to allow for the continuous production of silver seeds and/or silver nanoplates. For example, referring to FIG. 9, the device may comprise two spaced apart inlets 5, 6 in fluid communication with a mixing chamber 7 and an outlet 8. A suitable in-line continuous flow production shear mixer may have the following operating parameters: Flow rate range from 1 ml min⁻¹ to 10 L min⁻¹; Speed 16,000 rpm Gap size 0.15 mm, Radius of outer gap 15.05 mm, 15 cuttings/360° Shear rate 1.68×10⁵ s⁻¹; Shear frequency 3.36 Mio. Min⁻¹. A suitable shear mixer is sold by IKA process under item Magic Lab UTL 6F.

### Example 3H

### Flow Chemistry/Inline Production of Silver Nanoplates

In this example, silver nanoplates were grown from silver seeds produced in accordance with Example 3A. An in-line continuous flow production shear device in accordance with Example 3G was used in which AgNO₃ was pumped through inlet 5 at a rate of 170 mL/min using a peristaltic pump, and a mixture of ascorbic acid, silver seeds and water was pumped through inlet 6 at a rate of 170 mL/min using a peristaltic pump. The two solutions were mixed in the mixing chamber 7 at tip speed of 23 m/s.

The resultant solution was colourless which turned blue after about 20 minutes indicating that the silver nanoplates had been produced.

### Example 3I

### Flow Chemistry/Inline Production of Silver Nanoplates

In this example, silver nanoplates were grown from silver seeds produced in accordance with Example 3A. An in-line continuous flow production shear device in accordance with Example 3G was used in which AgNO₃ was pumped through inlet 5 at a rate of 170 mL/min using a peristaltic pump, and a mixture of ascorbic acid, silver seeds and water was pumped through inlet 6 at a rate of 170 mL/min using a peristaltic pump. The two solutions were mixed in the mixing chamber 7 at tip speed of 40 m/s.

The resultant solution was weakly pink which turned blue after about 20 minutes indicating that the silver nanoplates had been produced.

### Example 3J

### Flow Chemistry/Inline Production of Silver Nanoplates

In this example, silver nanoplates were grown from silver seeds produced in accordance with Example 3A. An in-line continuous flow production shear device in accordance with Example 3G was used in which AgNO₃ was pumped through inlet 5 at a rate of 23 mL/min using a peristaltic pump and a mixture of ascorbic acid, silver seeds and water was pumped through inlet 6 at a rate of 86 mL/min using a peristaltic pump. The two solutions were mixed in the mixing chamber 7 at tip speed of 23 m/s.

The resultant solution was weakly blue which turned blue after about 20 minutes indicating that the silver nanoplates had been produced.

We envisage that further optimization of the flow rates of the two components in the in-line continuous flow production shear device could result in the production of better quality silver nanoplates including a broader range of nanoplate shapes, shape mixtures, distributions in addition to single shaped, narrow single spectral band monodispersed high aspect ratio triangles. In the case of producing unstabilised nanoplates no further reagents are added on the completion of the addition of AgN0₃.

Furthermore, optimisation of the in-line continuous flow production parameters will lead to the production of triangular silver nanoplates for which the reaction will be completed as part of the inline process as will be indicated by no further colour change of the resultant solution.

It will be appreciated that the ultimate size of the nanoprisms can be tuned by controlling the ratio of silver ion: silver seed in the growth step. As the volume of silver seeds used is increased, the mean edge length of the triangular silver nanoprisms in the resultant solution is decreased and therefore the colour of the resultant solution can be tuned. This is because the silver ions present in the growth step have to be distributed over a greater number of particles (seeds). The mole ratio of silver seed: silver ion may be varied from about 1:8 to about 1:320 depending on the size of the silver nanoprisms required.

As can be seen from the above ratio, the volume of silver seed solution that is used to produce triangular silver nanoprisms is much less than the final volume of the nanoprisms produced. For example, in FIG. 3 the volume of seeds required to prepare 1 L of 17 ppm silver nanoplates is 10 mL. Therefore, in this whole process the volume of silver seed that needs to be produced is much lower than that of the grown triangular silver nanoplate solution.

The concentration of triangular silver nanoplate produced can also be varied. The number of triangular silver nanoplates produced is limited by the kinetic and thermodynamic equilibrium associated with the growth step. The concentration of silver ion introduced into the growth step can be varied from tens of ppm (such as 10 ppm) to a couple of hundred ppm, (such as 200 ppm) without inhibiting the reaction to such an extent that triangular silver nanoprisms cannot be produced. However, as the concentration of silver ion is increased other factors such as the ratio of silver seed: silver ion, the concentration of reducing agent and the rate at which the silver ion is introduced into the reaction need to be varied to accommodate the change in the concentration of silver ion. This variation is only necessary in the growth step process, the parameters for synthesising silver seeds remain unchanged.

The volume of triangular silver nanoprism solutions produced by the shear process described herein range from 1 L up to 10,000 L with concentrations of nanoprisms between about 17 ppm and about 200 ppm. The concentrations of reagents used may be varied accordingly.

Advantageously, the process described herein allows for the synthesis of a silver nanoplate solution at the highest possible concentration (ppm) in the highest possible volume within the limits imposed by the reaction chemistry involved.

### Example 4

### Aspect Ratio of Nanoplates

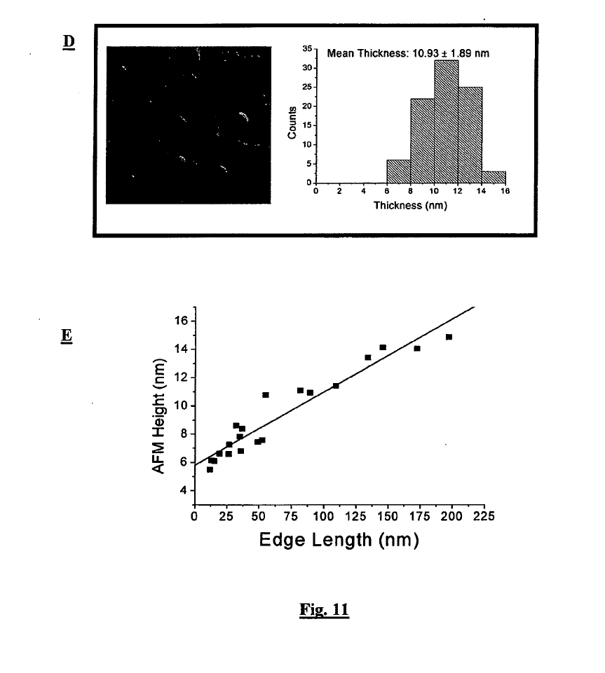

A series of TSNPs with increasing edge length from 11 nm to 197 nm were prepared. AFM and TEM images (FIG. 11 A-D) were recorded and analysed to assess the influence of the nanostructures geometry on the position of the LSPR. Using a statistically satisfactory number of nanoparticles (approx 150-200 particles) for each of the twenty ensembles, the mean thickness (height) (nm) and the mean edge length (nm) were calculated with the standard deviation of the distributions representing the experimental error. The AFM measurements show a gradual increase in the mean thickness of the TSNP ensembles with increasing edge length recorded via TEM (FIG. 11E).

It will be understood that the term “ensemble” as used herein means a collection of more than one silver nanoplates or coupled silver nanoplates.

The solution phase ensemble extinction spectra of the TSNP solutions were acquired using a UV-Vis-NIR spectrometer with the peak LSPR resonances ranging from wavelengths of about 500 nm in the visible up to 1090 nm in the NIR. The spectral position of a number of these samples is shown in FIG. 10 A. A linear dependence of the LSPR λₘₐₓ with edge length has been previously reported for gold triangular nanostructures of constant thickness²⁶. However due to the gradual increase in thickness of the TSNPs with edge length demonstrated in FIG. 11D the dependence of the LSPR λₘₐₓ on the structure is better examined using the aspect ratios for the different TSNP solutions. The aspect ratio of the TSNPs is found to increase from values of 2 to 13 with increasing edge length (FIG. 10B). The ensembles LSPR λₘₐₓ is observed to red-shift as the aspect ratio increases (FIG. 10C) for LSPRs within the range 500-1150 nm.

These TSNP exhibit distinct dipole, quadrupole and higher multipole plasmon resonances, and excitation of these resonances creates an E-field external to the particles that is important in determining normal and single molecule SERS intensities.

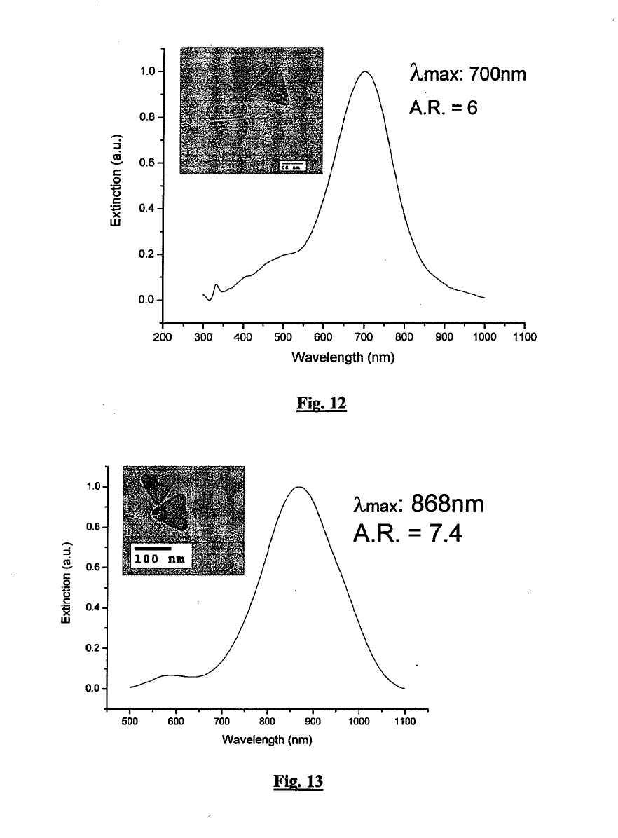

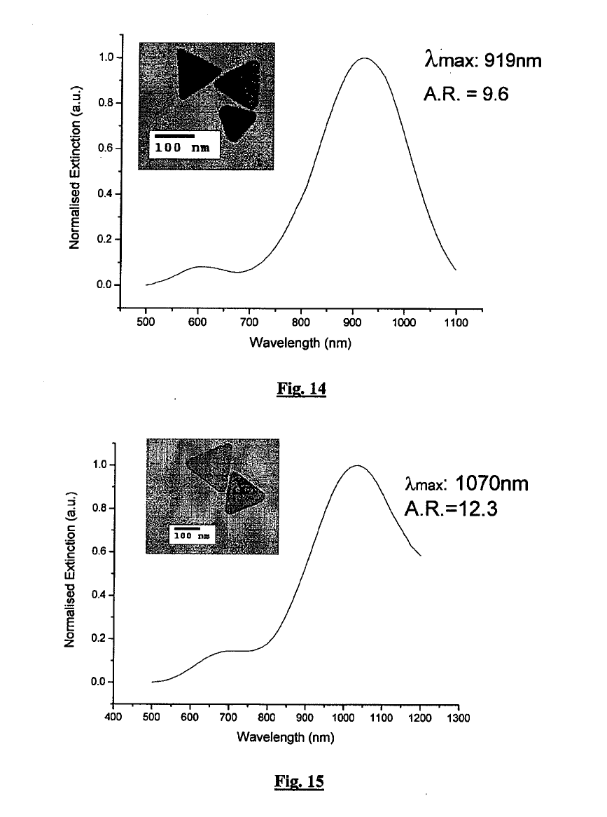

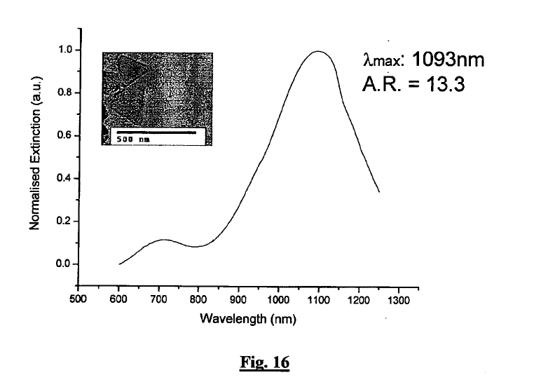

Referring to FIG. 12 the UV-VIS-NIR spectrum shown is for TSNPs with an aspect ratio of 6, the TEM inset image shows representive TSNP. The spectrum shows a signature out-of-plane quadrupole resonance located at 332 nm and the in-plane dipole peak located at 700 nm. The weaker peaks observed in between the in-plane dipole and the out-of-plane quadrupole peaks are indicative of higher order multipole resonances. FIG. 13 shows the UV-VIS-NIR spectrum for TSNPs with an aspect ratio of 7.4. The TEM image shows representative TSNP of larger edge length than those shown in FIG. 12 the increase in aspect ratio and edge length is signified by the red shift of the in-plane dipole peak located at 868 nm. FIG. 14 shows a UV-VIS-NIR spectrum for TSNP with an aspect ratio of 9.6, the TEM image shows representative TSNP of larger edge length than those shown in FIG. 13 the increase in aspect ratio and edge length is manifested by red shifting the in-plane dipole to 919 nm. FIG. 15 shows a UV-VIS-NIR spectrum for TSNP with an aspect ratio of 12.3 the TEM image shows representative TSNP of larger edge length than those shown in FIG. 14, the increase in aspect ratio and edge length is manifested by red shifting the in-plane dipole to 1070 nm. FIG. 16 shows a UV-VIS-NIR spectrum for TSNP with an aspect ratio of 13.3, the TEM image shows representative TSNP of larger edge length than those shown in FIG. 15, the increase in aspect ratio and edge length is manifested by red shifting the in-plane dipole to 1093 nm.

### Surface Area

For samples with aspect ratio less than 7 there is an optimal % surface area at which the TSNP exhibit optimal LSPR sensitivity. The maximum LSPR sensitivity occurs at a % surface area of ˜38-40%. This indicates that this is the optimal % surface area to prevent the onset of surface electron scattering dampening of the nanoparticle's LSPR absorption and LSPR sensitivity.

The volume and surface area of the TSNP can be calculated using equations 11 and 12 below.

Volume=½(Edge Length)(Diagonal)(Height)

Surface Area=└2(½(Edge Length)(Diagonal))┘+[3(Edge Length(Height))]

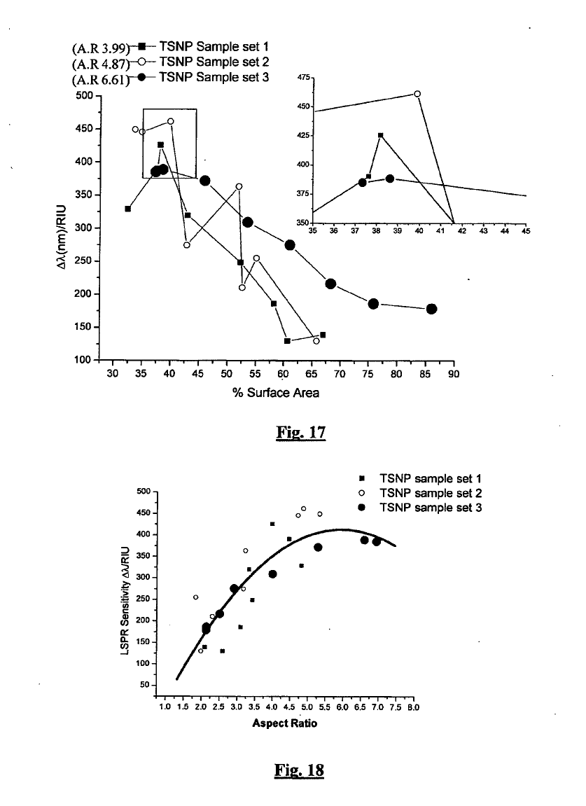

The Tables below detail the physical parameters of three different TSNP ensemble samples. Referring to FIG. 17 a local maximum in the ensemble local surface plasmon resonance sensitivity is observed in each of these three TSNP ensemble samples. When plotted against percentage surface area as in FIG. 17 it may be seen that the ensemble local surface plasmon resonance sensitivity coincides with percentage surface area within circa the 38% to 40% range. As the percentage surface area of the TSNP ensembles drops below this critical value, unless aspect ratio is sufficiently high. radiation damping factors come into play resulting in reduced LSPR sensitivity. It maybe noted that the dip following the maximum in the ensemble local surface plasmon resonance sensitivity is greatest for the TSNP ensembles having the lowest aspect ratio and least for the those having the largest aspect ratio at percentage surface areas less than circa 38% to 40%.

TABLE 2
Parameters of TSNP ensemble sample set 1

| Edge | | | Peak | | Surface | % | |
|---|---|---|---|---|---|---|---|
| Length | Height | Aspect | Wavelength | Volume | Area | Surface | Δλ |
| (nm) | (nm) | Ratio | (nm) | (nm³) | (nm²) | Area | (nm/RIU) |
| 12.51 | 6.27 | 1.99 | 511.57 | 981.0547 | 645.685 | 65.81539 | 129.95 |
| 14.38 | 7.83 | 1.84 | 532.63 | 1613.332 | 888.937 | 55.09943 | 254.94 |
| 17.92 | 7.76 | 2.31 | 573.60 | 1938.147 | 1019.831 | 52.61883 | 210.23 |
| 22.40 | 6.93 | 3.23 | 612.63 | 2342.34 | 1216.54 | 51.93695 | 363.17 |
| 26.88 | 8.46 | 3.18 | 315.33 | 4133.504 | 1770.566 | 42.83452 | 274.49 |
| 39.36 | 8.09 | 4.87 | 713.84 | 6346.411 | 2530.287 | 39.86957 | 461.47 |
| 42.88 | 9.09 | 4.72 | 786.12 | 9899.414 | 3450.78 | 34.85842 | 445.44 |
| 48.07 | 9.02 | 5.33 | 840.03 | 12359.76 | 4157.114 | 33.63427 | 449.15 |

TABLE 3
Parameters of TSNP ensemble sample set 2

| | | Aspect | | | | | |
|---|---|---|---|---|---|---|---|
| Edge | | Ratio | Peak | | Surface | % | |
| length | Height | (using | Wavelength | Volume | Area | Surface | Δλ |
| (nm) | (nm) | TEM) | (nm) | (nm³) | (nm²) | Area | (nm/RIU) |
| 12.10 | 5.75 | 2.1 | 481.33 | 996.7752 | 667.8994 | 67.00602 | 139.26 |
| 16.05 | 6.17 | 2.6 | 507.13 | 1387.831 | 842.4612 | 60.70345 | 129.91 |
| 19.11 | 6.25 | 3.1 | 544.33 | 1631.633 | 950.56 | 58.25821 | 185.96 |
| 23.40 | 6.82 | 3.43 | 576.89 | 2330.052 | 1218.124 | 52.27884 | 248.56 |
| 28.61 | 8.57 | 3.34 | 602.88 | 3983.508 | 1713.538 | 43.01581 | 319.71 |
| 36.99 | 9.26 | 3.99 | 662.93 | 6067.337 | 2316.076 | 38.17286 | 425.73 |
| 39.58 | 8.85 | 4.47 | 735.33 | 7048.176 | 2652.419 | 37.6327 | 390.29 |
| 48.07 | 9.98 | 4.82 | 795.49 | 11703.87 | 3795.459 | 32.4291 | 329.06 |

TABLE 4
Parameters of TSNP ensemble sample set 3

| Edge | | | Peak | | Surface | % | |
|---|---|---|---|---|---|---|---|
| Length | Height | Aspect | Wavelength | Volume | Area | Surface | Δλ |
| (nm) | (nm) | Ratio | (nm) | (nm³) | (nm²) | Area | (nm/RIU) |
| 11.77 | 5.48 | 2.14 | 504.54 | 390.2226 | 335.9158 | 86.08313 | 178.7 |
| 13.16 | 6.12 | 2.15 | 524.73 | 558.942 | 424.2784 | 75.9074 | 186.34 |
| 15.34 | 6.08 | 2.52 | 560.96 | 789.9732 | 539.6612 | 68.31386 | 216.41 |
| 19.33 | 6.61 | 2.92 | 588.21 | 1245.77 | 760.2489 | 61.02642 | 275.06 |
| 26.4 | 6.58 | 4.01 | 625.35 | 2254.782 | 1206.48 | 53.50762 | 309.17 |
| 35.86 | 6.77 | 5.29 | 700.52 | 4429.379 | 2036.848 | 45.98496 | 371.71 |
| 49.07 | 7.42 | 6.61 | 746.14 | 9353.714 | 3613.5148 | 38.63187 | 388.54 |
| 52.56 | 7.56 | 6.95 | 828.33 | 10947.09 | 4088.1168 | 37.34432 | 384.98 |

Referring to FIG. 18, the aspect ratio was not increased sufficiently with increasing edge length beyond the 4.5 aspect ratio region in order to prevent the onset of bulk volume radiation damping conditions which act to prevent the continued increase of the LSPR sensitivity and reduction is seen at aspect ratios greater than 6 nm which corresponds to TSNP with edge lengths of the order of the electron mean free path.

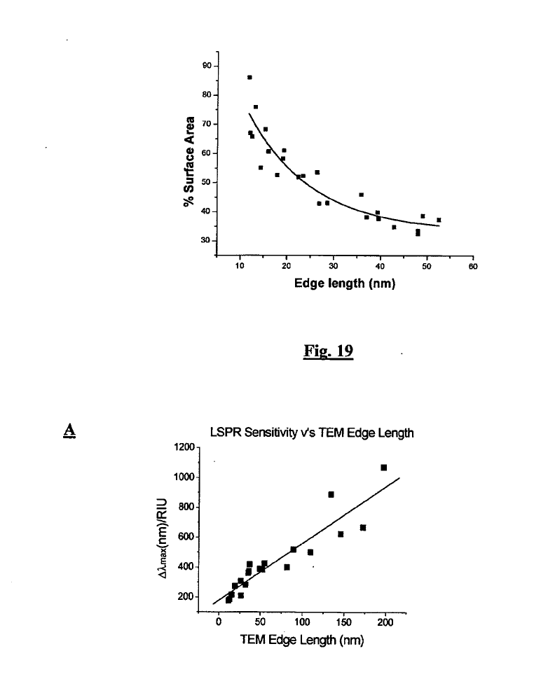

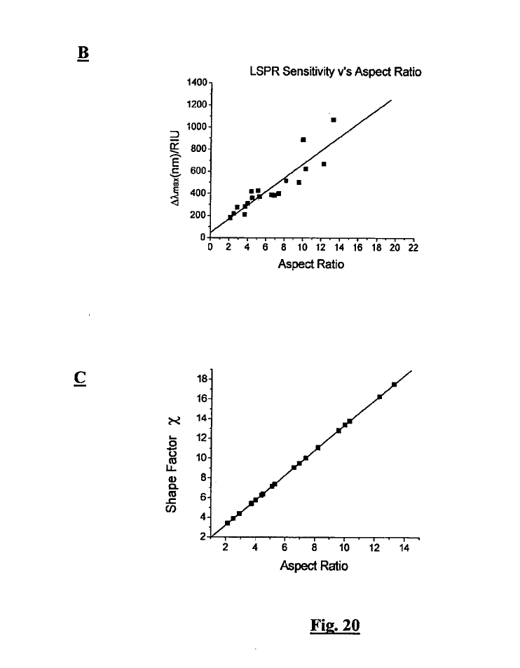

Referring to FIG. 19, the percentage surface area decreases in a exponential fashion with increasing edge length settling at a level of around 35% for TSNP with edge lengths of the same magnitude of the electron free path. TSNP of these edge lengths require increased aspect ratio in order to prevent the onset of radiation damping effects and the diminution of the optical and electronic properties.

### Example 5

### LSPR Sensitivity Measurement of TSNP

LSPR sensitivity scales with nanoparticle (including nanoplates) size up to the order of the electron mean free path. Larger high aspect ratio TSNP have longer λₘₐₓ which enables more free-electron like responses and contributes to the enhanced optical and physical properties of high aspect ratio TSNP.

The majority of LSPR sensitivities presented in the literature are for single nanostructures and not ensemble averages as in the case of the TSNP described herein. As a result of the nature of ensemble averaging, it is known to diminish and reduce LSRP sensitivity values compared to those calculated for individual single or coupled nanostructures. In the case of ensemble average LSPR sensitivities, Au nanorattles in solution, which have an aspect ratio of approximately 2 (length ˜60-65 nm, width ˜30-35 nm depending on initial rod length), were reported to have values ranging from 150 to 285 nm/RIU at wavelength of approximately 600 nm²⁹. In comparison, average LSPR sensitivity values for all TSNP ensemble are all greater 300 nm/RIU in the 600 nm spectral region. It is also significant that the TSNP ensemble average sensitivity values at LSPR peak wavelengths in the visible exceed those previously reported for single nanostructures within this wavelength band such as 204 nm/RIU for single Au triangles by Sherry et al¹⁷ (Table 1 below). It is evident that the highest sensitivities of the TSNP ensemble solutions examined here are greater than those recorded to date including those for single nanostructures such as nanorice²¹, gold nanorings²² and gold nanostars¹⁹ (see Table 1 below). Furthermore, unlike other reported high LSPR sensitive nanostructures the TSNP ensemble high LSPR sensitivities occur at wavelengths shorter than 1150 nm, this is important if the TSNP are incorporated into a biosensor as the high LSPR sensitivities occur at wavelengths before water and biomolecular absorptions can become limiting factors.

Full width at half maximum (FWHM) calculations were carried out manually. The FWHM calculation involved normalisation of the LSPR spectral peak, intersecting the halfway point and determining the wavelength on either side of the LSPR peak and calculating the difference.

TABLE 1
Comparison between the LSPR sensitivities reported to date in the
literature for various different single nanostructures fabricated and
tested using similar refractive index methods.

| | Peak λ (nm)/ | Δλ(nm)/ | FWHM |
|---|---|---|---|
| Sample | Shape | RIU | (eV) |
| Single silver | Pk 1: 459.3 | 93.99 | 0.284 |
| Nanoprisms¹⁷ | Pk 2: 630.6 | 204.9 | 0.246 |
| (2006) | Pk 1: 460.8 | 80.64 | 0.267 |
| | Pk 2: 634.6 | 182.9 | 0.195 |
| | Pk 1: 439.6 | 78.62 | 0.167 |
| | Pk 2: 631.4 | 196.4 | 0.166 |
| Single Silver | Sphere: | 161 | — |
| Nanoparticles | Triangle: | 197 | — |
| ²⁸(~35 nm) | Cube: | 235 | — |
| (2003) | | | |
| Nanorice | Longitudinal | 801/ | — |
| Length~366 nm | Plasmon | FDTD: | |
| Width~80 nm | Peak 1160 nm | 1060 | |
| (Shell Thickness | Transverse | 103/ | — |
| 13.7 nm)²¹ | Plasmon | FDTD: | |
| (2006) | Peak 860 nm | 115 | |
| Gold Nanoshells²⁰ | ~30 nm | 70.9 | — |
| (2002) | immobilised | | |
| | gold solid | | |
| | colloid | | |
| | ~50 nm gold | 60 | — |
| | solid colloid | | |
| | Nanoshells: | 408.8 | — |
| | Mean size | | |
| | 50 nm Wall | | |
| | thickness~4.5 | | |
| | nm | | |
| Gold Nanorings | Peak at | 880 | — |
| 150 nm Diameter | 1545 nm | | |
| (Gold: 20 nm | | | |
| thick)²² | | | |
| (2007) | | | |
| Au Nanohole Arrays | Infinite hole | 286 | 70 nm |
| 100 nm holes²⁹ | arrays | | |
| (2007) | Finite Hole | 313 | 0.032 |
| | Arrays | | |
| Rod-Shaped Gold | Dark Field | 199 ± 70 | — |
| Nanorattles~30-40 | Measurement: | | — |
| nm rods with | 50-100 single | | |
| 3-6 nm | particles per | | |
| shell (2009)²⁷ | measurement | | |
| Gold NanoBoxes* | Wall | 336 | ~127 nm |
| Inner edge length | thickness | | for 5.7 nm |
| 30 nm³⁰ | 5 nm | | thickness |
| (*These values were | Pk~600 nm | | |
| predicted | Varied wall | 210-565 | Peak |
| computationally) | thickness 15- | | broadens as |
| | 1.5 nm | | thickness is |
| | Pk: ~600 nm- | | increased |
| | 1000 nm | | |
| Ag/PVA | Peak: 600 mn | 377 | 0.89 |
| nanoparticles Edge | 55% shaped | | |
| Length 25 nm²⁵ | particles in | | |
| | ensemble, | | |
| | hexagons and | | |
| | triangles | | |
| TSNP Ensembles | Pk: 504 nm- | 178- | 0.297-0.6 |
| Edge Length 11.77- | 1093 nm | 1070 | |
| 197.23 nm | >95% Triangles | | |

We believe that the geometric structure may enhance the sensitivity and the dependence on the spectral location of the ensembles collective LSPR of the TSNP. Referring to FIG. 20 the maximum sensitivities recorded for TSNP solutions occurred in samples with a mean edge length of greater than 100 nm which is approximately twice the electron mean free path for bulk silver (˜52 nm)²⁷. In nanostructures of this size, bulk volume scattering and retardation effects of the electromagnetic field are expected to increase dampening of the LSPR band and incoherence in the plasmon resonance therefore meaning that the quasistatic approximation for dipolar LSPR resonance should not hold²⁷. Contrary to the experimental results obtained, this theoretical model would predict these TSNPs to be less sensitive to local refractive index changes than those of smaller dimensions and suggest the high sensitivities to be indicative of bulk refractive index change similar to thin film sensitivities²⁸.

The dependence of LSPR sensitivity with aspect ratio shown in FIG. 20 illustrates that the largest LSPR sensitivities recorded were for TSNP solutions with highest aspect ratios up to 13:1. We propose this geometric property (high aspect ratio) of the TSNPs to be the basis behind the enhanced response of the solution phase TSNP ensembles. The dependence of the resonance frequency on the aspect ratio and geometric parameters can be explained by Mie theory²⁹, where the extinction of a metallic sphere, i.e. the sum of the absorption and Rayleigh scattering can be represented by the equation

$$E = \frac{24 {π}^{2} {N}_{A} {a}^{3} {ɛ}_{m}^{\frac{3}{2}}}{λ ln ( 10 )} [ \frac{{ɛ}_{i}}{{( {ɛ}_{r} + χ {ɛ}_{m} )}^{2} + {ɛ}_{i}^{2}} ] \qquad ( Equation 2 )$$

where N_{A} is the areal density of nanoparticles,
a is the radius of the metallic nanosphere,
εₘ is the dielectric constant of the medium surrounding the metallic nanosphere,
λ is the wavelength of the absorbing radiation, and εᵢ, εᵣ the imaginary and real parts of the nanoparticle's dielectric function respectively.

The factor χ can be described as a shape factor which is determined by the depolarisation factors Pⱼ for the 3 axes A, B and C of the TSNPs, where

$$χ = \frac{1 - {P}_{j}}{{P}_{j}} {}^{30} .$$

The shape factor's dependence upon the aspect ratio of the TSNPs can be approximated by considering them as oblate spheroids structures with A (edge length)=B (diagonal)>C (thickness). For such a platelet type structure the depolarisation factor can be calculated as

$$\begin{matrix}{P}_{A} = \frac{g ( e )}{2 {e}^{2}} [ \frac{π}{2} - {tan}^{- 1} g ( e ) ] - \frac{{g}^{2} ( e )}{2} with & ( Equation 3 ) \\ e = \sqrt{1 - {( \frac{B}{A} )}^{2}} = \sqrt{1 - \frac{1}{{R}^{2}}} and & ( Equation 4 ) \\ g ( e ) = {( \frac{1 - {e}^{2}}{{e}^{2}} )}^{\frac{1}{2}} & ( Equation 5 )\end{matrix}$$

where

$$R = \frac{A}{B}$$

is the nanostructure aspect ratio.

Previous shape factor values of 2 for a sphere and greater than 17 for a 5:1 aspect ratio nanorod with a prolate spheroid geometry have been reported³¹. FIG. 20 illustrates the calculated shape factor values for the measured TSNPs aspect ratios, which range from 3 up to 18. As the oblate spheroid approximation does not take into account tip enhancement effects of the triangular geometry the calculated values are lower value estimates of the true shape factor. The dipolar plasmon resonance condition for equation 2 i.e. the occurrence of the extinction peak is satisfied when

εᵣ=−χεₘ (Equation 6)

or

εᵣ=−χn² (Equation 7)

where n is the refractive index of the surrounding medium.

This dependence of the position of this resonance condition can therefore be described as

$$\frac{Δ {λ}_{max}}{Δ n} ∝ \frac{d {ɛ}_{1}}{d n} = - 2 χ n \qquad ( Equation 8 )$$

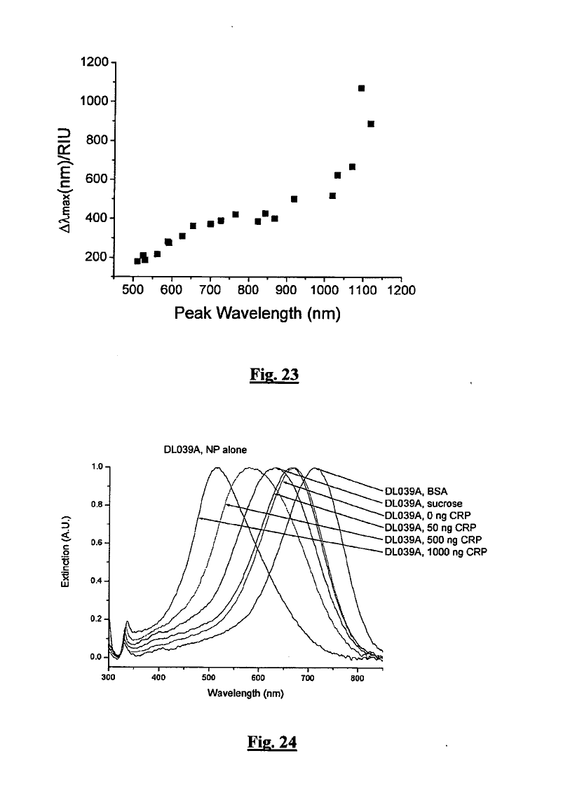

Equation 8 illustrates that as the aspect ratio is directly related to the shape factor x, and the sensitivity of the nanostructure's LSPR λₘₐₓ to the refractive index of the surrounding medium will increase accordingly with the aspect ratio. This increase is in agreement with the trend observed for the TSNPs shown in FIG. 20. Referring to FIG. 23, TSNP sensitivities are found to increase linearly with LSPR λₘₐₓ in agreement with previously reported models up to 800 nm³². However, surprisingly at wavelengths further into the near infrared (NIR) a deviation from the linear trend occurs and non-linear scaling is observed in which the LSPR sensitivity dramatically increases (FIG. 23). We believe that the high aspect ratio of these TSNP is sufficient to counteract the radiation damping effect on the LSPR band resulting in large TSNP which are highly sensitive at longer wavelengths. Referring to FIG. 23, it can be seen that there is a slight dip in the LSPR sensitivity at wavelengths between 800 to 900 nm as the radiation damping starts to take effect as the aspect ratio of the TSNPs at this wavelength is not large enough to counteract the effect of radiation damping. However as aspect ratio increases for the TSNPs, there is a dramatic increase in LSPR sensitivity above 900 nm LSPR λₘₐₓ. We believe that aspect ratio is the critical factor in overcoming radiation damping. Nanoplates having a high aspect ratio exhibit a longer wavelength LSPR. As aspect ratio increases, the LSPR and size of the nanoplates increases and the scaling of these factors enables the TSNPs to overcome the effects normally associated with radiation damping of large nanostructures.

The enhanced sensitivities observed for high aspect ratio nanoplates can be supported by examining the various electron scattering contributions to the LSPR bandwidth. The high aspect ratio platelet structure of the TSNP indicates that unlike lower aspect ratio nanostructures of similar edge length volume scattering effects are inhibited and surface effects remain dominant due to the high fraction of the metal atoms located near the surface compared to the case of thicker nanostructures. The high aspect ratio facilitates the continued dominance of surface effects over volume effects even at larger TSNP sizes leads to a strong enhancement of the LSPR sensitivity.

Due to the location of these TSNP ensembles LSPR λₘₐₓ peaks within the Vis-NIR wavelengths, interband transitions which occur for silver in the UV (˜330 nm)²⁷ can be neglected as the free electron processes dominate. In the classical theory of free electron metals the damping that determines the width γ of the dipole plasmon is due to scattering with phonons, electrons and lattice defects. The size and shape dependence of the width of the LSPR, taking into account all the relative contributions from bulk dephasing, electron-surface scattering and radiation damping, can be described as³³

$$γ = {γ}_{bulk} + \frac{{Av}_{f}}{{L}_{eff}} + \frac{ℏ κ V}{2} \qquad ( Equation 9 )$$

where γ_{bulk} is the bulk damping constant,
v_{f} is the fermi velocity of electrons in silver,
L_{eff} is the effective mean free path of the electrons,
V is the nanoparticle volume and
A and κ are constants describing the electron surface scattering and volume induced radiation damping contributions respectively.

This expression is valid when the LSPR corresponds to a single dipolar resonance and may be applied to the TSNPs due to strong dominance of the dipolar peak, over higher order resonances. The effective mean free path can be expressed in terms of the volume V and surface area S of the nanoparticles³⁴,

$${L}_{eff} = \frac{4 V}{S} \qquad ( Equation 10 )$$

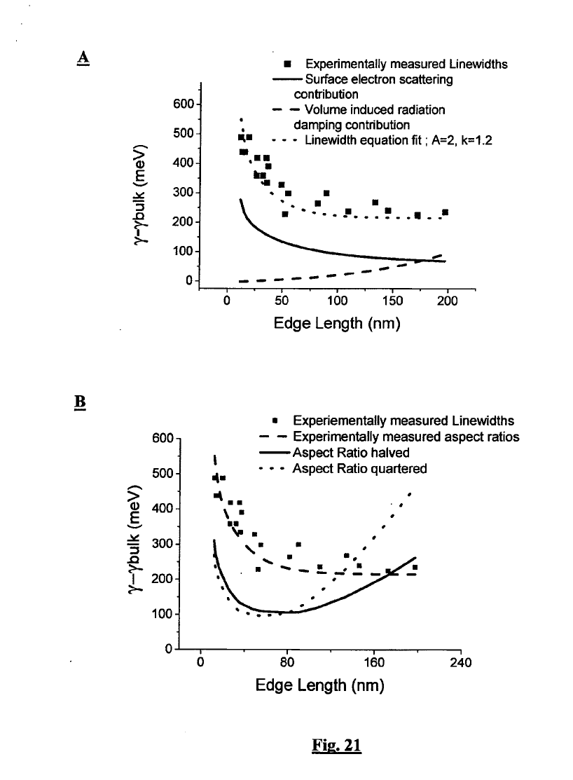

This effective mean free path though generally used for nanostructures with dimensions smaller than the mean free path of the conduction electron, can be extended to the case of the TSNP given their low thickness and their resultant high aspect ratio platelet like structure. The application of the linewidth equation using the experimentally measured structural parameters of the TSNPs shown in FIG. 21A illustrates the proposed contributions from the electron surface scattering and radiation damping parameters. It is apparent that the measured linewidths follow a trend similar to the electron scattering contribution indicating that this is the dominant factor and that volume contributions have a lower influence. This is the case even at larger diameters suggesting that the TSNPs continue to behave within the quasistatic regime due to the height aspects which are multiples less than the electron mean free path. The values of A and κ found to fit the experimental data best were A=2 and κ=1.2. which is in agreement with the κ value recently measured for silver nanoprisms³⁵. Further verification of the high aspect ratio explanation is provided by calculations of linewidths for TSNP which have multiples of the experimentally measured thickness (FIG. 21B). These calculations verify that as the thickness is increased larger contributions from the volume component are observed, in particular for larger edge length nanostructures, demonstrating the expected influence of the radiation damping parameter which would result in lower LSPR sensitivities for such larger edge length lower aspect ratio nanostructures. This is in agreement with values reported in the literature for single gold nanopyramids which showed a reduction in LSPR sensitivity with increased nanostructure height which was attributed to the thinner nanostructures exhibiting a higher volume fraction located near the nanostructure's surface³⁶.

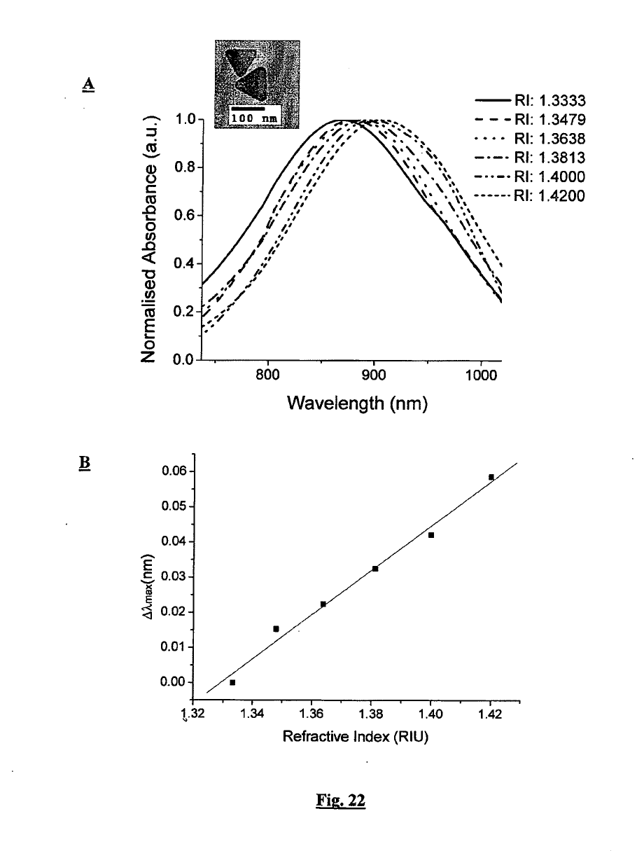

The sensitivity of TSNP preparations LSPR to changes in the external dielectric environment was investigated using a simple sucrose testing method whereby the refractive index of the solution surrounding the particles was changed through a variation in sucrose concentration. The sucrose method allows for a change in refractive index in the local surroundings without involving a change in the chemical environment of the solution, as may occur when using solvents, resulting in any shift in the nanoplates extinction spectrum being solely attributable to the refractive index change. The refractive indices of the sucrose concentrations used were measured after preparation on a temperature controlled AR-2008 Digital ABBE Refractometer with a 589 nm LED light source and compared to the universally known Brix scale for accuracy. FIG. 22A shows an example of the spectral shift observed for one of the TSNP in the various concentrations of sucrose. The sensitivity of the solution phase nanoparticles Δλ/RIU can be represented by plotting the shift observed in the peak plasmon wavelength Δλ against the corresponding refractive index of the sucrose FIG. 22B.

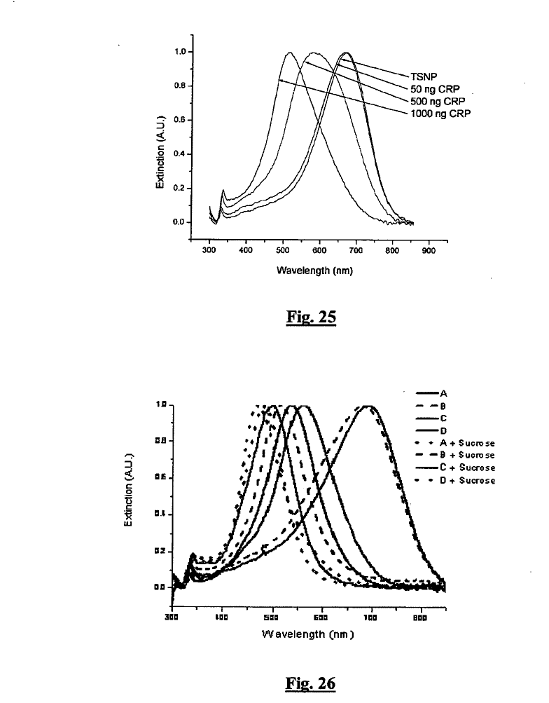

FIG. 24 shows an example of the spectral shift observed for a 100 nm edge length TSNP ensemble suspended in the various concentrations of sucrose. FIG. 25 shows that the LSPR sensitivity increases as λₘₐₓ is red-shifted throughout the visible to the NIR with a dramatic increase in sensitivity occurring at the longer wavelengths. It is apparent that the highest sensitivities occurred for the TSNP ensembles with the highest aspect ratios and correspondingly with LSPR wavelengths located in the NIR (Table 2).

TABLE 2
The highest LSPR sensitivities recorded for the
twenty ensemble samples tested in ascending order

| TEM Edge | Height | Aspect | Peak λ | Δλ(nm)/ |
|---|---|---|---|---|
| Length (nm) | (nm) | Ratio | (nm) | RIU |
| 145.72 | 14.12 | 10.32 | 1032.3 | 624.2 |
| 172.37 | 14.04 | 12.28 | 1070.9 | 668.5 |
| 134.07 | 13.39 | 10.01 | 1118.4 | 888.2 |
| 197.23 | 14.86 | 13.27 | 1093.1 | 1070.6 |

In this particular example, triangular silver nanoplates (TSNP) were produced by the two-step seed mediated method described in Example 1 above.

Blue Shifting of the TSNP was carried out as follows 1 mL of the functionalized TSNP is then centrifuged at 13,200 rpm for 30 minutes at 4° C. The colourless supernatant is then removed and the pellet is redispersed in 100 μL distilled H₂O.

The blue shifted TSNPs were used as biosensors in an assay for the acute phase protein C-reactive protein (CRP).

Fresh dilutions of CRP, in H₂O at pH 5.8 were prepared and kept on ice.

(Solution 1: CRP at 50 ng/uL, Solution 2, CRP at 12.5 ng/uL (¼ dilution of solution 1)).

A solution of CaCl₂ (1 mM) is also prepared.

In a black 96 well plate (flat, transparent bottom), the following solutions are all quoted:

1. 10 μL of 1 mM CaCl₂ per well
2. Variable amount of CRP (agent) (from 50 ng to 1 μg per well)
3. Water is added to a total volume of 290 μL per well (sigma)

### 4. Add 10 μL of TSNP.

5. Homogenise the contents of each well by pipetting.

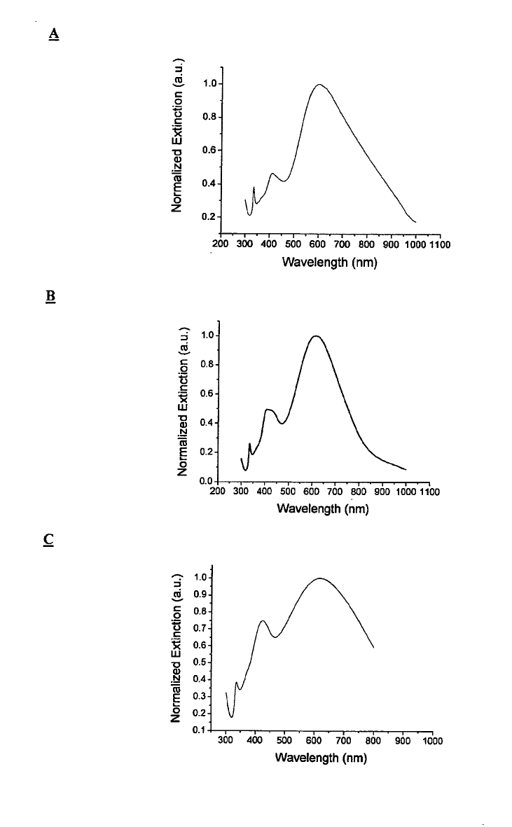

The spectra were then read. Referring to FIGS. 24 and 25, as the concentration of CRP is increased from 0 to 1000 ng/well, the spectral position of the in-plane dipole resonance is blue-shifted. Also shown in FIG. 24 is the red shifting of the TSNP using BSA in H₂O. FIG. 26 shows the blue shifting of TSNP using treatment with 50% w/v sucrose. Solution A is un coated, unfunctionalised TSNP, B is in situ PC functionalised TSNP; solution C is in situ hydrolysed-PC and un-hydrolised PC functionalised TSNP where the hydrolysed-PC has been exposed to water vapour and allowed to hydrolyse; and solution D is in situ hydrolysed-PC functionalised TSNP. In FIGS. 24 to 26 the out-of-plane quadrupole peak in the region of 330 to 345 nm remain consistently strong signifying that this is not just an etching process and that the geometric nature of the TSNP remains largely intact. The out-of-plane quadrupole peak is observed to red shift from about 330 nm to 340 nm as the in-plane dipole peak blue shifted through about 250 nm as observed in FIG. 26.

### Figure of Merit for Refractive Index Local Surface Plasmon Resonance Sensing

Figure of Merit (FOM) is a method of defining the overall sensitivity response of a plasmonic nanostructure. The FOM can be expressed as the ratio between the linear refractive index sensitivity of the nanostructure LSPR divided by its LSPR linewidth or full width half max (fwhm) signifying how narrow linewidths are desirable for optimum sensing. We compared the FOM for refractive index LSPR sensing of nanoparticles produced in accordance with the method described in PCT/IE2004/000047 (hereinafter referred to as PVA nanoparticles) and triangular silver nanoplates (TSNP) prepared in accordance with the methods described in Examples 1 to 3 above.

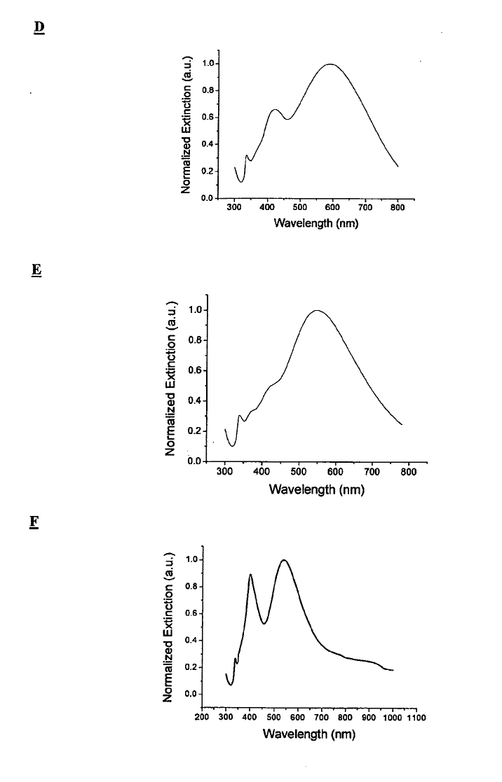

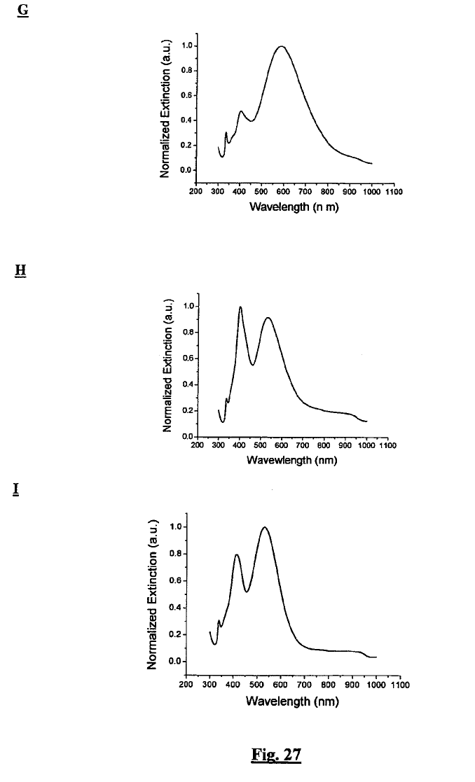

Referring to FIG. 27, the PVA nanoparticle spectra consist of 2 peaks, Peak 1 is the shorter wavelength peak (between about 410 to about 440 nm) which can be attributed to the presence of spherical particles within the distribution of particles within the sol and Peak 2 is the longer wavelength peak (about 600 nm), with higher intensity which can be attributed to the shaped particles within the sol. The properties of the different samples of particles are given in Tables 3 and 4 below.

TABLE 3
Properties of PVA nanoparticles produced according to the method described in
PCT/IE2004/000047

| | Diameter | Shape % | Height | Aspect | Peak λ | Δλ | FWHM | Γ | FOM |
|---|---|---|---|---|---|---|---|---|---|
| Sample | (nm) | (TEM) | (nm) | Ratio | (nm) | (nm)/RIU | (nm) | (eV) | (nm) |
| S22.2 | 25.39 | 55% | 12.67 | 2.01 | 412.17 | 88.26 | ~65 | 0.451 | 1.35 |
| (FIG. | | (22% | (STD | | 600.37 | 376.55 | 278.98 | 0.887 | 1.35 |
| 16A) | | Triangles, | Dev: | | | | | | |
| | | 33% | 5.35) | | | | | | |
| | | Hex) | | | | | | | |
| S31.2 | 28.27 | 59% | 18.29 | 1.55 | 409.05 | 87.02 | ~58 | 0.596 | 1.5 |
| (FIG. | | (26% | (STD | | 613.69 | 322.164 | 244.26 | 0.582 | 1.32 |
| 16B) | | Triangles, | Dev: | | | | | | |
| | | 33% | 8.6) | | | | | | |
| | | Hex) | | | | | | | |
| Sample 7 | 39.68 | 67% | 16.75 | 2.37 | 424.34 | 105.738 | ~56 | 0.205 | 1.89 |
| (FIG. | | (49% | (STD | | 616.88 | 327.164 | 287.15 | 0.442 | 1.14 |
| 16C) | | Triangle, | Dev | | | | | | |
| | | 18% | 6.22) | | | | | | |
| | | Hex) | | | | | | | |
| Sample 6 | 37.39 | 64% | 16.48 | 2.27 | 421.25 | 113.358 | ~62 | 0.314 | 1.83 |
| (FIG. | | (42% | (STD | | 588.57 | 271.123 | 205.07 | 0.559 | 1.32 |
| 16D) | | Triangle | Dev | | | | | | |
| | | 22% | 5.58) | | | | | | |
| | | Hex) | | | | | | | |
| Sample 2 | 30.46 | 57% | 17.16 | 1.76 | — | — | — | — | — |
| (FIG. | | (12% | (STD | | 547.33 | 259.092 | 229.35 | 0.414 | 1.13 |
| 16E) | | Triangle, | Dev | | | | | | |
| | | 45% | 5.31) | | | | | | |
| | | Hex) | | | | | | | |

TABLE 4
Properties of further PVA nanoparticles
produced in accordance with the methods
described in PCT/IE2004/000047

| Sample | Peak Wave | Sensitivity | FWHM | FOM |
|---|---|---|---|---|
| S21.1 | 538.39 | 199.04 | 202.09 | 0.98 |
| (FIG. | | | | |
| 16F) | | | | |
| S21.2 | 584.58 | 183.25 | 232.03 | 0.79 |
| (FIG. | | | | |
| 16G) | | | | |
| S22.1 | 533.91 | 173.19 | 172.78 | 1 |
| (FIG. | | | | |
| 16H) | | | | |
| S22.3 | 530.64 | 200.45 | 152.48 | 1.31 |
| (FIG. | | | | |
| 16I) | | | | |

Referring to FIG. 29, the highest linear refractive index sensitivities recorded for the PVA particles are similar to those recorded for the TSNPs, however there is a variation between sample batches of PVA nanoparticles.

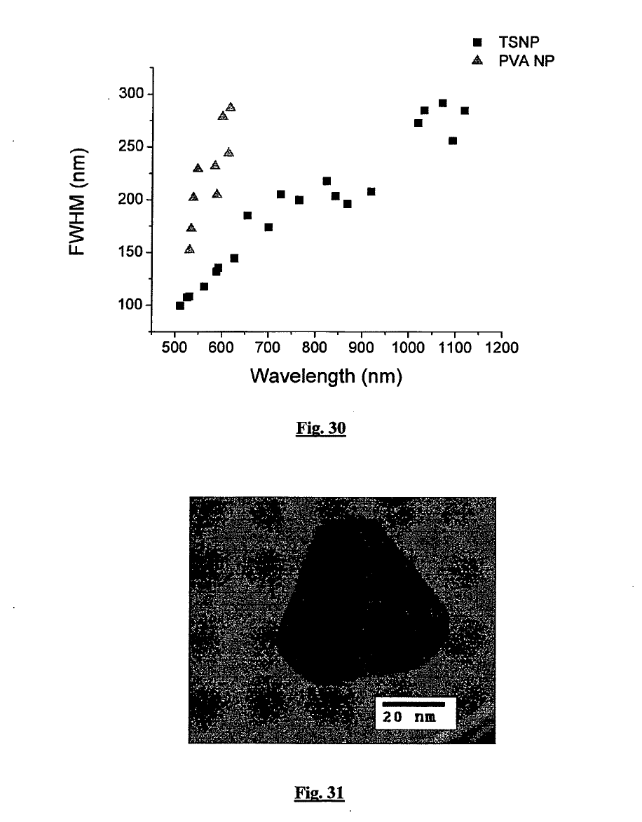

Referring to FIG. 30, the FWHM of the PVA particles are much broader than those for the TSNPs at similar wavelengths which is a result of the larger size and shape distributions and also possibly due to coupling between the particle.

TABLE 5
comparing the properties of TSNPs and PVA nanoparticles with
nanoparticles described in the literature

| | | Δλ(nm)/ | ΔE (eV)/ | |
|---|---|---|---|---|
| Sample | Peak λ (nm) | RIU | RIU | FOM |
| Single silver | 631 | 205 | 0.57 | 2.2 |
| Nanoprisms¹⁹ | 635 | 183 | 0.51 | 2.6 |
| | 631 | 196 | 0.55 | 3.3 |
| Nanorice²³ | Longitudinal: | 801 | — | — |
| | 1160 nm | | | |
| | Transverse: 860 | 103 | — | — |
| Gold Nanoshells²² | 720 | 409 | — | — |
| Gold Nanorings²⁴ | 1545 | 880 | — | 2 |
| Rod-Shaped Gold | Ensemble~650 | 150-285 | — | 2.1-3 |
| Nanorattles²⁹ | Single particle- | 199 ± 70 | — | 3.8 |
| Single silver | Pk 1: 351 | — | 0.79 | 1.6 |
| Nanocubes²⁰ | Pk 2: 444 | — | 0.69 | 5.4 |
| Single Gold | Pk 1: 650 | — | 0.65 | 3.8 |
| Nanostars²¹ | Pk 2: 700 | 643¹ | 1.41 | 10.7 |
| Single Gold | 600 | 174-199 | | 1.2-2.2 |
| Nanopyramids²⁷ | | | | |
| Ag/PVA | 547-616 | 259-377 | 0.9-1.2 | 1.13- |
| nanoparticles³⁶ | | | | 1.35 |
| TSNP Ensembles | Pk: 504-1093 | 188-1096 | 0.59-1.2 | 1.8-4.3 |

¹Estimated value from FIG. 6(b) in reference

### Example 6

### Optical Tunability

Increased aspect ratio enables systematic shifting of the LSPR peak wavelength through out the Visible and NIR region.

Snipping triangular silver nanoparticles can result in blue shifting of the LSPR keeping the spectrum within ranges required for biosensing. The corners (tips) of the TSNP can be deliberately snipped using chemical treatment or functionalisation. Snipped or truncated TSNP may be produced by a number of means including post synthetic treatment with chemical agents such as mercaptobenzoic acid or mercaptohexadecanoic acid or salts including sodium chloride, sodium bromide, sodium iodide or polymers such as polyvinyl alcohol or polyvinylpyrrolidone or sucrose or biological agents such as BSA or antibodies or C-reactive protein by alteration or adjustment of the surface chemistry or stabilisation of the TSNP on production, such as the reduction or increase in the amount of trisodium citrate (TSC) used and incubating the TSNP for a time from 10 minutes to several hours to several days. Another method for the creation of snipped or truncated TSNP is using centrifugation where the TSNP or functionalised TSNP may be centrifuged at 16,000 g.

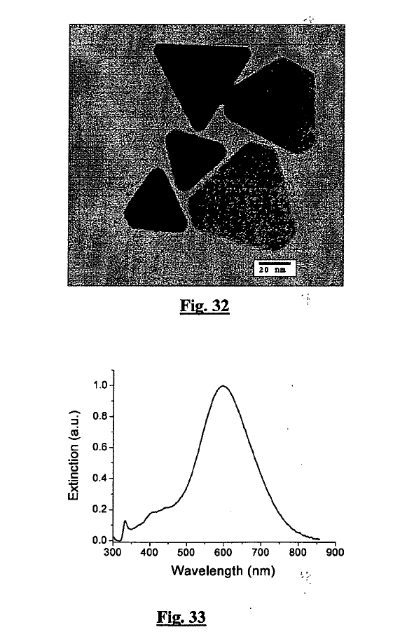

Referring to FIGS. 31 and 32, FIG. 31 is a transmission electron micrograph of a single snipped high aspect ratio triangular silver nanoprism and FIG. 32 is a transmission electron micrograph of a mixture of snipped and unsnipped high aspect ratio triangular silver nanoprisms. The snipped TSNP maintain their high aspect ratios and high LSPR sensitivity. The snipping of the corners (tips) has blue shifted the LSPR peak wavelength so that it remains within the 300 nm to 1150 nm spectral window appropriate for biosensing. Water and other organic molecules do not absorb in this spectral window.

Although the electrostatic fields for individual TSNP is found to decrease when the nanoparticle corners are snipped in the case of dimers the opposite is found where E-field enhancement is increased where the dimers are composed of snipped triangles as opposed to unsnipped triangles. This creates the “lightening rod” effect, which is a concept that comes from electrostatics less relevant for the plasmon resonant response of dimers. For SERS studies, the dimer of the snipped TNSP is better choice than unsnipped TSNP because the contact area at the interface is larger for that case, while the enhancement is the same. The electromagnetic field is larger for the unsnipped particles than for the snipped particles.

### Example 7

### In Situ Receptor Functionalisation of TSNP

In situ functionalisation of the surface of TSNP with antibodies, antibody fragments, proteins, peptides, nucleic acid, ligands and the like may produce in situ functionalised TSNP which are stable under ambient and/or assay conditions. The concentration of the functionalisation agent may be a factor in the degree of stabilisation of the in situ functionalised TSNP. For example, in situ IgG functionalised TSNP using 0.1 mg/ml IgG are highly stable under ambient and assay conditions. In the case of in situ phosphocholine functionalised TSNP using a 30 mM concentration of phosphocholine, the functionalised TSNP may be further stabilised by the addition of 25 mM TSC.

In the examples given below 200 μL seed solutions are used

### A) Antibody Functionalization:

1 mL of concentrations ranging from 0.1 mg mL⁻¹ to 1 mg·mL⁻¹ of freshly prepared aqueous solution of IgG from rabbit serum was added to the triangular silver nanoplates prepared as described in Example 1 in place of 0.5 ml of 25 mM Trisodium citrate. The total volume of the sol was then brought to 10 mL with distilled water and the sol was left undisturbed at 4° C. in the dark for overnight incubation. A typical UV-vis spectrum of such sol is shown in FIG. 23. TSNP solutions functionalized and stabilized by this method are stable for extended periods of time (in the order of months). Excess IgG may be removed by a centrifugation step (30 minutes at 20,000 g) and the resulting nanoplates may be easily re-dispersed back to their original volume or to a smaller volume (thus giving a more concentrated dispersion of nanoplates) with minimal loss of particles.

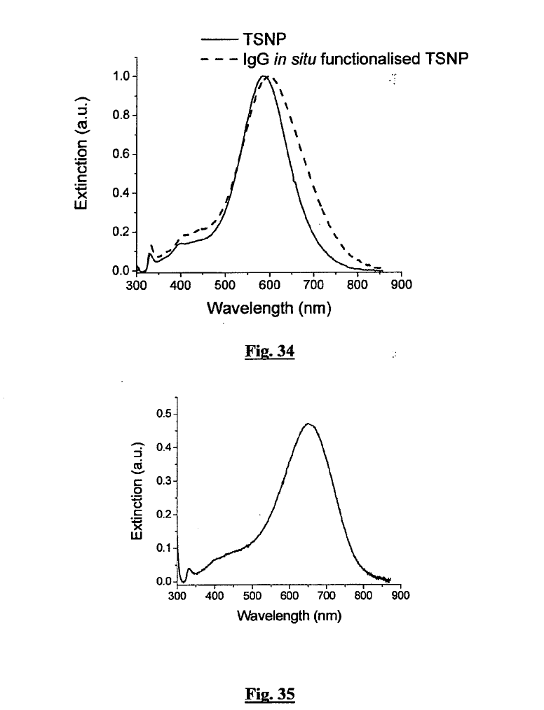

FIG. 34 shows a UV-vis spectrum of unfunctionalised TSNP stabilised by TSC and TSNP in-situ functionalised and stabilised by IgG; A red shift was observed for the case of the in-situ IgG functionalised TSNP compared TSC stabilised TSNP. This shift verifies the presence of the IgG on the surface of the TSNP as the larger physical size of the IgG compared to TSC will provide an increased refractive index change at the TSNP surface thereby inducing the red shift. The in-situ IgG functionalised TSNP were stable under both ambient and assay conditions

### B) Ligand Functionalization:

1 mL of a 30 mM freshly prepared aqueous solution of cytidine 5′-diphosphocholine (PC) was added to the triangular silver nanoplates prepared as described in Example 1 above. After an initial 30 minute incubation period, 500 μL of 25 mM trisodium citrate (TSC) was then added to sol for increased stabilization. The total volume of the sol was then brought to 10 mL with distilled water and the sol was left undisturbed at 4° C. in the dark for overnight incubation. A typical UV-vis spectrum of such sol is shown in FIG. 35. Sols stabilized/functionalized by this method are stable for extended periods of time (in the order of months). Excess PC/TSC may be removed by a centrifugation step (30 minutes at 20,000 g) and the resulting nanoplates may be easily redispersed back to their original volume or to a smaller volume (thus giving a more concentrated dispersion of nanoplates) with minimal loss of particles.

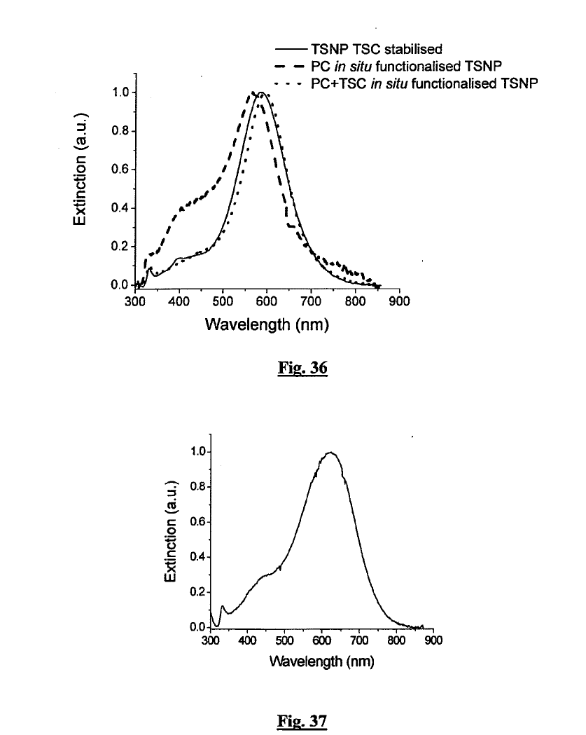

FIG. 36 shows a UV-vis spectrum of unfunctionalised TSNP stabilised by TSC and TSNP in-situ functionalised and stabilised by PC and TSNP stabilised by TSC and TSNP in-situ functionalised and stabilised by a combination of PC and TSC; A blue shift and spectral broadening was observed for in-situ PC only functionalised TSNP compared TSC stabilised TSNP. A slight red shift was observed for TSNP in-situ functionalised and stabilised by a combination of PC and TSC. This shift verifies the presence of the PC on the surface of the TSNP as the larger physical size of the PC induces the shift. The in-situ PC and TSC combination functionalised TSNP were stable under both ambient and assay conditions

### Oligonucleotide Functionalization:

Oligonucleotides structurally modified to contain a positively charged head group were sourced commercially. 200 μL of a 100 pM oligonucleotide was added to the triangular silver nanoplates prepared as described in Example 1 above. The total volume of the sol was then brought to 10 mL with distilled water and the sol was incubated with agitation at 4° C. in the dark overnight. A typical UV-vis spectrum of such sol is shown in FIG. 37. Sols stabilized/functionalized by this method are stable for extended periods of time (in the order of months). Particle purification can be carried out by a centrifugation step (30 minutes at 20,000 g) and/or by separation on MWCO membrane filtration devices commercial available for removal/isolation of free oligonucleotides (e.g. PALL, Millipore Systems). The resulting nanoplates may be easily redispersed back to their original volume or to a smaller volume (thus giving a more concentrated dispersion of nanoplates) with minimal loss of particles.

FIG. 40 is a set of UV-Visible spectra of unfunctionalised TSNP stabilised by TSC and in-situ nucleic acid probe functionalised and stabilised TSNP. A red shift is observed for TSNP in-situ functionalised and stabilised by nucleic acid probes. This shift verifies the presence of the nucleic acids on the surface of the TSNP as the larger physical size of the nucleic acid induces the optical shift. The in-situ nucleic acid functionalised TSNP were stable under both ambient and assay conditions.

### Unstabilised & In Situ Functionalised Nanoplates

According to the methods described herein, silver nanoplates are produced which enable intimate and direct contact of functionalisation agents and stabilization agents with the crystal lattice of the nanoplate surface. Stable silver nanoplates can be produced without any stabilization agent or functionalisation agent. To our knowledge, all the silver nanoplates and other nanostructures described in the literature are produced using a stabilization/capping/passivation agent. In the case of the production of the silver nanoplates without any stabiliser the same procedures are followed as given in the examples with one difference which is that no further reagents are added after the addition of the silver source.

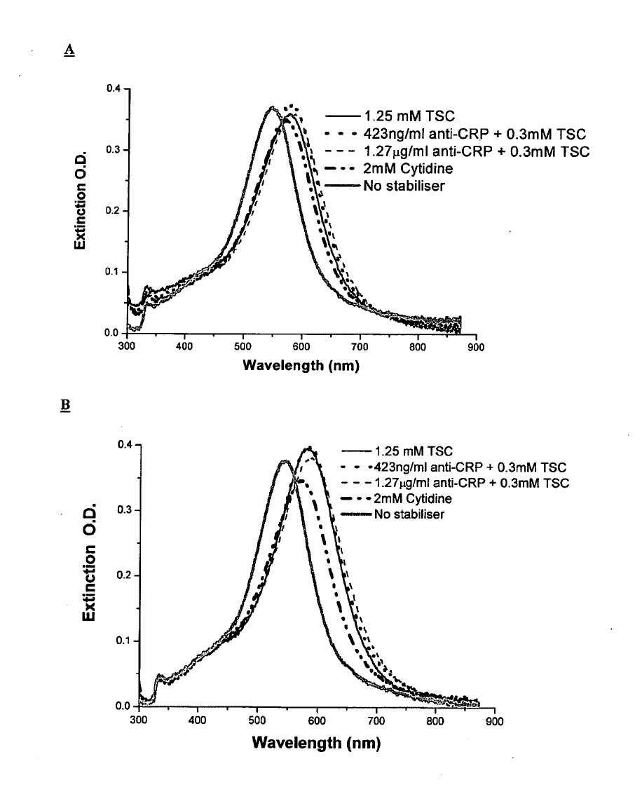

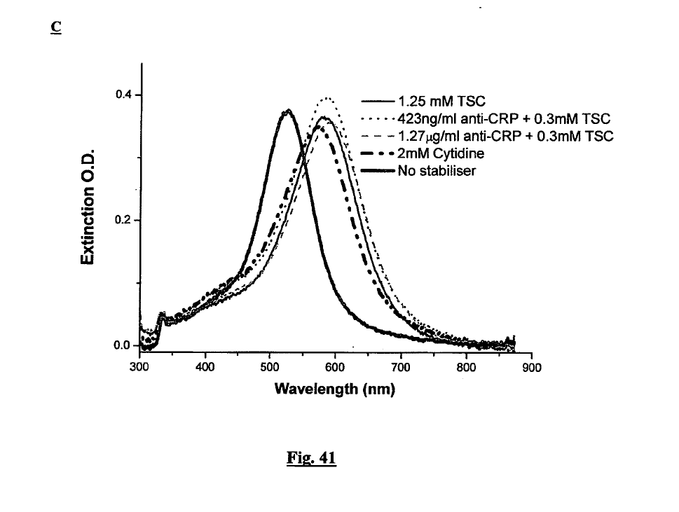

Referring to FIG. 41, the optical extinction spectra measured using UV-Visible-NIR spectroscopy of silver nanoplates produced with; 1.25 mM TSC stabilisation, stabilized by in-situ functionalized with 423 ng/ml anti-CRP antibody followed by the addition of 0.3 mM TSC, stabilized by in-situ functionalized with 1.27 μm/ml anti-CRP antibody followed by the addition of 0.3 mM TSC, stabilized with 2 mM Cytidine, no stabilization, show very little variation from 30 minutes after production (FIG. 41A) to 24 hours after production (FIG. 41B) to 1 week after production (FIG. 41C). The Table below lists the peak wavelength positions of each of these silver nanoplates each of which and including the silver nanoplates which are produced without a stabiliser are highly stable given the consistent profile of their LSPR spectra over time, including the presence of the out of plane quadrupole in the 340 nm region, little variation in the extinction optical density (O.D.) and the minimal shifting to the LSPR peak wavelengths.

This table lists peak wavelength spectral positions for nanoplates produced with; 1.25 mM TSC stabilisation, stabilized by in-situ functionalized with 423 ng/ml anti-CRP antibody followed by the addition of 0.3 mM TSC, stabilized by in-situ functionalized with 1.27 μm/ml anti-CRP antibody followed by the addition of 0.3 mM TSC, stabilized with 2 mM Cytidine, no stabilization

| | Peak wavelength λₘₐₓ (nm) | | |
|---|---|---|---|
| Stabilizer | Time 0 | 18 h | 1 week |
| 1.25 mM TSC | 577 | 581 | 581 |
| 423 ng/mL aCRP | 576 | 581 | 585 |
| 1.27 μg/mL aCRP | 578 | 585 | 594 |
| 2 mM Cytidine | 570 | 568 | 572 |
| No stabilizer | 546 | 543 | 527 |

TSC has previously been used to stabilize/Cap/passivate the nanoplates which results in TSC going directly on to the crystal lattice in direct contact with the Ag atoms aligned for example in a 111 plane⁵⁴. In the in-situ functionalisation methods described herein, the functionalisation agent (receptor) is deposited directly onto and in contact with the silver atomic crystal lattice such as the {111} face in a simple one pot method and no further intermediate agent or monolayer or chemical conjugation procedure is required. This not only acts to effectively stabilize/Cap/passivate the nanoplates it does this better than TSC alone. Furthermore, the optical/spectral signal of the in-situ functionalised nanoplates is improved as the functionalisation agent is in direct contact with the surface of the silver nanoplate and lies within the strongest regions of the electromagnetic field, rather than being spaced apart from the surface where the electromagnetic field intensity is weaker, which results in an extremely sensitive sensor.

### Example

Blue TSC stabilised TSNP, blue in situ PC functionalized TSNP and blue in situ anti-CRP functionalized TSNP were blocked with a 1 in 50 dilution of CRP free human serum. Each TSNP sample remained blue confirming the TSNP durability to the blocking process in each case. Subsequently full strength human serum was added to test the stability of each of the TSNP and the colour of the TSNP was observed over a 15 min period. The blocked TSC stabilised TSNP turned from blue to purple immediately indicating instability to the presence of full strength human serum. The blocked PC-TSNP and blocked in situ anti-CRP functionalized TSNP both remained blue over the 15 min time duration in presence of full strength human serum confirming the increased stability of in-situ receptor functionalized TSNP over TSC stabilised TSNP.

Direct in situ functionalisation enables increased binding of functionalisation agent to the surface of silver nanoplates compared to functionalisation by adsorption on to a surface coated with stabilising molecules. For example when the functionalisation agent is an antibody type receptor, the functionalisation agent can detach from the surface nanoparticle surface when an adsorption method is used. Furthermore, direct in situ functionalisation serves to preserve nanostructure geometry removing the need for chemical functionalisation which can act to degrade and damage the nanoparticle structure and hence the performance of its plasmon. Such chemical conjugation may also damage or interfere with the biological or chemical functionality of the receptor. The elimination of conjugation chemistries increased synthesis yields, avoiding issues such as nanomaterial losses through centrifugation and purification steps.

### Example 8

### Blocking of TSNP Sensors

Post synthetic stabilization of the as prepared triangular silver nanoplates can be carried out in a versatile manner which allows the surface chemistry of the nanoplates to be altered depending on their intended use.

For example, 1 mL of a 30 mM freshly prepared aqueous solution of cytidine 5′-diphosphocholine (PC) can be added to the triangular silver nanoplates prepared as described above. After an initial 30 minute incubation period, 500 μL of 25 mM trisodium citrate (TSC) can be added to sol for increased stabilization. The total volume of the sol is then brought to 10 mL with distilled water and the sol is left undisturbed at 4° C. in the dark for over night incubation, these nanoplates are the sensor.

The nanoplates may be blocked with an ethanolic solution of 16-mercaptohexadecanoic acid (MHA) by incubating the sensor with MHA at 4° C. for at least one hour to allow complexation of the MHA to the surface. Blocking the sensor with MHA reduces the level of non-specific binding of the analyte molecule to the nanoparticle (sensor) surface. The concentration of MHA used determines the extent to which the sensor is blocked. The concentration range studied in this Example was 20 nM to 20 μM. Other blocking agents which may be used include styrene, polyethylene glycol and other mercapto based agents. A mixture of more than one agent may also be used for blocking purposes.

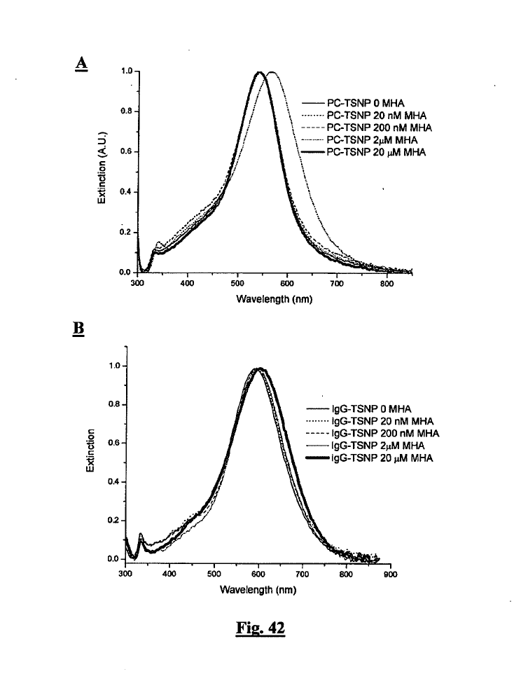

FIG. 42 shows the UV-Vis spectra for (A) in situ PC functionalized TSNP blocked with MHA concentration in the range of 0 to 20 μM; (B) is a UV-Vis spectra for in situ IgG functionalized TSNP blocked with MHA concentration in the range of 0 to 20 μM.

An important concern that needs to be addressed when designing high-sensitivity sensors is the ability of the sensor to achieve a response that is specific to the analyte in question. This requires the sensor to be of high specificity, capturing the analyte of interest while suppressing interactions of all other molecules. Thin film coatings of the receptor functionalized nanoplate sensor surface for example with molecular monolayers at thicknesses less than 10 nm can provide a steric repulsive barrier to non-specific adsorption. In the case of such coatings it is important that the coating is thin enough to enable efficient analyte receptor interaction at the nanoparticles surface.

Here we demonstrate blocking of a sensor using (i) a molecular blocker, MHA (16-mercaptohexadecanoic), which is used to fill in the gaps between the receptor molecules on the nanoplate sensor surfaces and (ii) serum which is a standard blocking agent for a bioreceptor and analyte interaction and binding studies.

MHA is a long-chain molecule which acts as a blocking agent that prevents non specific molecules from adsorbing to the nanoplate surface and nanoplate sensor surface while enabling specific binding of analyte molecules to receptors on the nanoplate sensor surface. The principle behind serum blocking is that non-immune serum from the host species of the receptor antibody is applied to the nanoplates and will adhere to protein-binding sites either by nonspecific adsorption or by binding of specific but unwanted, serum antibodies to antigens. The serum constituent will reposition to enable specific binding between receptors bound directly to the nanoplate sensor surface and target analytes. In addition blocking agents such as MHA and Serum act to protect the nanoplate from etching in harsh environment such as saline or serum solution.

### Example 8A

### Molecular Blocking of TSNP and PC In Situ Functionalised TSNP Using MHA

A series of studies were carried out on the impact of the MHA blocking on the LSPR sensitivity of bare nanoplates and nanoplates sensors produced by in situ functionalisation where the receptor, in this case phosphocholine (PC) which is specific for C-reactive protein, is directly bonded to the surfaces of the nanoplates.

### LSPR Sensitivity of TSNP Sols and Blocked TSNP Sols

Four different TSNP sots in total, two non-blocked TSNP sols and two blocked TSNP sols were prepared as follows

1) TSC stabilised TSNP
2) 16-mercaptohexadecanoic (MHA) blocked TSC stabilised TSNP
3) Phosphocholine (PC) stabilised TSNP i.e. PC in situ functionalised TSNP
4) MHA blocked PC stabilised TSNP. i.e. MHA blocked PC in situ functionalised TSNP

The MHA blocking was carried out by adding MHA to the sols at a given concentration

500 μL of each sol to be tested was centrifuged at 13,200 rpm for 20 minutes. The colourless supernatant was removed and the pellets were redispersed in 50 μL H₂O. 10 μL of this sol was then placed in the well of a 96 well plate to which 290 μL
1) H₂O
2) 10% w/v sucrose
3) 25% w/v sucrose
4) 50% w/v sucrose

The optical extinction spectra were recorded using UV-vis spectroscopy and are shown in FIG. 32.

Blocking of TSC Stabilised TSNP with Original Peak Wavelength in the Region of 541 nm

TSC stabilized TSNP were blocked at the following concentration of MHA

E: NP TSC stabilised+0 nM MHA
E1: NP TSC stabilised+20 nM MHA
E2: NP TSC stabilised+200 nM MHA
E3: NP TSC stabilised+2 μM MHA
E4: NP TSC stabilised+20 μM MHA

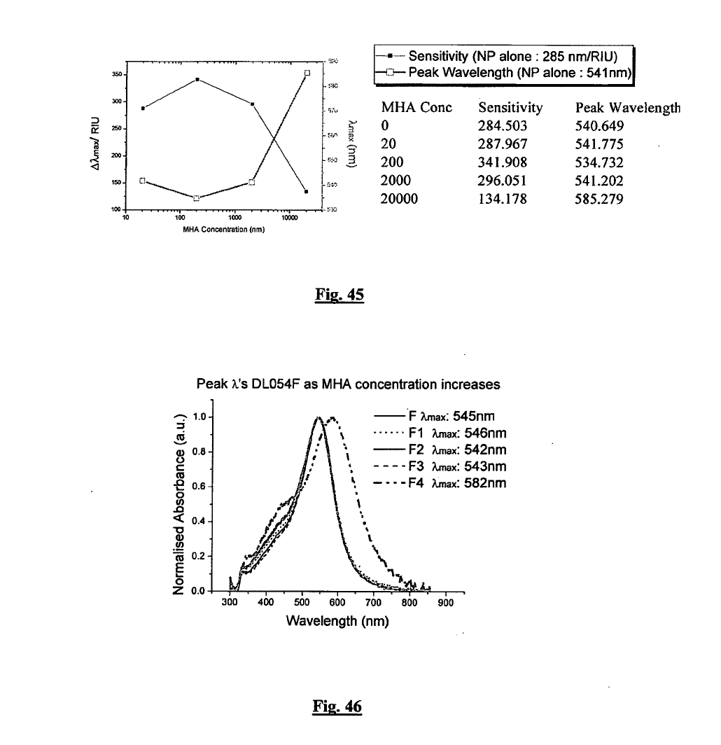

The optical extinction spectra of TSC stabilised TSNP after addition of MHA at concentrations, 20 nM, 200 nM, 2 μM and 20 μM were recorded using UV-vis spectroscopy and are shown in FIG. 44. The LSPR sensitivities and Peak wavelength dependence of TSC stabilised TSNP upon the nM concentration of MHA (log scale) are shown in FIG. 45.

Referring to FIG. 45, LSPR sensitivity of TSC stabilised TSNP with original peak wavelength in the region of 541 nm did not show any decrease on blocking with MHA up to concentrations of 2000 nM. Only a slight shifting of the peak wavelength was observed on blocking with MHA up to concentrations of 2000 nM. Furthermore, an increase in LSPR sensitivities is observed at MHA blocking concentrations between 200 nM and 2000 nM. This increase may correspond to coupling of the nanoplates e.g. in, pairs, triplets or short chains or it main correspond to sensitizing the surface of the nanoplates to facilitate a more responsive surface electric field which has increased receptiveness and susceptibility to the local surrounding environment and thereby provides for increased LSPR sensitivity. A decrease in the LSRP sensitivity was observed at an MHA blocking concentration of 20000 nM. Also a significant red shift of the order of 100 nm was observed on MHA blocking concentration of 20000 nM. This may correspond to large grouping of the nanoplates or to the fact that the concentration of MHA molecules is now high enough to shield the surface of the nanoplates to a certain extent from responding with its full capacity to the local environment thereby resulting in decreased LSPR sensitivity.

Blocking of PC Stabilised TSNP with Original Peak Wavelength in the Region of 545 nm

PC stabilized TSNP were blocked at the following concentration of MHA

F: NP PC stabilized+0 nM MHA
F1: NP PC stabilised+20 nM MHA
F2: NP PC stabilised+200 nM MHA
F3: NP PC stabilised+2 μM MHA
F4: NP PC stabilised+20 μM MHA

The Optical Extinction Spectra of PC stabilised TSNP after the addition of MHA (20 nM, 200 nM, 2 μM and 20 μM) are shown in FIG. 46. The LSPR sensitivities and Peak wavelength dependence of PC stabilised TSNP upon the nM concentration of MHA (log scale) are shown in FIG. 47.

Referring to FIG. 47, LSPR sensitivity of PC stabilised TSNP with original peak wavelength in the region of 545 nm demonstrated a similar pattern to that of the TSC stabilised TSNP with original peak wavelength in the region of 541 nm (FIG. 45) and did not show any decrease in blocking with MHA up to concentrations of 2000 nM. Only slight shifting of the peak wavelength was observed on blocking with MHA up to concentrations of 2000 nM. Furthermore an increase in LSPR sensitivities was observed at MHA blocking concentrations between 200 nM and 2000 nM. This increase may correspond to coupling of the nanoplates e.g. in pairs, triplets or short chains or it main correspond to sensitising the surface of the nanoplates to facilitate a more responsive surface electric field which has increased receptiveness and susceptibility to the local surrounding environment and thereby provides for increased LSPR sensitivity. A decrease in the LSRP sensitivity was observed at an MHA blocking concentration of 20000 nM. Also a significant red shift of the order of 100 nm was observed on MHA blocking concentration of 20000 nM. This may correspond to large grouping of the nanoplates or to the fact that the concentration of MHA molecules is now high enough to shield the surface of the nanoplates to a certain extent from responding with its full capacity to the local environment thereby resulting in decreased LSPR sensitivity.

Blocking of TSC Stabilized TSNP with Original Peak Wavelength in the Region of 577 nm

TSC stabilized TSNP were blocked at the following concentration of MHA

G: NP TSC stabilised
G1: NP TSC stabilised+20 nM MHA
G2: NP TSC stabilised+200 nM MHA
G3: NP TSC stabilised+2 μM MHA
G4: NP TSC stabilised+20 μM MHA

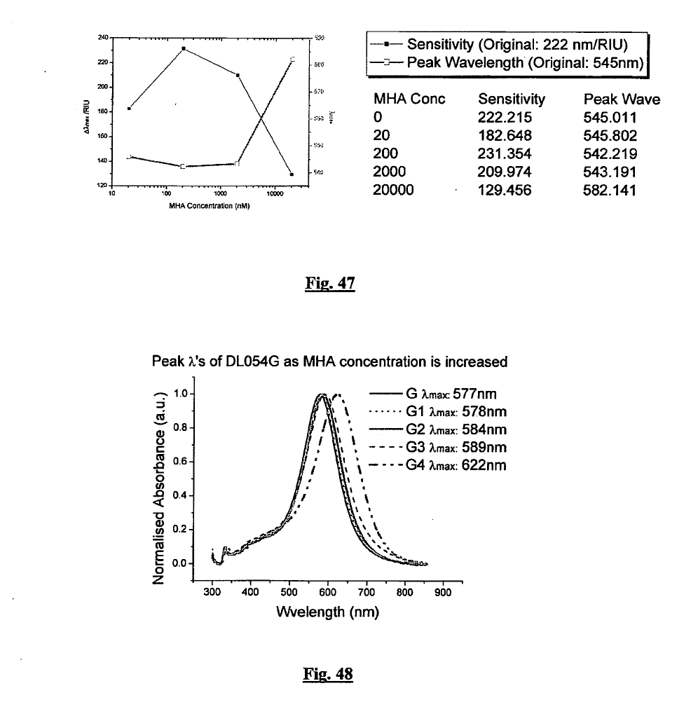

Optical extinction spectra of TSC stabilised TSNP after addition of MHA (20 nM, 200 nM, 2 μM and 20 μM) are shown in FIG. 48. The LSPR sensitivities and Peak wavelength dependence of TSC stabilised TSNP upon the nM concentration of MHA (log scale) are shown in FIG. 49.

Referring to FIG. 49, TSC stabilised TSNP with original peak wavelength in the region of 577 nm shows a constant LSPR sensitivity within experimental error on blocking with MHA up to concentrations of 200 nM. In fact an increase in LSPR sensitivities was observed at MHA blocking concentrations of 20 nM over the unblocked TSC stabilised TSNP. This increase may correspond to coupling of the nanoplates e.g. in pairs, triplets or short chains or it main correspond to sensitising the surface of the nanoplates to facilitate a more responsive surface electric field which has increased receptiveness and susceptibility to the local surrounding environment and thereby provides for increased LSPR sensitivity. A decrease in the LSRP sensitivity was observed at MHA blocking concentration of 2000 nM and above. This corresponds to significant red shifts at MHA blocking concentration of 2000 nM and above which may correspond to large grouping of the nanoplates or to the fact that the concentration of MHA molecules is now high enough to shield the surface of the nanoplates to a certain extent from responding with its full capacity to the local environment thereby resulting in decreased LSPR sensitivity.

Blocking of PC Functionalized TSNP with Original Peak Wavelength in the Region of 617 nm

PC stabilized TSNP were blocked at the following concentration of MHA

H: NP PC stabilized+0 nM MHA
H1: NP PC stabilised+20 nM MHA
H2: NP PC stabilised+200 nM MHA
H3: NP PC stabilised+2 μM MHA
H4: NP PC stabilised+20 μM MHA

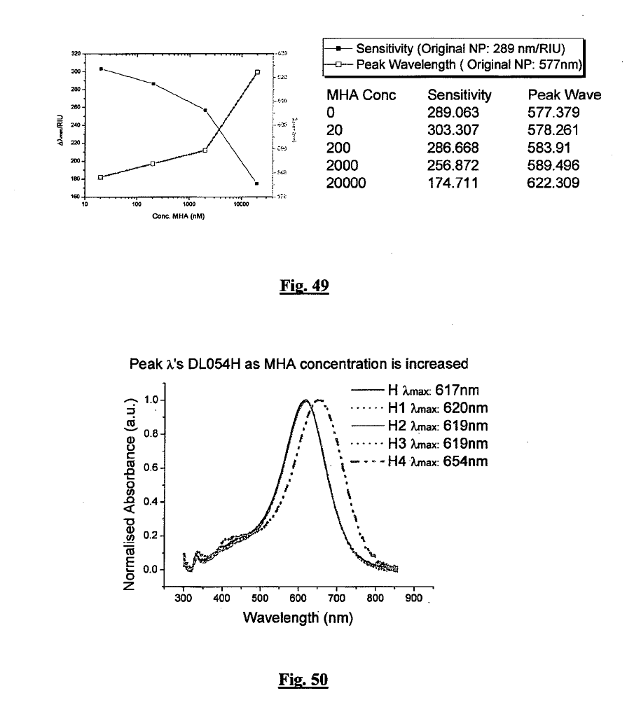

Optical Extinction Spectra of PC stabilised TSNP after addition of MHA (20 nM, 200 nM, 2 μM and 20 μM) are shown in FIG. 50. The LSPR sensitivities and Peak wavelength dependence of PC stabilised TSNP upon the nM concentration of MHA (log scale) are shown in FIG. 51.

Referring to FIG. 51, PC stabilised TSNP with original peak wavelength in the region of 617 nm show a very similar pattern to that of the TSC stabilised TSNP with original peak wavelength in the region of 577 nm (FIG. 49) showing a constant LSPR sensitivity within experimental error on blocking with MHA up to concentrations of 200 nM. An increase in LSPR sensitivities is observed at MHA blocking concentrations of 20 nM over the unblocked TSC stabilised TSNP. This increase may correspond to coupling of the nanoplates e.g. in pairs, triplets or short chains or it main correspond to sensitising the surface of the nanoplates to facilitate a more responsive surface electric field which has increased receptiveness and susceptibility to the local surrounding environment and thereby provides for increased LSPR sensitivity. A decrease in the LSRP sensitivity was observed at MHA blocking concentration of 2000 nM and above. This corresponds to significant red shifts at MHA blocking concentration of 2000 nM and above. This may correspond to large grouping of the nanoplates or to the fact that the concentration of MHA molecules is now high enough to shield the surface of the nanoplates to a certain extent from responding with its full capacity to the local environment thereby resulting in decreased LSPR sensitivity.

### Example 8B

### LSPR Biosensing for C-Reactive Protein Using MHA and Serum Blocked TSC Stabilised TSNP and PC Stabilised TSNP Sols

TSNP/sensors were aliquoted by 1 ml in eppendorf tubes. In the case of serum blocking 1 uL of serum was added to 1 mL of TSNP/sensors and in the case of MHA blocking, MHA was added to bring the concentration of MHA to 20 μM. The sample was vortexed 10 seconds, and immediately centrifuged (4° C.) for 10 minutes at a speed of 6-9K rpm for sensors (particularly antibody coated) or 30 minutes at a speed of 13.2K rpm for bare TSNP (TSC stabilised TSNP). Supernatant was discarded, and pellet was resuspended in 10% initial volume for TSNP (100 uL) and 5% to 10% initial volume for sensors. 10 uL of the blocked solutions were used in a 300 uL total volume assay, comprising: 50 uL serum, 240 uL water. Optical Extinction Spectra were recorded every minute for at least 3 minutes.

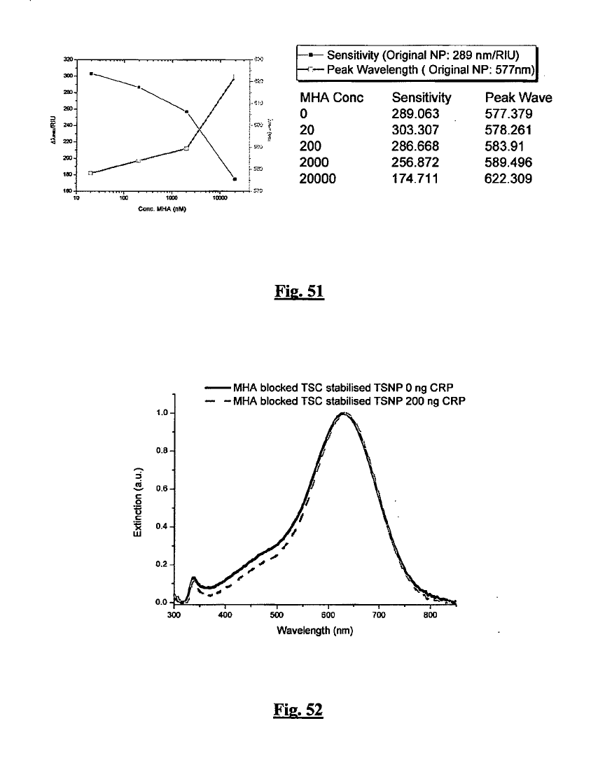

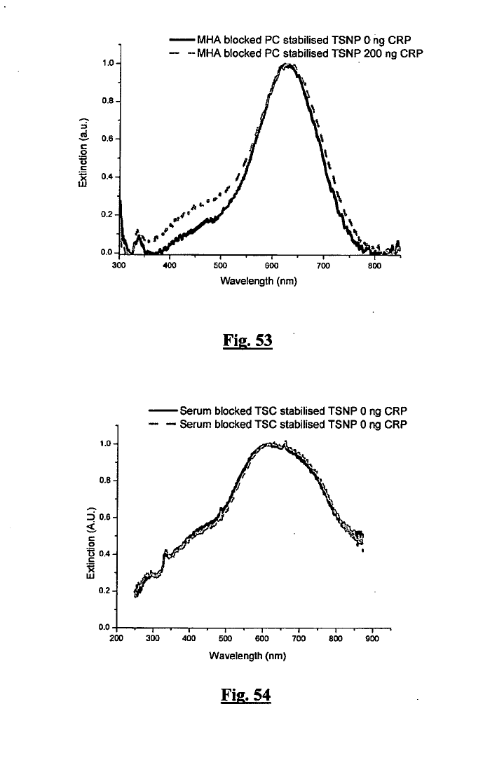

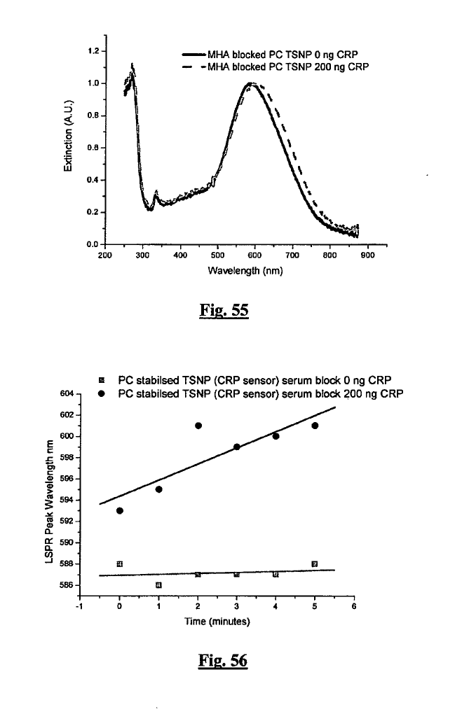

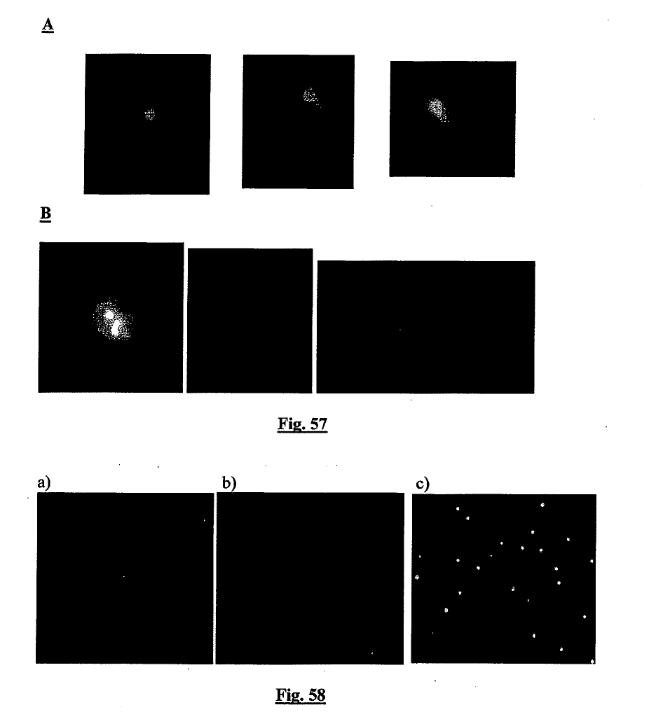

Referring to FIG. 52, Spectra of TSC stabilised TSNP blocked with 20 μM MHA show no clear LSPR red shift on the addition of 200 ng CRP. Referring to FIG. 53, Spectra of PC stabilised TSNP blocked with 20 μM MHA showing a clear LSPR red shift on the addition of 200 ng CRP. TSC stabilised TSNP and blocked with 20 μM MHA show no clear LSPR red shift on the addition of 200 ng CRP indicating the low occurrence of non-specific binding in the presence of the MHA blocking. A clear LSPR red shift was measured in the case of 20 μM MHA blocked PC stabilised TSNP on the addition of 200 ng CRP. This indicates that the presence of MHA blocking enables specific sensing of CRP with a the low occurrence of non-specific binding.

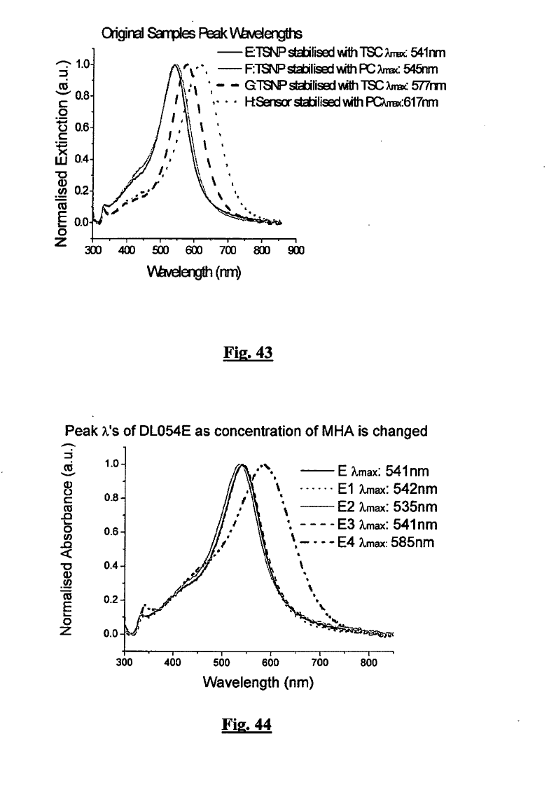

Referring to FIGS. 43 and 44, TSC stabilised TSNP and blocked with serum show no clear LSPR red shift on the addition of 200 ng CRP indicating the low occurrence of non-specific binding in the presence of the serum blocking. A clear LSPR red shift was measured in the case of serum blocked PC stabilised TSNP on the addition of 200 ng CRP. This indicates that serum blocking enables specific sensing of CRP with a low occurrence of non-specific binding

FIG. 45 shows that in the case of the addition of 0 ng of CRP to CRP sensor (PC stabilised TSNP) the LSPR peak position remains constant about 587 nm with time of 0 to 5 minutes. On the addition of 200 nm of CRP there is a constant increase in the LSPR peak wavelength over the 5 minute time period as more CRP molecules bind specifically to the PC receptors on the sensor surface.

From these results, it is clear that in the case of both MHA and Serum blocking, non-specific binding is dramatically reduced and specific LSPR sensing for CRP is achieved.

### Example 9

### Solution Phase Ensemble In Situ Receptor Functionalised TSNP Assay

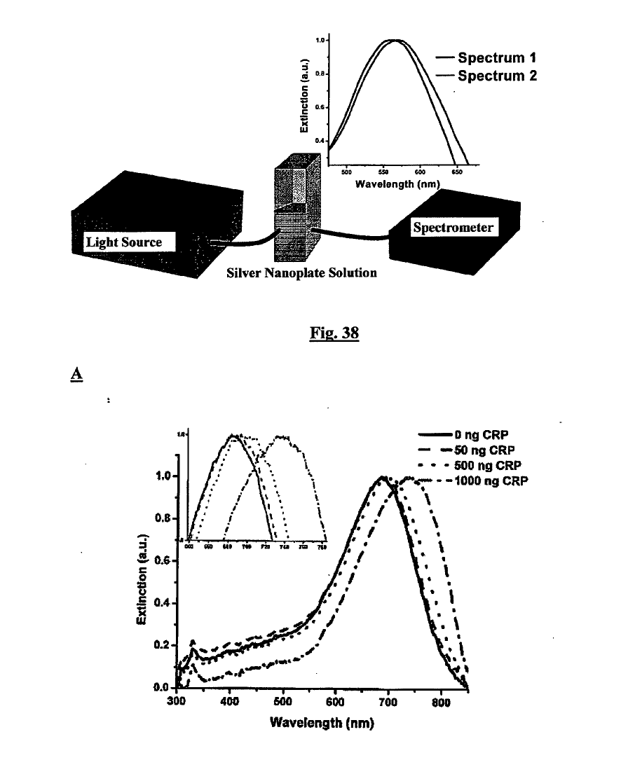

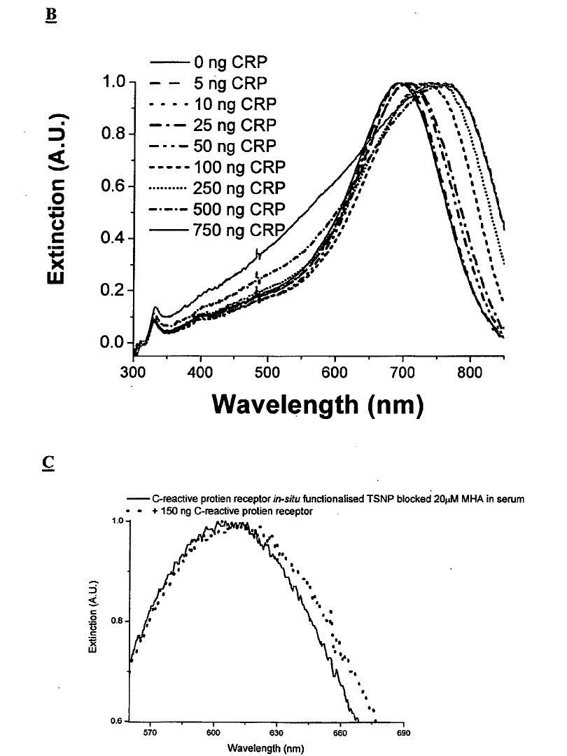

Referring to FIG. 38, a suitable detection system for a solution phase receptor functionalized TSNP assay involves a simple direct capture assay comprising a test solution, a light source and a spectrometer. In use, an initial UV-Visible spectrum of a solution of the in situ receptor functionalized TSNP (Spectrum 1) is recorded and following the addition of a sample containing a target analyte to the receptor functionalized TSP solution, a second UV-Visible spectrum is recorded (Spectrum 2). Analysis of the measured LSPR-shift will give an immediate (real-time) readout of the target concentration.

### (A) CRP Detection Assay Using Phosphocholine Functionalised TSNP

C-reactive protein (CRP) is a highly conserved plasma protein that participates in the systemic response to inflammation. CRP binds to a range of substances such as phosphocholine, fibronectin, chromatin, histones, and ribonucleoprotein in a calcium-dependent manner. It is a ligand for specific receptors on phagocytic leukocytes, mediates activation reactions on monocytes and macrophages, and activates complement. Plasma CRP is the classical acute-phase protein, increasing 1,000-fold in response to infection, ischemia, trauma, burns, and inflammatory conditions. It acts as a pattern recognition molecule that can bind to specific molecular configurations typically exposed during cell death or found on the surfaces of pathogens. Thus, CRP contributes to host defense and plays a crucial role in the first line of innate host defense.

In an assay for the acute phase protein C-reactive protein the biological capture agent was Phosphocholine which binds to C-reactive protein in the presence of CaCl₂.

Phosphocholine functionalised TSNP were held in microtubes tubes at 4° C. and centrifuged for 20 minutes at 16,000 g. The supernatant was removed and the TSNP were resuspended in 10% of initial volume, in water (from an ELGA purification system or HPLC grade purchased from Sigma Aldrich) and kept on ice/below room temperature. Fresh dilutions of human plasma or recombinant sourced CRP (Sigma Aldrich), in phosphate buffer 01=7.0, were used to make dilution standards; solution 1 CRP at [50 ng/uL] and solution 2 CRP at [12.5 ng/uL]. CaCl₂ solution was freshly prepared at 1 mM in water.

In a black 96 well plate, flat, transparent bottom, the solutions were aliquoted as follows:

1. 10 μL of CaCl₂ per well
2. Variable amount of analyte (0, ng and from 50 ng to 1 μg per well)

3. Make up to a total volume of 290 μL in water (sigma)

4. Add 10 μL of biosensor.

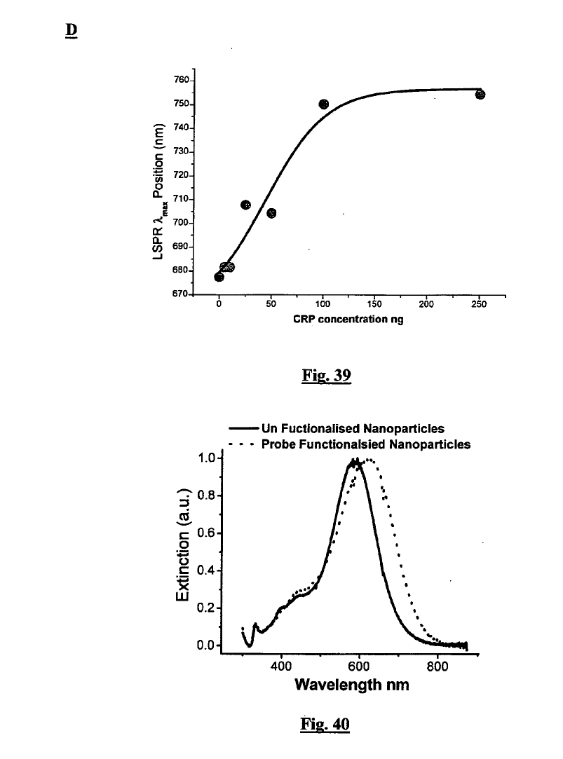

The UV-Vis spectra were then read. Referring to FIG. 39 (A) which is a UV-vis spectrum of a CRP Assay using total solution phase in-situ phosphocholine functionalised TSNP ensemble with an ensemble average in-plane dipole LSPR peak in the region of 680 nm. Systematic LSPR peak wavelength shift response on the presence of CRP is observed by the ensemble average LSRP of the in-situ phosphocholine functionalised TSNP.

Referring to FIG. 39 (B) which is a UV-vis spectrum of a CRP assay using in-situ phosphocholine functionalised TSNP and chemically blocked using 0.2 μM MHA. A systematic LSPR peak wavelength shift response on the presence of CRP is observed by the ensemble average LSRP of the in-situ phosphocholine functionalised and MHA blocked TSNP

Referring to FIG. 39 (C) which is a UV-vis spectrum of a CRP assay using in-situ phosphocholine functionalised TSNP, chemically blocked using 0.2 μM MHA in the presence of human serum. An LSPR peak wavelength shift response on the presence of CRP is observed by the ensemble average LSRP of the in-situ phosphocholine functionalised TSNP in human sera. (D) is a dose response curve for CRP in the range 0 ng/ml to 250 ng/ml

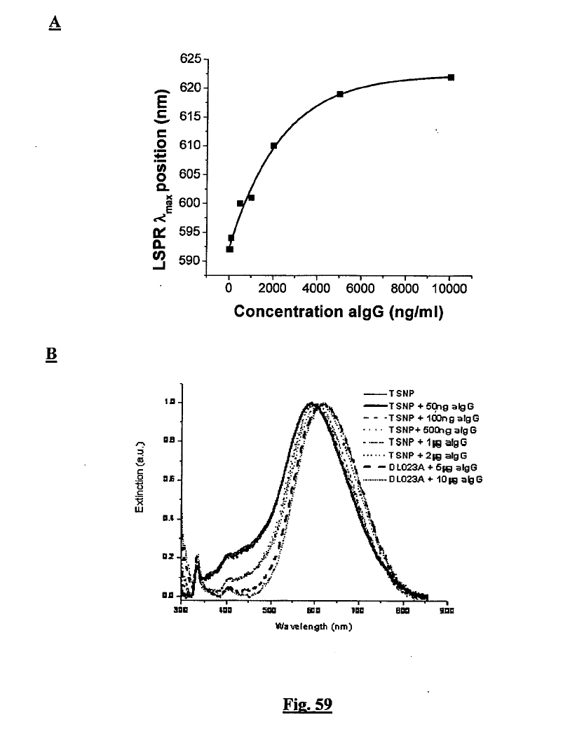

FIG. 57 (A) are Dark Field images of twinned, coupled and grouped TSNP. Note in the case of each group or twin coupled TSNP the entire group or twin appear same colour due to the sharing of the coupled plasmon. FIG. 57 (B) are Dark field images of a group of TSNP moving in solution with Brownian motion.

### (B) Anti-IgG Antibody Detection Assay Using Phosphocholine Functionalised TSNP

Centrifuge IgG functionalised TSNP in 1.5 mL microtubes, at 4° C. for 20 minutes at 18,500 g. Remove supernatant and resuspend in 10% of initial volume, in water (15.5 μΩ grade ELGA system or HPLC grade, Sigma Aldrich), keeping on ice. Prepare fresh dilutions of anti-IgG analyte (100 ng/uL) in water (Sigma Aldrich), keep on ice.

In a black 96 well plate, flat, transparent bottom, the solutions were aliqoted as follows:
1. Variably amount of analyte (0, ng and from 100 ng to 5 μg per well)
2. make up to a total volume of 290 μL in water (sigma)
3. Add 10 μL of biosensor.

The UV-Vis spectra were read. Referring to FIG. 59; which is a series of UV-vis spectra of in situ IgG functionalised TSNP in response to concentrations of aIgG in the range 0 to 10 μg/ml, b) a is an aIgG Assay response curve using in-situ IgG antibody functionalised TSNP.

### Example 10

### Individually Identifiable In Situ Receptor Functionalised TSNP Assays

Picolitre to microlitre drops of assay solutions prepared in Example 9 were drop-cast onto glass slides and examined under a darkfield microscope spectroscopy system at a range of magnifications (×10, ×40 and ×100) according to the following steps.
1. Drop 5 μl (a lower volume would be preferable) of each sample into the sample's designated space
2. Put on number 1 cover slip and turn the slide around
3. Put on sufficient dividers to create a small well to hold sufficient water for lens to make contact with.
4. Air dust and wipe sample with lens tissue before placing on microscope stage cover slip side down
5. Observe TSNP and TSNP sensors and TSNP sensors in the presence of analyte and record images using colour camera
6. Take spectra of individual TSPN and TSNP sensors and TSNP sensors in the presence of analyte using a grating of suitable ruling and blaze such as 300 g/mm blazed at 500 nm according to the following steps:
   Use eyepiece on microscope to align the particle roughly within the spectrograph's imaging region
   In the program select: Spectrograph→Move→Select settings: 1200 Mirror+Move to 0
   When mirror has aligned, select: Acquisition→Experimental set up→ROI set up→Imaging Mode→Use full chip→ok
   Press Focus
   While the images are being taken, open the slit wide enough to locate and identify the particle in question
   Move the particle to the vertical centre of the slit and close the slit until it touches the edges of the particle
   Zoom in on the particle
Take Spectra:
   Select: Acquisition→Experimental set up→ROI Set up
   Highlight the particle with the mouse on the screen
   Click mouse selection→Select start λ=1, end λ=1024→Store
   Adjust the start position and the height of the selected area until the lines surround the particle→Store
   Press ok
   Select: Spectrograph→Move→300 BLZ=500 nm+move to 600 nm→Ok
Repeating for next particle

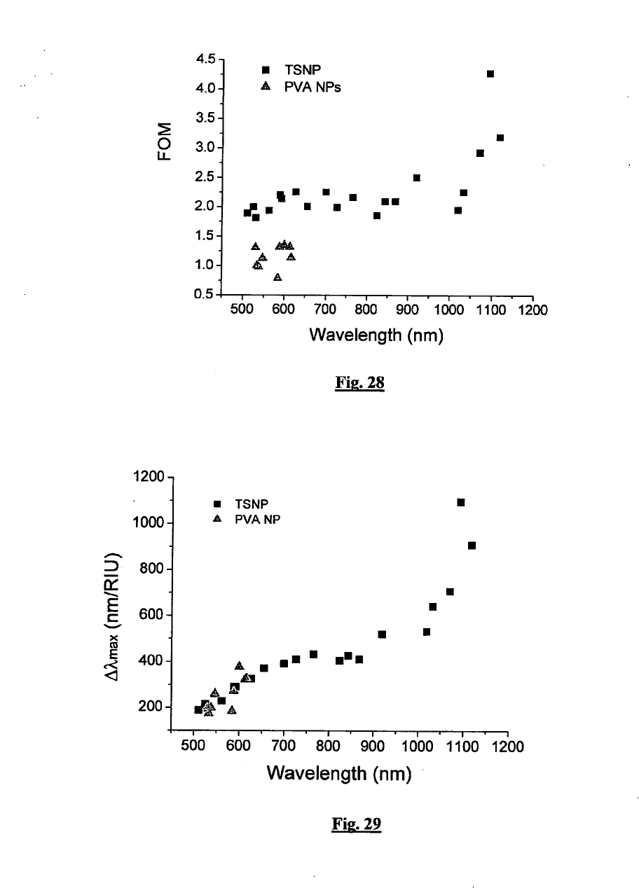

In the case of CRP detection assay using phosphocholine functionalised TSNP an average shift of 38 nm is found for the presence of 100 ng/ml C-reactive protein as shown in FIG. 28.

This method may be used to give a quantitative measure of the amount of analyte present in the sample.

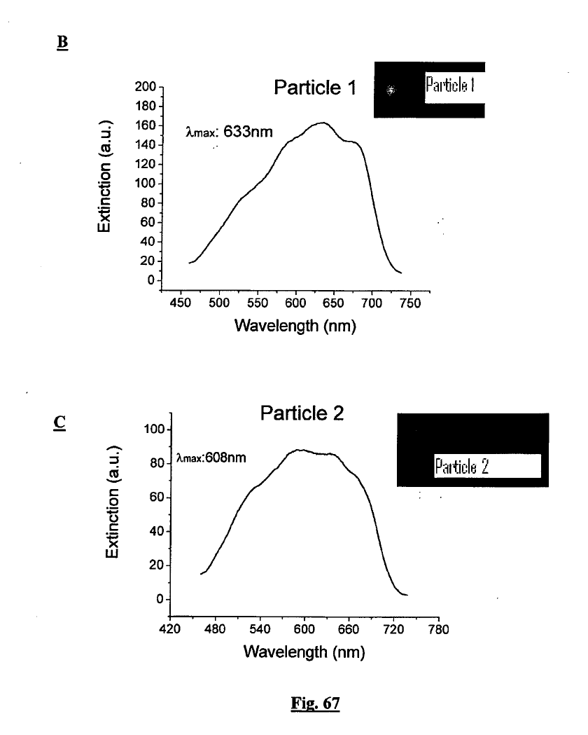

Referring to FIG. 67 which are Dark field images of a) individual and grouped C-reactive protein receptor in situ functionalised TSNP without the presence of C-reactive protein, b) Spectra of an individual C-reactive protein receptor in situ functionalised TSNP without the presence of C-reactive protein c) Spectra of another individual C-reactive protein receptor in situ functionalised TSNP without the presence of C-reactive protein.

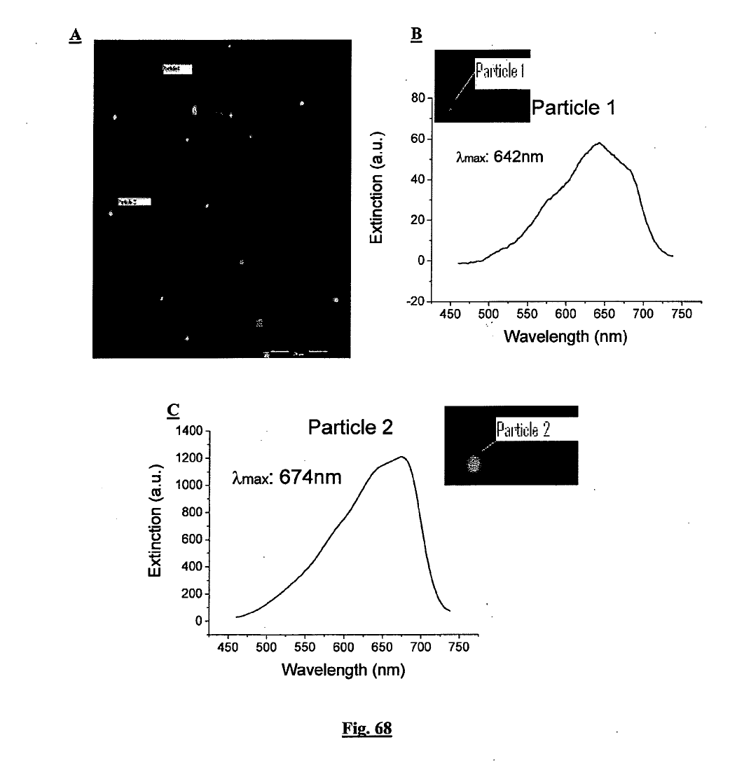

Referring to FIG. 68 which are Dark field images of a) individual and grouped C-reactive protein receptor in situ functionalised TSNP in the presence of 100 ng/ml C-reactive protein, b) Spectra of an individual C-reactive protein receptor in situ functionalised TSNP in the presence of 100 ng/ml C-reactive protein c) Spectra of another individual C-reactive protein receptor in situ functionalised TSNP in the presence of 100 ng/ml C-reactive protein An average shift of 38 nm is found for the TSNP CRP sensor in the presence of 100 ng/ml C-reactive protein.

### Example 11

### DNA Detection Assay Using Oligonucleotide Functionalised TSNP Using a Capture Immobilisation Format

Oligonucleotide functionalised TSNP are centrifuged at 4° C. for 20 minutes at 18,000 g. TSNP are resuspended in RNase/DNase free water and re-centrifuged under same conditions. Resuspend in 10% of initial volume, in RNase/DNase free water and held at 4° C.

Target antisense DNA functionalised with a biotin group is incubated on a streptavidin spotted segregated glass slide, for 4 hours in 0.1M phosphate buffer (PB) at 37° C. After which the slide is washed 3 times in 0.01M PB. The slide is then incubated with functionalised TSNP (a) with complimentary sense, and (b) unfunctionalised (as negative control) in 0.005M PB overnight in a hybridisation oven at 42° C. After which the slide is washed 3 times in 0.005M PB. Individual oligonucleotide spottings are then examined under dark-field microscopy according to the method described in Example 10 above.

Analysis of the spectral response such as LSPR wavelength shift of increased brightness or a combination or image profile may be used to give a quantitative measure of the target oligonucleotide.

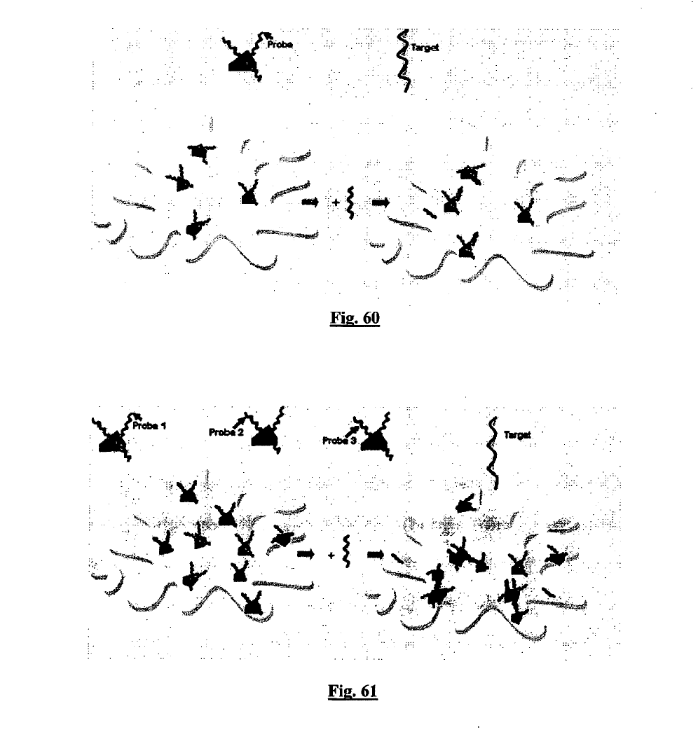

Referring to FIG. 58 which shows dark field images of a) individual in-situ probe functionalised TSNP, b) individual probe in-situ functionalised TSNP and negative target coated substrate and c) individual in-situ probe functionalised TSNP and positive target coated substrate.

### Example 12

### Assay Induced Enhanced Brightness and or Spectral Changes

In assays where the addition of an analyte changes such as increases the brightness of the TSNP sensors, images of the TSNP sensors with and without the presence of the analyte captured under the same luminosity conditions can be analysed using imaging software and the induced brightness and or colour changes may be determined as a quantitative measure of the amount of analyte present.

In the case of DNA detection assay using Oligonucleotide functionalised TSNP using a capture immobilisation format darkfield images of (a) probe functionalised TSNP and (b) probe functionalised TSNP and negative target coated substrate are significantly less bright than (c) probe functionalised TSNP and positive target coated substrate. There is also a significant spectral change comparing (a) and (b) with (c) which appears a distinctly a bright blue-green in colour. This may be used to give a quantitative measure of the target nucleotide.

### Example 13

### Total Solution Phase Individual Nanoplate Measurements

For the total solution phase nanoplate measurements, random TSNP immobilised on a slide were selected and aligned with the spectrometer slit and slit height. The position of the TSNP in the microscope field of view was noted and the spectrometer was set to setting s via spectrometer protocol.

An isolated TSNP moving in solution via Brownian motion was selected and this moving particle was aligned to the region in the microscope eyepiece where the immobilised TSNP was located. The spectrometer was focused and measurements were taken continuously within the selected region for a given time period. When the nanoplate moves into the selected region an increase in the intensity of the spectrum is recorded, a take spectrum is taken at this point.

Between 4 and 5 spectra were taken using this method for each solution phase TSNP being measured. A background spectrum is taken when the TSNP has left the selected region and the intensity has reduced again.

An example of spectral measurements of individual total solution phase TSNP moving in Brownian motion is shown in FIG. 70.

### Example 14

### Darkfield

Darkfield microscopy describes microscopy methods which exclude the unscattered light from the source beam from the image. The field around the specimen (i.e. where there is no specimen to scatter the beam) is therefore generally dark. Darkfield spectroscopy refers to measuring the optical spectrum under darkfield conditions where only scattered light is detected. This compares to UV-visible-NIR optical extinction where the absorption and scattering of light transmitted through a sample is measured.

In one embodiment of the invention it is useful to be able to compare the LSPR spectrum before and after a binding event and the degree of spectra shift provides a measurement of the quantity of the binding and corresponds to the amount of analyte present. Therefore representative before and after binding spectra which can be calibrated to provide standard binding concentration curves are required.

The potential sensitivity using a single nanoparticle is of the order of zeptamoles. However no matter how tight the size and shape distribution within a nanoparticle sample, one nanoparticle is not representative of the spectrum or the spectral sensitivity of a sample and therefore calibration to form a useable sensor is very difficult.

It would be more useful therefore to carry out sensing using low numbers of nanoparticles which provide a representative and reliable spectrum and LSPR refractive index sensitivity which may be calibrated for use as a quantitative sensor capable of measuring ultra high senstivities.

Measuring in solution phase is the most favourable phase for optimal binding kinetics facilitating increased sensing speed and sensitivity. Therefore solution phase measurements of a low number of nanoparticles which provide a representative spectrum and spectral response which can calibrated to provide a quantitative analyte detection at sensitivities orders of magnitude better that what can be achieved on using larger volumes of nanoparticles such as optical extinction measurements carried out using conventional UV-Vis spectroscopy. This can be achieved using dark field for example at high magnification such as 100× where nanoparticle number from single to ensembles containing of the order of 1 million nanoparticles.

The spectra obtained in such a fashion have a narrower fwhm signifying the reduce emsembled averaging effect one gets when carrying out UV-Vis spectroscopy where of the order of 10¹¹ nanoparticles are measured simultaneously. The spectra obtained by darkfield also show the LSPR responsivity as in the case of UV-Vis measurements.

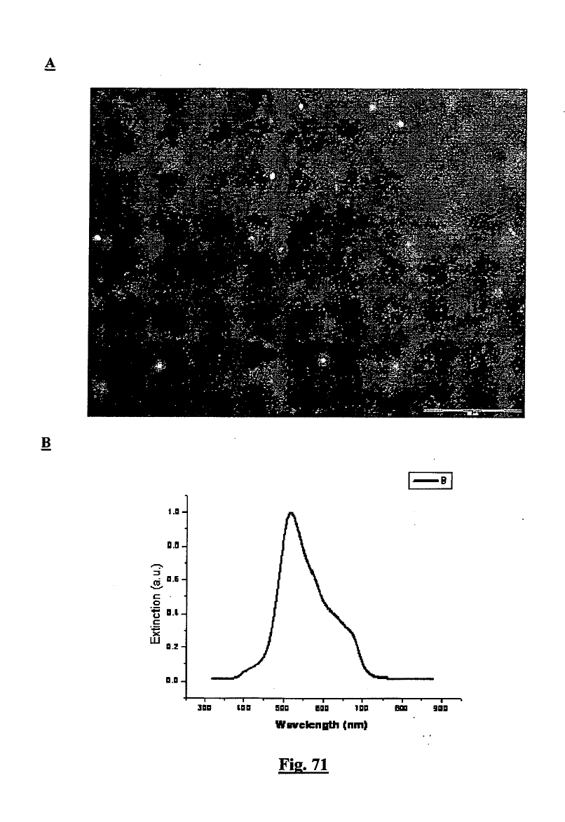

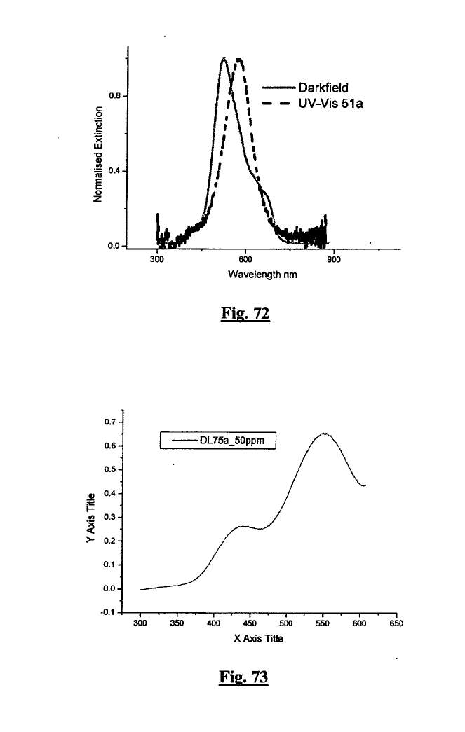

A Darkfield image at 100× magnification and is the corresponding dark field scattering spectrum of an ensemble collection of circa 5000 nanoparticles solution phase of TSNP moving freely in solution is shown in FIGS. 71(A) and (B). A Darkfield scattering spectrum of an ensemble collection of solution phase of TSNP moving freely in solution at 100× magnification and corresponding UV-Vis spectrum of nanoplates using a 1 cm path length is shown in FIG. 72. The difference in to location of the LSPR peak position between the UV-Vis spectrum measured for an ensemble collection of the order of 5×10¹¹ nanoparticles and the darkfield scattering spectrum for an ensemble collection of the order of 5×10³ nanoparticle occurs at two different wavelengths for the same TSNP sample.

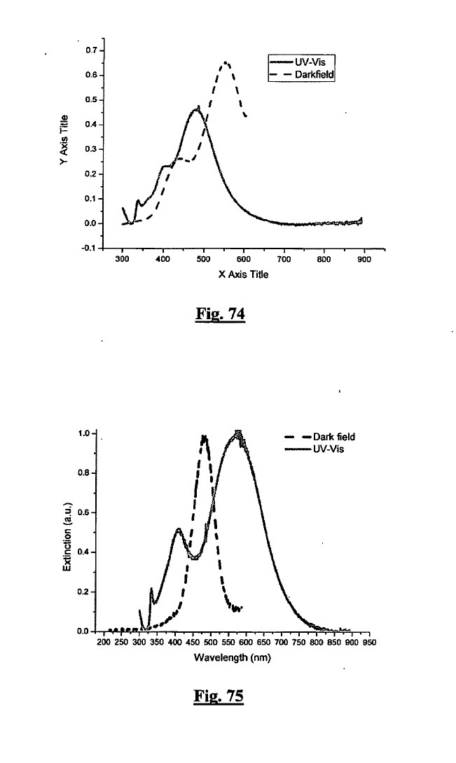

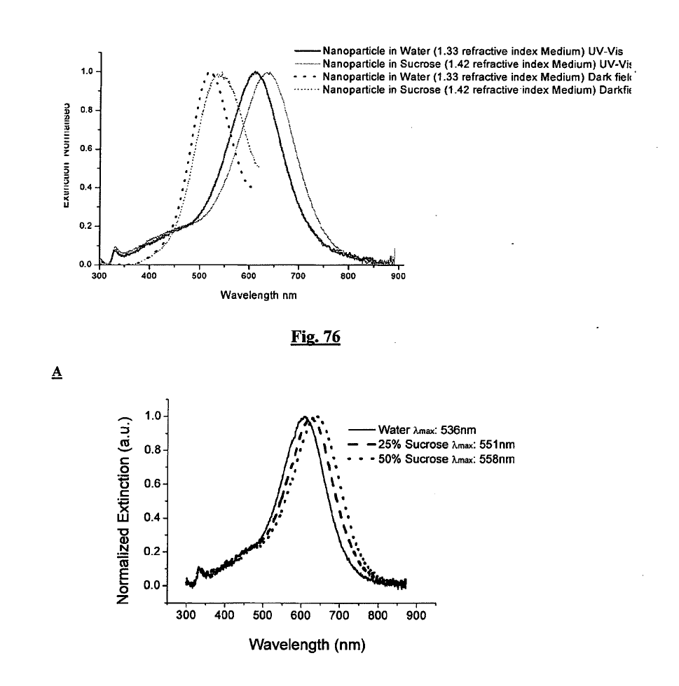

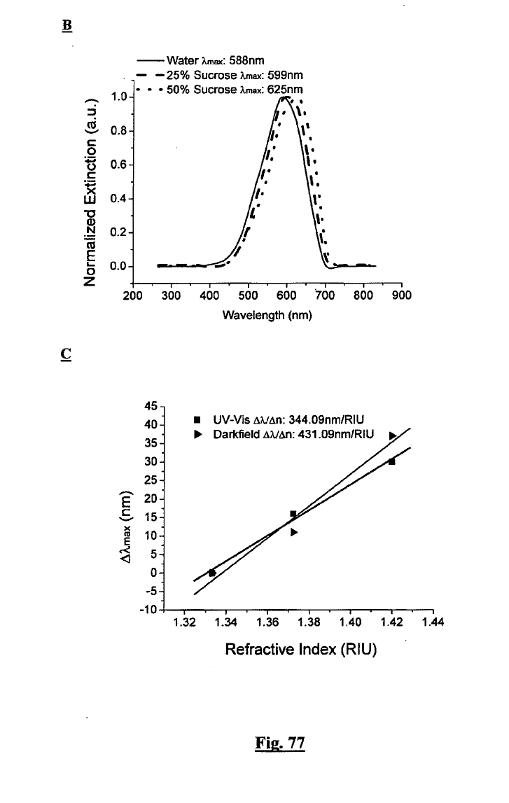

In FIG. 73 a Darkfield scattering spectrum of another collection of solution phase of TSNP have two LSPR peaks moving freely in solution is shown at 100× magnification. In FIG. 74 a Darkfield scattering spectrum of this collection of solution phase TSNP moving freely in solution and corresponding UV-Vis spectrum of nanoplates using a 1 cm path length are shown. Both the darkfield scattering and UV-Vis spectra show double peaks which are located at different spectral positions. In FIG. 75 the Darkfield scattering spectrum at 100× magnification of another collection of solution phase TSNP moving freely in solution which has a double corresponding UV-Vis spectrum of nanoplates using a 1 cm path length shows only one peak, which is due to the fact that the grating used the in the darkfield spectrometer limits the spectral detection in the region the second spectral peak would be expected.

FIG. 76 shows a Darkfield scattering spectrum at 100× magnification of anther collection of solution phase TSNP moving freely in solution in a 1.33 (water) and 1.42 (50% w/v sucrose solution) refractive index medium and corresponding UV-Vis spectrum of nanoplates using a 1 cm path length in a 1.33 (water) and 1.42 (50% w/v sucrose solution) refractive index medium. A significant spectral shift is observed in both the Dark field scattering and UV-Vis measurements. In addition the darkfield scattering spectra show narrower FWHM than in the case of the UV-Vis which will result in a higher figure of merit for the Darkfield scattering measured smaller ensemble collection of TSNP than in the case of the UV-Vis measurements.

FIG. 77(A) shows the UV-Vis extinction spectra for another solution phase ensemble of silver nanoplates in water, 25% sucrose and 50% sucrose, while B shows the darkfield scattering spectra for a collection of circa 5000 of the same silver nanoplates in solution phase. C shows the a linear plot of the peak wavelength shift as a function of refractive index in the case of both the UV-Vis extinction spectra and the darkfield scattering spectra for a collection of circa 5000 of the same silver nanoplates in solution phase.

| | | λₘₐₓ | λₘₐₓ | | | |
|---|---|---|---|---|---|---|
| | λₘₐₓ | 25% | 50% | Δλ/Δn | FWHM | |
| Detection | Water | Sucrose | Sucrose | (nm · RIU⁻¹) | (nm) | FOM |
| UV-Vis | 607 | 623 | 637 | 344.09 | 137.8 | 2.49 |
| Dark. Field. | 588 | 599 | 625 | 431.09 | 131.27 | 3.28 |

A significant wavelength shift is observed for the TSNP both in the case of the dark field scattering spectra and the UV-VIS extinction spectra and a significant increase the FOM is found in the case of the darkfield scattering spectra of the smaller ensemble silver nanoplates over the UV-Vis extinction spectra of the larger ensembled collection of silver nanoplates

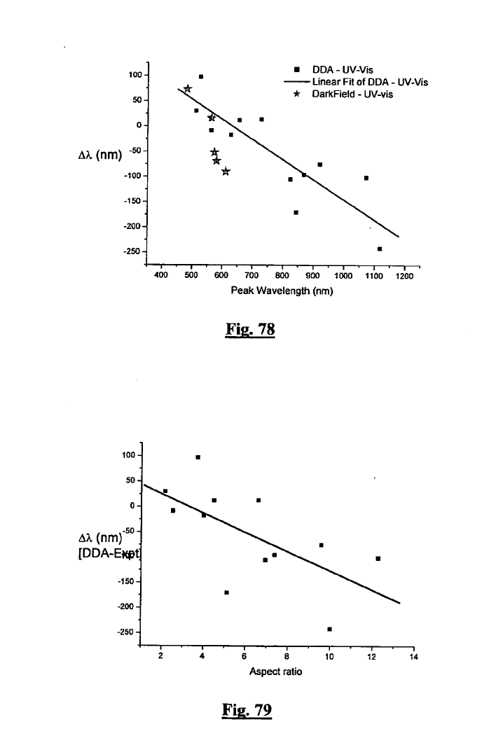

FIG. 78 is a plot showing the difference between the peak wavelength positions of DDA single TSNP calculated and the experimentally measured TSNP ensemble using UV-VIS peak wavelength position (black squares). Difference between the DDA single TSNP calculated and the experimentally measured TSNP ensemble using UV-VIS peak wavelength position (grey stars).

FIG. 79 is a plot showing the difference between the DDA single TSNP calculated and the experimentally measured TSNP ensemble using UV-VIS peak wavelength position (black squares) as a function of TSNP aspect ratio.

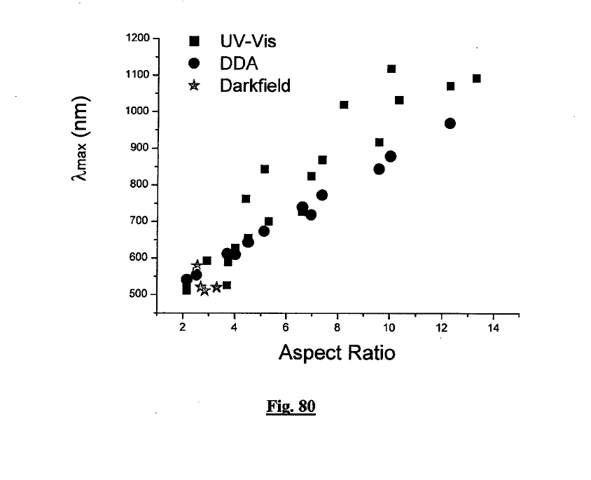

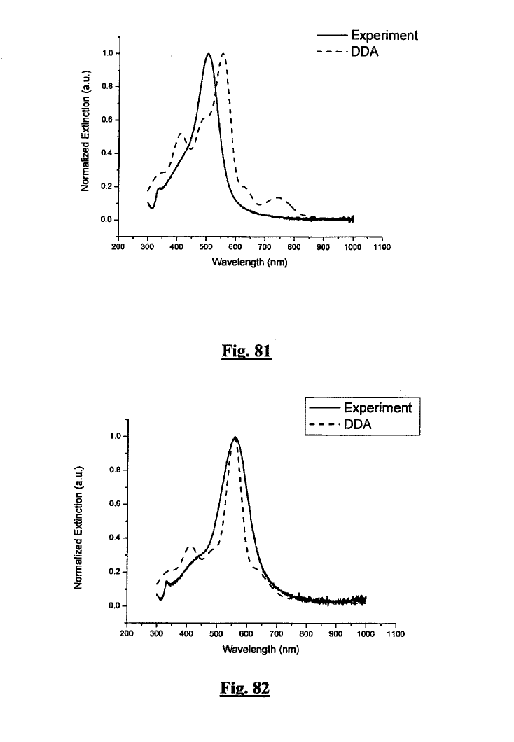

FIG. 80 is a plot showing the peak wavelength positions of nanoparticles measured using UV-Vis with a 1 cm optical path length (black squares) and darkfield (grey stars) and calculated using DDA (black circles).

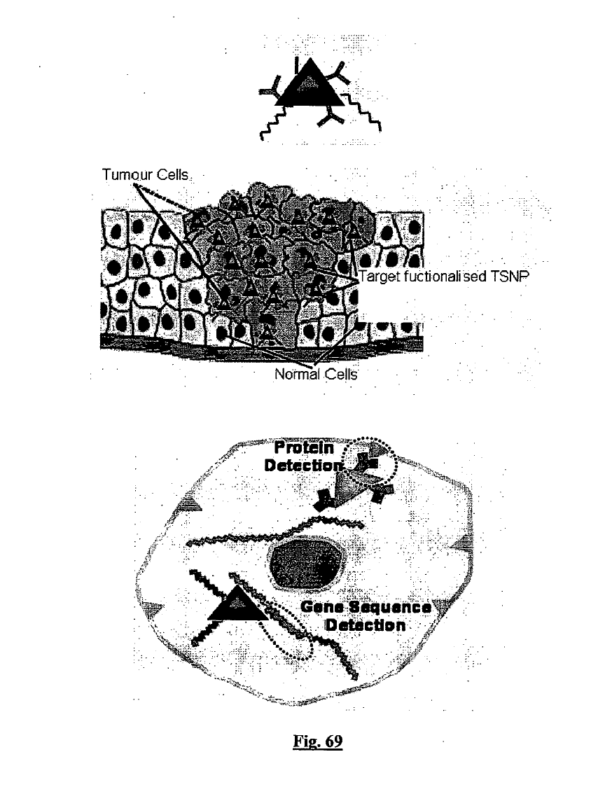

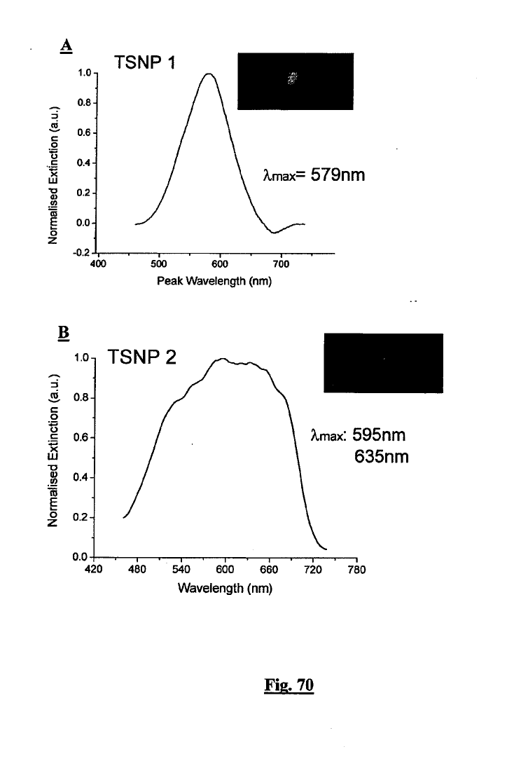

Discrete Dipole Approximations (DDA) were performed using the DDSCAT 7.0 code developed by Draine and Flatau,²³ to calculate the extinction, absorption and scattering spectra of the TSNPs in water. The 12 shapes used for the DDA calculation were based upon the samples in the experimental data, consisting of regular triangular prisms, made up of a simple cubic array of dipoles spaced ˜1 nm apart, as per the DDA method. It must be noted that the regular triangular prism is an approximation of shape measured for the experimental nanoplates. Therefore the key factors considered when calculating the DDA spectra were the aspect ratio and the volume of the nanoplates measured in the experimental studies. FIGS. 69 to 80 show the calculated spectra using DDA and corresponding UV-Vis experimental measurements of spectra for shape 1 to 19 nanoparticles listed in table 6;

TABLE 6
TSNP dimensions used for DDA thickness Analysis and comparison with experimental
parameters

| | | | | | | | | | ΔE |
|---|---|---|---|---|---|---|---|---|---|
| | Edge | | | | | | Δλ (nm) | | [DDA- |
| Shape | Length | Thickness | Aspect | Volume | Effective | λₘₐₓ | [DDA- | Peak E | Expt] |
| No | (nm) | (nm) | Ratio | (nm³) | Radius r* | (nm) | Expt] | (eV) | (eV) |
| 1 | 11.77 | 5.48 | 2.14 | 390.22 | 4.53 | 511.00 | 30.06 | 2.297 | −0.163 |
| 3 | 15.34 | 6.08 | 2.52 | 789.97 | 5.73 | 562.41 | −8.74 | 2.245 | 0.025 |
| 5 | 26.4 | 6.58 | 4.01 | 2254.78 | 8.13 | 627.22 | −17.57 | 2.039 | 0.049 |
| 7 | 49.07 | 7.42 | 6.61 | 9353.71 | 13.07 | 727.24 | 12.8 | 1.679 | −0.031 |
| 8 | 52.56 | 7.56 | 6.95 | 10947.09 | 13.77 | 824.2 | −105.48 | 1.729 | 0.229 |
| 9 | 26.75 | 7.23 | 3.70 | 2652.05 | 8.586 | 525.78 | 97.11 | 2.031 | −0.319 |
| 11 | 35.17 | 7.81 | 4.50 | 5236.76 | 10.77 | 655.07 | 11.95 | 1.933 | 0.043 |
| 13 | 55.02 | 10.74 | 5.12 | 16004.95 | 15.63 | 843.63 | −170.25 | 1.841 | 0.371 |
| 15 | 134.07 | 13.39 | 10.01 | 123949.4 | 30.93 | 1118.41 | −241.42 | 1.417 | 0.307 |
| 16 | 81.82 | 11.09 | 7.38 | 39326.01 | 21.09 | 868.48 | −95.731 | 1.61 | 0.18 |
| 17 | 109.46 | 11.43 | 9.58 | 67173.05 | 25.22 | 919.47 | −75.124 | 1.453 | 0.103 |
| 19 | 172.37 | 14.04 | 12.28 | 201495.4 | 36.37 | 1070.88 | −101.43 | 1.29 | 0.09 |

*Radius of particle if it were a sphere. Calculated from the TSNPs volume

### Example 15

### Assay Configurations

The nanoplate biosensors are highly versatile and may be used in a number of different assay configurations ranging from total solution phase assay configurations to immobilised assay configurations. These assays may be carried out using ultralow volumes in the nanoliter to picoliter range. Exemplary assay configurations are described below.

FIG. 60 is a schematic of total solution phase individual single TSNP assaying. The TSNP sensors/labels may exhibit a spectral response such as a shift, increase or decrease in optical scattering or a combination of these features upon the binding of an analyte molecule. This assay may be in total Solution phase format where the probe functionalised TSNP and the analytes remain in solution phase throughout the detection process. This assay may be carried out where the probe functionalised TSNP may be tethered or immobilised on a substrate or the analyte may be immobilised on a substrate. This assay may be carried out in a multiplex format wherein further different probe functionalised TSNP are employed, each having a distinct and different LSPR peak wavelength for each corresponding probe. A combination of image analysis and or spectral change analysis may provide a quantitative basis of the assay signal.

FIG. 61 is a schematic of an assay configuration involving TSNP functionalised with 3 different probes Probe 1 identifies and quantifies the target; Probe 2 recognises allele 1 (wild type); probe 3 recognises allele 2 (mutant). The TSNP sensors/labels may exhibit a spectral response such as a shift, increase or decrease in optical scattering or a combination of these features upon the binding of an analyte molecule. This change in the optical spectrum may be shared by all of the bound probe functionalised TSNP to a single analyte in that a uniform spectral profile may be exhibited by each of the TSNP in the bound group due to plasmon coupling. This configuration has potential for SNP typing. This assay may be in total solution phase format wherein each of the probe functionalised TSNP and the analytes remain in solution phase throughout the detection process. This assay may be carried out wherein one or more of the probe functionalised TSNP may be tethered or immobilised on a substrate or the analyte may be immobilised on a substrate. Twinned of grouped functionalised TSNP may be used which may serve to increase the scattering cross section and or LSPR sensitivity which would enable easier image of spectral detection and analysis. This assay may be carried out in a multiplex format wherein further different probe functionalised TSNP are employed, each having a distinct and different LSPR peak wavelength for each corresponding probe. A combination of image analysis and or spectral change analysis may provide a quantitative basis of the assay signal.

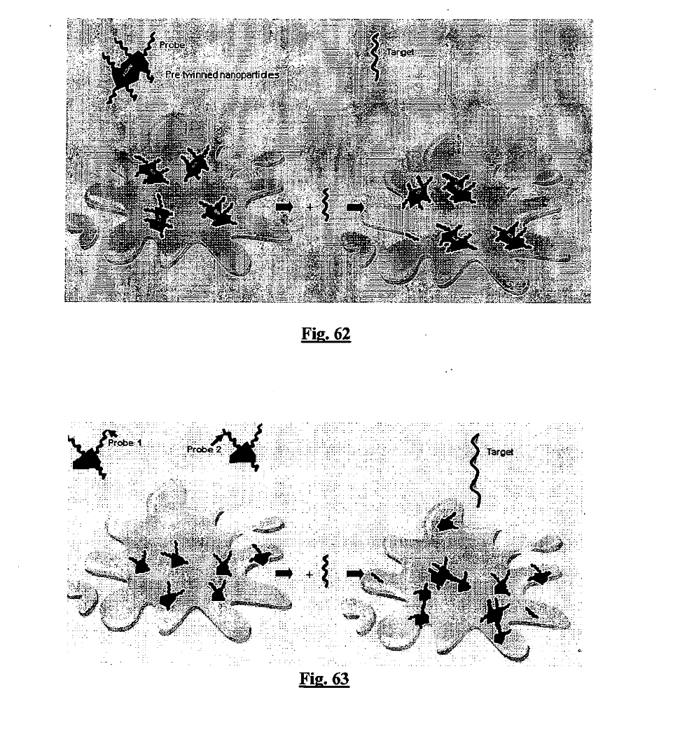

FIG. 62 is a schematic of twinned or pregrouped probe functionalised TSNP are used which may facilitate increased LSPR sensitivity and or enable increased optical extinction cross section than in the case of single probe functionalised TSNP. The twinned or pre grouped TSNP may exhibit a uniform spectral profile due to plasmon coupling. The twinned or pre-grouped TSNP sensors/labels may exhibit a spectral response such as a shift, increase or decrease in optical scattering or a combination of these features upon the binding of an analyte molecule. This change in the optical spectrum may be shared by all of the bound probe functionalised TSNP to a single analyte in that a uniform spectral profile may be exhibited by each of the TSNP in the bound group due to plasmon coupling. This assay may be in total solution phase format wherein each of the twinned or pre-grouped probe functionalised TSNP and the analytes remain in solution phase throughout the detection process. This assay may be carried out where one or more of the twinned or pre-grouped probe functionalised TSNP may be tethered or immobilised on a substrate or the analyte may be immobilised on a substrate. This assay may be carried out in a multiplex format wherein two or more further different twinned or pre-grouped probe functionalised TSNP are employed, each have a distinct and different LSPR peak wavelength for each corresponding probe. A combination of image analysis and or spectral change analysis may provide a quantitative basis of the assay signal.

FIG. 63 is a schematic of an assay configuration involving dual probe functionalised TSNP. Probe 1 is for target identification e.g. the presence or absence of analyte; Probe 2 acts to further characterise the analyte e.g. a subtyping of the analyte such as in the case of bacterial or protein isotyping. This combination of probes will also permit melting curve analysis for the determination of polymorphic DNA. The TSNP sensors/labels may exhibit a spectral response such as a shift, increase or decrease in optical scattering or a combination of these features upon the binding of an analyte molecule. This change in the optical spectrum may be shared by all of the bound probe functionalised TSNP to a single analyte in that a uniform spectral profile may be exhibited by each of the TSNP in the bound group due to plasmon coupling. Twinned or grouped functionalised TSNP may be used which may serve to increase the scattering cross section and or LSPR sensitivity which would enable easier image of spectral detection and analysis. This assay may be in total solution phase format wherein each of the probe functionalised TSNP and the analytes remain in solution phase throughout the detection process. This assay may be carried out wherein one or more of the probe functionalised TSNP may be tethered or immobilised on a substrate or the analyte may be immobilised on a substrate. This assay may be carried out in a multiplex format wherein two or more further different probe functionalised TSNP are employed, each have a distinct and different LSPR peak wavelength for each corresponding probe. A combination of image analysis and or spectral change analysis may provide a quantitative basis of the assay signal.

FIG. 64 is a schematic of the capturing and tethering or immobilising of probe functionalised TSNP sensors on the binding of target analyte with the solution phase TSNP sensors and substrate immobilised probes. The TSNP sensors/labels may exhibit a spectral response such as a shift, increase or decrease in optical scattering or a combination of these features upon the binding of an analyte molecule. Twinned or grouped functionalised TSNP may be used which may serve to increase the scattering cross section and or LSPR sensitivity which would enable easier image of spectral detection and analysis. This assay may be carried out in a multiplex format wherein two or more further different probe functionalised TSNP are employed, each have a distinct and different LSPR peak wavelength for each corresponding probe. A combination of image analysis and or spectral change analysis may provide a quantitative basis of the assay signal.

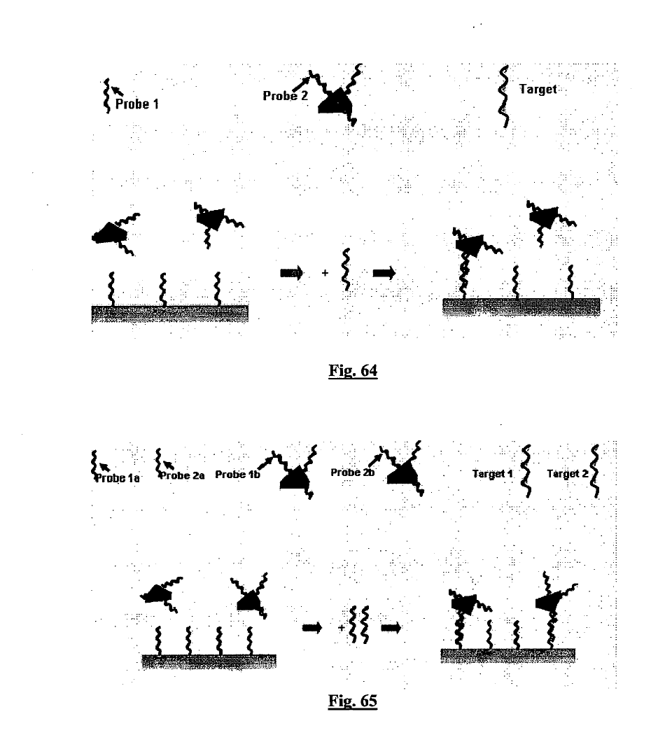

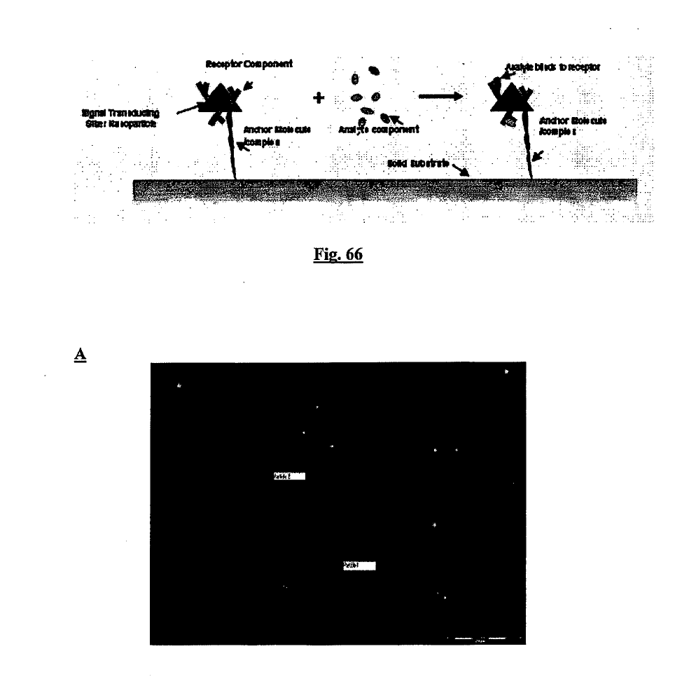

FIG. 65 is a schematic of multiplex TNSP sensors wherein two or more different probe functionalised TSNP, each have a distinct and different LSPR peak wavelength for each corresponding probe, Probe functionalised TSNP sensors are captured and tethered or immobilised on the binding of target analyte with the solution phase TSNP sensors and substrate immobilised probes. The TSNP sensors/labels may exhibit a spectral response such as a shift, increase or decrease in optical scattering or a combination of these features upon the binding of an analyte molecule. Distinctly different spectral responses may be measured for different probe functionalised TSNP sensors. Twinned or grouped functionalised TSNP may be used which may serve to increase the scattering cross section and or LSPR sensitivity which would enable easier image of spectral detection and analysis. A combination of image analysis and or spectral change analysis may provide a quantitative basis of the assay signal. A combination of image analysis and or spectral change analysis may provide a quantitative basis of the assay signal.

The probe may be a ligand, a protein, or a nucleic acid. The probe may by mono-species, di-species, or multi-species. Target analytes may be a protein, a nucleic acid, a bacterium or a viral body. Images may be captured using an optical reader such as a dark field microscope system. Spectral changes due to LSPR wavelengths shifts may be measured or image analysis which determines features such as brightness colour etc may be used to provide a quantitative signal Assaying using one or more individually identifiable TSNP, twinned or grouped TSNP

### Tethered Nanoparticle Configuration

We envisage nanoparticles, pre-coated or in situ functionalised with recognition molecules or receptors as the sensors. We envisage that in one embodiment these sensors may be “tethered” or anchored to a solid substrate by one or more anchor or tether molecules, which would be located among the receptor molecules and “tie” the sensor either directly or indirectly (through the formation of a complex with other molecules(s) or particle(s)) to the solid substrate. In this fashion these sensors maintain the feature that substantially all of the surfaces are available for interaction as shown in FIG. 66.

FIG. 66 is a schematic of a tethered probe arrangement wherein substantially all of the probe functionalised TSNP surfaces are available for binding. The TSNP sensors/labels may exhibit a spectral response such as a shift, increase or decrease in optical scattering or a combination of these features upon the binding of an analyte molecule. Distinctly different spectral responses may be measured for different probe functionalised TSNP sensors. Twinned or grouped functionalised TSNP may be used which may serve to increase the scattering cross section and or LSPR sensitivity which would enable easier image of spectral detection and analysis. This assay may be carried out in a multiplex format wherein two or more further different probe functionalised TSNP are employed, each have a distinct and different LSPR peak wavelength for each corresponding probe.

These anchor molecules/complexes may for part of “spacer” molecules which are often required in these types of configurations to avoid or reduce steric hindrance of the receptor components.

The TSNP sensors/labels may exhibit a spectral response such as a shift, increase or decrease in optical scattering or a combination of these features upon the binding of an analyte molecule

In this configuration the TSNP sensors may be tethered or embedded in a membrane with monitor a passing or surrounding fluid to which a target analyte binds if present in the fluid.

In addition to darkfield, confocal and TEM microscopies, spectrocopies ranging from fluorescence correlation spectroscopy (FCS) to stimulated emission depletion (STED), which enables subwavelength spatial resolution, can be used to read the assay configurations and provide means to provide TSNP facilitated detailed detection information including single molecule information.

### Nucleic Acid Detection

### Example

Three target sequences were used comprising 20 base pair oligonucleotides including one positive sequence (SEQ ID No. 1) and two negative sequences (SEQ ID No. 2 and SEQ ID No. 3) as follows;

| (SEQ ID No. 1) | |
|---|---|
| Positive Target: | TAG CCA TTT ATG GCG AAC CA |
| (SEQ ID No. 2) | |
| Negative Target 1: | CCC CAA GTC CTT GTG GCT TG |
| (SEQ ID No. 3) | |
| Negative Target 2: | TGG TTC GCC ATA AAT GGC TA |

SEQ ID No. 1 to 3 were immobilised on glass slides using a standard plotting method to form a nucleic acid array with individual spots of approximately 200 μm in diameter at concentrations of 20 μM, 2 μM, 200 nM, 20 nM and 2 nM.

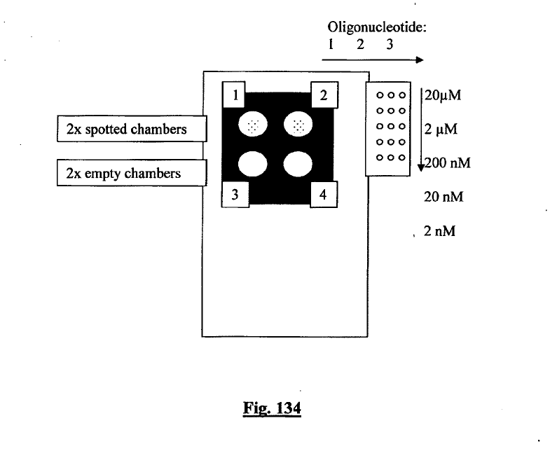

FIG. 134 is a schematic of a slide containing hybridisation chambers and a nucleic acid array. Oligonucleotide 1=SEQ ID No. 1 (positive nucleic acid Target) which is complementary to probe sequences functionalised on TSNP. Oligonucleotide 2 (SEQ ID No. 2) and 3 (SEQ ID No. 3) are negative controls. The spot diameter is approximately 200 μm and the hybridisation chamber volume is about 40 μl.

Probes included bare TSNP which were not functionalised with any nucleic acid sequences and TSNP functionalised with oligonucleotide sequence which were complementary to SEQ ID No. 1. It will be understood that by complementary we mean an oligonucleotide that binds to SEQ ID No. 1 in accordance with Watson-Crick binding i.e. G binds to C and A binds to T. The complimentary oligonucleotide sequence is as follows:

Complementary sequence:
(SEQ ID No. 4)
ATC GGT AAA TAC CGC TTG GT

The oligonucleotide sequences were modified with different end group chemistries at the 5′ end as follows: (i)No end group chemistry (unmodified sequence), (ii) DAPA, (iii) IDEA, (iv)Thiol and (v) Thiol A20. The modified and unmodified oligonucleotide sequences were used to functionalise the TSNPs. As an exemplary example the following oligonucleotides were used to fuctionalise the TSNPs:

(i) No end group chemistry:
(SEQ ID No. 3)
TGG TTC GCC ATA AAT GGC TA
(ii) DAPA:
(DAPA modified SEQ ID No. 3)
(DAPA)₄-4MOXT-TGG TTC GCC ATA AAT GGC TA

The end group chemistry for the DAPA modified nucleic acid sequence is four tertiary amino groups at the 5′-end with Spacer 9 (9 atoms) from Glen Research. The DAPA configuration is shown below

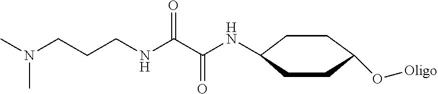

(iii) IDEA:
(IDEA modified SEQ ID No. 3)
(IDEA)₄-4MOXT-TGG TTC GCC ATA AAT GGC TA

The end group chemistry for the IDEA modified nucleic acid sequence is eight secondary amino groups at the 5′-end Spacer 9 (9 atoms) from Glen Research The IDEA configuration is shown below

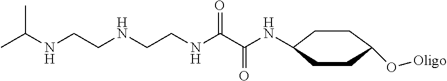

(iv)Thiol:
(Thiol modified SEQ ID No. 1)
THI-TAG CCA TTT ATG GCG AAC CA
(v) ThiolA20:
(SEQ ID No. 5
THI-AAA AAA AAA AAA AAA AAA AAA TAG CCA TTT ATG
GCG ACC A

—thiol modified SEQ ID No. 1 in which an additional 20 adenosine bases are added to the 5′ end of SEQ ID No. 1).

As a control, unfunctionalised TSNPs (TNSP Bare) were used. In addition, further controls for non-specific binding and background binding (unspotted chambers containing no target nucleic acids) were used.

### Assay Preparation.

10 uL of functionalised TSNP sensors and unfunctionalised TSNP were diluted in 90 uL of RNAse/DNAse and free phosphate buffer (Mono-di basic mix, 10 mM, pH=7.4)

The functionalised TSNP sensors and unfunctionalised TSNP and were incubated in denaturing conditions of 96° C. for 2 minutes, then placed on ice for a few minutes.

42 uL of functionalised TSNP sensors and unfunctionalised TSNP were distributed in each hybridisation chamber containing the spotted immobilized positive and negative target sequences at a range of concentrations as described above and the control hybridisation chamber containing no spotting and no nucleic acid sequences.

Hybridisation was carried out for 3 hours at 56° C.

Then two washes in phosphate buffer were performed to rinse off unbound functionalised TSNP sensors and unbound unfunctionalised TSNP.

A final wash was carried out to preserve samples, and slides were kept in the dark at 4° C. until examination.

Darkfield images and spectral profiles of the chambers and spotted arrays containing the in solution phase captured and tethered TSNP sensors on the binding with complementary target nucleic acids immobilised on a substrate were recorded as described above and analysed.

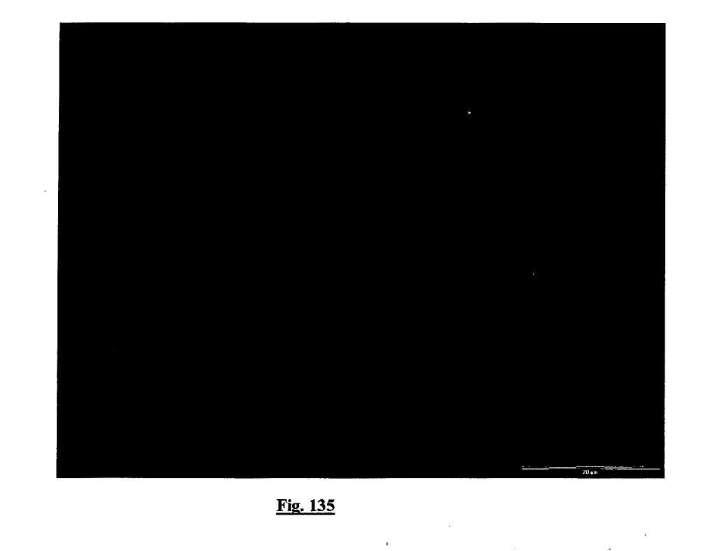

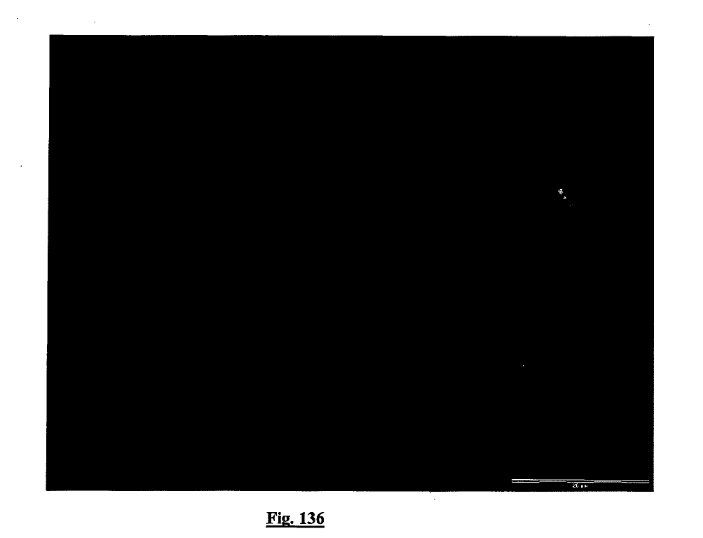

FIG. 135 shows a dark field image taken at a magnification of 100× of unfunctionalised TSNP on a spot containing immobilized positive target nucleic acid at a concentration of 20 μM. This image confirms negative unspecific binding of bare unfunctionalised TSNP with nucleic acid sequences and a very low background binding signal.

FIG. 136 shows a dark field image as representative of TSNP functionalized with SEQ ID No. 4 oligonucleotides which are complementary with the immobilized positive target sequence (SEQ ID No. 1). Specifically this case shows a dark field image taken at a magnification of 100× of thiol functionalised TSNP on a spot containing immobilized positive target nucleic acid at a concentration of 20 μM. This image confirms very low unspecific binding of functionalised TSNP with nucleic acid sequences and a very low background binding signal. Note that the one TSNP observable in the image is a group.

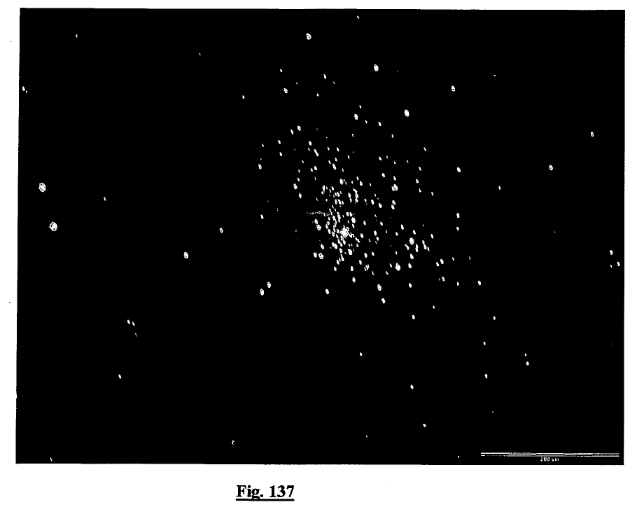

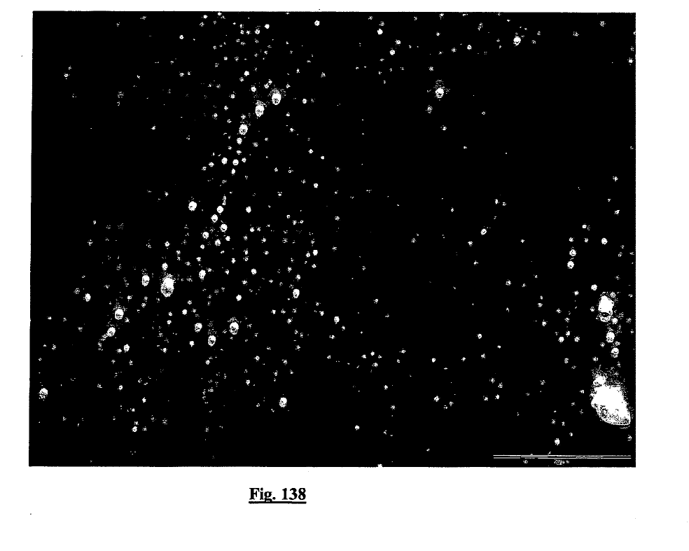

FIG. 137 shows a dark field image taken at a magnification of 10× of DAPA functionalised TSNP on a spot containing immobilized positive target nucleic acid (SEQ ID No. 1). This image confirms high binding of DAPA functionalised TSNP with complementary nucleic acid sequences (SEQ ID No. 4).

FIG. 138 shows a dark field image taken at a magnification of 100× of DAPA functionalised TSNP on a spot containing immobilized positive target nucleic acid (SEQ ID No. 1). This image confirms high binding of DAPA functionalised TSNP with complementary nucleic acid sequences (SEQ ID No. 4).

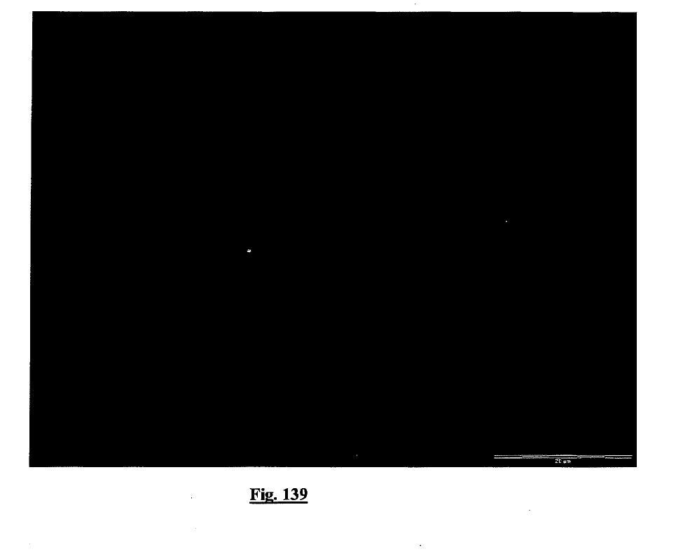

FIG. 139 shows a dark field image taken at a magnification of 100× of DAPA functionalised TSNP in a position between spots containing immobilized positive target nucleic acid (SEQ ID No. 1). This image confirms the very low unspecific binding of DAPA functionalised TSNP and very low background non-specific binding signal.

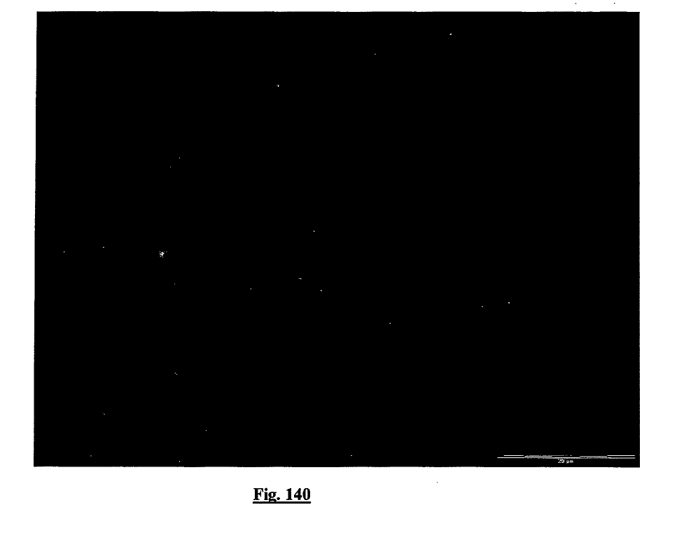

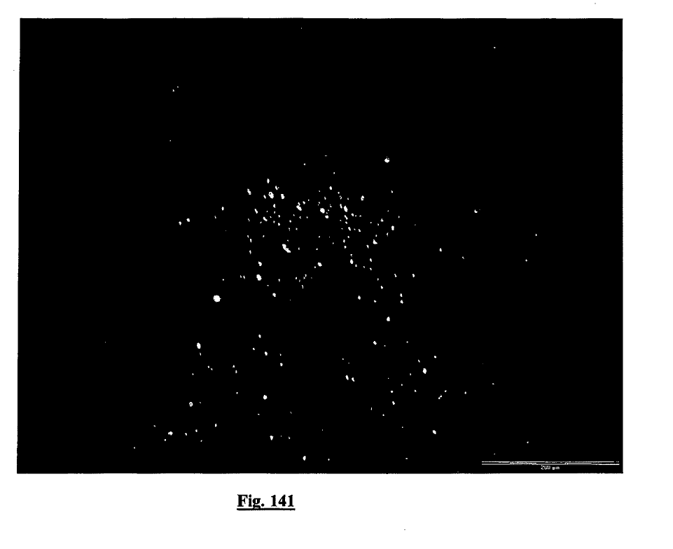

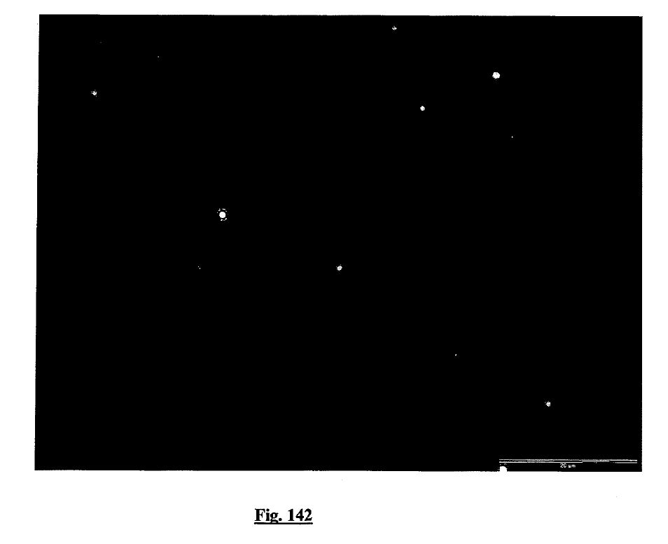

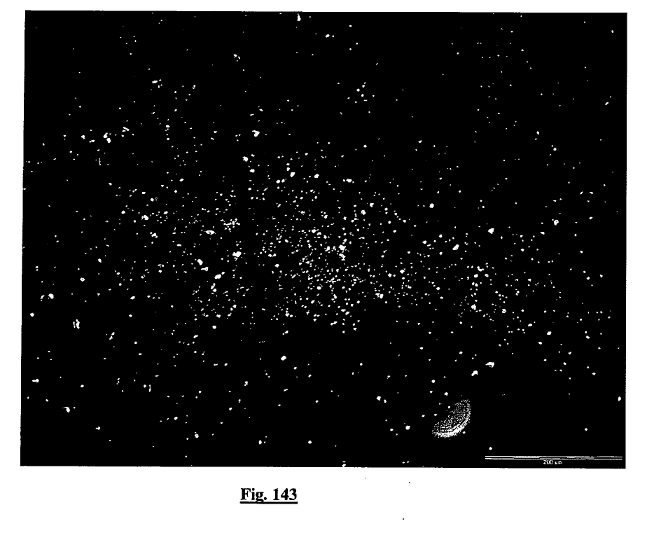

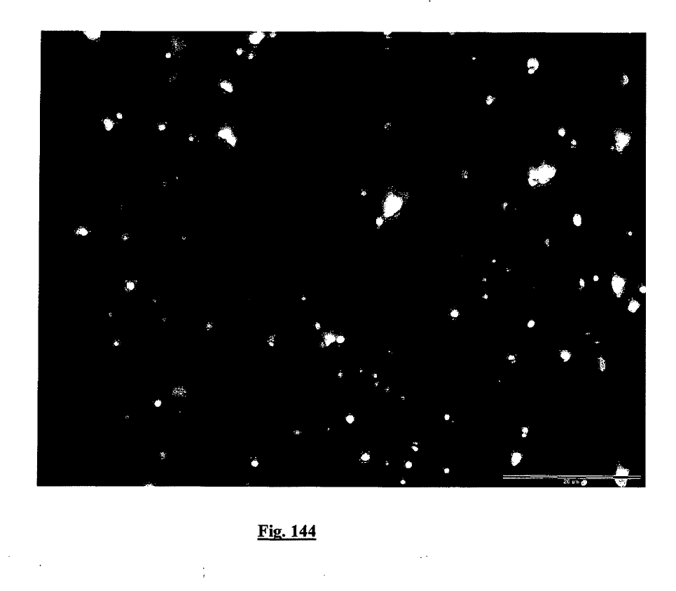

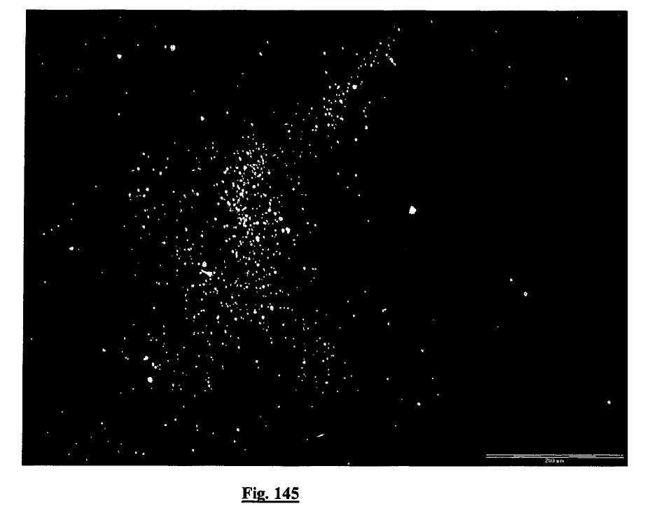

FIG. 140 shows a dark field image taken at a magnification of 100× of no end group chemistry functionalised TSNP on a spot containing immobilized positive target nucleic acid (SEQ ID No. 1). This image confirms the efficient binding of TSNP functionalised with complementary oligonucleotides (SEQ ID No. 4) with out any additional end group chemistry with complementary nucleic acid target sequences

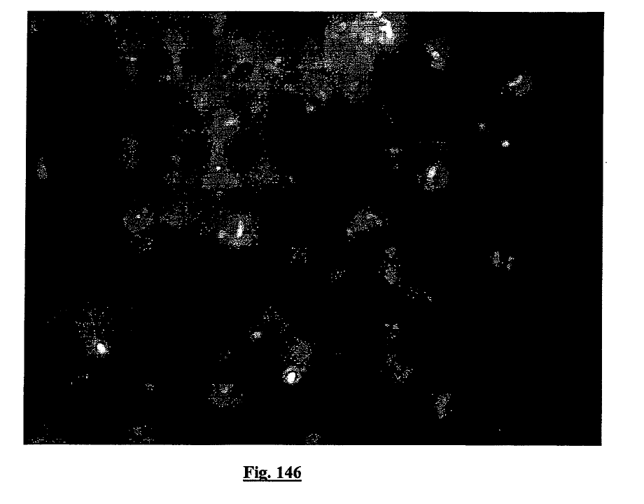

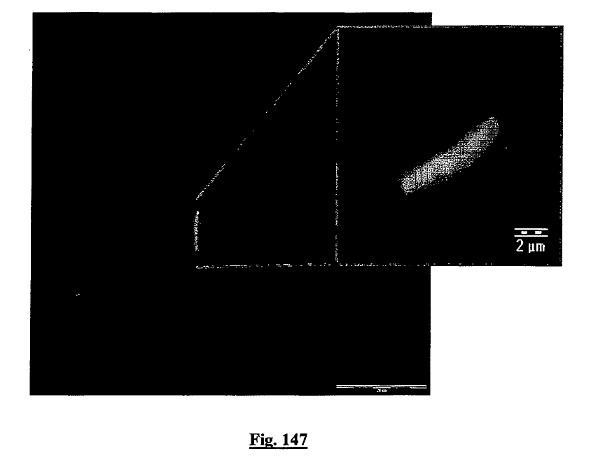

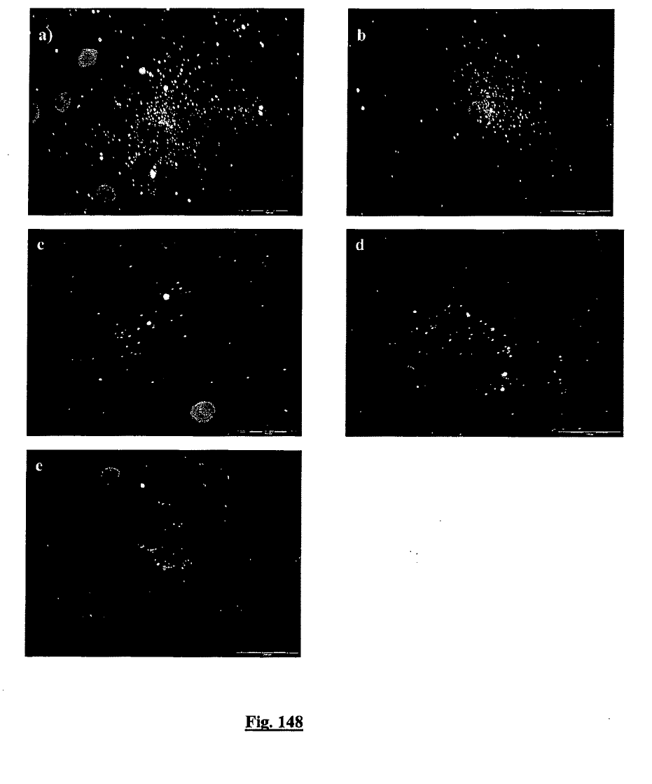

FIG. 141 shows a dark field image taken at a magnification of 10× of IDEA functionalised TSNP on a spot containing immobilized positive target nucleic acid (SEQ ID No. 1). This image confirms the binding of IDEA functionalised TSNP with complementary nucleic acid target sequences (SEQ ID No. 4).

FIG. 142 shows a dark field image taken at a magnification of 100× of IDEA functionalised TSNP on a spot containing immobilized positive target nucleic acid (SEQ ID No. 1). This image confirms the binding of IDEA functionalised TSNP with complementary nucleic acid target sequences (SEQ ID No. 4)

FIG. 143 shows a dark field image taken at a magnification of 10× of Thiol 20AA functionalised TSNP on a spot containing immobilized positive target nucleic acid (SEQ ID No. 1). This image confirms high binding of Thiol 20 AA functionalised TSNP with complementary nucleic acid sequences (SEQ ID No. 4)

FIG. 144 shows a dark field image taken at a magnification of 100× of Thiol 20 AA functionalised TSNP on a spot containing immobilized positive target nucleic acid (SEQ ID No. 1). This image confirms the very high binding of Thiol 20 AA functionalised TSNP with complementary nucleic acid sequences

FIG. 145 shows a dark field image taken at a magnification of 10× of Thiol functionalised TSNP on a spot containing immobilized positive target nucleic acid (SEQ ID No. 1). This image confirms the very high binding of Thiol functionalised TSNP with complementary nucleic acid sequences

FIG. 148 shows a dark field image taken at a magnification of 100× of Thiol functionalised TSNP on a spot containing immobilized positive target nucleic acid (SEQ ID No. 1). This image confirms the very high binding of Thiol functionalised TSNP with complementary nucleic acid sequences. In addition the darkfield image shows that the Thiol functionalised TSNP of consist of twinned, grouped and coupled TSNP. Note in the case of each group or twin coupled TSNP the entire group or twin are same colour which is uniformly distributed over the extent of the TNSP group. This is due to the sharing of the coupled plasmon. The TSNP group shows increased optical extinction cross section or brightness than in the case of single functionalised TSNP sensors and facilitates optical detection. To this end live observation of these tethered grouped TSNP sensors shows the vigorous movement of the TSNP group about their tethered position in solution. TSNP grouped sensor may also facilitate increased LSPR refractive index sensitivity over single TSNP sensors.

FIG. 147 shows a darkfield image of a grouped or precoupled TSNP coupled TSNP in solution phase. Note entire TSNP group is the same colour which is uniformly distributed over the extent of the TNSP group. This is due to the sharing of the plasmon among coupled TSNP. The TSNP group shows increased optical scattering which is observed as increase brightness than in the case of single probe functionalised TSNP facilitating optical detection and may also facilitate increased LSPR refractive index sensitivity. Increased LSPR refractive index sensitivity of coupled TSNP may be achieved by presenting the receptors such that they binding with the analyte occurs within the E-field.

FIG. 148 shows a sequence of dark field images taken at a magnification of 10× of DAPA functionalised TSNP corresponding to spots containing immobilized positive target nucleic acid (SEQ ID No. 1) at a concentrations of a) 20 μM, b) c) 200 nM, d) 20 nM and e) 2 nM. These images confirm the high binding of DAPA functionalised TSNP with complementary nucleic acid sequences across the spotting concentration range from 20 μM to 2 nM.

### Example 16

### Labeling, Mapping and Assaying the Distribution of Receptors

Oligonucleotide, peptide, antibody, protein or ligand functionalised TSNP labels/sensors targeted to cell surface marker or internal cell markers are centrifuged at 4° C. for 20 minutes at 18,000 g. TSNP labels/sensors are resuspended in RNase/DNase free water and re-centrifuged under same conditions. Resuspend in 10% of initial volume, in RNase/DNase free water and held at 4° C. TSNP labels/sensors are exposed to target cells in situ or in culture where they are then incubated under standard conditions. In the case of in situ visualisation of cultured or isolated cells can be performed under a range of microscopy techniques including TEM, confocal, darkfield etc.

Darkfield images and spectral profiles of the individual TSNP labels/sensors are recorded as described above and analysed to give a profile, map and distribution of the target receptors which may also permit biosignaturing.

### Example 17

### Real Time Monitoring of Processes Such as Cellular Processes and Events

A cellular process may include mitochondrial protein synthesis wherein mitochrondial target sequence functionalized TSNP show LSPR responses which are associated with protein levels. Further to this on the synthesis of mutant proteins which for example may be associated with the onset of cancerous conditions may be detected, characterized and monitored using dual, treble and multi probe configuration method described above as diagnostic and prognostic tools. These events can be localized through in vivo imaging.

Referring to FIG. 69 which is a schematic of target functionalised TSNP, targets may be nucleic acids, proteins, antibodies, peptides, ligands. Cancer cell target functionalised TSNP act in a homing fashion and are delivered with a large degree of exclusivity to cancer cells in a cancer tumour located within healthy normal cell tissue.

Cells with specific protein target functionalised TSNP and specific gene sequence target functionalised TSNP can act in a homing fashion to be delivered to target locations for in situ detection, monitoring, characterisation, labelling and mapping of events and process of target bodies. The target functionalised TSNP sensors/labels may exhibit a spectral response such as a shift, increase or decrease in optical scattering or a combination of these features upon the binding of an analyte molecule resulting from the activity of the body under surveillance.

Other cellular events which may be monitored are proliferation, apoptosis and angeogenisis. Functionalised TSNP target to markers specific for each of these events and pathways involved in these events may be detected, characterized and monitored using these methods

In a further embodiment a pathway or a cascade of cellular events may be switched off for example in the case of a particular organism (e.g. ribosome) its activity may be stalled by exposure to a particular biochemical reagent (e.g. ricin) and target functionalized TSNP may be used to monitor such events, prior during and after stalling.

This embodiment may further be used in combination with monitoring for example cell surface marker which may intermediately or permanently be altered by the event stalling episode or downstream of the event stalling episode. For example stalling a cellular cascade may in turn alter a cancerous profile (identified by the presence of specific cell markers at the cell surface) to a noncancerous profile (identified by the absence of associated cell markers at the cell surface) may be detected, characterized and monitored using these methods.

### Example 18

### Carbohydrate Profiling Free Solution Proteins and Surface Bound Structures

Oligonucleotide or ligand functionalised TSNP labels/sensors targeted to carbohydrates are centrifuged at 4° C. for 20 minutes at 18,000 g. TSNP labels/sensors are resuspended in RNase/DNase free water and re-centrifuged under same conditions. Resuspend in 10% of initial volume, in RNase/DNase free water and held at 4° C.

TSNP labels/sensors are exposed to target molecules or cells in situ or in culture where they are then incubated under standard conditions. In the case of in situ visualisation of cultured or isolated cells can be performed under a range of microscopy techniques including TEM, confocal, darkfield etc.

Darkfield images and spectral responses of the individual TSNP labels/sensors are recorded as described above and analysed to give a quantitative measure, profile, map and distribution of the target carbohydrates which may also permit biosignaturing.

An example of an application for this method includes downstream analysis of recombinant protein production.

### Raman Enhancement

High aspect ratios allow the continuation of electric field (E-field) scaling i.e. E² scaling with nanoparticle radii beyond the size limits at which radiative damping effects would otherwise become significant such that a further increase would no longer be observed and a reduction in E² would occur. In the case of Surface Enhanced Raman Spectroscopy (SERS) it is well known that enhancement is greater for aggregated or coupled nanoparticles such as dimers. The E-field enhancements for dimers can be increased for dimers composed of larger particles i.e. which have longer wavelength dipole plasmon resonances. Therefore larger edge length TSNP will provide the basis for high Raman enhancing substrates. Snipping the tips of large edge TSNP maybe used to blue shift their LSPR peaks in order that they are resonant with the Raman excitation laser line as required. Red shifting of the TSNP LSPR peaks may be carried out by decreasing the thickness of a TSNP, i.e. by increasing the aspect ratio of a particular edge length TSNP. Aggregation of the TSNP is required to deliver optimal Raman enhancement signals. Though E² is diminished for single TSNP which are snipped, the opposite is the case for aggregated TSNP used as Raman substrates as the increased surface area for plasmon coupling achieved by the snipping as a stronger contributor to E-field enhancement than factors such as the light rod effect of sharp TSNP tips.

### Electromagnetic Field Enhancement

Continuation of E² scaling with nanoparticle radii beyond the size limits at which radiative damping effects would otherwise become significant such that a further increase would no longer be observed and a reduction in E² would occur may be enabled by having nanoplates of high aspect ratios.

### Electrical Conductivity

The electrical conductivity is dependent on the surface area of the nanoparticles. This means the electrical conductivity is along the surface of a nanoparticle with the internal volume of a nanoparticles being effectively redundant. TSNP with large aspect ratio, which maximise the surface area while minimising the internal volume, compared to the case of lower aspect ratio TSNP will lead a lower loading requirement (lower concentration of nanoplates required) to achieve the same conductivity levels associated with conventional nanostructures.

### Optical Extinction Enhancement

Optical extinction is the combination of absorption and scattering. Generally for nanoparticle below 10 nm absorption dominates. As nanoparticle size increases, the optical scattering cross section increases and therefore optical extinction scales with TSNP edge length up to the onset of radiation damping effects at large TSNP edge lengths. Very high optical extinction can therefore be exhibited by very large TSNP with high aspect ratios that prevent the onset of radiative damping which acts to reduce optical scattering enabling the continuation of the increased optical extinction scaling beyond the case for lower aspect ratio TSNP of the same large edge lengths.

### Example 19

### Surface Enhanced Raman Spectroscopy (SERS)

Raman spectroscopy is concerned with the study of molecular vibrations. When radiation of a particular frequency falls on a molecule, some radiation is scattered. The Raman effect is a relatively weak one. Light that is not absorbed by the molecule of interest is only weakly inelastically scattered off the vibration in the molecule. A Raman spectrum is very informative as it provides a good vibrational fingerprint of the molecule. Also one major advantage that it has over the more commonly used infrared spectroscopy is that the O—H bond is only weakly Raman active so spectra can be recorded in aqueous solution with less interference from water. For SERS, the presence of nanoscale features on a metallic surface and in particular the ability of a surface to support surface plasmons creates the SERS effect. SERS has not become a routinely used analytical tool because the reproducibility of the technique is poor due to a lack of control over the fabrication of suitable SERS substrates and the equipment required is costly. However, in recent years there has been resurgence in the development of SERS as the cost of optoelectronic equipment has fallen and the development of nanofabrication techniques such that well defined substrates can be produced consistently.

Zou and Dong have demonstrated the SERS activity of aggregated silver nanoplates in aqueous solution that the addition of the analyte 2-aminothiophenol (2-ATP) to silver nanoplates slightly dampened the absorption maximum but was unable to aggregate them³⁷. Zou and Dong³⁷ required the addition of an additional agent to aggregate their silver nanoplates using NaCl to induce aggregation so that detectable SERS of 2-ATP was observed. However, the action of an aggregation agent such as NaCl would serve to alter the morphology such that the SERS substrate may not resemble the original nanoprisms in anyway.

The intensity of Raman scattering is directly proportional to the square of the induced dipole. As a consequence of exciting the local surface plasmon resonance (LSPR), the local electromagnetic field is enhanced. It has been shown, that for a metal sphere the Raman scattering scale as E⁴. Therefore if the local electric field is enhanced by a factor of 10 by the nanoparticle, the Raman scattering will be enhanced by 10^{4 38}. It is now widely accepted that the presence of ‘hot spots’ gives rise to enormous enhancement of the electromagnetic field³⁹. These ‘hot spots’ have been attributed to two basic phenomena
1) Lightning rod effect
2) Coupling of SPRs

The lightening rod effect is not associated with surface plasmons. It occurs when the incident electromagnetic field does not penetrate inside the metal nanoparticles that are next to each other. In essence the electric field is compressed or focussed into the gap between nanostructures. As this event is purely dependent on the geometry of the nanoparticles concerned it is no surprise that it has been reported as the key to SERS for nanoparticles such as nanorods. The coupling of SPRs occurs when the SPRs on adjacent nanoparticles interact and hybridise giving rise to extremely intense electromagnetic fields.

The most important aspects of the electromagnetic model are
1) Excitation of a SPR of nanoparticles or aggregates of nanoparticles
2) The position of the plasmon resonances as determined by various factors such as size, shape, dielectric properties of the metal and dielectric properties of the medium surrounding the nanoparticles.
3) The E⁴ enhancement discussed above has been calculated from theory based on a spherical metal nanoparticle model. However, as the shape of the nanoparticle is changed the number of plasmon resonances is also changed so in practice, multiple plasmon resonances must be considered.

In general SERS is dependent on a number of factors. These include the size of the nanoparticle; shape of the nanoparticle; dielectric function of the nanoparticle; dielectric function of the surrounding medium; surface coverage of the analyte; adsorption of the target molecule; metal-molecule interactions; molecular orientation of the analyte; and polarization effects. However two generic factors should always be optimized in any SERS experiment. Firstly, the plasmon resonance of the nanoparticles (usually aggregates) should be in tune with the laser line used for excitation of Raman scattering. And secondly, the adsorption of the target molecule on the surface must be maximised.

TSNP with LSPR λₘₐₓ Between 485-615 nm for SERS

Monodisperse, well-defined TSNP of varying edge length were used. The SERS spectra were recorded on an Avalon Instruments RamanStation with an excitation wavelength of 785 nm. The laser power was 100 mW and the resolution of the Raman instrument was 4 cm⁻¹. An exposure time of 10 s was used with two exposures to record each spectrum. All experiments were carried out in a 96 well polypropylene microtitre plate. The final volume in each of the wells was 300 μL, consisting of 200 μL TSNP+50 μL analyte+MgSO₄ (1 M, 50 μl).

TSNP can be prepared according to the seed mediated methods described in PCT/IE2008/000097. In this example, TSNP were prepared as follows: in a typical experiment, silver seeds are produced by combining aqueous trisodium citrate, aqueous poly(sodium styrenesulphonate) and aqueous NaBH₄ followed by addition of aqueous AgNO₃ while stirring vigorously. The nanoprisms were produced by combining 5 mL distilled water, aqueous ascorbic acid and various quantities of seed solution, followed by addition of aqueous AgNO₃. After synthesis, aqueous trisodium citrate is added to stabilize the particles. Referring to FIG. 93 the optical tuning of TSNP as a result of the quantity of seeds used in growing the TSNPs is shown. TSNPs grown from the smallest quantity of seeds (G) have a larger LSPR peak (615 nm) compared to TSNPs grown from the largest quantity of seeds (A).

SERS Using TSNP with Crystal Violet as the Analyte

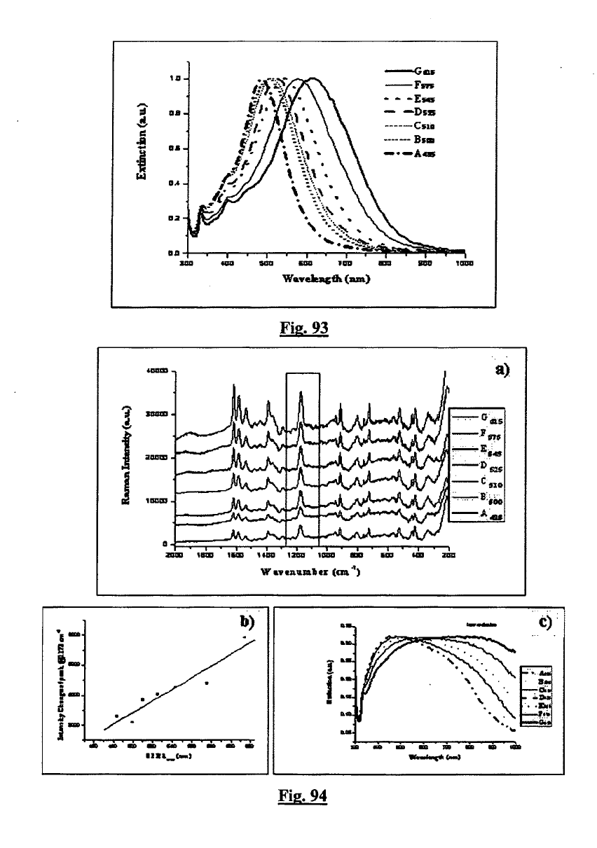

Crystal violet is a common SERS analyte. It is positively charged and will easily stick to the negatively charged (zeta potential −39±5 mV) TSNP. In this example, each well contained TSNP (200 μL), MgSO₄ (0.1 M) followed by crystal violet CV (5 μM). Aggregation was carried out using magnesium sulphate MgSO₄. In the case of true aggregation of the silver nanoplates as induced here by MgSO₄ the out of plane dipole at 340 nm is significantly diminished as shown in FIG. 94 C SERS spectra were obtained using crystal violet (5 μM) as an analyte and silver nanoprisms of varying edge length as substrates (FIG. 94A). The intensity of the SERS signal was dependent on the wavelength, λ_{max,} of the TSNP. As the LSPR λₘₐₓ is red shifted the intensity of the SERS spectrum increases (FIG. 94B). Referring to FIG. 94B it can be noted that as the LSPR λₘₐₓ of the aggregates becomes more red-shifted, the TSNP absorbance increase at 785 nm as a consequence and, the SERS spectrum is further enhanced.

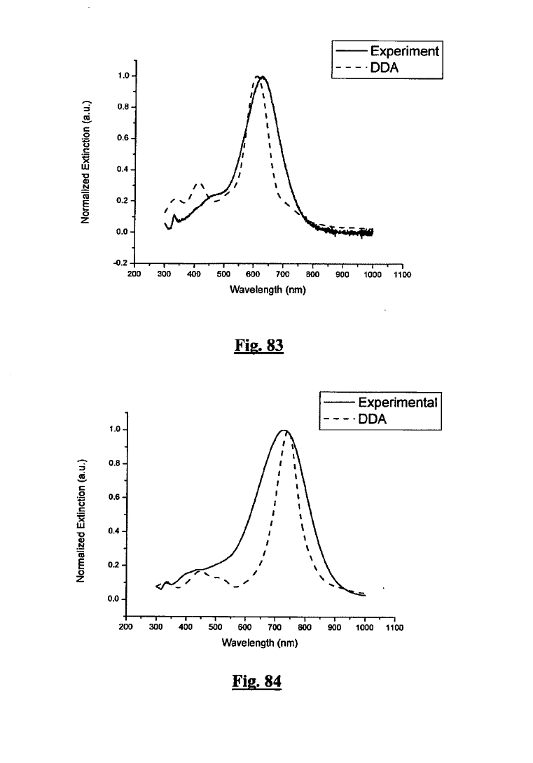

Aggregation or coupling acts to produce electromagnetically coupled plasmon bands that are localized in the junctions between TSNP aggregates or TSNP couples. These junctions act as ‘hot-spots’. It is therefore advantageous to have the analyte present during the coupling or aggregation process so that the analyte molecules have a higher chance of adsorbing onto these hot-spots. The SERS spectra shown in FIG. 83 are a result of adding the crystal violet (5 μM) to the TSNP prior to the addition of MgSO₄.

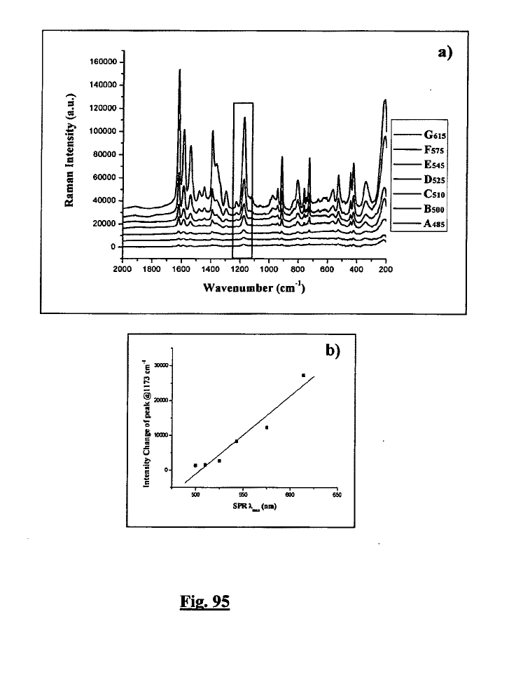

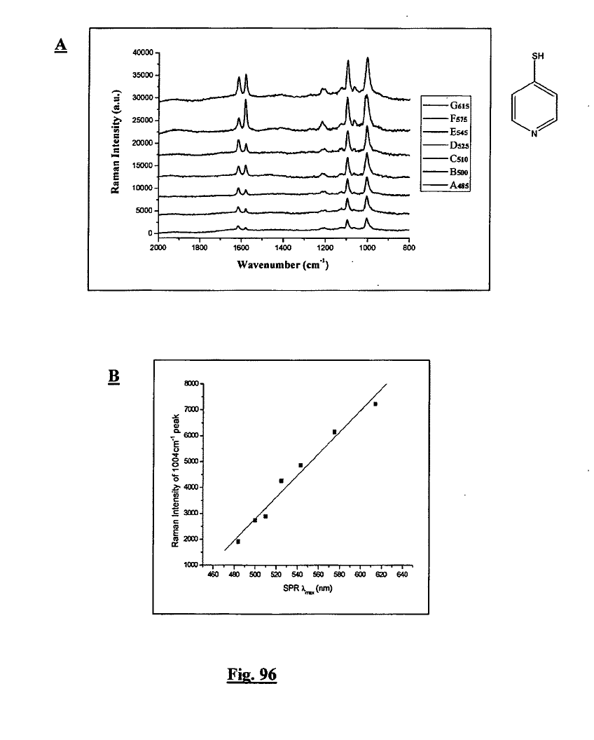

The presence of extremely intense electromagnetic fields is required for SERS to be observed. Theoretical calculations have been reported in which these intense electromagnetic fields have been shown to exist in the junctions between nanoparticles⁴⁰. If the analyte is present before the aggregation or coupling of the nanoparticles, it is likely that more of the analyte molecules will get trapped in these junctions and will therefore be SERS active compared to if the analyte is introduced after the coupling or aggregation process. Including the analyte before aggregation or coupling increases the likelihood of the analyte adsorbing onto the hot spots created during aggregation or coupling. As can been seen from FIG. 95A the intensity of the SERS bands increased when the analyte was present prior to aggregation. To quantify the importance of adding the analyte before coupling or aggregation, a direct comparison was carried out between the SERS intensities in FIG. 94A and FIG. 95A. The results confirm that the intensity of the SERS signal is increased by 66±10% if the analyte is present during the coupling or aggregation process.

Referring to FIG. 95 and Table 7 when mercaptopyridine is used as a SERS analyte molecule, a similar trend to that for crystal violet is observed. As the LSPR λ_{max,} of the coupled TSNP is further red shifted the intensity of the observed SERS spectrum is increased, there is also a charge in the relative intensities of the bands at 1580 and 1615 cm⁻¹.

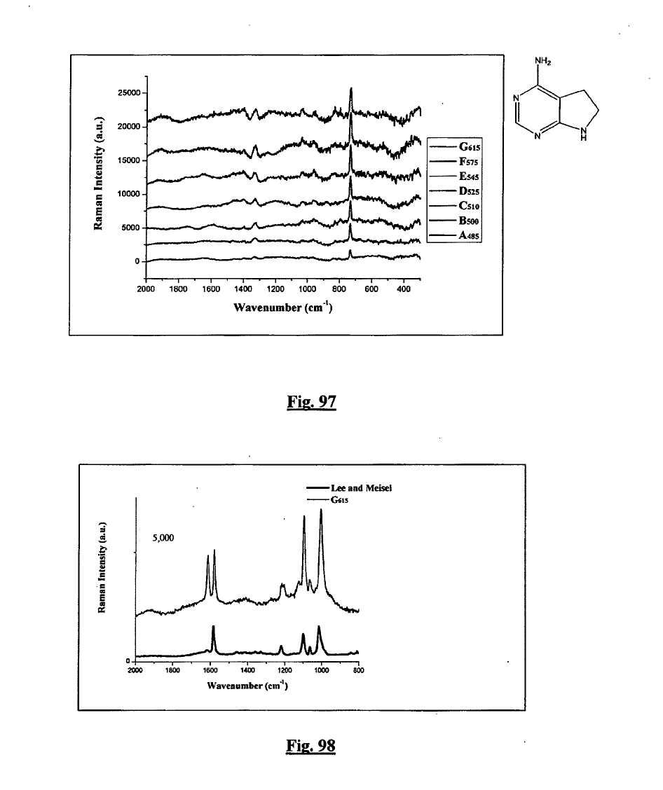

Referring to FIG. 97 and Table 8, when adenine is used as a SERS analyte molecule, a similar trend to that observed for the crystal violet and mercaptopyridine analytes is observed. We also observe a shift in the position of the ring breathing mode from 734-738 cm⁻¹ which has previously only been reported when the concentration of the analyte has been varied⁴⁴.

Example Comparison of TSNP with Lee and Meisel Colloids as SERS Substrates

One of the standard SERS substrates commonly used is silver colloid prepared by the Lee and Meisel method⁴⁰. This involves the reduction of silver nitrate by a boiling solution of trisodium citrate. A batch of Lee and Meisel colloid was prepared and was tested with 4-mercaptopyridine. So a direct comparison could be made, the TSNP was diluted so that the initial Ag ion concentration was the same for both TSNP and the Lee and Meisel colloids.

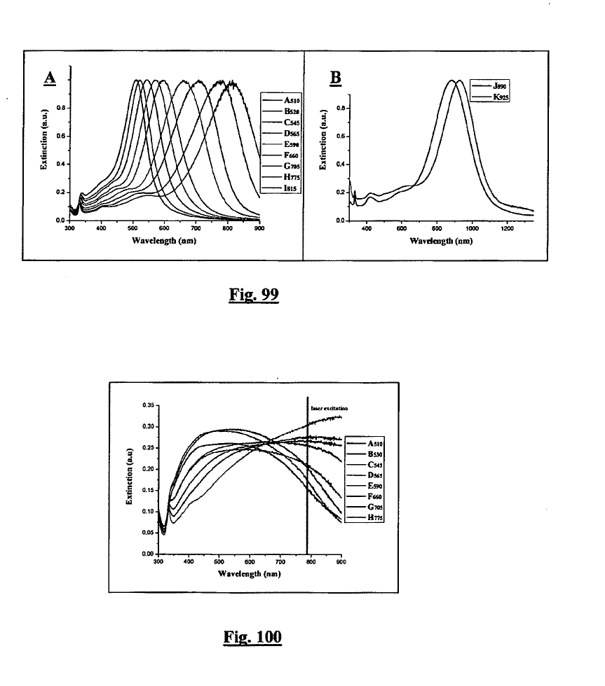

Referring to FIG. 98 it can be seen that TSNP can act as a good alternative to the Lee and Meisel colloid as the intensity of the peaks observed when TSNP G₆₁₅ was used as the SERS substrate were up to three times as intense as when standard colloid was used.

### Example TSNP as SERS Substrates Under 532 nm Laser Excitation

Crystal violet was tested with 532 nm laser excitation using a Raman microscope. Experiments were carried out using the same microtitre plate as that used for the 785 nm excitation experiments described above. The laser excitation wavelength overlaps with an electronic absorption band of the crystal violet dye. (Crystal violet λₘₐₓ=590 nm). The intensities of the Raman scattering of the vibrational modes of the crystal violet are enhanced resulting in Surface Enhanced Resonance Raman scattering (SERRS).

We have found that the intensity of the SERS spectrum is increased (by ˜66%) when the analyte is added before the coupling or aggregation process, probably due to the increased probability of it adsorbing onto the hot spots as they are formed. Furthermore, as the λₘₐₓ of the SPR is shifted further into the red (as the λₘₐₓ of the coupled TSNP is shifter further in to the NIR) the enhancement factor increases. As the nanoprisms are negatively charged, crystal violet adsorbs electrostatically to the nanoparticles giving rise to the enhanced spectrum whereas 4-Mercaptopyridine chemisorbs to the nanoparticles through a Ag—S bond giving rise to the enhanced spectrum.

TSNP with λₘₐₓ between 510-925 nm

The range of the LSPR of the TSNP prepared (FIG. 93) was varied from 510-920 nm (FIG. 99). We investigated if the SERS intensities of the analytes could be increased even further by pushing the λₘₐₓ of the SERS substrate further into the near infrared region of the spectrum. This provides larger edge length TSNP which can provide further increase E-field enhancement, which scales with nanoparticle size and which is enabled by the high aspect ratio of these large edge length TSNP. This will also apply to the case of TSNP dimers and TSNP couples as Raman substrates.

### Example Study of the Aggregation and Coupling Process

The aggregation or coupling process of the TSNPs, is key to the observation of SERS and was monitored by both UV-vis spectroscopy and TEM. FIG. 100 shows the UV-vis spectra of TSNP samples A-H, from FIG. 9910 minutes after aggregation with MgSO₄ (0.1 M).

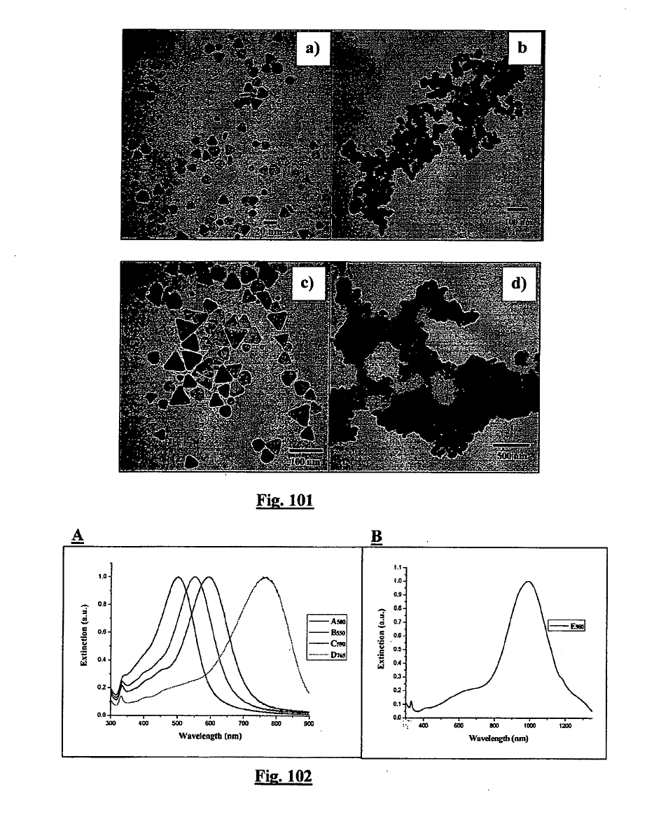

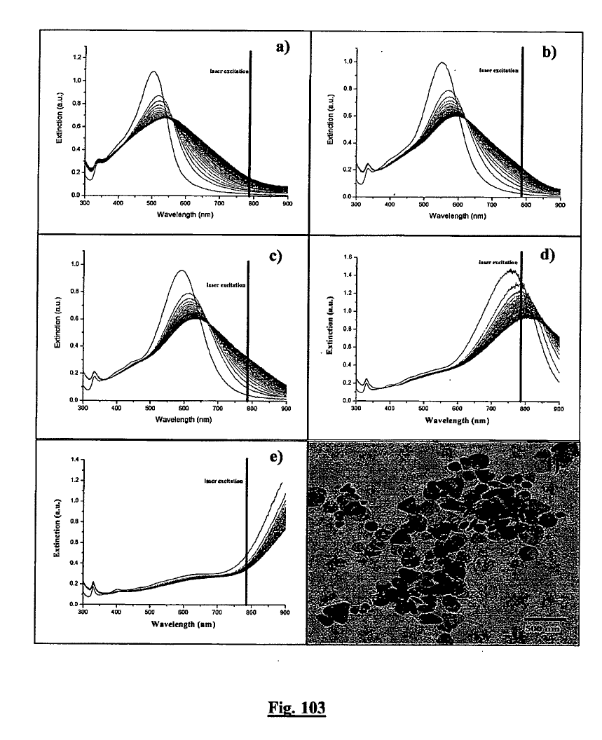

TEM images of TSNP were taken before and after aggregation with 0.1 M MgSO₄ (FIG. 101). The images taken of the TSNP before aggregation (A and C) are a result of centrifuging 1 mL of TSNP, removing the supernatant and redispersing the pellet in 100 μL distilled water. The TSNP after aggregation (B and D) were not preconcentrated before being dropped on the TEM grid.

On aggregation of the nanoprisms with MgSO₄, the original morphology of the particles was not maintained. As can been seen from FIG. 98, following aggregation the nanoprisms have ‘melted’ and the resulting nanostructures do not appear to have a specific morphology. This is an important aspect to consider when choosing an aggregating agent, for the SERS substrate as in this case after aggregation with MgSO₄ the nanostructure does not resemble the initial nanoplates in any way and this could negate the advantage of using anisotropically shaped silver nanoparticles over the standard Lee and Meisel colloid.

### Coupling of Nanoplates by Analytes

We noted that some coupling of the nanoplates was evident after the addition of the analyte alone. For this reason it was decided to monitor the aggregation or coupling of the TSNP by the analytes alone without the addition of MgSO₄. The analytes chosen for these series of experiments were thiophenol, 4-methylthiophenol, and 4-aminothiophenol. The structures of these analytes are shown below:

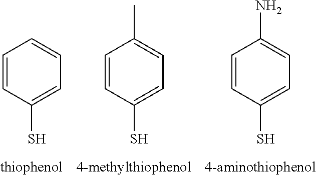

Referring to FIG. 102, the coupling of the five TSNP using 30 μm 4-aminothiophenol was examined by UV-vis spectroscopy. 200 μL of the TSNP to be analyzed was placed in a 1 mm quartz cuvette. The spectrum was recorded. The analyte was then added to the required concentration and the contents of the cuvette were agitated with the pipette. Spectra were then recorded every 30 seconds for 15 minutes. No additional aggregating agent was used. Referring to FIG. 103F, a TEM image of TSNP E₅₉₀ is shown, the TSNP were not concentrated prior to dropping on the TEM grid. Analytes caused sufficient coupling, without causing the nanoparticles to crash out of solution, for analysis by TEM without preconcentrating the sample.

Referring to FIG. 103A to E, the two dominant peaks observed in the UV-vis spectra of the nanoprisms shown above are the in-plane dipole resonance (at lower energy) and the out-of-plane quadrupole resonance, typically at 334 nm. Both the out-plane dipole and in-plane quadrupole resonances are present but are only seen as shoulder, in between the two main resonances but confirm the occurrence of coupling as opposed to aggregation. Upon coupling with the 4-aminothiophenol, the main change in the spectrum is associated with the in-plane dipole. The out-of-plane quadrupole does experience a small red-shift, accompanied by a small change in intensity however these are not remarkable when compared with the movement of the in-plane dipole. During the coupling process, the in-plane dipole resonance, shifts to longer wavelengths and broadens out significantly. This broadening also occurs mainly on the longer wavelength side of the resonance.

This coupling process, initiated by the presence of the 4-aminothiophenol alone, is different to the aggregation process that occurs in the presence of MgSO₄. From FIG. 100, it can be seen that 10 minutes after the addition of MgSO₄ to TSNP a broad absorption of the whole of the visible spectrum is recorded. The TEM images of the TSNP dried in the solid phase upon coupling with 4-aminothiophenol (FIG. 103) and MgSO₄ (FIGS. 101B and D) are also remarkably different. Upon coupling with 4-aminothiophenol, while some change in morphology of the particles is evident i.e. truncation from prisms to disks, in general the particles are merely brought closer to each other and remain individually distinct. This is in contrast to aggregation with MgSO₄ when the integrity of the initial nanoprisms is not maintained.

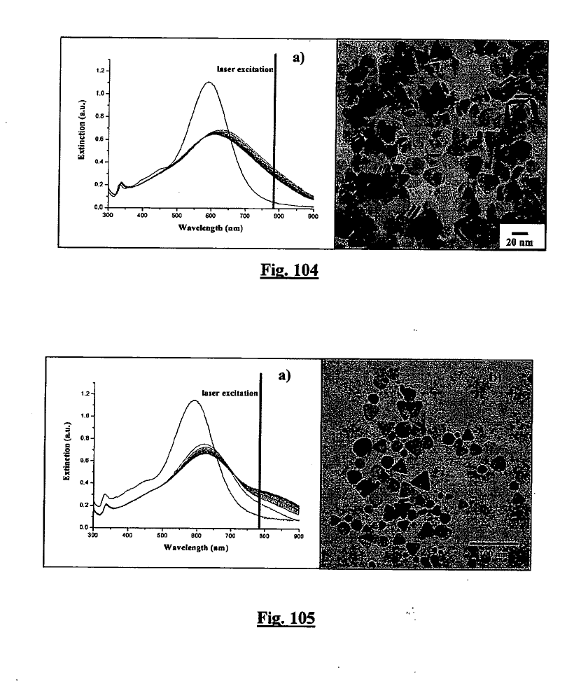

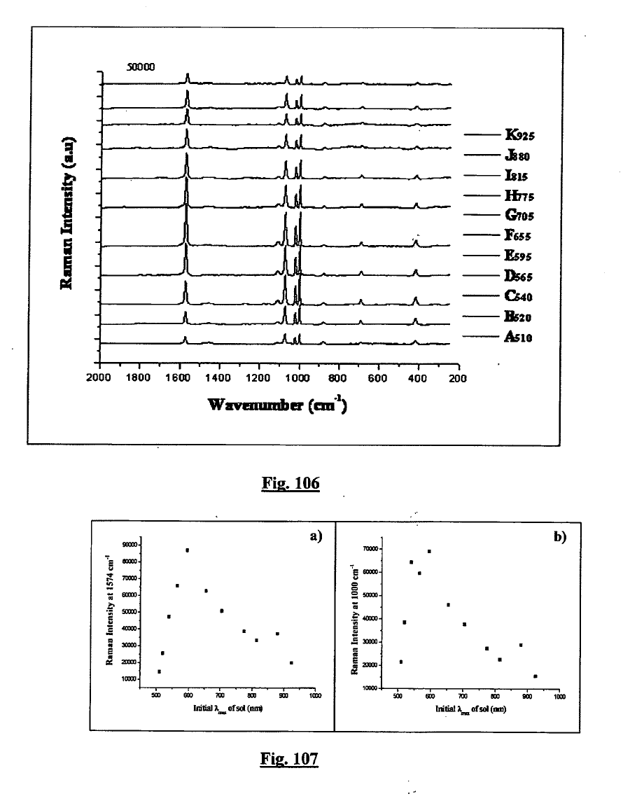

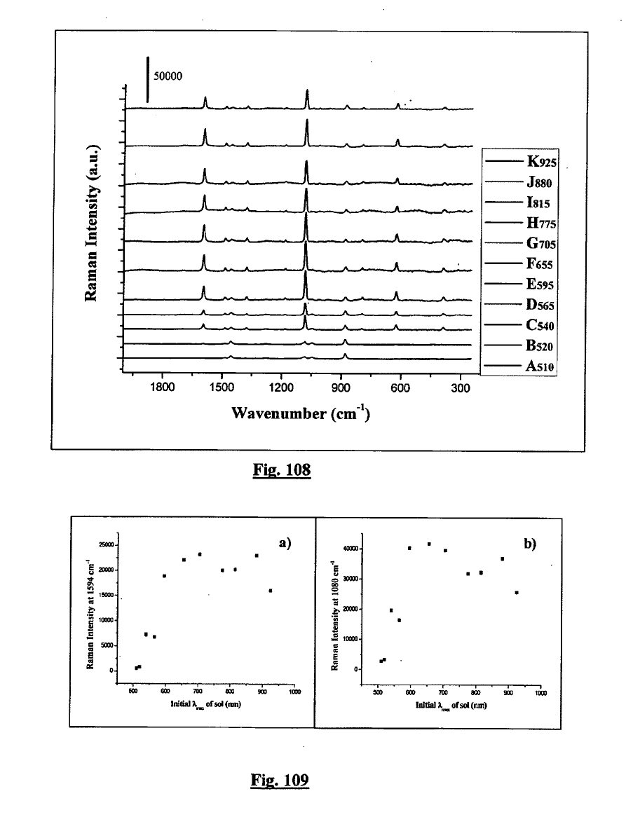

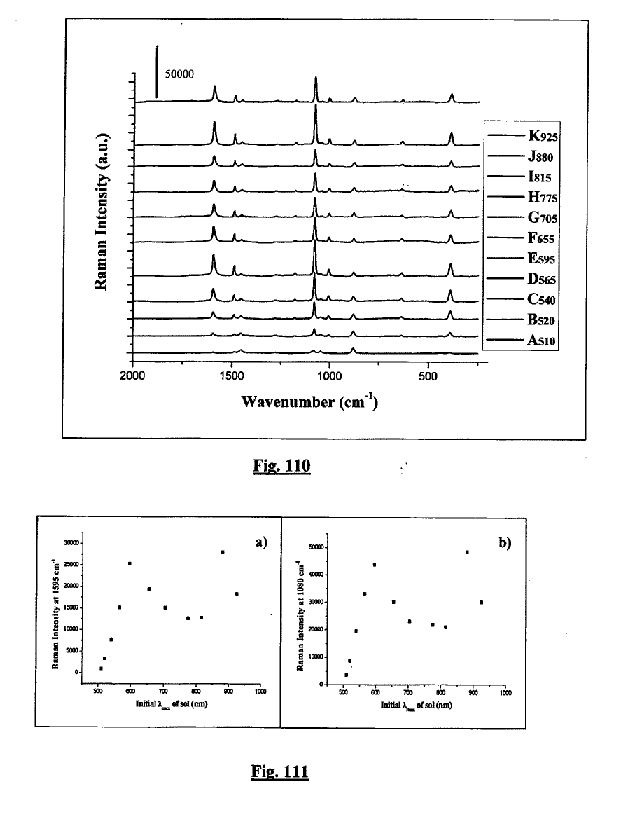

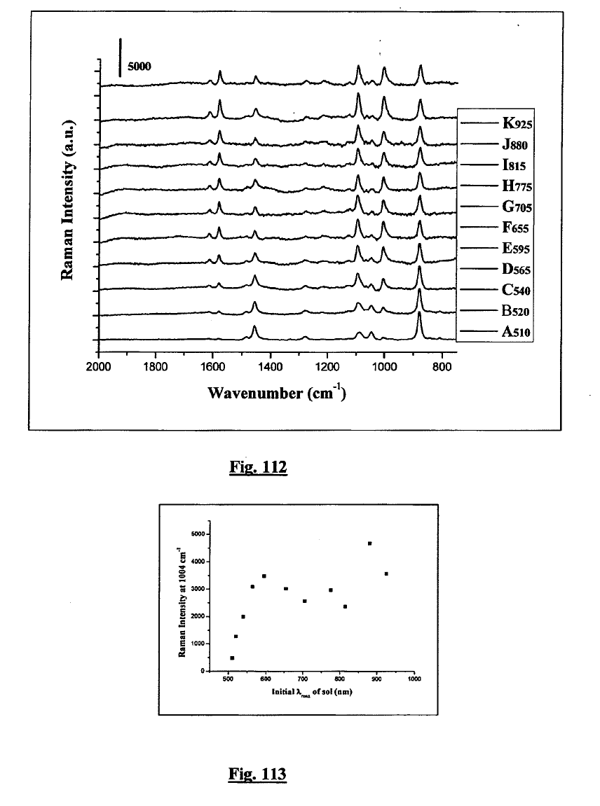

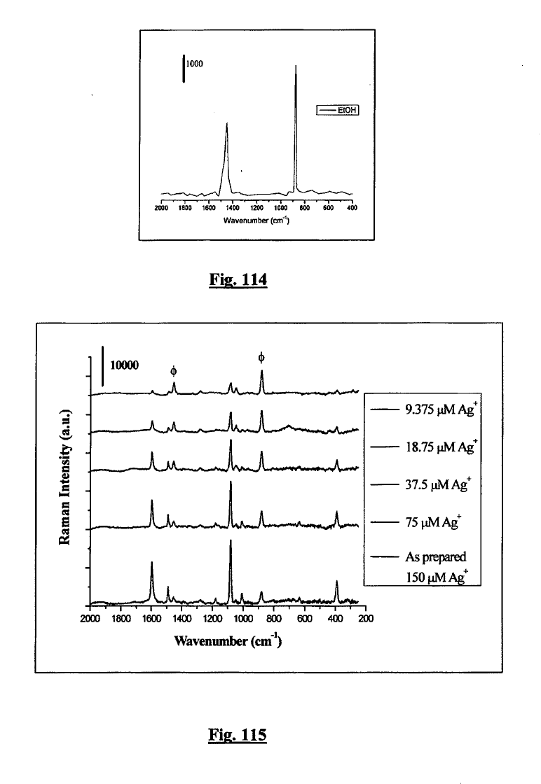

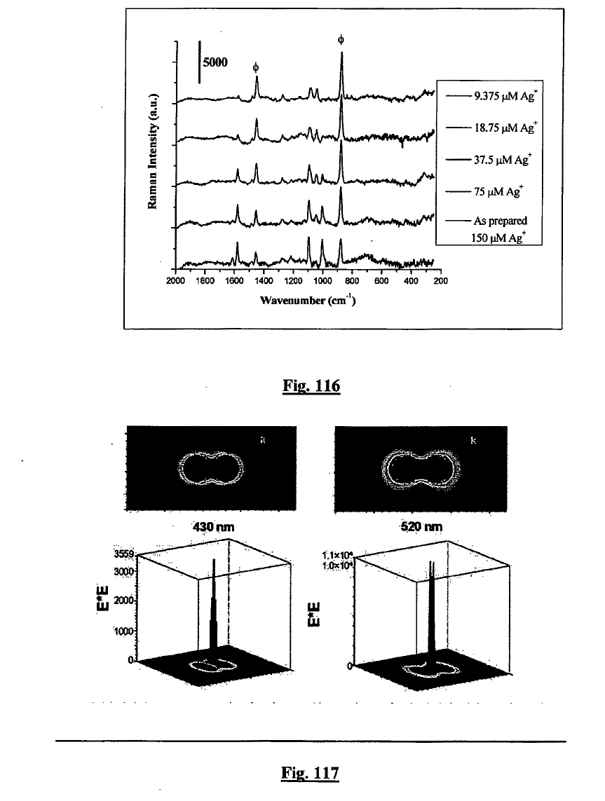

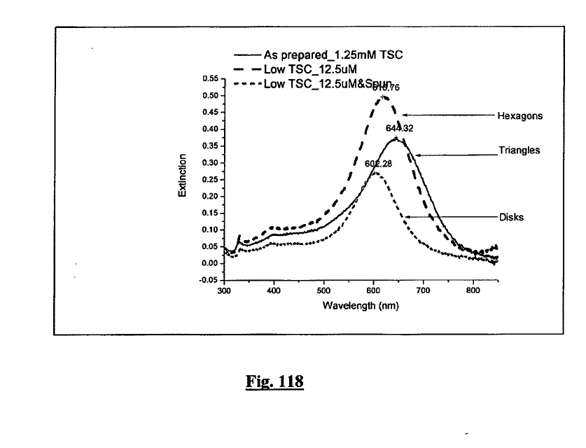

The couling of TSNP C₅₉₀ from FIG. 102 with 4-methylthiophenol and thiophenol are shown in FIGS. 104 and 105 respectively.

It can be seen that the two coupling processes shown above are slightly different to that of 4-aminothiophenol shown in FIG. 103. Firstly, the extent of coupling (from the UV-vis spectra) is less for 4-methylthiophenol and thiophenol. Also in the case of thiophenol, as the coupling proceeds a new absorption band at the longest wavelength side appears. It must be noted that the extent of coupling and aggregation cannot be ascertained from the TEM images as in some cases the drying to the solid phase process alone is enough to induce coupling and aggregation. The purpose of the TEM analysis is to confirm the morphology of the SERS substrates and it can be seen that the integrity of the TSNP is, on the whole, maintained during the coupling process.

### SERS Studies

SERS spectra were recorded on an Avalon Instruments RamanStation with an excitation wavelength of 785 nm. The laser power was 100 mW and the resolution of the instrument was 4 cm⁻¹. An exposure time of 10 s was used with two exposures to record each spectrum. All experiments were carried out in a 96 well polypropylene microtitre plate. The final volume in each of the wells was 250 μL (200 μl, TSNP+50 μL analyte). It was found that the addition of an external aggregating agent, such as MgSO₄ was unnecessary, as the analytes alone induced enough coupling for a SERS spectrum to be recorded.

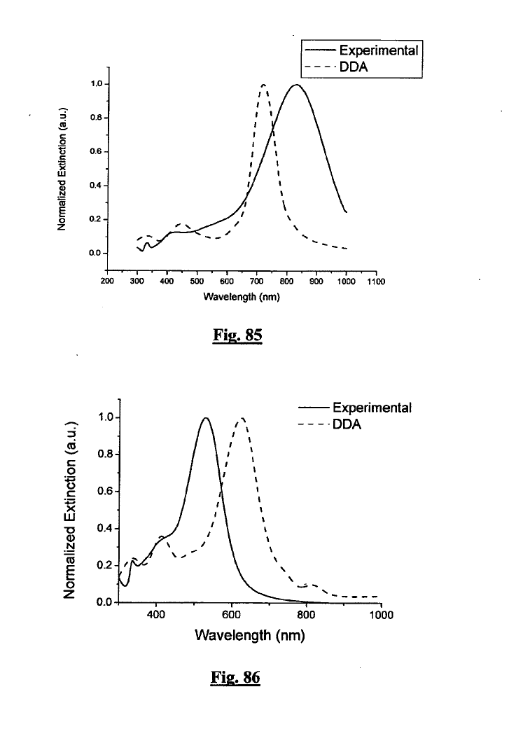

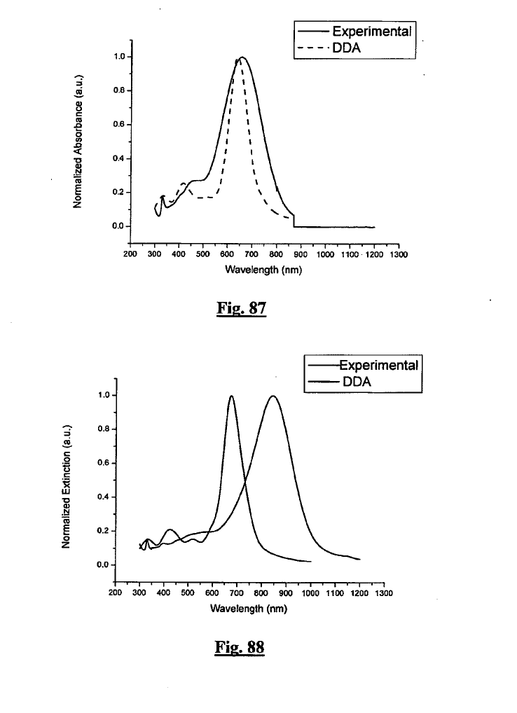

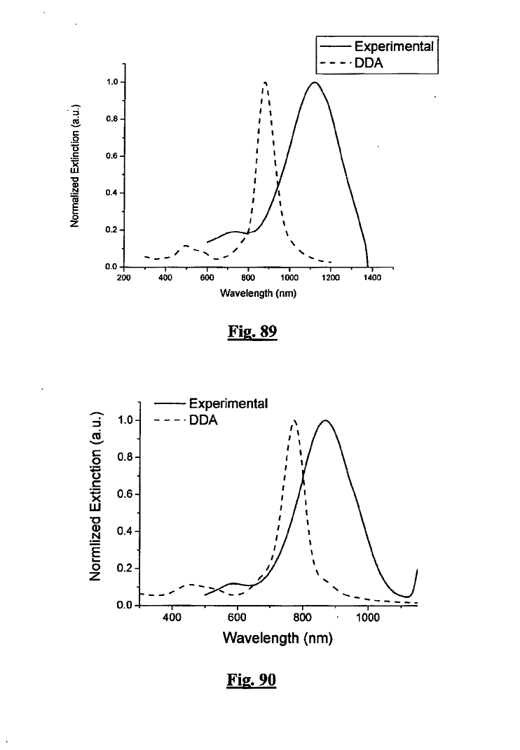

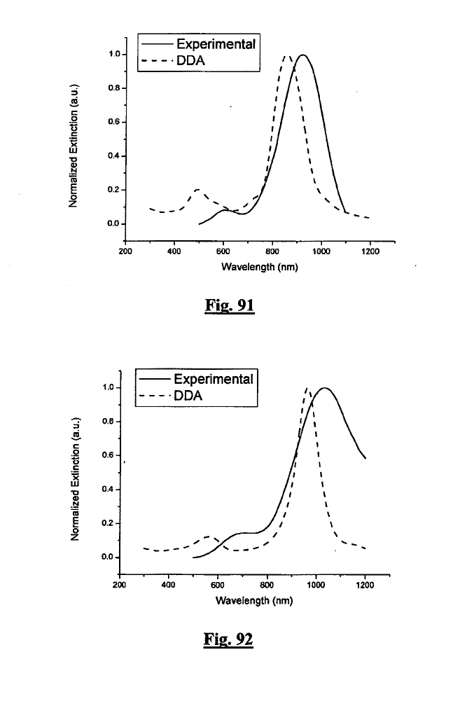

We investigated if the SERS intensities of the analytes could be increased even further by pushing the λₘₐₓ of the SERS substrate further into the near infrared region of the spectrum. From the spectra shown in FIG. 100, 105, 110, 112 a similar trend to that seen for TSNP with LSPR λₘₐₓ between 485 and 615 (FIG. 85) was observed for TSNP with λₘₐₓ less than 600 nm. As the λₘₐₓ of the SPR is shifted further into the red (therefore as the λₘₐₓ of the coupled sol is shifted further in to the NIR) the enhancement factor increases. However, as the λₘₐₓ is pushed out further than 600 nm the enhancement decreases again or at best a levelling off is observed. This phenomenon is independent of the analyte used. This can be seen clearly in FIG. 101 for methylthiophenol, FIG. 101 for 4-aminothiophenol and FIG. 113 for 4-mercaptopyridine.

The increase and subsequent decrease in the SERS intensities observed as the in-plane dipole resonance is shifted from 510 to 925 nm. The correlation between the surface plasmon resonance and laser excitation wavelength reveals that in general higher SERS intensities can be achieved when the excitation wavelength is coincident or slightly to the red side of the absorption maximum of the aggregated sols^{52, 37, 53}. As the position of the in-plane dipole resonance is shifted further into the red region of the spectrum, the position of the coupled absorption maximum is also shifted in a similar manner. Thus the degree of overlap of the absorption band with the excitation wavelength first increases and then decreases with the threshold position of the in-plane dipole of the original TSNP at ˜600 nm. If the laser excitation was varied to 1064 nm (another common laser excitation wavelength) this observed trend would also change.

TABLE 7
Assignments of SERS signals of 4-mercaptopyridine from refs⁴¹⁻⁴³

| Position of | |
|---|---|
| band (cm⁻¹) | Assignment |
| 1003 | ν(C—C)_{ring} |
| 1065 | δ(C—H) |
| 1096 | ν(C—C)_{ring,} C—S |
| 1217 | δ(C—H), δ(N—H) |
| 1580 | ν(C—C)_{ring} |
| 1615 | ν(C—C)_{ring} |

δ = bending,
ν = stretching,
ring = ring breathing.

TABLE 8
Assignment of SERS signals of adenine.

| Position of | |
|---|---|
| band (cm⁻¹) | Assignment |
| 734-738 | Ring breathing |

TABLE 9
Assignment of the Raman intensities of thiophenol of FIG. 95 from
references⁴⁶⁻⁴⁸

| Position of | |
|---|---|
| band (cm⁻¹) | Assignment |
| 419 | ν(CS), δ(CC)ring |
| 691 | ν(CS), δ(CC)ring |
| 878 | EtOH |
| 1000 | δ(CC)ring |
| 1020 | δ(CH) |
| 1073 | δ(CH) |
| 1111 | δ(CH) |
| 1456 | EtOH |
| 1575 | ν(CC)ring, ν(CS) |

The assignments of the Raman bands listed in tables 7 to 9 may be used to identify the positions of the Raman peaks in the spectra for 4-mercaptopyridine, adenine and thiophenol.

All of the analytes were in ethanolic solution and ethanol peaks observed in the SERS spectra. The spectrum of EtOH is shown in FIG. 114. Raman signals from EtOH can be used as an internal standard. The relative signal intensities of the analyte SERS spectra can be normalised against the EtOH Raman in order to obtain absolute SERS intensities and thereby allowing the calculation of SERS enhancement factors.

### Varying the TSNP Substrate Concentration

We investigated the effect of varying the concentration of TSNP as substrates on the SERS spectra for the different analytes. Referring in FIGS. 115 and 116 the analyte concentration remained at 30 μm, whilst the concentration of TSNPs was varied.

It can be seen from FIGS. 115 and 116 that as the concentration of nanoprism was decreased by dilution, the intensity of peaks associated with the analytes decreased. However, the intensity of the peaks attributed to EtOH was enhanced. As the surface area of the particles was reduced, the intensity of the EtOH peaks was increased, to such an extent that in the 4-mercaptopyridine case the EtOH peaks dominate the spectra.

### Calculation of the SERS Enhancement Factor

The SERS enhancement factor (EF) arguably one of the most important numbers for characterizing the SERS effect, however the wide discrepancies in quoted EF arises from the wide variety of definitions of the EF and also the many assumptions and estimates that are involved in its calculation. The relative enhancement factors for the thiophenol from FIG. 104 are shown below in Table 10 below with the caveat that these values can only be compared truly with EF values calculated by the same method.

The following equation was used^{37, 53}:

$$EF = \frac{( {I}_{SERS} / {C}_{SERS} )}{( {I}_{normal} / {C}_{bulk} )} \qquad ( Equation 11 )$$

where C_{CERS} is the concentration of the adsorbed molecules on the silver surface;
C_{bulk} is the concentration of molecules in the bulk samples; and
I_{CERS} and Iₙₒᵣₘₐₗ are the intensities of a certain vibration in SERS and normal Raman respectively.

The total surface area of the nanoprisms in each sol is assumed to remain constant as the same concentration of silver ion is used to prepare all of the sols and it has been found that the thickness does not vary with edge length⁵⁴. Therefore the total surface area was estimated to be 7.56 nm²/10 mL sol. The footprint of thiophenol was estimated to be 0.28 nm², similar to that of 2-aminothiophenol from reference³⁷. Considering that 200 μL sol was used for each experiment, the concentration of thiophenol required to achieve monolayer coverage is calculated to be 0.45 μM. Using equation 11 above, the enhancement factors for thiophenol on the different substrates were calculated (Table 10). These values are an order of magnitude greater than those reported for 2-ATP on similar aggregated silver nanoplates³⁷.

TABLE 10
Enhancement factors calculated by equation above for the band
at 1000 cm⁻¹ in the SERS spectrum of thiophenol

| TSNP | Enhancement Factor |
|---|---|
| A₅₁₀ | 7.4 × 10⁶ |
| B₅₂₀ | 1.3 × 10⁷ |
| C₅₄₀ | 2.2 × 10⁷ |
| D₅₆₅ | 2.0 × 10⁷ |
| E₅₉₅ | 2.4 × 10⁷ |
| F₆₅₅ | 1.6 × 10⁷ |
| G₇₀₅ | 1.3 × 10⁷ |
| H₇₇₅ | 9.4 × 10⁶ |
| I₈₁₅ | 7.8 × 10⁶ |
| J₈₈₀ | 9.9 × 10⁶ |
| K₅₁₀ | 5.3 × 10⁶ |

Using thiophenol as the analyte, large enhancement factors was obtained for coupled silver nanoprisms in solution. The ease with which the in-plane dipole resonance of the silver nanoprisms can be tuned across the visible into the near-infrared region of the spectrum makes nanoprisms prepared by this method desirable as substrates for SERS measurements with varying laser excitation wavelength.

One of the advantages that the TSNP present over the system examined by Zou and Dong³⁷ is that coupling of the TSNP may be induced by the analyte on its own such that an additional aggregation or coupling agent and coupling or aggregation step may not be required. The TEM analysis confirms in the TSNP coupling the morphology of the TSNP remain largely intact upon coupling. Therefore after coupling is presented the nature of the substrate giving rise to SERS is well characterized and on the whole the integrity of the TSNP is maintained throughout the SERS experiment. Maintaining the morphology of the nanoparticles while coupled can serve to give a larger SERS signal compared with the case where the morphology of the aggregates nanoparticle is not maintained. Also, as an additional coupling or aggregating agent is not required, there is one less variable to be considered when designing a successful SERS experiment. The TSNP SERS enhancement factors an order of magnitude greater than those reported for the same analytes on similar aggregated silver nanoplates³⁷. The narrow nature of the LSPR and the ease with which the LSPR in-plane dipole resonance of the TSNP can be tuned across the visible into the near-infrared region of the spectrum makes TSNP desirable as substrates for SERS measurements with varying laser excitation wavelength.

### Example 20

### SERS of Triangular, Hexagonal and Disk Nanoplates

Three sets of nanoplate sols were prepared (1) Triangular, (2) hexagonal and (3) disk. The triangular sols were prepared as described herein with no deprivation of passivation. Hexagons were prepared by preparing triangles but depriving the passivation which was reduced from 1.25 mM TSC to 12.5 μM TSC. Disks were prepared by preparing hexagons and centrifuging. Both the hexagons and disks are under passivated.

The preparation conditions for the different sols can be summarised as follows:

Triangles: Stabilized with 1.25 mM TSC, no centrifugation
Hexagons: Stabilized with 12.5 μM TSC, no centrifugation
Disks: Stabilized with 12.5 μM TSC, centrifuged.

These samples are denoted as:
i. Pristine Triangles,
ii. Hexagons
iii. Disks

### Coupling

4-aminothiophenol (4-ATP) in EtOH was added to 200 μL, aliquots of each of the triangle, hexagon and disk sols described above to give final concentrations of 30, 3 and 0.3 μM. In the case of the hexagon and disk sols, coupling was carried out on the ‘as prepared’ samples and also aliquots of the samples where the concentration of TSC was raised back to 1.25 mM TSC before the addition of the analyte (added TSC sols). Each sol is coupled to a greater or lesser degree at 3 different concentrations of 4-ATP.

Coupled nanoplates can be defined as linked individual nanoplates which are discrete and not physically touch but whose electromagnetic fields (E-Field) overlap. The degree of coupling may vary wherein the nanoplates may form simple dimers, trimers or other multimers where the individual nanoplates are spaced between a number of nanometers apart. They may form larger chains or groups within which each discrete nanoplate is completely identifiable. In all cases electromagnetic fields and LSPR of the coupled nanoplates can combine, becoming shared among the individual nanoplates within the coupled group, (note in many cases coupled nanoplates are found to share the same colour and spectrum) or they may exhibit modes which add or multiply together in areas or conversely subtract in other areas. E-field contours for the head to head configuration of two silver nanoplates 2 nm apart at wavelengths that correspond to modes such as the dipole and quadrupole plasmon resonances show large enhancements at the tips and the interface. Three dimensional plots show that the maximum enhancement occurs at the interface between the two triangular nanoplates. This is key to many electromagnetic field dependent phenomena such as LSPR refractive index biosensing and SERS. Coupling is distinct from aggregation which refers to a state wherein individual nanoplates within a group are no longer completely discrete and individually identifiable. Aggregation refers to a state where nanoplates in a group physically touch and merge. In the case of TSNP the presence of the out of plane quadrupole peak in the UV-Vis optical extinction spectrum in the 340 nm spectral region is a strong indicator of the retention of the physical characteristics and discreteness of the TSNP when in a coupled configuration. The UV-Vis optical extinction spectrum provides a measure of the degree of coupling of the TSNP wherein a simple red shift of the TSNP LSPR is associated with short chain coupling of the nanoplates. The greater the degree of the LSPR red shift the great the coupling, which means the greater the number of TSNP that is contained within each individual couple. The continued presence of an out of out of plane quadrupole peak in the LSPR spectrum in the 340 nm spectral region indicates the discreteness of the individual nanoplates within the couples. Coupling of TSNP can be facilitated by a range of molecules such as thiols, proteins, ligands and nucleic acids.

In the case of SERS an enhanced E-field (E)near a nanoparticle leads to enhanced Raman excitation and emission of analyte molecules. Two types of enhancements are of interest: The average of E² over the particle surface, which is relevant to conventional SERS measurements, and the peak value of E², which is important in single molecule SERS. Peak E² values are relatively modest for isolated spheres ˜100, however, they are significantly higher 10³ for spheroids and nanoprisms, due in part to red-shifted plasmon excitation, which gives the metal a more free-electronlike response! and to sharp points that produce lightening-rod effects. In many theoretical studies it is recognized that the fields between two spheres are strongly enhanced, areas know as hot spots E² enhancements greater than 10⁵ have been detected. Hao et al (reference 55 and FIG. 117) have shown that for a dimer of nanoparticles E² values close to 10⁵ occurs at hotspot areas in the region of the interface between silver nanoprisms where the separation is 2 nm between the nanoprisms. The enhancement is a strong function of separation distance, and it scales with nanostructure size such that larger nanostructures give the same enhancements for larger separations. Hao et al⁵⁵ also suggest that not all of the single molecule SERS enhancement factor of 10¹² can be ascribed to purely electromagnetic effects.

We describe SERS using nanoplates which are coupled. Presentation of the analyte molecules within the E-fields of the coupled TSNP is an important feature as is the presentation of the analyte molecules in E-field hot spots. In one embodiment of the invention, presentation of the analyte molecules in E-field hot spots is achieved through the use of under passivated nanoplates. The analyte molecule are in this case used to complete the passivation of the nanoplates and also to couple the nanoplates. In so doing the analyte molecules present themselves within the E-field hot spots at the interface region between the coupled nanoplates in more optimal configuration for SERS.

### EXPERIMENTAL

### Preparation of the Sols:

### Preparation of Seed Particles:

Aqueous TSC (5 mL, 2.5 mM), poly(sodium styrenesulphonate) (PSSS; 0.25 mL, 500 mg/L; 1,000 kDa) and NaBH4 (0.3 mL, 10 mM) were combined with vigorous stirring followed by addition of AgNO3 (5 mL, 0.5 mM) at a rate of 2 mL/min using a syringe pump, while stirring continuously. The seeds were aged for 4 h prior to use in the growth step.

Growth from Seeds of Triangles (1), Hexagons (2) and Disks (3)

10 mL distilled water, ascorbic acid (150 μL, 10 mM) and various quantities of seed solution were combined followed by addition of AgNO3 (6 mL, 0.5 mM) at a rate of 2 mL/min with vigorous stirring. After synthesis, the as prepared sol was split into two aliquots of equal volume.
1. The first aliquot was stabilized by the addition of TSC (0.5 mL, 25 mM) to give a final TSC concentration of 1.25 mM. These are triangular nanoplate sols.
2. The second aliquot was stabilized by the addition of TSC (5 μL, 25 mM) to give a final TSC concentration of 12.5 μM. These are hexagonal nanoplate sols.
3. The second aliquot was stabilized by the addition of TSC (5 μL, 25 mM) to give a final TSC concentration of 12.5 μM was centrifuged at 13,200 rpm for 40 minutes and the pellets were redispersed back to their original volume with H₂O. These are disk nanoplate sols.

In summary, three sets of nanoplate sols were prepared Triangular, hexagonal and disk. The sols with initial λₘₐₓ at approx. 600 nm were chosen for the SERS study and the UV-vis spectra are shown in FIG. 118 in which: Triangles are Stabilized with 1.25 mM TSC, no centrifugation; Hexagons are Stabilized with 12.5 μM TSC, no centrifugation; and Disks are Stabilized with 12.5 μM TSC, centrifuged.

### Triangles

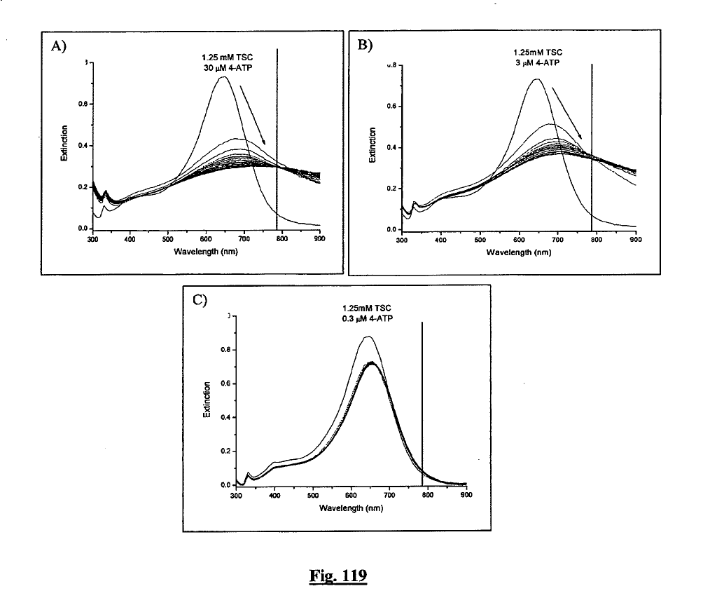

Upon addition of 4-ATP to the sols, the in-plane dipole LSPR gradually shifted to longer wavelengths and in the case of 30 μM and 30 μM 4-ATP the LSRP was observed to broaden out significantly (mainly on the longer wavelength side of the resonance) which corresponds to significant coupling of the triangular nanoplates (FIG. 119). In the case of 30 μM 4-ATP a clear isosbectic point at 795 nm was observed (FIG. 119A). As the concentration of the analyte was reduced to 3 μM, the isosbectic point was not as well defined but occurs at approximately 860 nm (FIG. 119B). At 0.3 μM 4-ATP a shift in the in-plane dipole LSPR (Δλ=10 nm) was observed, but no aggregation was noted (FIG. 119C). This shift is associated with coupling of the triangular nanoplates

### Hexagons

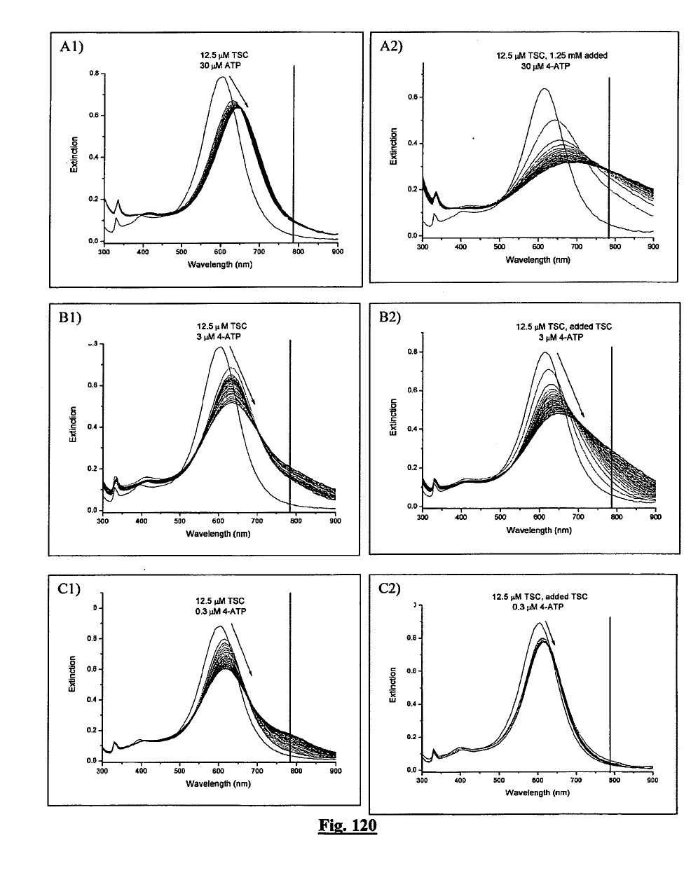

Upon addition of 30 μM 4-ATP to sols stabilized with 12.5 μM TSC (hexagons), the in-plane dipole LSPR gradually shifted to a longer wavelength over a 15 minute period (FIG. 120A1). This shift was also accompanied by a small decrease (˜6%) in intensity. However significant broadening of the LSPR was not observed, indicating the adsorption of the analyte onto the surface of the nanoplates with causing extensive coupling of the nanoplates

Upon addition of TSC (1.25 mM) to the hexagnonal sol prior to the addition of the 4-ATP (FIGS. 120 A2, B2 and C2), a similar trend to that observed in FIGS. 119A and B was observed. A clear isosbectic point at 770 nm was observed. This is consistent with significant coupling of the hexagonal sols with a final TSC concentration of 1.25 mM in the presence of 30 μM 4-ATP

Upon addition of 3 μM 4-ATP to hexagnonal sols stabilized with 12.5 μM TSC 9 FIG. 120 B2), the in-plane dipole LSPR shifted to a longer wavelength (Δλ=29 nm) before experiencing a decrease in intensity. This was also accompanied by broadening of the LSPR but not to the same extent as that observed in FIGS. 119 A and B. A clear isosbectic point at 700 nm was observed.

Upon addition of TSC (1.25 mM) to the hexagnonal sol prior to the addition of the 4-ATP, a similar trend to that observed in FIG. 119 was observed. A clear isosbectic point at 700 nm was observed. This is associated with coupling of the hexagonal nanoplates However, the extent of coupling (as judged by the intensity of the LSPR at the isosbectic point) was not as great as that observed in FIG. 119.

Upon addition of 0.3 μM 4-ATP to hexagnonal sols stabilized with 12.5 μM TSC, the in-plane dipole LSPR shifted to a longer wavelength (Δλ=18 nm) before experiencing a decrease in intensity (FIG. 108 C2). This was also accompanied by broadening of the LSPR but not to the same extent as that observed in FIG. 107. A clear isosbectic point at 675 nm was observed.

Upon addition of TSC (1.25 mM) to the sol prior to the addition of the 4-ATP, a shift in the in-plane dipole LSPR (Δλ=12 nm) was observed, indicating a low degree of coupling was noted. This is associated with coupling of the hexagonal nanoplates

### Disks:

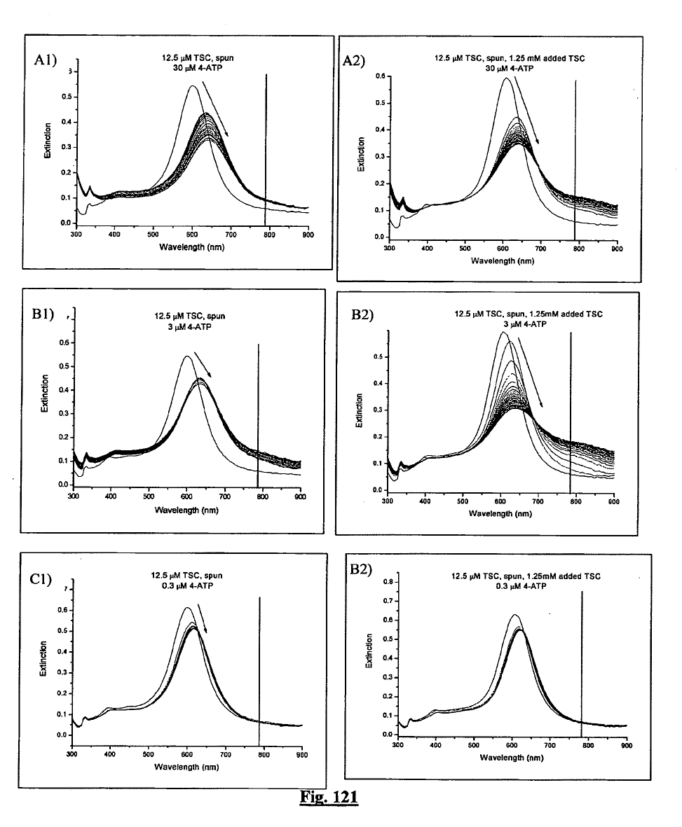

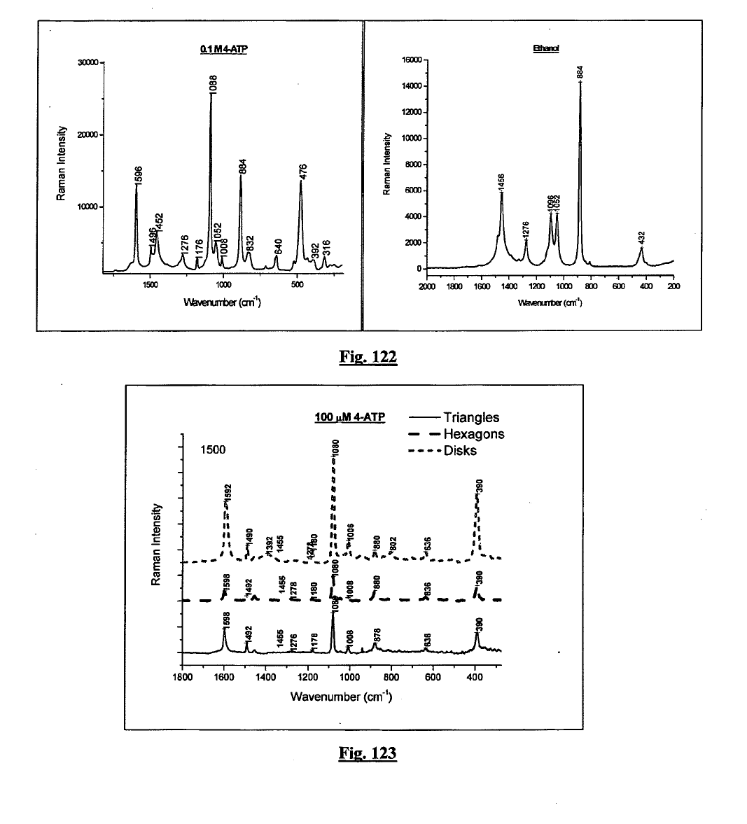

Upon addition of 30 μM 4-ATP to sols stabilized with 12.5 μM TSC and then centrifuged to from disk sols, the in-plane dipole LSPR shifted to a longer wavelength (Δλ=30 nm) before experiencing a decrease in intensity (FIG. 121 A1). This was not accompanied by broadening of the LSPR. No isosbectic point was observed. This is associated with coupling of the hexagonal nanoplates.

Upon addition of TSC (1.25 mM) to the sol prior to the addition of the 4-ATP, a shift in the in-plane dipole LSPR (Δλ=30 nm) was observed (FIG. 121). This was also accompanied by broadening of the LSPR but not to the same extent as that observed in FIG. 119 A clear isosbectic point at 695 nm was observed.

Upon addition of 3 μM 4-ATP to sols stabilized with 12.5 TSC and then centrifuged, the in-plane dipole LSPR shifted to a longer wavelength (Δλ=30 nm). This was not accompanied by broadening of the LSPR. No isosbectic point was observed. (FIG. 121 B1) This is associated with coupling of the hexagonal nanoplates.

Upon addition of TSC (1.25 mM) to the sol prior to the addition of the 4-ATP, a gradual redshift in the in-plane dipole LSPR (Δλ=30 nm) was observed. This was also accompanied by broadening of the LSPR (to greater extent to that observed in FIG. 120). A clear isosbectic point at 695 nm was observed.

Upon addition of 0.3 μM 4-ATP to sols stabilized with 12.5 μM TSC and then centrifuged, the in-plane dipole LSPR shifted to a longer wavelength (Δλ=18 nm) indicating coupling but no aggregation was noted (FIG. 121 C1).

Upon addition of TSC (1.25 mM) to the sol prior to the addition of the 4-ATP, a shift in the in-plane dipole LSPR (Δλ=18 nm) was observed indicating coupling, but again no aggregation was noted.

### Summary

Triangles: As the concentration of 4-ATP was reduced from 30 to 3 to 0.3 μM, the extent of plasmon broadening and shifting of the nanoplates was also decreased and is consistent with reduced degrees of coupling of the triangular nanoplates.

Hexagons: Coupling was induced on the addition of the 4-ATP analyte at each concentration, however not to the same extent as observed for the triangles. This is consistent with the 4 ATP analyte also playing a role in further passivating the hexagonal surfaces in addition to inducing coupling.

Disks: Coupling and not aggregation was induced by 4-ATP.

### SERS

The SERS spectra were recorded on an Avalon Instruments RamanStation-FS with an excitation wavelength of 785 nm. The laser power was 100 mW and the resolution of the instrument was 4 cm⁻¹. An exposure time of 10 s was used with two exposures to record each spectrum. All experiments were carried out in a 96 well polypropylene microtitre plate. The final volume in each wells was 250 μL (200 μL sol+50 μL analyte).

FIG. 122 is a Raman spectra for 4-aminothiophenol and ethanol which shows where the Raman peaks of the analyte and the solvent are located

### Morphology Comparison:

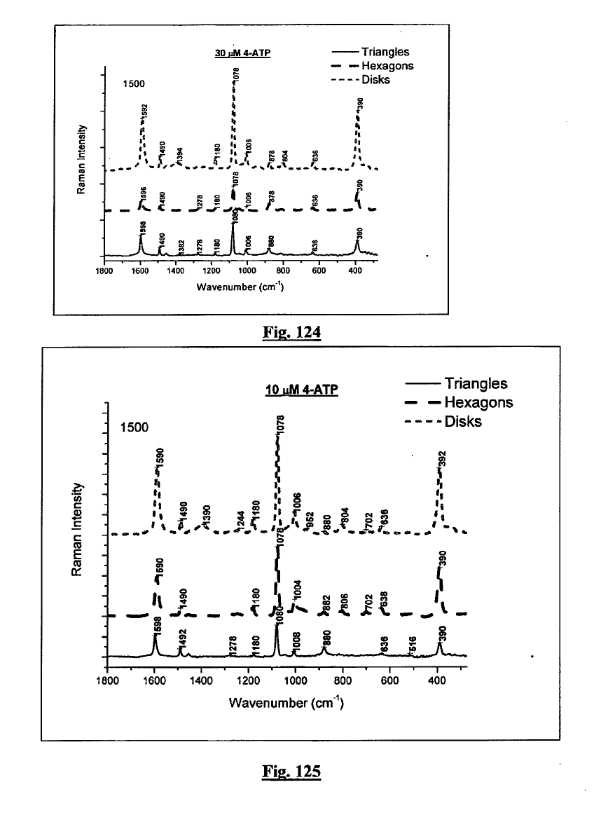

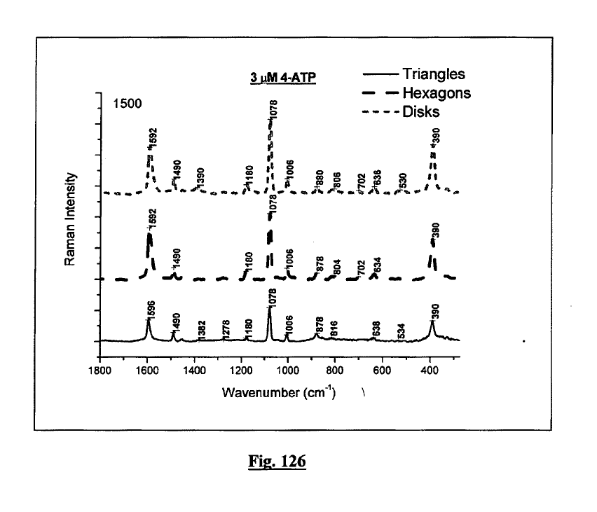

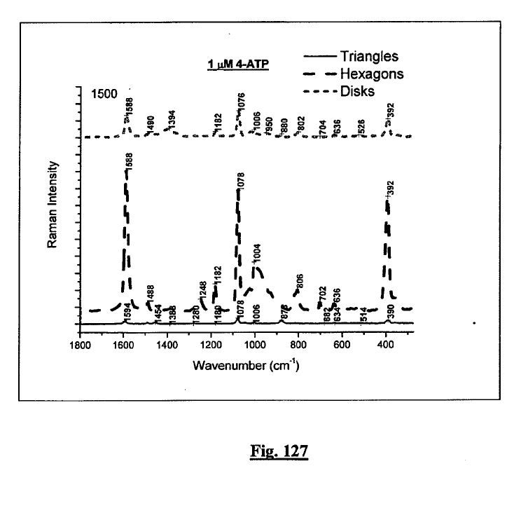

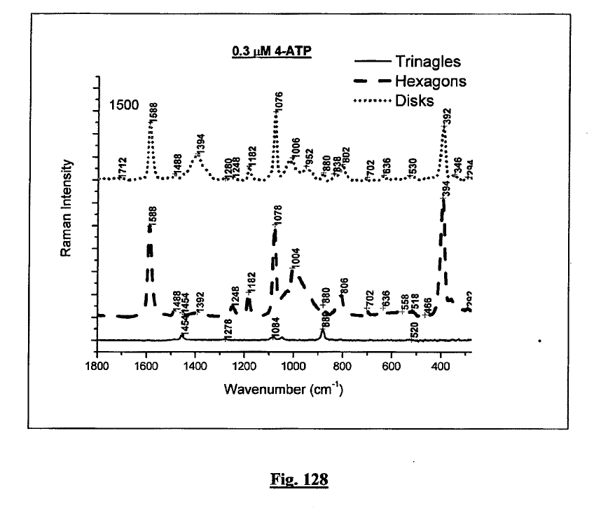

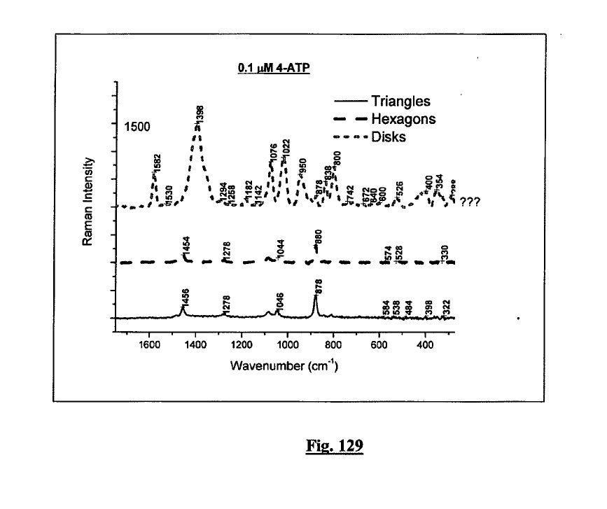

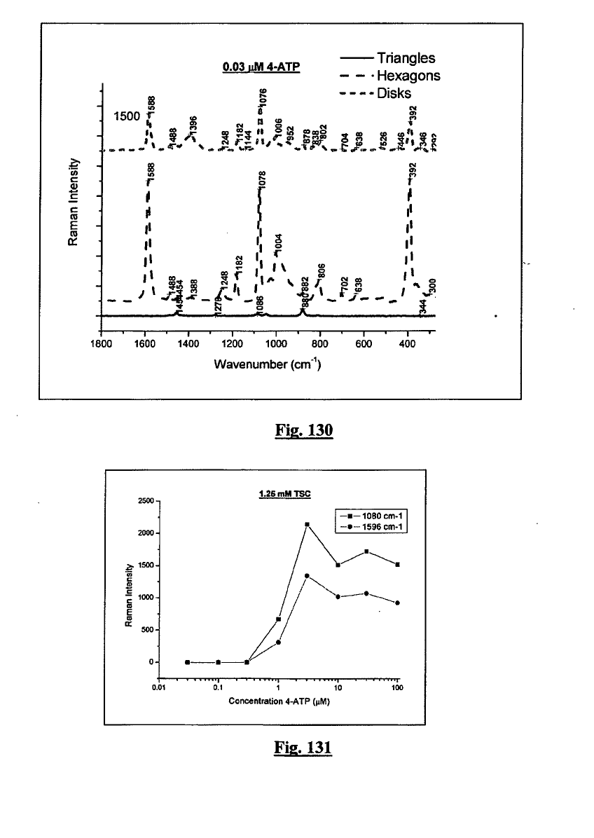

FIG. 123 to FIG. 130 show SERS of triangular, hexagonal and disk shaped nanoplates in the presence of 4-ATP at a concentration of 100 μM, 30 μM, 3 μM, 1 μM, 0.3 μM, 0.1 μM, and 0.03 μM. FIG. 118 shows SERS peak intensities of 4-ATP at a concentration range of 100 μM to 0.03 μM on triangular nanoplates; FIG. 119 is SERS peak intensities of 4-ATP at a concentration range of 100 μM to 0.03 μM on hexagonal nanoplates; FIG. 120 shows the SERS peak intensities of 4-ATP at a concentration range of 100 μM to 0.03 μM on hexagonal nanoplates. FIG. 121 shows SERS peak intensities of 4-ATP at a concentration range from 100 μM to 0.03 μM on disk nanoplates.

### Triangles

Referring to FIG. 131 and Table 11, as the concentration of 4-ATP was decreased from 100-3 μM an increase in SERS intensity was observed for the triangular nanoplates. A decrease was then observed between 3 μM and 1 μM analyte. Below this concentration, only EtOH Raman signals were detected. The intensity of the signals observed are comparable and exceed those reported in the literature particularly in the case of 3 μM analyte concentration.

TABLE 11
SERS peak positions of 4-ATP (bold) and Raman peak position of ethanol at a
concentration range from 100 μM to 0.03 μM on triangular nanoplates

| ATP | | 100 uM | 30 uM | 10 uM | 3 uM | 1 uM | 0.3 uM | 0.1 uM | 0.03 uM | EtOH |
|---|---|---|---|---|---|---|---|---|---|---|
| 316 | | | | | | | | | | |
| 392 | δCS | 390 | 390 | 390 | 390 | 390 | | | | |
| | | | | | | | | | | 432 |
| 476 | | | | | | | | | | |
| 640 | δCC | 636 | 636 | 636 | 638 | 634 | | | | |
| 712 | | | | | | | | | | |
| | | | | | | | 810 | 810 | 810 | 808 |
| 832 | | | | | | | | | | |
| 884 | | 878 | 880 | 880 | 878 | 878 | 880 | 878 | 880 | 884 |
| 1008 | δCH | 1008 | 1006 | 1008 | 1006 | 1004 | | | | |
| 1052 | | | | | | 1045 | 1045 | 1046 | 1046 | 1052 |
| 1088 | νCC, νCS | 1080 | 1080 | 1080 | 1078 | 1078 | | | | |
| | | | | | | | 1084 | 1088 | 1086 | 1096 |
| 1176 | δCH | 1178 | 1180 | 1180 | 1180 | 1180 | | | | |
| 1276 | | 1276 | 1278 | 1278 | 1278 | 1280 | 1278 | 1278 | 126 | 1276 |
| 1452 | | | 1452 | | | 1454 | 1454 | 1456 | 1454 | 1456 |
| 1496 | νCC | 1492 | 1490 | 1492 | 1490 | 1489 | | | | |
| 1596 | νCC | 1598 | 1598 | 1598 | 1598 | 1594 | 1594 | | | |

Hexagons, (12.5 μM TSC (λmax=617 nm)):

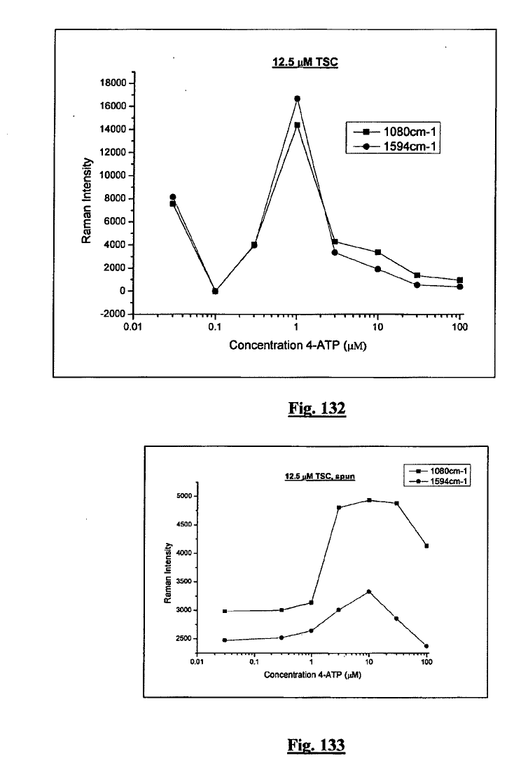

Referring to FIG. 132 and Table 12, as the concentration of 4-ATP was decreased from 100-1 μM an increase in SERS intensity was observed most notably between 3 and 1 λM. A decrease was then observed using 0.3 μM analyte. At 0.1 μM analyte, only EtOH Raman signals were detected. However 4-ATP SERS signals were then observed using 0.03 μM analyte. Another point to note is that as the concentration of the analyte was reduced the vC-C signal shifts 10 cm⁻¹ from 1598 to 1588 cm⁻¹, and becomes the most dominant signal in the SERS spectrum. This can be associated with the analyte orientation and changing of the analyte orientation. It is also associated with the increased binding of the analyte to different crystal faces or the different loading of the analyte on to different crystal faces of the nanoplates than is found in for example the case of the pristine triangles. These results are indicative that under these conditions the analyte molecule is in more optimal configuration for SERS. This is evidence that under these conditions in its role to increase the passivation of the nanoplates and also to couple the nanoplates the analyte molecules present themselves, by varying orientation, loading, or a combination of both within the E-field hot spots at the interface region between the coupled nanoplates in format that generates a SERS signal where no SERS is produced for other samples such as the pristine triangles.

TABLE 12
SERS peak positions of 4-ATP (bold) and Raman peak position of ethanol at a
concentration range from 100 μM to 0.03 μM on hexagon nanoplates

| ATP | | 100 uM | 30 uM | 10 uM | 3 uM | 1 uM | 0.3 uM | 0.1 uM | 0.03 uM | EtOH |
|---|---|---|---|---|---|---|---|---|---|---|
| 316 | | | | | | | | | | |
| 392 | δCS | 390 | 390 | 390 | 390 | 392 | 394 | | 392 | |
| | | | | | | | | | | 432 |
| 476 | | | | | | | | | | |
| 640 | δCC | 636 | 636 | 638 | 634 | 636 | 636 | | 636 | |
| 712 | | | | 702 | 702 | 702 | 702 | | 702 | |
| | | 818 | 803 | 806 | 804 | 806 | 806 | | 806 | 808 |
| 832 | | | | | | | | | | |
| 884 | | 880 | 878 | 882 | 878 | 880 | 879 | 880 | 880 | 884 |
| 1008 | δCH | 1008 | 1006 | 1004 | 1006 | 1004 | 1004 | | 1004 | |
| 1052 | | | | | | | | 1044 | | 1052 |
| 1088 | νCC, νCS | V1080 | 1078 | 1078 | 1078 | 1078 | 1078 | 1086 | 1078 | |
| | | | | | | | | | | 1096 |
| 1176 | δCH | 1180 | 1180 | 1180 | 1180 | 1182 | 1182 | | 1182 | |
| 1276 | | 1278 | 1278 | 1277 | 1277 | | | 1278 | | 1276 |
| 1452 | | 1454 | 1454 | 1454 | 1454 | | 1454 | 1454 | 1454 | 1456 |
| 1496 | νCC | | 1490 | 1490 | 1490 | 1488 | 1488 | | 1488 | |
| 1596 | νCC | 1598 | 1596 | 1590 | 1590 | 1588 | 1588 | | 1588 | |

Disks, (12.5 μM TSC, Spun (λmax=602 nm)):

Referring to FIG. 133 and Table 13, as the concentration of 4-ATP was decreased from 100 to 10 μM a small increase in SERS intensity was observed. Upon further lowering of the analyte concentration a small decrease in SERS intensity was observed.

TABLE 13
SERS peak positions of 4-ATP (bold) and Raman peak position of ethanol at a
concentration range from 100 μM to 0.03 μM on disk nanoplates

| ATP | | 100 uM | 30 uM | 10 uM | 3 uM | 1 uM | 0.3 uM | 0.1 uM | 0.03 uM | EtOH |
|---|---|---|---|---|---|---|---|---|---|---|
| 316 | | | | | | | | | | |
| 392 | δCS | 390 | 390 | 392 | 390 | 392 | 392 | 400 | 392 | |
| | | | | | | | | | 446 | 432 |
| 476 | | | | | | | | | | |
| 640 | δCC | 636 | 636 | 636 | 636 | 636 | 636 | 640 | 638 | |
| 712 | | 702 | 704 | 702 | 702 | 704 | 702 | | 704 | |
| | | 802 | 804 | 804 | 806 | 802 | 802 | 800 | 802 | 808 |
| 832 | | | | | | | | | | |
| 884 | | 880 | 878 | 880 | 880 | 880 | 880 | 878 | 878 | 884 |
| 1008 | δCH | 1006 | 1006 | 1006 | 1006 | 1006 | 1006 | | 1006 | |
| 1052 | | | | | | | | | | 1052 |
| 1088 | νCC, νCS | 1080 | 1078 | 1078 | 1078 | 1076 | 1076 | 1076 | 1076 | |
| | | | | | | | | | | 1096 |
| 1176 | δCH | 1180 | 1180 | 1180 | 1180 | 1182 | 1182 | 1182 | 1182 | |
| 1276 | | 1278 | 1278 | | 1276 | | | | | 1276 |
| 1452 | | 1456 | 1494 | | | 1454 | | | | 1456 |
| 1496 | νCC | 1490 | 1490 | 1490 | 1490 | 1490 | 1488 | | 1488 | |
| 1596 | νCC | 1592 | 1592 | 1590 | 1592 | 1588 | 1588 | 1592 | 1588 | |

### DISCUSSION

Disks which are produced by deprivation of passivation to nanoplates give rise to the biggest SERS enhancements up to an analyte concentration of between 10 and 3 μM.
At 3 μM both hexagons and disks give rise to the approximately the same enhancement, which is greater than that of the fully passivated triangles (pristine triangles).
At an analyte concentration of 1 μM and below, hexagons give rise to the biggest enhancement, No SERS spectrum recorded when the pristine triangles were used as the substrate at these concentrations.

At the lowest concentration (0.03 μM analyte 4-ATP) SERS signals were observed for hexagons. Note that for the hexagons as the concentration of the analyte was reduced from 100 μM to 0.03 μM the vC-C signal shifts 10 cm⁻¹ from 1598 to 1588 cm⁻¹, and becomes the most dominant signal in the SERS spectrum. This is associated with the analyte orientation and changing of the analyte orientation. It is also associated with the increased binding of the analyte to different crystal faces or the different loading of the analyte on to different crystal faces of the nanoplates than is found in for example the case of the pristine triangles. These results are indicative that under these conditions the analyte molecule is in more optimal configuration for SERS. This is evidence that under these conditions in its role to increase the passivation of the nanoplates and also to couple the nanoplates the analyte molecules present themselves, by varying orientation, loading, or a combination of both within the E-field hot spots at the interface region between the coupled nanoplates in format that generates a SERS signal where no SERS is produced for other samples such as the pristine triangles

We have demonstrated the dependence of the sensitivity of the LSPR of tunable TSNP within the Vis-NIR wavelength bands upon their structural parameters over a late range of aspect ratios. We have observed strong enhancement of the LSPR sensitivity for TSNP solutions with high aspect ratios. The accentuation of the LSRP sensitivity was found to be directly dependent on TSNP aspect ratio with the largest sensitivities recorded to date, a value of 1070 nm/RIU, measured for the highest aspect ratio 13:1 TSNP solution. LSPR linewidth studies reveal that the low thickness of these TSNP facilitates of the dominance of surface over volume electron scattering contributions despite edge lengths multiples larger than the bulk electron mean free path thereby providing a mechanism for the enhancement of the LSPR sensitivities. These results suggest that the TSNP ensembles may be the optimal silver nanostructures for biosensing as they encompass aspect ratios large enough to provide high LSPR sensitivity yet low enough that the LSPR λₘₐₓ remains within the spectral range appropriate for biosensing. Upon comparison with LSPR sensitivities recorded both for single substrate bound and solution phase nanostructures reported in literature it is apparent that solution phase high aspect ratio TSNP can provide the optimum sensing response determined to date throughout the biosensing relevant spectral range.

### Electromagnetic Coupling

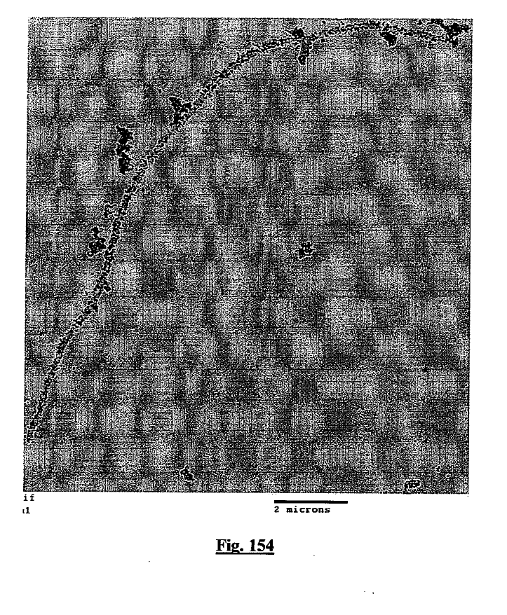

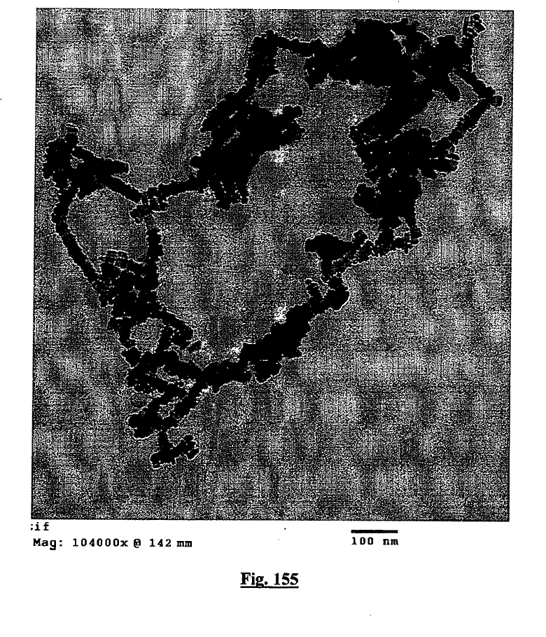

The electromagnetic coupling of adjacent triangular (or other apexed polygonal) silver nanoplates either in solution or suspension or else when deposited on a substrate is a contributing factor to their electrical conductivity. These high aspect ratio silver nanoplates have been shown to form wires and networks of wires, and quasi-solid films (FIGS. 154 to 157), within which the silver nanoplates are either in direct contact or in proximity by a distance which is of the order of 1 to 10 nm. These interparticle distances are therefore on the length scale over which quantum mechanical tunnelling currents are significant. It is also physically reasonable to conclude that the formation of a so-called metallic bond, i.e. the delocalisation of valence electrons of the metal atoms over the extent of the metal nanoplate, quantum mechanically described by a Bloch wavefunction, extends over two or more such electromagnetically coupled nanoplates in close proximity to each other. It is further physically reasonable to conclude that the proximity or coupling of another silver nanoplate to a silver nanoplate will disturb the surface plasmon of that silver nanoplate following the same reasoning that any other functionalising entity bonded, attached, or coupled to it would affect the surface plasmon of the silver nanoplate.

Triangular silver nanoplates are particularly advantageous for the formation of such electromagnetically coupled assemblies of metal nanoparticles, and by extension for the formation of electrically conducting wires and wire networks and solid films. The electric charge on the surface of the triangular silver nanoplate concentrates near the apices, and the electric field strength near the apices is increased due to this locally increased concentration of charge carriers. This effect can act to enhance the electrical conductivity of the wires, wire networks, and solid films.

We have described how these triangular silver nanoplates are of particularly high aspect ratio, and can be made of particularly long dimensions in the plane, while preserving their local surface plasmon resonance due to their thickness remaining under the mean free path length of an electron. The local surface plasmon resonance which we have described, is the only significant optical absorption mechanism of these silver nanoplates. When the edge length of the silver nanoplates is increased (by means of the selection of suitable process variables and process chemistry), their local surface plasmon resonance is shifted well beyond the visible part of the spectrum into the near infrared, and the particle suspension is rendered optically translucent as a result. The morphology of the wire network formed when the high aspect ratio nanoplate suspension is deposited on a substrate is such that most of the network comprises particle-free fields. This attribute give the network a high degrees of optical transparency.

It is therefore possible to make dense wire networks, which appear at low magnification as quasi-solid films, which are electrically conducting while also exhibiting a high degree of translucency and transparency, by depositing formulations of these predominantly triangular silver nanoplates on a substrate.

We have also observed that the silver nanoplates remain discrete when formed into solid wires and wire networks on a substrate. This, combined with the electromagnetic coupling and enhancement mechanisms associated with high aspect ratio triangular silver nanoplates, is of particular advantage when these wires and wire networks are formed on flexible substrates, wherein the substrates may be bent or flexed, with relative movement of the silver nanoplates, while sufficient electrical conductivity is preserved.

Similar arguments apply to hexagonal and other polygonal silver nanoplates, wherein there is concentration of electric charge and electric field strength at apices.

Production of Silver Nanoplates Suspensions without a Stabilising Agent

As described above, table silver nanoplates can be produced without any stabilising agent. To our knowledge, all the silver nanoplates and other nanostructures described in the literature are produced using a stabilising/capping/passivation agent. In the case of the production of the silver nanoplates without any stabiliser the same procedures are followed as given in the examples with one difference which is that no further reagents are added after the addition of the silver source.

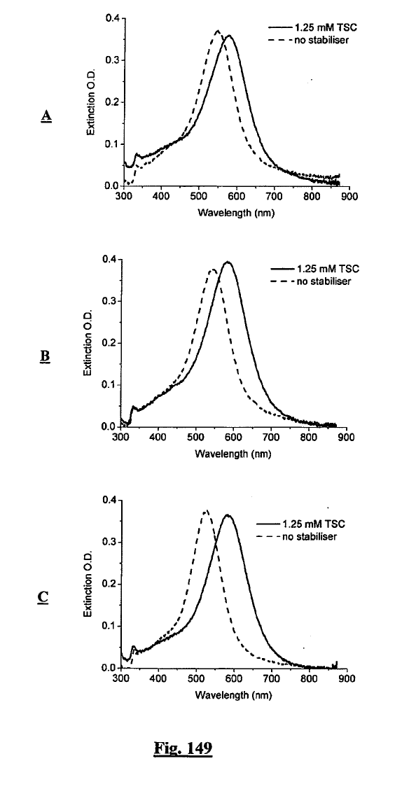

Referring to FIG. 149, the optical extinction spectra measured using UV-Visible-NIR spectroscopy of silver nanoplates produced with 1.25 mM TSC stabilisation and no stabilization, show very little variation from 30 minutes after production (FIG. 149) to 18 hours after production (FIG. 148) to 1 week after production (FIG. 148). The Table below lists the peak wavelength positions of each of these silver nanoplates each of which and including the silver nanoplates which are produced without a stabiliser are highly stable given the consistent profile of their LSPR spectra over time, including the presence of the out of plane quadrupole in the 340 nm region, little variation in the extinction optical density (OD.) and the minimal shifting to the LSPR peak wavelengths.

List of peak wavelength spectral positions for nanoplates produced with 1.25 mM TSC and no stabilization

| | Peak wavelength λₘₐₓ (nm) | | |
|---|---|---|---|
| Stabilizer | Time 0 | 18 h | 1 week |
| 1.25 mM TSC | 577 | 581 | 581 |
| No stabilizer | 546 | 543 | 527 |

### Cross Flow Filtration Concentration

Concentrating the silver nanoplates inks was achieved using cross flow ultrafiltration membranes. These cartridges are operated in a cross flow mode. In sharp contrast to single pass filtration, cross flow involves recirculation of the feed stream pumped across the membrane surface. The “sweeping action” created by fluid flow across the membrane surface promotes consistent productivity over the long term. In operation, as the feed stream is pumped through the membrane cartridge, the retentate, including species excluded by the membrane pores, continues through the recirculation loop while the permeate, including solvent and solutes transported through the membrane pores, is collected on the shell side of the cartridge.

As a convention, flux is recorded in terms of litres per square meter of membrane surface area per hour (lmh). Flux in l.m.⁻².h.⁻¹ (“lmh”) is:

Flux(lmh)=(Permeate Flow(ml/min)/Cartridge Area(m²))×0.06

Typical flux observed is of the order of 100-150 lmh, which shows promise of a fast densification process, considering also that this concentration process is close to being linearly scalable. Average flux does vary from batch to batch. However there is no appreciable decrease in the flux as the concentration factor is increased.

A low void volume allowed us to achieve a concentration factor of minimum 10, with starting concentration of 100 ppm.

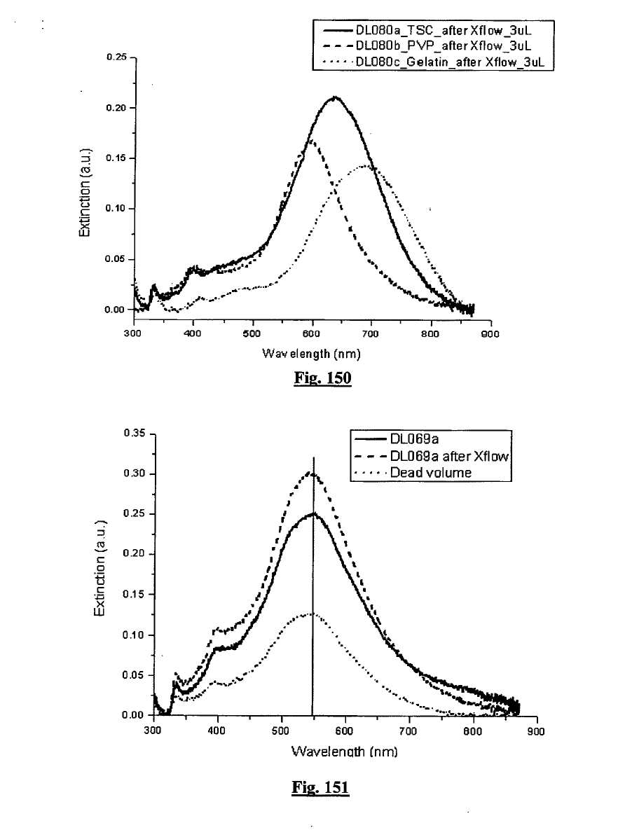

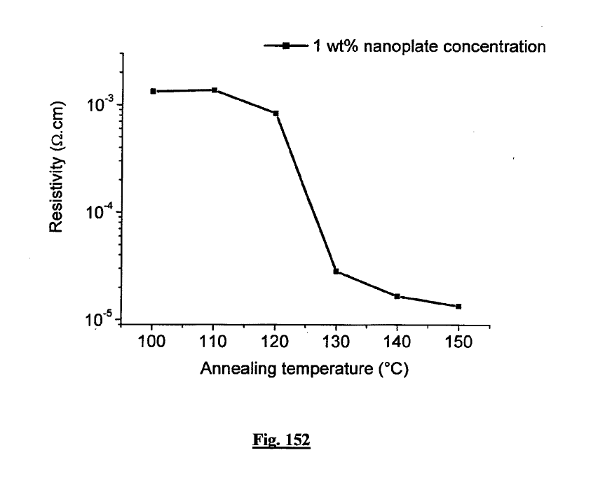

FIG. 150 shows the optical transmission spectra in the ultraviolet-visible-infrared region of the spectrum of Trisodium citrate (TSC), Polyvinylpyrrolidone (PVP) and gelatine stabilised (capped) silver nanoplates after densification using cross flow ultrafiltration. The stabilising agent was added before cross-flow filtration, demonstrating the compatibility of the cross-flow filtration processes even with stabilised formulations made using the process. The surface chemistry of these silver nanoplates has been unexpectedly found to be compatible with this membrane ultrafiltration technology, allowing the as-produced low silver weight content nanoplate suspension to be densified into a conductive ink. Generally, membrane cassette technologies of this type are not compatible with the densification of suspensions of these stable, well-dispersed, discrete nanoplates which have an inherent surface charge.

FIG. 151 shows the optical transmission spectra in the ultraviolet-visible-infrared region of the spectrum of silver nanoplates before and after densification using cross flow ultrafiltration. Also shown is the spectrum of the dead volume. There is no spectral peak shift upon densification using this process, showing that the nanoplate plasmonic properties are preserved. It made be concluded that the particle size, shaped, and discrete character are unaffected by this process of concentration by membrane ultrafiltration.

### Low Concentration Resistivity and Thermal Curing

A Jandel Universal Four Point Probe together with a Jandel RM3 test unit was used to determine the conducting properties of silver nanoplate thin films. The RM3 unit can give the resulting voltage in either mV or the sheet resistance expressed in units of Ω/□ (Ohms per dimensionless square). Four point probing is a technique which measures the average sheet resistance and bulk resistivity (expressed in Ohm·cm). The four point probe contains four thin linear placed tungsten wire probes, which once contact is made with the sample, a known current (I) is applied across the two outer probes and voltage (V) is measured by the two inner probes.

Sheet resistance is calculated using Rs (Ω/□)=4.5324 V/I

The volume resistivity is estimated by multiplying the sheet resistance value obtained by the four point probe measurement and the thickness value obtained by the profilometry measurements.

Volume resistivity(Ω·cm)=Surface resistance(Ω/□)×Film thickness(m)×100

A series of thin films of silver nanoplates with silver concentration of 0.1 wt %, 0.5 wt %, and 1 wt % were prepared by the drop casting method on glass substrates in order to estimate their resistivity. Thickness measurements were carried out using a 3D optical surface profiler. Thickness varied on average from 0.75 μm, 1.01 μm and 1.48 μm for the 0.1 wt %, 0.5 wt %, and 1 wt % samples respectively.

The annealing temperature was varied from room temperature to 200° C. in 50° C. intervals for 30 minutes for all the samples and from 100° C. to 150° C. in 10° C. intervals for 30 minutes for the 1 wt % sample.

A volume resistivity of 1.37×10⁻⁵ Ω·cm for a silver content of 1 wt % was achieved (bulk silver is 1.6×10⁻⁵ Ω·m). The best annealing temperature is found to be around 130° C.

FIG. 152 shows a graph of the resistivity of a film made by depositing a 1 wt % aqueous suspension of silver nanoplates on a substrate, as a function of curing temperature. The resistivity drops dramatically between 120° C. and 130° C. and drops gradually at higher temperatures.

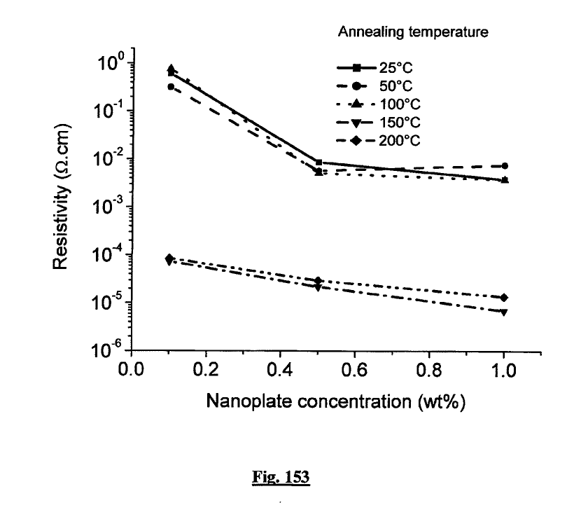

FIG. 153 shows a graph of the resistivity of a film made by depositing an aqueous suspension of silver nanoplates on a substrate, at different silver contents by weight, as a function of curing temperature.

### Curing Temperature, Printing Compatibility and Thermal Stability

FIG. 152 provides conclusive evidence that the curing temperature for these formulations as deposited on substrates is between 120° C. and 130° C. This makes the formulations compatible with ink-jet and gravure printing, and with printing on most flexible substrate materials. FIG. 153 shows temperature stability of the films for 30 minutes at 200° C. The films are also compatible with shorter thermal exposures to higher temperatures, for example during lead-free solder reflow processes.

Transparency Using Functionalisation and Transparency with Conductivity

FIG. 154 is a micrograph showing the alignment of functionalised triangular nanoplates over 15 microns.

FIG. 155 is a micrograph showing the assembled network of chemically functionalised triangular nanoplates

Alignment over 15 μm and an assembled network of phosphocholine functionalised triangular nanoplates were achieved for increased connectivity. With reference to FIG. 155, it is clear that a dense, wire network has been formed on the substrate, with substantial particle-free fields. This is the basis for an optically translucent or transparent, electrically conductive, film.

Hexagonal nanoplates produced using a low citrate concentration (12.5 μM) were also produced and a similar wire network was made from them.

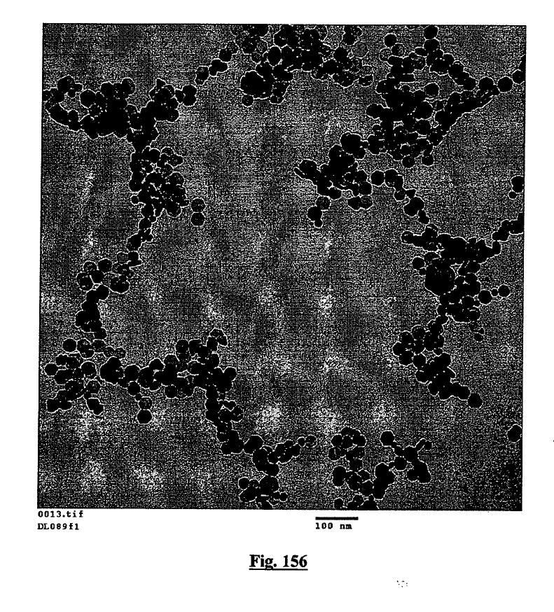

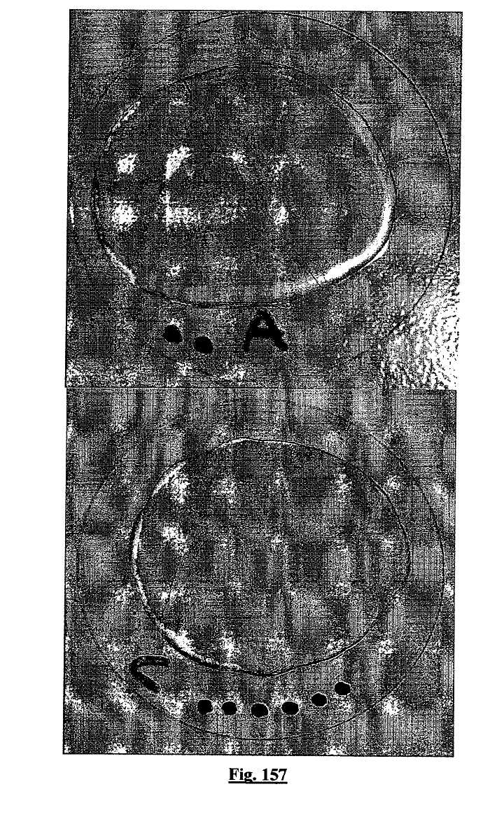

FIG. 156 is a micrograph showing an assembled network of hexagonal silver nanoplates which result in better packing than triangular nanoplates.

FIG. 157 shows two photographs of silver thin films, post thermal curing, made with (a) 0.1 wt % and (b) 1 wt % of silver nanoplates. This is further evidence of optical transparency.

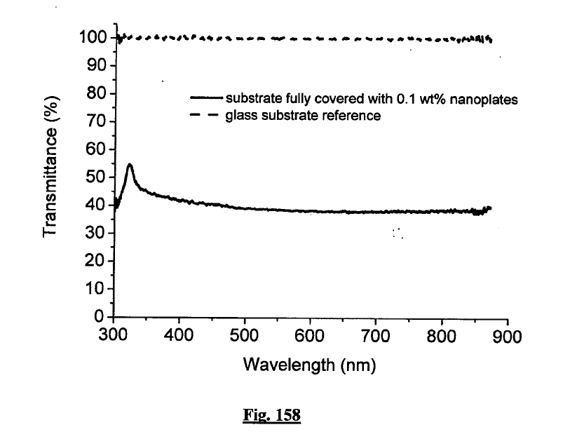

FIG. 158 shows a graph of the thin film transmittance of a 0.1 wt % silver nanoplate coated glass substrate, in the ultraviolet-visible-infrared spectral region. This is evidence for the transparency of these electrically conducting films.

The invention is not limited to the embodiments hereinbefore described, with reference to the accompanying drawings, which may be varied in detail.

### REFERENCES

(1) Willets, K. A.; Van Duyne, R. P. Annual Review of Physical Chemistry 2007, 58, 267.
(2) Jensen, T. R.; Malinsky, M. D.; Haynes, C. L.; Van Duyne, R. P. Journal of Physical Chemistry B 2000, 104, 10549.
(3) Ledwith, D.; Aherne, D.; Kelly, J. M. Approaches to the Synthesis and Characterization of Spherical and Anisotropic Silver Nanomaterials. In Metallic Nanomaterials; Kumar, C. S. S. R., Ed.; Wiley-VCH: Weinheim, Germany 2009; Vol. 1; pp 99.
(4) Johnson, R. C.; Li, J.; Hupp, J. T.; Schatz, G. C. Chemical Physics Letters 2002, 356, 534.
(5) Chan, G. H.; Zhao, J.; Hicks, E. M.; Schatz, G. C.; Van Duyne, R. P. Nano Letters 2007, 7, 1947.
(6) Doremus, R. H.; Rao, P. Journal of Materials Research 1996, 11, 2834.
(7) Zhang, X. Y.; Hicks, E. M.; Zhao, J.; Schatz, G. C.; Van Duyne, R. P. Nano Letters 2005, 5, 1503.
(8) Mock, J. J.; Smith, D. R.; Schultz, S. Nano Letters 2003, 3, 485.
(9) Jensen, T. R.; Duval, M. L.; Kelly, K. L.; Lazarides, A. A.; Schatz, G. C.; Van Duyne, R. P. Journal of Physical Chemistry B 1999, 103, 9846.
(10) Malinsky, M. D.; Kelly, K. L.; Schatz, G. C.; Van Duyne, R. P. Journal of Physical Chemistry B 2001, 105, 2343.
(11) Sonnichsen, C.; Reinhard, B. M.; Liphardt, J.; Alivisatos, A. P. Nature Biotechnology 2005, 23, 741.
(12) Sharma, P.; Brown, S.; Walter, G.; Santra, S.; Moudgil, B. Advances in Colloid and Interface Science 2006, 123-126, 471.
(13) Wang, C. Y.; Cimalla, V.; Kups, T.; Rohlig, C. C.; Stauden, T.; Ambacher, O.; Kunzer, M.; Passow, T.; Schirmacher, W.; Pletschen, W.; Kohler, K.; Wagner, J. Applied Physics Letters 2007, 91, 103509.
(14) Zhao, J.; Zhang, X. Y.; Yonzon, C. R.; Haes, A. J.; Van Duyne, R. P. Nanomedicine 2006, 1, 219.
(15) Haes, A. J.; Van Duyne, R. P. Journal of the American Chemical Society 2002, 124, 10596.
(16) Kelly, K. L.; Coronado, E.; Zhao, L. L.; Schatz, G. C. Journal of Physical Chemistry B 2003, 107, 668.
(17) Sherry, L. J.; Jin, R. C.; Mirkin, C. A.; Schatz, G. C.; Van Duyne, R. P. Nano Letters 2006, 6, 2060.
(18) Sherry, L. J.; Chang, S. H.; Schatz, G. C.; Van Duyne, R. P.; Wiley, B. J.; Xia, Y. N. Nano Letters 2005, 5, 2034.
(19) Nehl, C. L.; Liao, H. W.; Hafner, J. H. Nano Letters 2006, 6, 683.
(20) Sun, Y.; Xia, Y. Anal. Chem. 2002, 74, 5297.
(21) Wang, H.; Brandl, D. W.; Le, F.; Nordlander, P.; Halas, N. J. Nano Letters 2006, 6, 827.
(22) Larsson, E. M.; Alegret, J.; Kall, M.; Sutherland, D. S. Nano Letters 2007, 7, 1256.
(23) Kreibig, U., Vollmer, M. Optical Properties of Metal Clusters; Springer: Berlin, 1995.
(24) Voisin, C.; Del Fatti, N.; Christofilos, D.; Vallee, F. J. Phys. Chem. B 2001, 105, 2264.
(25) Lee, P. C.; Meisel, D. The Journal of Physical Chemistry 1982, 86, 3391.
(26) Hao, E.; Schatz, G. C. The Journal of Chemical Physics 2004, 120, 357.
(27) Khalavka, Y.; Becker, J.; Sonnichsen, C. Journal of the American Chemical Society 2009, 131, 1871.
(28) McFarland, A. D.; Van Duyne, R. P. Nano Letters 2003, 3, 1057.
(29) Henzie, J.; Lee, M. H.; Odom, T. W. Nat Nano 2007, 2, 549.
(30) Cao, M.; Wang, M.; Gu, N. The Journal of Physical Chemistry C 2009, 113, 1217.
(31) Charles, D.; Fournet, P.; Cunningham, S.; Ledwith, D.; Kelly, J. M.; Blau, W.; Fournet, M. B. Plasmonics: Metallic Nanostructures and Their Optical Properties VI, 2008, San Diego, Calif., USA.
(32) Charles, D. E.; Aherne, D.; Gara, M.; Ledwith, D. M.; Gun'ko, Y. K.; Kelly, J. M.; Blau, W. J.; Brennan-Foumet, M. E. ACS Nano 2009, 4, 55.
(33) Shuford, K. L.; Ratner, M. A.; Schatz, G. C. Journal of Chemical Physics 2005, 123, 114713.
(34) Jung, L. S.; Campbell, C. T.; Chinowsky, T. M.; Mar, M. N.; Yee, S. S. Langmuir 1998, 14, 5636.
(35) Mie, G. Ann Phys 1908, 25, 377.
(36) Haes, A. J.; Van Duyne, R. P. Analytical and Bioanalytical Chemistry 2004, 379, 920.
(37) Miller, M. M.; Lazarides, A. A. The Journal of Physical Chemistry B 2005, 109, 21556.
(38) Hu, M.; Novo, C.; Funston, A.; Wang, H.; Staleva, H.; Zou, S.; Mulvaney, P.; Xia, Y.; Hartland, G. V. Journal of Materials Chemistry 2008, 18, 1949.
(39) Coronado, E. A.; Schatz, G. C. Journal of Chemical Physics 2003, 119, 3926.
(40) Munechika, K.; Smith, J. M.; Chen, Y.; Ginger, D. S. Journal of Physical Chemistry C 2007, 111, 18906.
(41) Lee, J.; Hasan, W.; Odom, T. W. The Journal of Physical Chemistry C 2009, 113, 2205.
(42) Draine, B. T.; Flatau, P. J. J. Opt. Soc. Am. A 1994, 11, 1491.
(43) Zou, X.; Dong, S. The Journal of Physical Chemistry B 2006, 110, 21545.
(44) Campion, A.; Kambhampati, P. Chemical Society Reviews 1998, 27, 241.
(45) Lal, S.; Grady, N. K.; Kundu, J.; Levin, C. S.; Lassiter, J. B.; Halas, N. J. Chemical Society Reviews 2008, 37, 898.
(46) Xu, H.; KM, M. Physical Review Letters 2002, 89, 246802.
(47) Sang Kyu, K.; Tai Ha, J.; Se Won, S.; Myung Soo, K. Journal of Raman Spectroscopy 1986, 17, 381.
(48) Banholzer, M. J.; Millstone, J. E.; Qin, L. D.; Mirkin, C. A. Chemical Society Reviews 2008, 37, 885.
(49) Le Ru, E. C.; Blackie, E.; Meyer, M.; Etchegoin, P. G. The Journal of Physical Chemistry C 2007, 111, 13794.
(50) Orendorff, C. J.; Gearheart, L.; Jana, N. R.; Murphy, C. J. Physical Chemistry Chemical Physics 2006, 8, 165.
(51) Baldwin, J. A.; VlcÍ(EkovÃᵢ, B.; Andrews, M. P.; Butler, I. S. Langmuir 1997, 13, 3744.
(52) Baldwin, J.; SchÃ¼hler, N.; Butler, I. S.; Andrews, M. P. Langmuir 1996, 12, 6389.
(53) Szafranski, C. A.; Tanner, W.; Laibinis, P. E.; Garrell, R. L. Langmuir 1998, 14, 3570.
(54) Carron, K. T.; Hurley, L. G. The Journal of Physical Chemistry 1991, 95, 9979.
(55) Joo, T. H.; Kim, M. S.; Kim, K. Journal of Raman Spectroscopy 1987, 18, 57.
(56) Aherne, D.; Ledwith, D. M.; Gara, M.; Kelly, J. M. Advanced Functional Materials 2008, 18, 2005.

1-92. (canceled)

93. A sensor for detecting of an analyte in a solution phase, the sensor comprising a plurality of functionalised silver nanoplates wherein a functionalising agent is directly bonded to the surfaces of the nanoplates and whereby the nanoplates provide a detectable wavelength shift change in their local surface plasmon resonance spectrum in response to the binding of an analyte.

94. The sensor as claimed in claim 93 wherein two or more of the nanoplates are electromagnetically coupled.

95. The sensor as claimed in claim 93 wherein at least three or more of the nanoplates are electromagnetically coupled.

96. The sensor as claimed in claim 93 wherein at least four or more of the nanoplates are electromagnetically coupled.

97. The sensor as claimed in claim 96 wherein the coupled nanoplates form a chain-like structure.

98. The sensor as claimed in claim 93 wherein the nanoplates are dispersed in a solvent system.

99. The sensor as claimed in claim 93 wherein the nanoplates are tethered to a support substrate such that substantially all of the surfaces of the nanoplate are available for interaction with an analyte.

100. The sensor as claimed in claim 93 wherein the sensor comprises from 10¹ to 10¹³ nanoplates.

101. The sensor as claimed in claim 93 wherein the sensor comprises at least 10⁹ to 10¹³ nanoplates.

102. The sensor as claimed in claim 93 wherein the sensor comprises from 10¹ to 10⁹ nanoplates.

103. The sensor as claimed in claim 93 wherein the sensor comprises from 10² to 10⁴ nanoplates.

104. The sensor as claimed in claim 93 wherein the functionalised nanoplates remain stable in the solvent system for a period of at least one week at atmospheric pressure and at a temperature of 20° C.

105. The sensor as claimed in claim 93 wherein when the functionalised nanoplates are exposed to a light source at a wavelength range within the ultraviolet-visible-infrared spectrum or part thereof, and an optical spectrum of an ensemble of the functionalised nanoplates is measured over a wavelength range within the ultraviolet-visible-infrared spectrum or part thereof, at least one optical spectral peak is observed due to the local surface plasmon resonance (LSPR) of the functionalised nanoplates with incident light from said light source, and the said functionalised nanoplates have, for a specific method of light exposure and optical spectrum measurement, a specified minimum sensitivity or ensemble sensitivity figure of merit (FOM) (defined as the ratio of the linear local surface plasmon resonance (LSPR) refractive index sensitivity or ensemble sensitivity, to the local surface plasmon resonance linewidth being the full width at half peak maximum (FWHM) of the optical spectral peak due to the local surface plasmon resonance (LSPR)) at least at one specified wavelength in the spectrum.

106. The sensor as claimed in claim 105 wherein the ensemble sensitivity figure of merit is at least 1.75 at a wavelength of 450 nm

107. The sensor as claimed in claim 105 wherein the ensemble sensitivity figure of merit is at least 1.75 at wavelengths between 450 nm and 930 nm.

108. The sensor as claimed in claim 105 wherein the ensemble sensitivity figure of merit is at least 2.25 at wavelengths above 900 nm.

109. The sensor as claimed in claim 105 wherein the ensemble sensitivity figure of merit is at least 3.0 at wavelengths above 1100 nm.

110. The sensor as claimed in claim 93 wherein the nanoplates have an ensemble sensitivity value of between 281 nm and 1400 nm per unit change in the (dimensionless) refractive index and with a local surface plasmon resonance (LSPR) peak in the 400 nm to 1200 nm wavelength region of the spectrum when measured by optical extinction spectroscopy.

111. The sensor as claimed in claim 93 wherein the nanoplates have an ensemble sensitivity value of at least 300 nm per unit change in the (dimensionless) refractive index with a local surface plasmon resonance (LSPR) peak in the 600 nm region of the spectrum when measured by optical extinction spectroscopy.

112. The sensor as claimed in claim 93 wherein the light from a light source traverses a volume or part thereof containing the functionalised nanoplates in a dark field imaging or light collection arrangement, and the optical reflection and/or scattering and/or emission spectrum of an ensemble of the functionalised nanoplates thereof is measured by dark field spectroscopy.

113. The sensor as claimed in any of claim 112 wherein the ensemble sensitivity figure of merit is greater than 1.9 at a wavelength of 450 nm when measured by dark field spectroscopy.

114. The sensor as claimed in claim 112 wherein the ensemble sensitivity figure of merit is greater than 3.0 at a wavelength of 600 nm when measured by dark field spectroscopy.

115. The sensor as claimed in claim 112 wherein the ensemble sensitivity figure of merit is greater than 3.5 at a wavelength of 750 nm when measured by dark field spectroscopy.

116. The sensor as claimed in claim 112 wherein the ensemble sensitivity figure of merit of the functionalised nanoplates when measured by dark field spectroscopy is greater than the sensitivity or ensemble sensitivity figure of merit (respectively) of the functionalised nanoplates when measured by optical extinction spectroscopy performed at a wavelength range within the ultraviolet-visible-infrared spectrum or part thereof.

117. The sensor as claimed in claim 93 wherein the functionalising agent is selected from a ligand, a peptide, a polypeptide, a glycan, an antibody, or a nucleic acid.

118. The sensor as claimed in claim 93 wherein the functionalising agent is selected from a mono-species, a di-species, and a multi-species functionalising agent.

119. The sensor as claimed in claim 93 wherein the silver nanoplates have an aspect ratio of between 2 and 20.

120. The sensor as claimed in claim 93 wherein the nanoplates are triangular in shape.

121. The sensor as claimed in claim 93 wherein the nanoplates are of truncated triangular shape.

122. The sensor as claimed in claim 121 wherein the apices of the triangles have been snipped with a chemical agent or by deprivation of a passivation agent

123. The sensor as claimed in claim 122 wherein the chemical agent is one or more of an acid, a base, a salt, a polymer, or a biological agent.

124. The sensor as claimed in claim 93 wherein the nanoplates are blocked with a blocking agent.

125. The sensor as claimed in claim 124 wherein the blocking agent is selected from a mercapto based agent, such as mercaptobenzoic acid or mercaptohexadecanoic acid or 16-mercaptohexadecanoic acid, or a serum, or an immuno stripped serum, or a non-immuno antibody or a non-specific protein, or a nucleic acid sequence or styrene, or polyethylene glycol.

126. The use of a sensor as claimed in claim 93 in an assay based on the principle of local surface plasmon resonance (LSPR) optical spectral peak wavelength shift due to a refractive index change or other optical property change in response to the attachment of a species to at least some of the functionalised nanoplates.

127. The use of a sensor as claimed in claim 93 as a contrast agent for cellular imaging.

128. A process for functionalising the surface of a silver nanoplate with a functionalising agent comprising the steps of:
a. forming silver seeds from an aqueous solution comprising a reducing agent, a stabilising agent, a water soluble polymer and a silver source; and
growing the thus formed seeds into silver nanoplates in an aqueous solution comprising silver seeds, a reducing agent, a silver source, and a functionalising agent selected from a ligand, a peptide, a polypeptide, a glycan, an antibody, or a nucleic acid.


SEQUENCE LISTING (5 sequences)

SEQ ID NO: 1 - DNA, 20 bp, Artificial Sequence
  synthesised sequence
tagccatttatggcgaacca

SEQ ID NO: 2 - DNA, 20 bp, Artificial Sequence
  synthesised sequence
ccccaagtccttgtggcttg

SEQ ID NO: 3 - DNA, 20 bp, Artificial Sequence
  synthesised sequence
tggttcgccataaatggcta

SEQ ID NO: 4 - DNA, 20 bp, Artificial Sequence
  synthesised sequence
atcggtaaataccgcttggt

SEQ ID NO: 5 - DNA, 40 bp, Artificial Sequence
  synthesised sequence
aaaaaaaaaaaaaaaaaaaatagccatttatggcgaacca